SEQUENCE LISTING (44 sequences)

SEQ ID NO: 1 - DNA, 190 bp, Homo sapiens
ggacccaggatatgagcggaaaacactttctctacttagatacaactttttcctgtgcgcatgcctgtaatcccagctactcaggaggctgaggcaggagaatcccttgaacccaggaggcagaggttgcggtgagccaagatctcaccattgcactccagcctgggcaataagaacaaaactccgtctc

SEQ ID NO: 2 - DNA, 783 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagctatgtaactgagtaagacagggagaaatattaatccgtgagtggctcccagtaagaccatggccaaatacatcctgaagtagaatatctggaaaatttgagatt

SEQ ID NO: 3 - DNA, 1649 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagccttcaaatcatctgggcccaagttaaaacagaaggaatttaaaaaaaaaacacagtcactgtcttagaagatgactcatatgctaagacaggtctgcctccctgactcagaatgctgagtgactcctgacattattagttggaatgggaagtgtaaggtcaagttggggtctttacctgcatgacgaaaccacttcttgtaatgacagacttttactgtgttggttagaatagccagtccttggggagcctctagtctgttgtagctgaatgatttggaagtgttctttcactttttacttttgtcctcagcattacctacatgaacttgttatcaaaacccttgtccagcacaacctcttttatatgctgcatcagttcctgcagtaccacgtcctcagcgactccaaacctttggcttgtctgctgttatccctagagagtttctatcctcctgctcatcagctatctctggacatgctgaaggtaactctgatgtgtgaggttttagactatggaaactaactctgttcctgttgtttgcactgacctggacttctctcccttactgctagcgactttcaacagcaaatgatgaaatagtagaagttctcctttccaaacaccaagtgttagctgccttaaggtttatccggggcattggtggccatgacaacatttctgcacgaaaatttttagatgctgcaaagcagactgaagacaacatgcttttctatacaatattccgcttttttgaacagcgaaaccagcgtttgcgagggagccccaatttcacaccaggggaacactgtgaagaacatgttgcttttttcaaacagatttttggagaccaagctctaatgaggcctacaacattctgaaatcacttgctgtttttttatataaaaatgtgtacaaagttaatttattgcattaataaagctctttaaactataaaatgttaaaaagtg

SEQ ID NO: 4 - DNA, 1861 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagccttcaaatcatctgggcccaagttaaaacagaaggaatttaaaaaaaaaacacagtcactgtcttagaagatgactcatatgctaagacaggtctgcctccctgactcagaatgctgagtgactcctgacattattagttggaatgggaagtgtaaggtcaagttggggtctttacctgcatgacgaaaccacttcttgtaatgacagacttttactgtgttggttagaatagccagtccttggggagcctctagtctgttgtagctgaatgatttggaagtgttctttcactttttacttttgtcctcagcattacctacatgaacttgttatcaaaacccttgtccagcacaacctcttttatatgctgcatcagttcctgcagtaccacgtcctcagcgactccaaacctttggcttgtctgctgttatccctagagagtttctatcctcctgctcatcagctatctctggacatgctgaaggtaactctgatgtgtgaggttttagactatggaaactaactctgttcctgttgtttgcactgacctggacttctctcccttactgctagcgactttcaacagcaaatgatgaaatagtagaagttctcctttccaaacaccaagtgttagctgccttaaggtttatccggggcattggtggccatgacaacatttctgcacgaaaatttttagatgctgcaaagcagactgaagacaacatgcttttctatacaatattccgcttttttgaacagcgaaaccagcgtttgcgagggagccccaatttcacaccaggtgagaatgcaatgaaaagacttggggtaaccatagcctcaaagagtagcagagggcactggcagctggtgggcgaggaccctgggttagcatttttgtaaacaacacaatttgataacagcccacctagcccttggcccattatttgtagtagagtgaattcagtatactgacagaatctggattatgctctggaactcaccgaggaggtgtgttttgagtcaagacacatttaggacccagatcaggcacagcccatctcttatagcagatcttggaatatctcttaaagccaggaataagacggcaaatggtggctaagggttttaaagggtctggggcttattaaggtttcagttttatgaagtatacattggttgat

SEQ ID NO: 5 - DNA, 214 bp, Homo sapiens
gtaagggaactttggcgacttagtgcgatcactgggagaattgtagagtccactggagagaaagaaaaatggtcaaaaagagcccagagagttcctgggggaaaacacaccgcagcccagacctattcataactgcacagctggtacttccagaggcacatgcaccaggggcacgtggttctctttgctgacaagatttattaaaagaaaagag

SEQ ID NO: 6 - DNA, 1934 bp, Homo sapiens
aagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgtgagtaatccattctaattcttcaattgtgttccagacagtgctttaatttgtctttacattttaaccaaaactaggtgacagtagcgaaagaggaagaaaagtgtgcattaaagctacttattctacactataatcactatcatctcttattagccacctctttgtacttggtaggtacaagggggcttttcctgattaatgtcagttttaaaataaattcttttctgagattctcactgaaaaaat

SEQ ID NO: 7 - DNA, 2353 bp, Homo sapiens
aagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 8 - DNA, 2500 bp, Homo sapiens
gacatgggctgtttctgcgctgttccggaagaattttactgcgaagttttgctcctggatgaatccaagttaacccttaccacccagcagcagggcatcaagaagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 9 - DNA, 947 bp, Homo sapiens
gaaagatgatactaggtcaggaaatagcatttgaaagtcattctcatctggagggatgaagccaagataaggcggaaccagggaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccattaacagtggtgaagtttcagatcttaaggcaaagggagggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgtgagtaatccattctaattcttcaattgtgttccagacagtgctttaatttgtctttacattttaaccaaaactaggtgacagtagcgaaagaggaagaaaagtgtgcattaaagctacttattctacactataatcactatcatctcttattagccacctctttgtacttggtaggtacaagggggcttttcctgattaatgtcagttttaaaataaattcttttctgagattctcactgaaaaaat

SEQ ID NO: 10 - DNA, 1366 bp, Homo sapiens
gaaagatgatactaggtcaggaaatagcatttgaaagtcattctcatctggagggatgaagccaagataaggcggaaccagggaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccattaacagtggtgaagtttcagatcttaaggcaaagggagggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 11 - DNA, 422 bp, Homo sapiens
aatcttcataatccccatgtgtcaaaggagagaccaggtggaggtaactgaatcatgggggtggtttccccaggctgtttttgtgatagtgagtgagttctcatgagatctgatggttttataaggggctcttccctcctttgcttgtgaagaaggtgccttttttcccctttgccttctgccatgattgtaagtttcctgaggcctccccagccaagctgaactgtgagtcaattaaacctcttttcttcgtaaattacccagtcttgagcagttctttacagcagtgtgaaaacagaggaatacacccatacatgctattctctgcccagaagccagggggagcctgccattaaaatgaaagtcactccttgactcagaaccctcaaatagctttcatctcacccagaaaaaaaagaaaa

SEQ ID NO: 12 - DNA, 1532 bp, Homo sapiens
aggtttctgcacaggaatatcgagagcgtcatgaacccgagctatagagaaaggagatgaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 13 - DNA, 1753 bp, Homo sapiens
tttcttagggtttttttttgagttggagcctcgctctgtcccccaggctggagtgcagtgatgtgatctcggctcactgcaacctctgcctcccaggttcaagtgattctcctgcctcagcctccctagtagctgcgactacaggcatgtgccaccatgcctggctaacgttttgtatttttgagtagagacagggtttcaccatgttggccaggctattctcgaactcctgacctcaagtgatccacctgcctcggcttcccaaagtttctgggattacaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 14 - DNA, 1832 bp, Homo sapiens
gggttttgcgggtataattacattcaggatctcaggatactgcattatctgtgtgacccctaaatctgatgacaagtgtctgttttttgtttttgtttttgagacagagcctcgctctgtcacccaggctggagtgctgtggtgtgatctcggctcactgcaacctccgcctcccaggttcaagcaattctctgcctcagcctcccgagtaaatgtgattacaggcaggcgcctgccagcacacccagctgattttagtatttttagtagagatggggtttcaccatcttggccaggctggtcttgaattcctgacctcgtgatccacccacttcagcttcccaaagttctgggattacaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 15 - DNA, 10394 bp, Homo sapiens
cgttgtttggcgtgtttttttttttgttttttgtcactgcctgcctgggtcctgcccgaggtctccatcctcggtttccctgtccttgccccgggccctgggagtgctctggaaggctgcgcagtattggaggggacagaatgaccttccggccttgagtccctggggagcagatggaccctactggaagtcagttggattcagatttctctcagcaagatactccttgcctgataattgaagattctcagcctgaaagccaggttctagaggatgattctggttctcacttcagtatgctatctcgacaccttcctaatctccagacgcacaaagaaaatcctgtgttggatgttgtgtccaatcctgaacaaacagctggagaagaacgaggagacggtaatagtgggttcaatgaacatttgaaagaaaacaaggttgcagaccctgtggattcttctaacttggacacatgtggttccatcagtcaggtcattgagcagttacctcagccaaacaggacaagcagtgttctgggaatgtcagtggaatctgctcctgctgtggaggaagagaagggagaagagttggaacagaaggagaaagagaaggaagaagatacttcaggcaatactacacattcccttggtgctgaagatactgcctcatcacagttgggttttggggttctggaactctcccagagccaggatgttgaggaaaatactgtgccatatgaagtggacaaagagcagctacaatcagtaaccaccaactctggttataccaggctgtctgatgtggatgctaatactgcaattaagcatgaagaacagtccaacgaagatatccccatagcagaacagtccagcaaggacatccctgtgacagcacagcccagtaaggatgtacatgttgtaaaagagcaaaatccaccacctgcaaggtcagaggacatgccttttagccccaaagcatctgttgctgctatggaagcaaaagaacagttgtctgcacaagaacttatggaaagtggactgcagattcagaagtcaccagagcctgaggttttgtcaactcaggaagacttgtttgaccagagcaataaaacagtatcttctgatggttgctctactccttcaagggaggaaggtgggtgttctttggcttccactcctgccaccactctgcatctcctgcagctctctggtcagaggtcccttgttcaggacagtctttccacgaattcttcagatcttgttgctccttctcctgatgctttccgatctactccttttatcgttcctagcagtcccacagagcaagaagggagacaagataagccaatggacacgtcagtgttatctgaagaaggaggagagccttttcagaagaaacttcaaagtggtgaaccagtggagttagaaaacccccctctcctgcctgagtccactgtatcaccacaagcctcaacaccaatatctcagagcacaccagtcttccctcctgggtcacttcctatcccatcccagcctcagttttctcatgacatttttattccttccccaagtctggaagaacaatcaaatgatgggaagaaagatggagatatgcatagttcatctttgacagttgagtgttctaaaacttcagagattgaaccaaagaattcccctgaggatcttgggctatctttgacaggggattcttgcaagttgatgctttctacaagtgaatatagtcagtccccaaagatggagagcttgagttctcacagaattgatgaagatggagaaaacacacagattgaggatacggaacccatgtctccagttctcaattctaaatttgttcctgctgaaaatgatagtatcctgatgaatccagcacaggatggtgaagtacaactgagtcagaatgatgacaaaacaaagggagatgatacagacaccagggatgacattagtattttagccactggttgcaagggcagagaagaaacggtagcagaagatgtttgtattgatctcacttgtgattcggggagtcaggcagttccgtcaccagctactcgatctgaggcactttctagtgtgttagatcaggaggaagctatggaaattaaagaacaccatccagaggaggggtcttcagggtctgaggtggaagaaatccctgagacaccttgtgaaagtcaaggagaggaactcaaagaagaaaatatggagagtgttccgttgcacctttctctgactgaaactcagtcccaagggttgtgtcttcaaaaggaaatgccaaaaaaagaatgctcagaagctatggaagttgaaaccagtgtgattagtattgattcccctcaaaagttggcaatacttgaccaagaattggaacataaggaacaggaagcttgggaagaagctacttcagaggactccagtgttgtcattgtagatgtgaaagagccatctcccagagttgatgtttcttgtgaacctttggagggagtggagaagtgctcagattcccagtcatgggaggatattgctccagaaatagaaccatgtgctgagaatagattagacaccaaggaagaaaagagtgtagaatatgaaggagatctgaaatcagggactgcagaaacagaacctgtagagcaagattcttcacagccttccttacctttagtgagagcagatgatcctttaagacttgaccaggagttgcagcagccccaaactcaggagaaaacaagtaattcattaacagaagactcaaaaatggctaatgcaaagcagctaagctcagatgcagaggcccagaagctggggaagccctctgcccatgcctcacaaagcttctgtgaaagttctagtgaaaccccatttcatttcactttgcctaaagaaggtgatatcatcccaccattgactggtgcaaccccacctcttattgggcacctaaaattggagcccaagagacacagtactcctattggtattagcaactatccagaaagcaccatagcaaccagtgatgtcatgtctgaaagcatggtggagacccatgatcccatacttgggagtggaaaaggggattctggggctgccccagacgtggatgataaattatgtctaagaatgaaactggttagtcctgagactgaggcgagtgaagagtctttgcagttcaacctggaaaagcctgcaactggtgaaagaaaaaatggatctactgctgttgctgagtctgttgccagtccccagaagaccatgtctgtgttgagctgtatctgtgaagccaggcaagagaatgaggctcgaagtgaggatccccccaccacacccatcagggggaacttgctccactttccaagttctcaaggagaagaggagaaagaaaaattggagggtgaccatacaatcaggcagagtcaacagcctatgaagcccattagtcctgtcaaggaccctgtttctcctgcttcccagaagatggtcatacaagggccatccagtcctcaaggagaggcaatggtgacagatgtgctagaagaccagaaagaaggacggagtactaataaggaaaatcctagtaaggccttgattgaaaggcccagccaaaataacataggaatccaaaccatggagtgttccttgagggtcccagaaactgtttcagcagcaacccagactataaagaatgtgtgtgagcaggggaccagtacagtggaccagaactttggaaagcaagatgccacagttcagactgagagggggagtggtgagaaaccagtcagtgctcctggggatgatacagagtcgctccatagccagggagaagaagagtttgatatgcctcagcctccacatggccatgtcttacatcgtcacatgagaacaatccgggaagtacgcacacttgtcactcgtgtcattacagatgtgtattatgtggatggaacagaagtagaaagaaaagtaactgaggagactgaagagccaattgtagagtgtcaggagtgtgaaactgaagtttccccttcacagactgggggctcctcaggtgacctgggggatatcagctccttctcctccaaggcatccagcttacaccgcacatcaagtgggacaagtctctcagctatgcacagcagtggaagctcagggaaaggagccggaccactcagagggaaaaccagcgggacagaacccgcagattttgccttacccagctcccgaggaggcccaggaaaactgagtcctagaaaaggggtcagtcagacagggacgccagtgtgtgaggaggatggtgatgcaggccttggcatcagacagggagggaaggctccagtcacgcctcgtgggcgtgggcgaaggggccgcccaccttctcggaccactggaaccagagaaacagctgtgcctggccccttgggcatagaggacatttcacctaacttgtcaccagatgataaatccttcagccgtgtcgtgccccgagtgccagactccaccagacgaacagatgtgggtgctggtgctttgcgtcgtagtgactctccagaaattcctttccaggctgctgctggcccttctgatggcttagatgcctcctctccaggaaatagctttgtagggctccgtgttgtagccaagtggtcatccaatggctacttttactctgggaaaatcacacgagatgtcggagctgggaagtataaattgctctttgatgatgggtacgaatgtgatgtgttgggcaaagacattctgttatgtgaccccatcccgctggacactgaagtgacggccctctcggaggatgagtatttcagtgcaggagtggtgaaaggacataggaaggagtctggggaactgtactacagcattgaaaaagaaggccaaagaaagtggtataagcgaatggctgtcatcctgtccttggagcaaggaaacagactgagagagcagtatgggcttggcccctatgaagcagtaacacctcttacaaaggcagcagatatcagcttagacaatttggtggaagggaagcggaaacggcgcagtaacgtcagctccccagccacccctactgcctccagtagcagcagcacaacccctacccgaaagatcacagaaagtcctcgtgcctccatgggagttctctcaggcaaaagaaaacttatcacttctgaagaggaacggtcccctgccaagcgaggtcgcaagtctgccacagtaaaacctggtgcagtaggggcaggagagtttgtgagcccctgtgagagtggagacaacaccggtgaaccctctgccctggaagagcagagagggcctttgcctctcaacaagaccttgtttctgggctacgcatttctccttaccatggccacaaccagtgacaagttggccagccgctccaaactgccagatggtcctacaggaagcagtgaagaagaggaggaatttttggaaattcctcctttcaacaagcagtatacagaatcccagcttcgagcaggagctggctatatccttgaagatttcaatgaagcccagtgtaacacagcttaccagtgtcttctaattgcggatcagcattgtcgaacccggaagtacttcctgtgccttgccagtgggattccttgtgtgtctcatgtctgggtccatgatagttgccatgccaaccagctccagaactaccgtaattatctgttgccagctgggtacagccttgaggagcaaagaattctggactggcaaccccgtgaaaatcctttccagaatctgaaggtactcttggtatcagaccaacagcagaacttcctggagctctggtctgagatcctcatgactggtggtgcagcctctgtgaagcagcaccattcaagtgcccataacaaagatattgctttaggggtatttgatgtggtggtgacggacccctcatgcccagcctcggtgctgaagtgtgctgaagcattgcagctgcctgtggtgtcacaagagtgggtgatccagtgcctcattgttggggagagaattggattcaagcagcatccaaaatataaacacgattatgtttctcactaaagatacttggtcttactggttttattccctgctatcgtggagattgtgttttaaccaggttttaaatgtgtcttgtgtgtaactggattccttgcatggatcttgtatatagttttatttgctgaacttttatgataaaataaatgttgaatctctttggttgtagtaactgggatttcttcatctgtttttttgagcttaatctcagaacaaatgacaagacatagtactttctctgagtctttcaacaggcttattcacttacggaggacagctcaccaaggaaattgaaaagttaagagtgaactttattctgtggcatcattcccaaaaggttattccagggtgtctaaaatgctatgcttgcagaaactcagtttaaggtaggtgaaggcccagattaacagttgtgccaaaagttgagtggaattgggcacagctctgtttcctgacagttaaaaaagacctcatgctctctctctgagctgagatcacagctcacctgtgggtactccccaactcttagagctaaagggagaacgaaaggaccaactgccatgaagggacagtgaccataagcttgatggaatgaccttccgtaagataaacatgggaagcacaagtgagaacacctggaaatgttacacgttctagtcaaagacccaatattattattattattattgtcacaatagctggaagcagttccttcccttcctctggcatcactgatccctgcatggcttctcattctctaaagcaggggtcaacaaggtttttttctgtaaagggtcaaagagtaaatatttcaggctttgtgggccatttgatccatcacaactactcgcctttgctgtgagggcatgaaagcaaccatagacaatgagtaaacaaatgggcacggctgtgtttcagtaaaactgtacaaaaacagacagcaggccatagtttgccagctcctgctccagagacagcagtggaaagggtgatctttagttgataatagcagggaataagttgtcagagcttcccagtgtgtgtagaatatgtagtgatgaaaaccagatgcagtgactataacctgatgccagaacactgcattctttttcagtttggagggcgttgttcagtgaatatttctttttacttacactgatatgaatattgattaccagtgatggctgggccatattaagataacttcaacccctatggtttgtgtaagatgggtaattgggcctgcaatcttcagtatttaaaaatctaacaacttgatctcaattttttcttaaggacctttttcttggagaataatacttttttttttttttttttttttgagacggaatttcgctcttgttgcccaggctggaatgcaatggcacaatctcagctcactgcagcgtctgcttcccaggttcaagcaattctcctgtctcagcctcctgagtagctgggattacaggcacatgccaccacacctggctaatttttgtatttttagtagaatcgaggtttcatcatgttggtcaggctggtctcaaactcctgacttcaggtgatccgcccgcctcggcctcccaaagtgctgggattacaggtgtgagccaccatgcccggcctaagaaatacttttaagtatattttcattagctagaattgcccaatctgtgtaggtataaattacttggtatagggagagagaaagcctatcttacctgttgctttcttacttggtggtaacatccagcagttagtctatttataaacataattactttttcacatatgaaccataaaatatttaactttctgctctatattgtttgtttaccgctgtatctcccacagcttgaacagtaccaaggtacgtagtaggtgctcaataaatgactattgaataaatgaacatatccaacaaatgttctcaatgtaaaggatcagagatgccacatgttctccttgatgggagagacccttccacatgggaatgatgggaaggagttgtactcctggatgttcagtaactgcttctaggagaaaaggtagagtcctatcactaagccgcagatatttatttgtgtgtggctagaatgggatgttttgaatcttctgttacaaccttgggaacgtggctgttatttcaatttatgagccagaaattttcacatcccgaaactacaaaagagaaaaagagccttattaagtgtcatgctttcccaagactaccttcaaagaaatatgaatcaggataacctgtgatctaaataatgtcatcttaaaactgaagagtttcttttgactcttctgctacaatagcttagaaaaaaatctgcttgcagacattttagagagaaaggacaatgaagtgattttctgaatgggaatgacagacctctgggaagccagctaccactgaatctcggtatcagttttttttaaagtttagagttagaaggggtggtcgcctcctttcacagatgcggaagctagggaccagcaaggcggggtgcccacggctgcacagctagttcatatcagaattgggagtggaaggcccactgcctcccagcatagcaatacataacctagcaaaggacttaacacctatctcactgtcaggttttttagtattttatgatgatgatgacttctactagaaaataacctccattaaaattattaaagatggtcacacctctatctctaagccttacttataaaatgagggtatttggactaaagtcttcttccagttctagaattctacaactcattaaaaagccaccttaaaaagtctactgagttacccaagggttgctcctacctgcccagagttccaccagcctgggtatagtatttgttataatctagtcgtaacagtagttgagccaaatctgagttgatctgatgattccgaacactggagagaatcttgaacaggagtgaagactggcggctaaagccctgcagagagaaggactcagctgtcattccacttcagctcaccaactctccatatggaggatgggggcggagggaggagtttcttggaaaagccttgttcaaaattctacagaaccacctggccttcccacattcctatttctattagttcctaaaatgacttgtaccaaatccatacatgcatgacttcctatgaaagtactctttcatcagtaggaatttagtagctggtttccagttaatgtattttgtcaagtactggggttggggagaacccgttttgattacaagcagataattatctcagtgagatgggggttagttcaaggaagtaaggaggggggaggatgtgaggaagttagaacaacccaatgcttatttgatgggctgaataaactattcaggactgaactatttttgagcactgtgaggtggcacagtaattacctgcttcaaaatcaactgataccaacatttttatctttgtatcttatctctgtacgtgtgtgtattgaggaaatgctttactgactcagaggaaagatcatgaattctccattggcaaaaccacctctgtcctttcggcaaggctgcatacttccaggcagacgcaccttcacgagaatgctcagctgggcggctccacgctcatccagtgggcctaggttctgactgaccagcgaacaaaaactgtgacagagatctaggatttcattcaggcagtgaaacacctacccgggaaacagagttggcattaggaaaggaaggaaggtacatccatgaagttaaagtgttaggagaacagtctgattaatagctgatctaattaatagctgacctcccaaatctgacaggatagacactgccacgtgcaaggcctgccagcccctcagacgcacaaaatgcgtaaaacaaatgcatcctttcctggctaagcgagtattactctcttagccctgcaccaaacctccaatctagccacatttaactcttcatttcttagacccgcagagtgtcttcctgcctctgagctgtgagtgttgttccctttgcccgggatgctcttgtttttaataccagttcaagtcccactctctcagtgaagcactcccttccccactatagcctttagtgaaccctcgtttcttgcttctttattatctgtactgttgtccacttggcaattgttcaggcctctgtgttgttactgatttttgtatgtatatatatatatatgtcttgtttttccaactagattgtgagctccttaagggcagagccatgaattatacctctttgtatccccagtgccttgcatacagtaagcactcaata

SEQ ID NO: 16 - DNA, 6837 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaagtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgtttgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttagtgataggactctaccttttctcctagaagcagttactgaacatccaggagtacaactccttcccatcattcccatgtggaagggtctctcccatcaaggagaacatgtggcatctctgatcctttacattgagaacatttgttggatatgttcatttattcaatagtcatttattgagcacctactacgtaccttggtactgttcaagctgtgggagatacagcggtagacaaacaatatagagcagaaagttaaatattttatggttcatatgtgaaaaagtaattatgtttataaatagactaactgctggatgttaccaccaagtaagaaagcaacaggtaagataggctttctctctccctataccaagtaatttatacctacacagattgggcaattctagctaatgaaaatatacttaaaagtatttcttaggccgggcatggtggctcacacctgtaatcccagcactttgggaggccgaggcgggcggatcacctgaagtcaggagtttgagaccagcctgaccaacatgatgaaacctcgattctactaaaaatacaaaaattagccaggtgtggtggcatgtgcctgtaatcccagctactcaggaggctgagacaggagaattgcttgaacctgggaagcagacgctgcagtgagctgagattgtgccattgcattccagcctgggcaacaagagcgaaattccgtctcaaaaaaaaaaaaaaaaaaaaaagtattattctccaagaaaaaggtccttaagaaaaaattgagatcaagttgttagatttttaaatactgaagattgcaggcccaattacccatcttacacaaaccataggggttgaagttatcttaatatggcccagccatcactggtaatcaatattcatatcagtgtaagtaaaaagaaatattcactgaacaacgccctccaaactgaaaaagaatgcagtgttctggcatcaggttatagtcactgcatctggttttcatcactacatattctacacacactgggaagctctgacaacttattccctgctattatcaactaaagatcaccctttccactgctgtctctggagcaggagctggcaaactatggcctgctgtctgtttttgtacagttttactgaaacacagccgtgcccatttgtttactcattgtctatggttgctttcatgccctcacagcaaaggcgagtagttgtgatggatcaaatggcccacaaagcctgaaatatttactctttgaccctttacagaaaaaaaccttgttgacccctgctttagagaatgagaagccatgcagggatcagtgatgccagaggaagggaaggaactgcttccagctattgtgacaataataataataataatattgggtctttgactagaacgtgtaacatttccaggtgttctcacttgtgcttcccatgtttatcttacggaaggtcattccatcaagcttatggtcactgtcccttcatggcagttggtcctttcgttctccctttagctctaagagttggggagtacccacaggtgagctgtgatctcagctcagagagagagcatgaggtcttttttaactgtcaggaaacagagctgtgcccaattccactcaacttttggcacaactgttaatctgggccttcacctaccttaaactgagtttctgcaagcatagcattttagacaccctggaataaccttttgggaatgatgccacagaataaagttcactcttaacttttcaatttccttggtgagctgtcctccgtaagtgaataagcctgttgaaagactcagagaaagtactatgtcttgtcatttgttctgagattaagctcaaaaaaacagatgaagaaatcccagttactacaaccaaagagattcaacatttattttatcataaaagttcagcaaataaaactatatacaagatccatgcaaggaatccagttacacacaagacacatttaaaacctggttaaaacacaatctccacgatagcagggaataaaaccagtaagaccaagtatctttagtgagaaacataatcgtgtttatattttggatgctgcttgaatccaattctctccccaacaatgaggcactggatcacccactcttgtgacaccacaggcagctgcaatgcttcagcacacttcagcaccgaggctgggcatgaggggtccgtcaccaccacatcaaatacccctaaagcaatatctgcaaggagcaagggaaagtgaagaaggaaaggacactcaacttagccctccattagaaagagagatttgattctaaccaatacatcccactctgcacaaaccaaagccctattatgtcaaacacactgctactgatcatgaccaaaggcagagttataatcactatgtgctgaccttgtagaaatatttaacaaatatacgtccagtgcttcacttatgttgactcacctcttgaaggtggtacttttcttctctaagaaacatggatacggtcaacctattaggcctgagccttggaccacaaggcctaacacctacaggtctaaggagatccctggaacaaagacactacacacactctttcaggtacctttgttatgggcacttgaatggtgctgcttcacagaggctgcaccaccagtcatgaggatctcagaccagagctccaggaagttctgctgttggtctgataccaagagtaccttcagattctggaaaggattttcacggggttgcctatgaaggagacaggaaaggaccttagcatgacaagtaatatccaacaaactgcctttctgcaaagggactcatgtacatctgaatgctttcaaaaataaatgccccatcagacatagtgtctcaagcctgtaatcccagcactttgggaggctgtcgtggttggatctcttgggcctgggagttcgagaccagcctgggcaatgtggtgagaccccatctctacaaaagacaacaaaaaaattagctgggtgtggtggcgagtgcctgtagtcccagcagcttgggaggctgaggtagggggatcacttcagcctgggaggttgaggctgcagtaagtcgtcactgcgccactgtactccagcctaggtgacagagcaagacttcatcttaaaaaactaagccctatattagggtcccccttctcttccttctttctatgaatgatctgtattccttgcattcctggctttctaatttccatgtttgttctggggctgagaataatccaaatcatgctcctgagcctatatatttttaatgcttgcttaaaacttagttctctgactttacaggttgagaatattgaacctatatacaaatcttcacacatttgcaaaaggttcctagccaatgtaacctagggaaataaactagataaactcctgaagtcatttcaaacccactcaaatttatcccacagacattccaatttctagaaagctttactctctcacctagattctcttccctccaaagcttgctgtcctcctgcctatacaattctggatgggcttcaaatacttaccagtccagaattctttgctcctcaaggctgtacccagctggcaacagataattacggtagttctggagctggttggcatggcaactatcatggacccagacatgagacacacaaggaatcccactggcaaggcacaggaagtacttccgggttcgacaatgctgatccgcaattagaagacactggtaagctgtgttacactgcaagaaaagaagcagagccaatgggtttggtgacttctgtggaaagctcctaagcagcagccataatgagccatgaagagcagatctgaagactcccaactactacccaaaatgtgatttagtctatcctgcccaaggccactcttctcactggaaggcccaagtaatttccatagatgttctctctgcctcacctgcagcatactgaggacctaaatcctcaacggacaaccaaaacctatgaactcagcctttcaggctaaaaatcagcaaccctaataggggtttctactactaaacataaacatcaatcttcttttgtcccagcaacagaaccatagccattaactaacccaaggtcctaccttctcttccctatacacaacaaaaattctatttcatgcaaaaacattttggcagtttctcagttcctgaaatctctggctactttatccaggttccccaacccctcccaggcctcttctcaacacagcaagttggctcttatcattgccactatattaggttacacaaagaaactcctcacctgggcttcattgaaatcttcaaggatatagccagctcctgctcgaagctgggattctgtatactgcttgttgaaaggaggaatttccaaaaattctatattaaaaaaaaaaaccaagata

SEQ ID NO: 17 - DNA, 733 bp, Artificial sequence
  Probe
cacaatctccacgatagcagggaataaaaccagtaagaccaagtatctttagtgagaaacataatcgtgtttatattttggatgctgcttgaatccaattctctccccaacaatgaggcactggatcacccactcttgtgacaccacaggcagctgcaatgcttcagcacacttcagcaccgaggctgggcatgaggggtccgtcaccaccacatcaaatacccctaaagcaatatctgcaaggagcaagggaaagtgaagaaggaaaggacactcaacttagccctccattagaaagagagatttgattctaaccaatacatcccactctgcacaaaccaaagccctattatgtcaaacacactgctactgatcatgaccaaaggcagagttataatcactatgtgctgaccttgtagaaatatttaacaaatatacgtccagtgcttcacttatgttgactcacctcttgaaggtggtacttttcttctctaagaaacatggatacggtcaacctattaggcctgagccttggaccacaaggcctaacacctacaggtctaaggagatccctggaacaaagacactacacacactctttcaggtacctttgttatgggcacttgaatggtgctgcttcacagaggctgcaccaccagtcatgaggatctcagaccagagctccaggaagttctgctgttggtctgataccaagagtaccttcagattctgg

SEQ ID NO: 18 - DNA, 734 bp, Artificial sequence
  Probe
gctagaattgcccaatctgtgtaggtataaattacttggtatagggagagagaaagcctatcttacctgttgctttcttacttggtggtaacatccagcagttagtctatttataaacataattactttttcacatatgaaccataaaatatttaactttctgctctatattgtttgtctaccgctgtatctcccacagcttgaacagtaccaaggtacgtagtaggtgctcaataaatgactattgaataaatgaacatatccaacaaatgttctcaatgtaaaggatcagagatgccacatgttctccttgatgggagagacccttccacatgggaatgatgggaaggagttgtactcctggatgttcagtaactgcttctaggagaaaaggtagagtcctatcactaagccgcagatatttatttgtgtgtggctagaatgggatgttttgaatcttctgttacaaccttgggaacgtggctgttatttcaatttatgagccagaaattttcacatcccgaaactgcccagagttccaccagcctgggtatagtatttgttataatctagtcgtaacagtagttgagccaaatctgagttgatctgatgattccgaacactggagagaatcttgaacaggagtgaagactggcggctaaagcccttcacgagaatgctcagctgggcggctccacgctcatccagtgggcctaggttctgactgaccagca

SEQ ID NO: 19 - DNA, 2289 bp, Homo sapiens
tcgcggccgcgtcracgcgtggtagggggcccagagcaagccgaaggcaagcacgatggcgctcaccagccggcccacccgcgccccgtgccgcccggagccccagcgggcgccccgcagccgtgccagcgtcacgctgtagcagccgagcatcagccgaaaggaagcacgaaagcggtcagagtctccaggctcaggtgggcggcggcgtggaccggcgacgggtggcacagctggcatacgcggtccctccacaggtggcggtagacggcggccgggacggcgagcaacagggcggccagccagaccgccagcagcaggcggcgggccagggccgggctgcgcagccgaggcgccaggaaggggcgggtgactgcgaggcagcgctgcaggctgagcaggccggtgagcagcacgcttggcgtacatgctgagcgcgcacacgtagtacaccgccttgcagcccgcctggcccagcggccaggcctgccggtcaggaaggccacaaagagcggcgtgagcagcagcaccgcgccgtcggccagcgccaggtgcagcacaagcgtggccgccagcggtcgcccccgtgcaggctgccagcccgccaagctccacaccacgaagccgttgccaggcagccccagcagcgccgccagcagcaggaaggctgtgcctgtggcccgcgaagtcttccagctcagcagtgtctcgttccctgggggacggtagcagaccgacatccttctgggcctacaggacacagaaaaaaagtggggaagctgggggaccctacaaggatccttggcaggaaagcagggattgtgttcattttgagggtttcactgtcagtgagagtctcagcttccatgcaactgtccatcacggctgcaactgaaatcagagctgggacacagcgcaccagaagctaaagtcttgatgccatcaaaggacatcccctgccccattcacayattcacatctctgtcacgtccactaatcggcaaaaggagaaaagtgagagaagatgacctaagtgtgactgcagcaggcagctctggaaaatgaagccagagcagtgagccagcccctcctccgaccaaggaggaaggaaagagcagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatgggcctggacgggagagccccaagcacagcaaggacatcggccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctagcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaccgtcaaccc

SEQ ID NO: 20 - DNA, 1511 bp, Homo sapiens
cacatttcatccttttacatggttcccatctaccctcacaacacatgtcatcaccaaagacacacatacaagctccaatggcttttgccaggcaattcttcctccaggaccccatctggcccctccctcatccctccccttggactttgcccttcttactggccaggcaggggggccagagtccaggcttgactcattcccaccttgtcctgggctgagatcccaggtttgtaacagaaaacaccactaaagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatggcctgggacgggagaggcccaagcacagcaaggacatcgcccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctggcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaccactaagacttctgactccaatttaaaccaggaaagggatggggtggataccccattttgccctcccccatcaacacccagtcccagatccaaagcc

SEQ ID NO: 21 - DNA, 6530 bp, Homo sapiens
ttttgttagtttgaggggaagggtatgaagacagatctcaaggtaaagtcagagagggctgtcatcagtatgctggggagtttagggacaggaggcattggtaggggattagatgtagcagcagtcaggctgggatcaagatgcctgggggacatcttgatcttggcctttcagggcaagtgggaggccagaaaggtggctaggaaagaacagcattcttcaggtaagggtatagacttgggatgtgaggcgttatgctgaaaggttctgtcacgaggggatcagaggacagtggggaaattgggtgggttatctagcctgtactgtctgcaggtcctgaaatttgatgctgtcatagtctttgcagtgggtcggttggaatgattctgggggcagaagctcagagccccttagtaggaatggaggcggcccttctgctgccactgctcagccccctccactgcatgacgaagggtggaggaaattcccagcaacatatggcccaggccttgcagcagtgtggaggtccaacgaaggagctccctgaatggcagagacaagaggaaatcagatgatttggaaaacttgggaggaagccatcaagctgggagatgaggactttccacaagcaagagctaactaggggtaggtgggtgcaagaggacgaattatggggactatccaactgtaggggatggggcagtatgacatgttgatttctgacctgagtactttctttgggccaagtccttgaaagtcacaactcatagagtagagcccgtagaatgtggctttgacattcaggctgccaaagaggtctcgagggttttgcttgtacacgtcaaaggtgaatcgggcgatgtccttgctgtgcttgggcctctcccgtcccaggccatatgacagcactccactctgtaggacacccttgtcagtgcagtagatcctcataccagacacccaccactaatctccatcagcactgggtcagaccctccctcgcttggactttctgtccactgtgtgacatccttgacaattccacaactcctcctgcacctggtccccaggatcagggttaagctagagaggaagcccgggaaagctctaaaggacaggcattggaagcagccccagtataggcctcttacccttgtagggctccagctctgaccagactgcaacaccatcaggcacgtgtcatcctccagcagctggaagaagtcctcactgtccactgcagttccatcctcctctagcaccagggttagcactccattcagcagtagggtctccaatgcctgcccaatggcaagaagcaagaagggcaggtcttatcccatgccccttccctctttagctgcccaacatccatcagttggctctagacattggtcgatgtcccactttgactttccggcactttgatacctcctaaaggttgcagctctccgtgttcttcagtttttgggggatcctagctagaggctgacctttttcctctttgctcctaccatgtcattggcatctccccttgctcccctccaagtcacttctggtttggaattggaaagcaagccaggttctcacgaagtccacccttctgtcttatctacaatgctgcacctcacttcccacaccctcaagagttctccagaagtgttttcagtaatagtgtttaacctttttgagtccttactctgtgccaggtatgaggactttacctacattatcctcttactcctttcaacaaccctaggaggtgatgtattattattgcctttttatagttgaagaaactgaggttttggtaggttgaacaacttcccaaggtttgacaggcaggaagtggcagaatcagaatttgaacttgatttgtcacacaaatcacctttccatactagcttctgaattctgtccctcgaactctccctatctcctgctaacccctgctcccatagaaaagctcactcggtggaaaatgaacaaattgaccagagctcattaggcccactccgctgcttttagccctcagagggaggggcagctgtgtgacttcagccctctgctccatcatcacaagttgccactgttgtggagccccttggctacccctgctataggaaccgaggaacttggcctacttactttggctagcagctcctggcgggtggcagctgtcaggcctttccggatggtccgcttgtgatcacagacacggaaaggtcgctggggtggtggagctgaggtccagaccctccgtccaaactccgagcttatattagatactgacctggtagttgagaagaaaagtcaagaaggggcgaggaggggcttggtgagtgtaaagggcatgatgagggtagagtggctagagggctagggagggagagatctaggtttatcgattagggatgagggagagaccatggagtgcaggtgggggcgggtggctcaggagcttgacaagcccactgtggagtggggagcaggagaggaaggggtactggttagtctcctaggggctgagtggagtattgttgccctgcctatatcccctaaaggtggagggtagagcggagggttagcagtcacctgagtaagtcactggggttcagagctgagaggtactccatggtggaccggagagttccttccctggaacttctgggctgggtggttctctcctgtgctggggctttagtggtgttttctgttacaaacctgggatctcagcccaggacaaggtgggaatgagtcaagcctggactctggcccccctgcctggccagtaagaagggcaaagtccaaggggagggatgagggaggggccagatggggtcctggaggaagaattgcctggcaaaagccattggagcttgtatgtgtgtctttggtgatgacatgtgttgtgagggtagatgggaaccatgtaaaaggatgaaatgtgacttctggtgtttttttatttctatggagggaatttctggggacggtttctggctctcaggctctgagaagctgcagtttatgagtggctctgtgtgtgctgccacctactggagaagccataagctgcagctttaggaaaagggaacccggggcagagtgtggggaagtgggatggcagcatggcagggctttggaaaatgagaggtgagactgtgtccaggaagggtgtaaggagaggatggatcctgatacatggattcaggatcattagggtcctgtctgggacactggccttcctgcttacctgctctttccttcctccttggtcggaggaggggctggctcactgctctggcttcattttccagagctgcctgctgcagtcacacttaggtcatcttctctcacttttctccttttgccgattagtggacgtgacagagatgtgaatggggcagggatgtcctttgatggcatcaagactttagcttctggtgcgctgtgtcccagctctgatttcagttgcagccgtgatggacagttgcatggaagctgagactctcactgacagtgaaaccctcaaatgaacacaatccctgctttcctgccaaggatccttgtagggtcccccagcttccccactttttttctgtgtcctgacaaagaaacacagagtaacttgattgccctgtgacctggccagttgcatttcccctgcaggcttgagcccaagccagagccttgaaaaggtattcaggttgttgcccaaaacactgaaaaaaactggccctggccctgaaccaaataccttgaaccctcgtaaactccataccctgacccccttgttttggatatacccaggtagaacaactctctctcactgtctgttgtgaggatacgctgtagcccactcattaagtacattctcctaataaatgctttggactgatcaccctgccagtcttttgtcttgggcaatctatacttttctcagaggttcccaaggcctactgaagggacttaacatactcttaatggctttcctctctcttgttttaccttatgccctcacttcctgagttaacctcccaaatacaggatcacctgtacccaagcccttagctcaagaatacaggatcacctgtacccaagcccttagctcaagctctgctttggaagaacccaaactaagacagtgctcctggtgccctccccaagcaacctcaagttctggctgttacttgagcagaggcctttcttttcccttcccccagctctatccatctgccaggcccccctcaaatctcttcatttccaagttttgcttgacttttccaagaggagagggctgcttcttagtatgtccctactcatcctttcctttcttgtcttgtatcctggtgcagcctggtaatggggcctcttcatggttgtgtgtcatgactccctaaccattatgcctccatgcatcccctgttcctcctggaacctagcaccatgccttacatggaaaagctgtcattgacagcccggtgagagccctgagggtggagtgactggggcagggcctgaggcaagaggtgggaggaggtaggaggccaggggctcagccggaccaggagactggaaacaggcaaggataaggcaggtgggggactgagttgtttgggtcacctctgcaggccagagagaccaggcaacatacacactgcagaaggtgggctgggaggattggggccagagctgggggagggatgagaacagaagcaggaccaggattcagcagagtcctcctatttccttccaccaccagggaatcttactgccccacttcagcttgtgctgtttcctggcaaggcaggctctcacatgcctggacgcctgggtgcgttggtgatgggaaggagcagggtgagggaggggccccaggagaggcccaggatgagcctcatcttgtccctccccattcttgtcttaccctctgcaaatgtgataggcacaggacaggagtaggcacctcgcctactgctgcttaacctttcagcttctccaggcccccaatcctgcttgctcccagcttggtaagtagatctgtgcacgtccctttacaccccaccatccagttttgcccagatgtgctagaatggggctggacaaagaaggaggggccagactagaggagtggtggtagagatagtgacagcctggggtgatgactttatgcctgtttaccactgagctctgggaaggaggccaggagtggggcaggtcaactgactgggagcaggggatctgggttccaagaaggagttgtgtttgaggtggggtctgggtcctcgtggaagtcaggactcccaggcagaaaagaggcaggctgcagggaagtaaggaggaggcatggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttgacagcagaccct

SEQ ID NO: 22 - DNA, 424 bp, Artificial sequence
  Probe
ggattagatgtagcagcagtcaggctgggatcaagatgcctgggggacatcttgatcttggcctttcagggcaagtgggaggctagaaaggtggctaggaaagaacagcattcttcaggtaagggtatagacttgggatgtgaggcgttatgctgaaaggttctgtcacgaggggatcagaggacagtggggaaattgggtgggttatctagcctgtactgtctgcaggtcctgaaatttgatgctgtcatagtctttgcagtgggtcggttggaatgattctgggggcagaagctcagagccccttagtaggaatggaggcggcccttctgctgccactgctcagccccctccactgcatgacgaagggtggaggaaattcccagcaacatatggcccaggccttgcagcagtgtggaggtcc

SEQ ID NO: 23 - DNA, 424 bp, Artificial sequence
  Probe
ggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctagcctcccacttgccctgaaaggccaagatcaagatgtcccccaggcatcttgatcccagcctgactgctgctacatctaatcc

SEQ ID NO: 24 - DNA, 7042 bp, Homo sapiens
aagaagaggtagcgagtggacgtgactgctctatcccgggcaaaagggatagaaccagaggtggggagtctgggcagtcggcgacccgcgaagacttgaggtgccgcagcggcatccggagtagcgccgggctccctccggggtgcagccgccgtcgggggaagggcgccacaggccgggaagacctcctccctttgtgtccagtagtggggtccaccggagggcggcccgtgggccgggcctcaccgcggcgctccgggactgtggggtcaggctgcgttgggtggacgcccacctcgccaaccttcggaggtccctgggggtcttcgtgcgccccggggctgcagagatccaggggaggcgcctgtgaggcccggacctgccccggggcgaagggtatgtggcgagacagagccctgcacccctaattcccggtggaaaactcctgttgccgtttccctccaccggcctggagtctcccagtcttgtcccggcagtgccgccctccccactaagacctaggcgcaaaggcttggctcatggttgacagctcagagagagaaagatctgagggaagatggatgcaaaagctcgaaattgtttgcttcaacatagagaagctctggaaaaggacatcaagacatcctacatcatggatcacatgattagtgatggatttttaacaatatcagaagaggaaaaagtaagaaatgagcccactcaacagcaaagagcagctatgctgattaaaatgatacttaaaaaagataatgattcctacgtatcattctacaatgctctactacatgaaggatataaagatcttgctgcccttctccatgatggcattcctgttgtctcttcttccagtgtaaggacagtcctgtgtgaaggtggagtaccacagaggccagttgtttttgtcacaaggaagaagctggtgaatgcaattcagcagaagctctccaaattgaaaggtgaaccaggatgggtcaccatacatggaatggcaggctgtgggaagtctgtattagctgcagaagctgttagagatcattcccttttagaaggttgtttcccagggggagtgcattgggtttcagttgggaaacaagacaaatctgggcttctgatgaaactgcagaatctttgcacacggttggatcaggatgagagtttttcccagaggcttccacttaatattgaagaggctaaagaccgtctccgcattctgatgcttcgcaaacacccaaggtctctcttgatcttggatgatgtttgggactcttgggtgttgaaagcttttgacagtcagtgtcagattcttcttacaaccagagacaagagtgttacagattcagtaatgggtcctaaatatgtagtccctgtggagagttccttaggaaaggaaaaaggacttgaaattttatccctttttgttaatatgaagaaggcagatttgccagaacaagctcatagtattataaaagaatgtaaaggctctccccttgtagtatctttaattggtgcacttttacgtgattttcccaatcgctgggagtactacctcaaacagcttcagaataagcagtttaagagaataaggaaatcttcgtcttatgattatgaggctctagatgaagccatgtctataagtgttgaaatgctcagagaagacatcaaagattattacacagatctttccatccttcagaaggacgttaaggtgcctacaaaggtgttatgtattctctgggacatggaaactgaagaagttgaagacatactgcaggagtttgtaaataagtctcttttattctgtgatcggaatggaaagtcgtttcgttattatttacatgatcttcaagtagattttcttacagagaagaattgcagccagcttcaggatctacataagaagataatcactcagtttcagagatatcaccagccgcatactctttcaccagatcaggaagactgtatgtattggtacaactttctggcctatcacatggccagtgccaagatgcacaaggaactttgtgctttaatgttttccctggattggattaaagcaaaaacagaacttgtaggccctgctcatctgattcatgaatttgtggaatacagacatatactagatgaaaaggattgtgcagtcagtgagaattttcaggagtttttatctttaaatggacaccttcttggacgacagccatttcctaatattgtacaactgggtctctgtgagccggaaacttcagaagtttatcagcaagctaagctgcaggccaagcaggaggtcgataatggaatgctttacctggaatggataaacaaaaaaaacatcacgaatctttcccgcttagttgtccgcccccacacagatgctgtttaccatgcctgcttttctgaggatggtcagagaatagcttcttgtggagctgataaaaccttacaggtgttcaaagctgaaacaggagagaaacttctagaaatcaaggctcatgaggatgaagtgctttgttgtgcattctctacagatgacagatttatagcaacctgctcagtggataaaaaagtgaagatttggaattctatgactggggaactagtacacacctatgatgagcactcagagcaagtcaattgctgccatttcaccaacagtagtcatcatcttctcttagccactgggtcaagtgactgcttcctcaaactttgggatttgaatcaaaaagaatgtcgaaataccatgtttggtcatacaaattcagtcaatcactgcagattttcaccagatgataagcttttggctagttgttcagctgatggaaccttaaagctttgggatgcgacatcagcaaatgagaggaaaagcattaatgtgaaacagttcttcctaaatttggaggaccctcaagaggatatggaagtgatagtgaagtgttgttcgtggtctgctgatggtgcaaggataatggtggcagcaaaaaataaaatctttttgtggaatacagactcacgttcaaaggtggctgattgcagaggacatttaagttgggttcatggtgtgatgttttctcctgatggatcatcatttttgacatcttctgatgaccagacaatcaggctctgggagacaaagaaagtatgtaagaactctgctgtaatgttaaagcaagaagtagatgttgtgtttcaagaaaatgaagtgatggtccttgcagttgaccatataagacgtctgcaactcattaatggaagaacaggtcagattgattatctgactgaagctcaagttagctgctgttgcttaagtccacatcttcagtacattgcatttggagatgaaaatggagccattgagattttagaacttgtaaacaatagaatcttccagtccaggtttcagcacaagaaaactgtatggcacatccagttcacagccgatgagaagactcttatttcaagttctgatgatgctgaaattcaggtatggaattggcaattggacaaatgtatctttctacgaggccatcaggaaacagtgaaagactttagactcttgaaaaattcaagactgctttcttggtcatttgatggaacagtgaaggtatggaatattattactggaaataaagaaaaagactttgtctgtcaccagggtacagtactttcttgtgacatttctcacgatgctaccaagttttcatctacctctgctgacaagactgcaaagatctggagttttgatctccttttgccacttcatgaattgaggggccacaacggctgtgtgcgctgctctgccttctctgtggacagtaccctgctggcaacgggagatgacaatggagaaatcaggatatggaatgtctcaaacggtgagcttcttcatttgtgtgctccgctttcagaagaaggagctgctacccatggaggctgggtgactgacctttgcttttctccagatggcaaaatgcttatctctgctggaggatatattaagtggtggaacgttgtcactggggaatcctcacagaccttctacacaaatggaaccaatcttaagaaaatacacgtgtcccctgacttcaaaacatatgtgactgtggataatcttggtattttatatattttacagactttagaataaaatagttaagcattaatgtagttgaactttttaaatttttgaattggaaaaaaattctaatgaaaccctgatatcaactttttataaagctcttaattgttgtgcagtattgcattcattacaaaagtgtttgtggttggatgaataatattaatgtagctttttcccaaatgaacatacctttaatcttgtttttcatgatcatcattaacagtttgtccttaggatgcaaatgaaaatgtgaatacataccttgttgtactgttggtaaaattctgtcttgatgcattcaaaatggttgacataattaatgagaagaatttggaagaaattggtattttaatactgtctgtatttattactgttatgcaggctgtgcctcagggtagcagtggcctgctttttgaaccacacttaccccaagggggttttgttctcctaaatacaatcttagaggttttttgcactctttaaatttgctttaaaaatattgtgtctgtgtgcatagtctgcagcatttcctttaattgactcaataagtgagtcttggatttagcaggcccccccaccttttttttttgtttttggagacagagtcttgctttgttgccaggctggagtgcagtggcgcgatctcggctcaccacaatcgctgcctcctgggttcaagcaattctcctgcctcagcctcccgagtagctgggactacaggtgtgcgcacatgccaggctaatttttgtatttttagtagagacggggtttcaccatgttggccgggatggtctcgatctcttgacctcatgatctacccgccttggcctcccaaagtgctgagattacaggcgtgagccaccgtgcctggccaggccccttctcttttaatggagacagggtcttgcactatcacccaggctggagtgcagtggcataatcatacctcattgcagcctcagactcctgggttcaagcaatcctcctgcctcagcctcccaagtagctgagactgcaggcacgagccaccacacccagctaatttttaagttttcttgtagagacagggtctcactatgttgtctaggctggtcttgaactcttggcctcaagtaatcctcctgcctcagcctcccaaagtgttgggattgcagatatgagccactggcctggccttcagcagttctttttgtgaagtaaaacttgtatgttggaaagagtagattttattggtctacccttttctcactgtagctgctggcagccctgtgccatatctggactctagttgtcagtatctgagttggacactattcctgctccctcttgtttcttacatatcagacttcttacttgaatgaaacctgatctttcctaatcctcacttttttcttttttaaaaagcagtttctccactgctaaatgttagtcattgaggtggggccaattttaatcataagccttaataagatttttctaagaaatgtgaaatagaacaattttcatctaattccatttacttttagatgaatggcattgtgaatgccattcttttaatgaatttcaagagaattctctggttttctgtgtaattccagatgagtcactgtaactctagaagattaaccttccagccaacctattttcctttcccttgtctctctcatcctcttttccttccttctttcctttctcttcttttatctccaaggttaatcaggaaaaatagcttttgacaggggaaaaaactcaataactagctatttttgacctcctgatcaggaactttagttgaagcgtaaatctaaagaaacattttctctgaaatatattattaagggcaatggagataaattaatagtagatgtggttcccagaaaatataatcaaaattcaaagattttttttgtttctgtaactggaactaaatcaaatgattactagtgttaatagtagataacttgtttttattgttggtgcatattagtataactgtggggtaggtcggggagagggtaagggaatagatcactcagatgtattttagataagctatttagcctttgatggaatcataaatacagtgaatacaatcctttgcattgttaaggaggttttttgtttttaaatggtgggtcaaggagctagtttacaggcttactgtgatttaagcaaatgtgaaaagtgaaaccttaattttatcaaaagaaatttctgtaaatggtatgtctccttagaatacccaaatcataattttatttgtacacactgttaggggctcatctcatgtaggcagagtataaagtattaccttttggaattaaaagccactgactgttataaagtataacaacacacatcaggttttaaaaagccttgaatggcccttgtcttaaaaagaaattaggagccaggtgcggtggcacgtgcctgtagtcccagctccttgggaggctgagacaggaggattccttgagccctggagtttgagtccagcctgggtgacatagcaagaccctgtcttaaaagaaaaatgggaagaaagacaaggtaacatgaagaaagaagagatacctagtatgatggagctgcaaatttcatggcagttcatgcagtcggtcaagaggaggattttgttttgtagtttgcagatgagcatttctaaagcattttcccttgctgtatttttttgtattataaattacattggacttcatatatataatttttttttacattatatgtctcttgtatgttttgaaactcttgtatttatgatatagcttatatgatttttttgccttggtatacattttaaaatatgaatttaaaaaatttttgtaaaaataaaattcacaaaattgttttgaaaaacaaaaaaaaaaaaaa

SEQ ID NO: 25 - DNA, 3019 bp, Artificial sequence
  Probe
tttttttttttttttttgaaaaacatttttggattgtttcattctttgcttgtcatttatctgttgattagaccactaaagtgaaggattcaagctaaatacatcaacctttctatttaggctttatcagctatatgtaaattcaattctatcaaaattttctgagtgcctcctcagtgtgtctctctgatggttcctgcccggtatggctggcatgaagaagatccacggacttgcgaatgctaacgcggggcttggggatgggtttggagggtttgttttcaaagctttctggaagtgtggaggagtgtcccccttttcttgcttgtagtgctagctggtaagcgacttcgaatgcctgtcccagggttaggatgatttcataggctaaattcacatcaaaggcagtaaacacatgacagtagtggtgattagacttcaaatcttttgtgatataggcaaatgttgagaggtcttctgggtcctgggcagcacaggagatattacgaatttcatgctcagcaattatgttcttatttgttgcatcaataaatttgactcctttatatgagacagaaagaataatagtagggaccttcttcatttgctctgtagacttctgacagttagcccgcatttttgcacaagcatcttgggttgattctgtccccctaagctcttttatcagcatagaacctaaataaaaagctttgtaatcacacgactggaagataagcttttctgggtgatgctgccagtactgtaccggggtagaggctgtggcttcattcggaggtcgcaaggtaatggaaggttctccccagtctcctgtctgagccatctgcctctccagttttgatcggggaatatcatcaaagtagttttcatttcttctcctccttgcatcgccctgcatgataatgtgaggaacgtctagggagccaccagtggtgtaagtgctttggctaagtgatggagacaactgaggaggagtgtgattaccactgggttccctgagggtgatggaccgagggggcttctgtgggggatcgtcgtgcagcctgtctcccagagatgccaaaatacgtttcctgtggccaatcaaattgatttttaaaacattaataagttcaacctcccagatttttttcaacaggtccatcgaagtgtagccattaattagaaaggctttggtgtagtcgcccagttcaatggaatccagccactcagctacagaggtgggatggtagccatcatgcccaatgggtctcatctttggaaggagctggattgcctgtagaattctttgtctgtgcccagaattaaggattccaatttccaacaaatcctgatcttccataacattgcttcccataaactgcacattgtcaaatccattagccatcaggtggttctcgtactgaggtagcccaatgctttccaaccattgtcccactgtttggacagggcatctgggtcttgtggtctcaccattcatctctttaagttcgttgttgattccaacatctatggaactcattattttgtcaatttcttcccattccgatgtgaaggatggtgttctttcagaattccctttagaactgtgttcagcagtggaagattcactccagttaactcttgatgttttctcattggaaggataggcaatgagatcagaatcagatttagagacactttttgacaaatgcatgtcgatcaaggctttaggcaatgaccttattctccccagagtacaggctctctccacaaatccccctgcgttcataacccactggtccccattccgagatccactcctggttgatcttgtgccaacaatggtatggttttcgagttggttgctttttttgtgaaaaattgttctactgaccactttgggtttaattttctttaccaaaggttctgagttattttctattggagactgcttgaaaggcaaaggactgtttgccaaactgacttcatcttgtcctttttcacattgttctcttttcccatagagatgaaatggattttcaggggactcacaggctggagaggatccatggagcaggcctgcaaattgcccaggatcatattcttttgggggatcattgtcatcctgtcgggagaggtcatctgtatggttagttccctcattcttgacttctgtggttcccacagaagaggtgggtggactagcaggaggactggcagtaaagcttgtgcaccctgtagatgtgttgatttcaaaatattcttggttgtgattcattcggtgaaaatccagagaagacacaatggatgttcgctgtttaggctggggtcgaatgacttttacaatatttttgagggcagtatcaggggatggaggtgaacaatcaggtgttgggcctgttgagctgtttctatggttactagttcctggagtagtaactgctacctcagaggcattatcagttcttggggaaggtgcccttgcaatttctaaggagcaaggtttctttgtaacagctgtgtccatgagatcacacagaaagttctcattttctgaaggaaatgtatccagagaagcagatggtacaatttccatagtgtaatttctcttctttggataggactcctgggcaagcatggggaagccaaggttcctacatccattacacggagttaatgcttcccaaagtcctgatggcccacacgtattttcatcatcatcctcttcttccacttctcctggtgacaaattgattgtagatgaggttcttacactctggcttccatttttcccaagttcttcctctgaaatcttgctcaaattatctaagtagtggtctgatatngtgtggcacaagtcttcaaacgaataatccttttcttgacagagttttatttcatccaagagttttgataattctccagtgacggtttcacttttggtcttttgggaaggagactcaacaggagatgaaatgtgtgtttcttgtgttgcatcttcctgtacaggctcttcgag

SEQ ID NO: 26 - DNA, 1752 bp, Artificial sequence
  Probe
agaacgcagaccagcccaagctgacagcttgagtatgccttcttctgctgcctggttttgggggctgtatgacgtactggtcggtagtaaagattaatatgtaagaaatgtggagctaggatcaagtcatactccacagcctgcctggcaaactatgttttacttctgactttgctctctcgctgagaacattaatctgtcaagctggcgggctcctttgatagcaactttcccaggggcatgatgtggcaatgccacctctcagcccaggactaccgctattaccccgtggacggctactccctgcttaaacgcttccctcttcatcctcttacaggacccagatgccctgtccaaacagtgggacaatggttggaaagcattgggctacctcagtacgagaaccacctgatggctaatggatttgacaatgtgcagtttatgggaagcaatgttatggaagatcaggatttgttggaaattggaatccttaattctgggcacagacaaagaattctacaggcaatccagctccttccaaagatgagacccattgggcatgatggctaccatcccacctctgtagctgagtggctggattccattgaactgggcgactacaccaaagcctttctaattaatggctacacttcgatggacctgttgaaaaaaatctgggaggttgaacttattaatgttttaaaaatcaatttgattggccacaggaaacgtattttggcatctctgggagacaggctgcacgacgatcccccacagaagccccctcggtccatcaccctcaggacaggagactggggagaaccttccattaccttgcgacctccgaatgaagccacagcctctaccccggtacagtactggcagcatcacccagaaaagcttatcttccagtcgtgtgattacaaagctttttatttaggttctatgctgataaaagagcttagggggacagaatcaacccaagatgcttgtgcaaaaatgcgggctaactgtcagaagtctacagagcaaatgaagaaggtccctactattattctttctgtctcatataaaggagtcaaatttattgatgcaacaaataagaacataattgctgagcatgaaattcgtaatatctcctgtgctgcccaggacccagaagacctctcaacatttgcctatatcacaaaagatttgaagtctaatcaccactactgtcatgtgtttactgcctttgatgtgaatttagcctatgaaatcatcctaaccctgggacaggcattcgaagtcgcttaccagctagcactacaagcaagaaaagggggacactcctccacacttccagaaagctttgaaaacaaaccctccaaacccatccccaagccccgcgttagcattcgcaagtccgtggatcttcttcatgccagccataccgggcaggaaccatcagagagacacactgaggaggcactcagaaaattttgatagaattgaatttacatatagctgataaagcctaaatagaaaggttgatgtatttagcttgaatccttcactttagtggtctaatcaacagataaatgacaagcaaagaatgaaacaatccaaaaatgtttttcaaaacaattttgtgaattttatttttacaaaaattttttaaattcatattttaaaatgtataccaaggcaaaaaaatcatataagctatatcataaatacaagagtttcaaaacatacaagagacatataatg

SEQ ID NO: 27 - DNA, 367 bp, Artificial sequence
  Probe
ccgcgttagcattcgcaagtccgtggatcttcttcatgccagccataccgggcaggaaccatcagagagacacactgaggaggcactcagaaaattttgatagaattgaatttacatatagctgataaagcctaaatagaaaggttgatgtatttagcttgaatccttcactttagtggtctaatcaacagataaatgacaagcaaagaatgaaacaatccaaaaatgtttttcaaaacaattttgtgaattttatttttacaaaaattttttaaattcatattttaaaatgtataccaaggcaaaaaaatcatataagctatatcataaatacaagagtttcaaaacatacaagagacatataatg

SEQ ID NO: 28 - DNA, 367 bp, Artificial sequence
  Probe
cattatatgtctcttgtatgttttgaaactcttgtatttatgatatagcttatatgatttttttgccttggtatacattttaaaatatgaatttaaaaaatttttgtaaaaataaaattcacaaaattgttttgaaaaacatttttggattgtttcattctttgcttgtcatttatctgttgattagaccactaaagtgaaggattcaagctaaatacatcaacctttctatttaggctttatcagctatatgtaaattcaattctatcaaaattttctgagtgcctcctcagtgtgtctctctgatggttcctgcccggtatggctggcatgaagaagatccacggacttgcgaatgctaacgcgg

SEQ ID NO: 29 - DNA, 2457 bp, Homo sapiens
cacgcagcaggatggcaagggctccgcttggggtcctgctcctcttggggcttctcggcaggggtgtggggaagaacgaggaactgcgtctttatcaccatctcttcaacaactatgacccaggaagccggccagtgcgggagcctgaggatactgtcaccatcagcctcaaggtcaccctgacgaatctcatctcactgaatgaaaaagaggagactctcaccactagcgtctggattggaatcgattggcaggattaccgactcaactacagcaaggacgactttgggggtatagaaaccctgcgagtcccttcagaactcgtgtggctgccagagattgtgctggaaaacaatattgatggccagttcggagtggcctacgacgccaacgtgctcgtctacgagggcggctccgtgacgtggctgcctccggccatctaccgcagcgtctgcgcagtggaggtcacctacttccccttcgattggcagaactgttcgcttattttccgctctcagacgtacaatgccgaagaggtggagttcacttttgccgtagacaacgacggcaagaccatcaacaagatcgacatcgacacagaggcctatactgagaacggcgagtgggccatcgacttctgcccgggggtgatccgccgccaccacggtggcgccaccgacggcccaggggagactgacgtcatctactcgctcatcatccgccggaagccgctcttctacgtcattaacatcatcgtgccctgtgtgctcatctcgggcctggtgctgctcgcctacttcctgccggcgcaggccggcggccagaaatgcacggtctccatcaacgtcctgctcgcccagaccgtcttcttgttcctcattgcccagaaaatcccagagacttctctgagcgtgccgctcctgggcaggttccttattttcgtcatggtggtcgccacgctcattgtcatgaattgcgtcatcgtgctcaacgtgtcccagcggacgcccaccacccacgccatgtccccgcggctgcgccacgttctcctggagctgctgccgcgcctcctgggctccccgccgccgcccgaggccccccgggccgcctcgcccccaaggcgggcgtcgtcggtgggcttattgctccgcgcggaggagctgatactgaaaaagccacggagcgagctcgtgtttgaggggcagaggcaccggcaggggacctggacggctgccttctgccagagcctgggcgccgccgcccccgaggtccgctgctgtgtggatgccgtgaacttcgtggccgagagcacgagagatcaggaggccaccggcgaggaagtgtccgactgggtgcgcatggggaatgcccttgacaacatctgcttctgggccgctctggtgctcttcagcgtgggctccagcctcatcttcctcggggcctacttcaaccgagtgcctgatctcccctacgcgccgtgtatccagccttagctcgcaccgacttcaatttcccacccatctccagtaggaaattgattttgaaaaagtaggctgccgccaccacggcattatgatcccttccccctgctgatcaatctgcagtttgtgaacttcacaagaatggtgtgtgcccgttccctggcgtgtgtaggcctggccgcagtccaggggtcagcaggaggaaagggttcacataggctctcaggtgccagtcttccagaaagcaaggactgcccttcattcagccttgctgacctcccagcctttctaaggctcagccccacgggactctggtggctgccagcttgtgagctatctatctatattcatttcatagccaaacaggagacccctttgcaggacttgcacacagggaggctgtagccaggaaaccctcttcttccctggtctggctctgctggagcgggtgggaaccaaacaccttcagtgctggtggccctcaggcccacaggtttaaggctgaggctgccctgacccttccacagtcatttcttctaggttttcttggcccagcactgcccatcccaccccatgaggctcactcattgcagatcccagcccaccctgcccctttcttccccaccctggaggctctctctgcctagtctacagtactgacagaaagcaaggacatgcggcctgcatggtgggagctggttgaattgtctttattaacaaacaggatatccaaggccactacattgaggaggggggaggggggagggaggagaagggttacttgctgctcacactatatacagatgcaagcaaggggcgtggagagtgagggctccctgctccctccctccaccggggaagggcatgggctagaagaggagaggggggtcgggaatggggggaatgttttggctgcggggtcccccctccattccctggagtttgggggaaggggaatcattaaagtgctttcaga

SEQ ID NO: 30 - DNA, 4863 bp, Homo sapiens
ggagatagcgcctgtcagtcggtgggtcggtcctcgcgccggccctccccctccccggtctccgggggaggcgcggtggagtccgcccccggggttctccgatgggggagaagcggcgacggcggcagtggagtaaccgagccggagcgtgagcggccccggtgccccgttccccacggaggccatgggcgacccagcccccgcccgcagcctggacgacatcgacctgtccgccctgcgggaccctgctgggatctttgagcttgtggaggtggtcggcaatggaacctacggacaggtgtacaagggtcggcatgtcaagacggggcagctggctgccatcaaggtcatggatgtcacggaggacgaggaggaagagatcaaacaggagatcaacatgctgaaaaagtactctcaccaccgcaacatcgccacctactacggagccttcatcaagaagagccccccgggaaacgatgaccagctctggctggtgatggagttctgtggtgctggttcagtgactgacctggtaaagaacacaaaaggcaacgccctgaaggaggactgtatcgcctatatctgcagggagatcctcaggggtctggcccatctccatgcccacaaggtgatccatcgagacatcaaggggcagaatgtgctgctgacagagaatgctgaggtcaagctagtggattttggggtgagtgctcagctggaccgcaccgtgggcagacggaacactttcattgggactccctactggatggctccagaggtcatcgcctgtgatgagaaccctgatgccacctatgattacaggagtgatatttggtctctaggaatcacagccatcgagatggcagagggagccccccctctgtgtgacatgcaccccatgcgagccctcttcctcattcctcggaaccctccgcccaggctcaagtccaagaagtggtctaagaagttcattgacttcattgacacatgtctcatcaagacttacctgagccgcccacccacggagcagctactgaagtttcccttcatccgggaccagcccacggagcggcaggtccgcatccagcttaaggaccacattgaccgatcccggaagaagcggggtgagaaagaggagacagaatatgagtacagcggcagcgaggaggaagatgacagccatggagaggaaggagagccaagctccatcatgaacgtgcctggagagtcgactctacgccgggagtttctccggctccagcaggaaaataagagcaactcagaggctttaaaacagcagcagcagctgcagcagcagcagcagcgagaccccgaggcacacatcaaacacctgctgcaccagcggcagcggcgcatagaggagcagaaggaggagcggcgccgcgtggaggagcaacagcggcgggagcgggagcagcggaagctgcaggagaaggagcagcagcggcggctggaggacatgcaggctctgcggcgggaggaggagcggcggcaggcggagcgcgagcaggaatacaagcggaagcagctggaggagcagcggcagtcagaacgtctccagaggcagctgcagcaggagcatgcctacctcaagtccctgcagcagcagcaacagcagcagcagcttcagaaacagcagcagcagcagctcctgcctggggacaggaagcccctgtaccattatggtcggggcatgaatcccgctgacaaaccagcctgggcccgagaggtagaagagagaacaaggatgaacaagcagcagaactctcccttggccaagagcaagccaggcagcacggggcctgagccccccatcccccaggcctccccagggcccccaggacccctttcccagactcctcctatgcagaggccggtggagccccaggagggaccgcacaagagcctggtggcacaccgggtcccactgaagccatatgcagcacctgtaccccgatcccagtccctgcaggaccagcccacccgaaacctggctgccttcccagcctcccatgaccccgaccctgccatccccgcacccactgccacgcccagtgcccgaggagctgtcatccgccagaattcagaccccacctctgaaggacctggccccagcccgaatcccccagcctgggtccgcccagataacgaggccccacccaaggtgcctcagaggacctcatctatcgccactgcccttaacaccagtggggccggagggtcccggccagcccaggcagtccgtgccagtaaccccgacctcaggaggagcgaccctggctgggaacgctcggacagcgtccttccagcctctcacgggcacctcccccaggctggctcactggagcggaaccgcgtgggagtctcctccaaaccggacagctcccctgtgctctcccctgggaataaagccaagcccgacgaccaccgctcacggccaggccggcccgcaagctataagcgagcaattggtgaggactttgtgttgctgaaagagcggactctggacgaggcccctcggcctcccaagaaggccatggactactcgtcgtccagcgaggaggtggaaagcagtgaggacgacgaggaggaaggcgaaggcgggccagcagaggggagcagagatacccctgggggccgcagcgatggggatacagacagcgtcagcaccatggtggtccacgacgtcgaggagatcaccgggacccagcccccatacgggggcggcaccatggtggtccagcgcacccctgaagaggagcggaacctgctgcatgctgacagcaatgggtacacaaacctgcctgacgtggtccagcccagccactcacccaccgagaacagcaaaggccaaagcccaccctcgaaggatgggagtggtgactaccagtctcgtgggctggtaaaggcccctggcaagagctcgttcacgatgtttgtggatctagggatctaccagcctggaggcagtggggacagcatccccatcacagccctagtgggtggagagggcactcggctcgaccagctgcagtacgacgtgaggaagggttctgtggtcaacgtgaatcccaccaacacccgggcccacagtgagacccctgagatccggaagtacaagaagcgattcaactccgagatcctctgtgcagccctttggggggtcaacctgctggtgggcacggagaacgggctgatgttgctggaccgaagtgggcagggcaaggtgtatggactcattgggcggcgacgcttccagcagatggatgtgctggaggggctcaacctgctcatcaccatctcagggaaaaggaacaaactgcgggtgtattacctgtcctggctccggaacaagattctgcacaatgacccagaagtggagaagaagcagggctggaccaccgtgggggacatggagggctgcgggcactaccgtgttgtgaaatacgagcggattaagttcctggtcatcgccctcaagagctccgtggaggtgtatgcctgggcccccaaaccctaccacaaattcatggccttcaagtcctttgccgacctcccccaccgccctctgctggtcgacctgacagtagaggaggggcagcggctcaaggtcatctatggctccagtgctggcttccatgctgtggatgtcgactcggggaacagctatgacatctacatccctgtgcacatccagagccagatcacgccccatgccatcatcttcctccccaacaccgacggcatggagatgctgctgtgctacgaggacgagggtgtctacgtcaacacgtacgggcgcatcattaaggatgtggtgctgcagtggggggagatgcctacttctgtggcctacatctgctccaaccagataatgggctggggtgagaaagccattgagatccgctctgtggagacgggccacctcgacggggtcttcatgcacaaacgagctcagaggctcaagttcctgtgtgagcggaatgacaaggtgttttttgcctcagtccgctctgggggcagcagccaagtttacttcatgactctgaaccgtaactgcatcatgaactggtgacggggccctgggctggggctgtcccacactggacccagctctccccctgcagccaggcttcccgggccgcccctctttcccctccctgggcttttgcttttactggtttgatttcactggagcctgctgggaacgtgacctctgacccctgatgctttcgtgatcacgtgaccatcctcttccccaacatgtcctcttcccaaaactgtgcctgtccccagcttctggggagggacacagcttccccttcccaggaattgagtgggcctagcccctccccccttttctccatttgagaggagagtgcttggggcttgaaccccttaccccactgctgctgactgggcagggccctggacccctttatttgcacgtcaggggagccggctccccccttgaatgtaccagaccctggggggggtcactgggccctagatttttggggggtcaccagccactccaggggcagggaccatttcttcattttctgaaagcactttaatgattccccttcccccaaactccagggaatggaggggggaccccgccagccaaaacattccccccattcccgacccccctctcctcttctagcccatgcccttccccggtggagggagggagcagggagccctcactctccacgccccttgcttgcatctgtatatagtgtgagcagcaagtaacccttctccctccccccccacccctcctcaatgtagtggccttggatatcctgtttgttaataaagacaattcaaccagct

SEQ ID NO: 31 - DNA, 283 bp, Artificial sequence
  Probe
agctggttgaattgtctttattaacaaacaggatatccaaggccactacattgaggaggggggaggggggagggaggagaagggttacttgctgctcacactatatacagatgcaagcaaggggcgtggagagtgagggctccctgctccctccctccaccggggaagggcatgggctagaagaggagaggggggtcgggaatggggggaatgttttggctgcggggtcccccctccattccctggagtttgggggaaggggaatcattaaagtgctttcaga

SEQ ID NO: 32 - DNA, 283 bp, Artificial sequence
  Probe
tctgaaagcactttaatgattccccttcccccaaactccagggaatggaggggggaccccgcagccaaaacattccccccattcccgacccccctctcctcttctagcccatgcccttccccggtggagggagggagcagggagccctcactctccacgccccttgcttgcatctgtatatagtgtgagcagcaagtaacccttctcctccctcccccctcccccctcctcaatgtagtggccttggatatcctgtttgttaataaagacaattcaaccagct

SEQ ID NO: 33 - DNA, 2714 bp, Homo sapiens
ggcacagggcgaggttttatacacctgaaagaagagaatgtcaagacgaagtagccgtttacaagctaagcagcagccccagcccagccagacggaatccccccaagaagcccagataatccaggccaagaagaggaaaactacccaggatgtcaaaaaaagaagagaggaggtcaccaagaaacatcagtatgaaattaggaattgttggccacctgtattatctggggggatcagtccttgcattatcattgaaacacctcacaaagaaataggaacaagtgatttctccagatttacaaattacagatttaaaaatctttttattaatccttcacctttgcctgatttaagctggggatgttcaaaagaagtctggctaaacatgttaaaaaaggagagcagatatgttcatgacaaacattttgaagttctgcattctgacttggaaccacagatgaggtccatacttctagactggcttttagaggtatgtgaagtatacacacttcatagggaaacattttatcttgcacaagacttttttgatagatttatgttgacacaaaaggatataaataaaaatatgcttcaactcattggaattacctcattattcattgcttccaaacttgaggaaatctatgctcctaaactccaagagtttgcttacgtcactgatggtgcttgcagtgaagaggatatcttaaggatggaactcattatattaaaggctttaaaatgggaactttgtcctgtaacaatcatctcctggctaaatctctttctccaagttgatgctcttaaagatgctcctaaagttcttctacctcagtattctcaggaaacattcattcaaatagctcagcttttagatctgtgtattctagccattgattcattagagttccagtacagaatactgactgctgctgccttgtgccattttacctccattgaagtggttaagaaagcctcaggtttggagtgggacagtatttcagaatgtgtagattggatggtaccttttgtcaatgtagtaaaaagtactagtccagtgaagctgaagacttttaagaagattcctatggaagacagacataatatccagacacatacaaactatttggctatgctggaggaagtaaattacataaacaccttcagaaaagggggacagttgtcaccagtgtgcaatggaggcattatgacaccaccgaagagcactgaaaaaccaccaggaaaacactaaagaagataactaagcaaacaagttggaattcaccaagattgggtagaactggtatcactgaactactaaagttttacagaaagtagtgctgtgattgattgccctagccaattcacaagttacactgccattctgattttaaaacttacaattggcactaaagaatacatttaattatttcctatgttagctgttaaagaaacagcaggacttgtttacaaagatgtcttcattcccaaggttactggatagaagccaaccacagtctataccatagcaatgtttttcctttaatccagtgttactgtgtttatcttgataaactaggaattttgtcactggagttttggactggataagtgctaccttaaagggtatactaagtgatacagtactttgaatctagttgttagattctcaaaattcctacactcttgactagtgcaatttggttcttgaaaattaaatttaaacttgtttacaaaggtttagttttgtaataaggtgactaatttatctatagctgctatagcaagctattataaaacttgaatttctacaaatggtgaaatttaatgttttttaaactagtttatttgccttgccataacacattttttaactaataaggcttagatgaacatggtgttcaacctgtgctctaaacagtgggagtaccaaagaaattataaacaagataaatgctgtggctccttcctaactggggctttcttgacatgtaggttgcttggtaataacctttttgtatatcacaatttgggtgaaaaacttaagtaccctttcaaactatttatatgaggaagtcactttactactctaagatatccctaaggaatttttttttttaatttagtgtgactaaggctttatttatgtttgtgaaactgttaaggtcctttctaaattcctccattgtgagataaggacagtgtcaaagtgataaagcttaacacttgacctaaacttctattttcttaaggaagaagagtattaaatatatactgactcctagaaatctatttattaaaaaaagacatgaaaacttgctgtacataggctagctatttctaaatattttaaattagcttttctaaaaaaaaaatccagcctcataaagtagattagaaaactagattgctagtttattttgttatcagatatgtgaatctcttctccctttgaagaaactatacatttattgttacggtatgaagtcttctgtatagtttgtttttaaactaatatttgtttcagtattttgtctgaaaagaaaacaccactaattgtgtacatatgtattatataaacttaaccttttaatactgtttatttttagcccattgtttaaaaaataaaagttaaaaaaatttaactgcttaaaagtaaagttttgccattgcttggagaaacttttttttccttctctgcgctgccagctgta

SEQ ID NO: 34 - DNA, 6773 bp, Homo sapiens
caagcatgtgatgttcttgtaccttcttctgatagtacatctcaacagttgactccatatagtcaagtccatatttgtttgagatctggcaactatcaggaggtaatacagattttcattgaagacaacttaaccttgagtttacctgtccagttccgacagtcagtcctaagagaactctttaagaaagctcaacagggaaatgaagctctagatgaaatctgttttaaagtttgtgcctgtaatacagtccgtgatatactggaaggcagaacaattagtgttcaatttaaccagctatttcttagaccaaataaagagaaaatagactttcttcttgaggtatgttcaagatcagtaaatttagaaaaagcttcagagtctttgaaaggaaacatggctgcttttctaaagaatgtgtgtctggggttggaagatctgcagtatgttttcatgatttcttcacatgagcttttcattacattgttgaaagatgaagaacgaaagctacttgttgatcagatgaggaagagatcccctagagtaaatctgtgcattaaacctgtaacttcattttatgatatcccagcttcagcaagtgtcaacattggtcagttagagcatcaacttatattgtcagtggatccttggaggattagacaaattttaattgaattacatggtatgacttcagagcgccagttctggacagtgtctaataagtgggaagtaccttctgtctatagtggtgttatcctgggaattaaagacaatttaacaagagatttggtttatattcttatggccaaaggtttgcactgcagtactgttaaggacttttcccatgctaaacagctctttgctgcttgtttggagttggtaacagagttctcaccgaagcttcgtcaggtcatgctgaatgagatgttgcttttggatattcatacacacgaagctgggacagggcaggcaggagagagaccgccatccgaccttataagtagagtacgaggctatctggaaatgaggcttcctgatattcctcttcgtcaagttatagctgaggaatgtgttgcctttatgttaaactggagagaaaatgaataccttacactccaagttcctgcatttttgcttcagagtaatccatatgtaaagcttggacagcttttagcagctacatgcaaagaacttccaggccctaaagaaagcagacggactgccaaagacctttgggaagttgttgttcaaatctgtagtgtgtccagtcagcacaaacgaggaaatgatggcagagttagtttaataaaacagagggaatctacgttaggtatcatgtatcggagtgaactgctttcttttatcaaaaaattacgagaaccactcgttttgactattattttatcactctttgtgaaacttcacaatgttcgggaggacattgtgaatgatattacagctgaacacatttctatttggccatcttccattcccaacctccagtctgtggactttgaagctgtggcaatcacagtgaaagagctagttcgatatacactcagtataaatccaaataaccattcttggttaattatccaggcagatatttactttgcaacgaatcagtattcagcagctcttcactattacctccaggcaggagctgtgtgttctgacttctttaacaaggctgtgccccctgatgtttatacagaccaggtaataaaacgaatgataaaatgttgttctttgctgaattgccacacacaggtggctattttatgtcagttcctcagagaaattgactacaaaacagcgtttaaatctctgcaagaacaaaacagtcatgatgctatggactcctactacgactacatatgggatgttaccattttggaatacttgacttatcttcatcataaaagaggagaaacagataaaagacaaattgcaatcaaagccatcggccagacagagttgaatgcaagcaatccagaagaagtgttacagctggcagcgcagagaaggaaaaaaaagtttctccaagcaatggcaaaactttacttttaagcagttaaatttttttaacttttattttttaaacaatgggctaaaaataaacagtattaaaaggttaagtttatataatacatatgtacacaattagtggtgttttcttttcagacaaaatactgaaacaaatattagtttaaaaacaaactatacagaagacttcataccgtaacaataaatgtatagtttcttcaaagggagaagagattcacatatctgataacaaaataaactagcaatctagttttctaatctactttatgaggctggattttttttttagaaaagctaatttaaaatatttagaaatagctagcctatgtacagcaagttttcatgtctttttttaataaatagatttctaggagtcagtatatatttaatactcttcttccttaagaaaatagaagtttaggtcaagtgttaagctttatcactttgacactgtccttatctcacaatggaggaatttagaaaggaccttaacagtttcacaaacataaataaagccttagtcacactaaattaaaaaaaaaaattccttagggatatcttagagtagtaaagtgacttcctcatataaatagtttgaaagggtacttaagtttttcacccaaattgtgatatacaaaaaggttattaccaagcaacctacatgtcaagaaagccccagttaggaaggagccacagcatttatcttgtttataatttctttggtactcccactgtttagagcacaggttgaacaccatgttcatctaagccttattagttaaaaaatgtgttatggcaaggcaaataaactagtttaaaaaacattaaatttcaccatttgtagaaattcaagttttataatagcttgctatagcagctatagataaattagtcaccttattacaaaactaaacctttgtaaacaagtttaaatttaattttcaagaaccaaattgcactagtcaagagtgtaggaattttgagaatctaacaactagattcaaagtactgtatcacttagtataccctttaaggtagcacttatccagtccaaaactccagtgacaaaattcctagtttatcaagataaacacagtaacactggattaaaggaaaaacattgctatggtatagactgtggttggcttctatccagtaaccttgggaatgaagacatctttgtaaacaagtcctgctgtttctttaacagctaacataggaaataattaaatgtattctttagtgccaattgtaagttttaaaatcagaatggcagtgtaacttgtgaattggctagggcaatcaatcacagcactactttctgtaaaactttagtagttcagtgataccagttctacccaatcttggtgaattccaacttgtttgcttagttatcttctttagtgttttcctggtggtttttcagtgctcttcggtggtgtcataatgcctccattgcacactggtgacaactgtcccccttttctgaaggtgtttatgtaatttacttcctcctatacatgggaagaaatcatgcactgatttcataaatcaaagtcaaaccagacttctgggtacttatttgagattatttaggcctaattttaatagtctttttatgtctttgcaagtgtgaagggtcatattctgaaagtttctgtaacgttatatattttttaaactctttatctagactgttggcattgatcttgagacacttcacaaatcttgctttgatttcaaagtaattttattaacttttctacattttgaaatcagtgtgccccttagaactttctttcccctgaaactgcctgaaggagtactctattcctaccatcagttttggtgacttactagattcagatagcaaagccaaaaaactcacaaaaaaacataccagcatagccaaatagtttgtatgtgtctggatattatgtctgtcttccataggaatcttcttaaaagtcttcagcttcactggactagtactttttactacattgacaaaaggtaccatccaatctacacattctgaaatactgtcccactccaaacctagatagatagaaaaaagttagaaaagcatgaaggttgtacatcagaaactatcttacatatgtctgatgtacttgttgctgtttttgagatattttaaaagaaaccaaatcataaccaagaagtttagcatgtcaaaacagattatcactctcaaactatttacatgactatgttgaagggaaaaaggacttcagaacttcttaaccagtaccttctacatatgaaattgaaatggtcaaatcccaaagaactcttaaagcagaactataatgttgattcatttcaactgtatttaaattccatttggtctttttgttgatacacattcaggattggaaagtacttctaacagaaagataattactgaacagctaattttttttttgccaaagttttaaatgcatgtttagcagaatgttaaagttcagagactgtagtcccattagaagttgtgaaaaggtaagaagacaacaaatagagagtcttacctgaggctttcttaaccacttcaatggaggtaaaatggcacaaggcagcagcagtcagtattctgtactggaactctaatgaatcaatggctagaatacacagatctaaaagctaacaggaaaaacaaaagtacaagcaatttaggagaaagatgagtactaaatgtctcttgctaaaaccttagggatctagagataaataagccaccacccggccaggcgcggtggctcacgcctgtaatcccagcactttgggaggccgaggcgggtggatcacaaggtcacgagatcgagaccatcctggctaacacggtgaaaccccgcctctactaaaaaatacaaaaaattagctgggcttggtggcacgcgcctgtagtcccagctactcgggaggctgaggcaggagaatggcgtgaacccgggatgcagagcttgcagtgagcagagatcgcgccactgcactccagcctgggcgaaagagcgagactccgtctcaaaaaaaaaaaaaacaagccaccaacctgaaggaagtagacaaggaaggactgttgcaatacagtgtgacatgtactagcaggaagggcacctaatccagattggaaaagatagtgatggcctcaaattgccataaatgggtcttaaaagataagggagccaggaagagtaggaggcagagaatgttctaggtatagggacattacttggaactcagttcacagttcagaactcctaaggtgaaaaataaataaggagtaccttcatttcttatcaagaaagatgaggggtggtggctagaaagaggcatggtctagattggatcacaaagggtctttaagaagtcagaattttataggctgattcttgaagctactggaagatttttaaatcaaagttccattttaagaaagataccttagaatgcagtgaagcagacagactagaagaaaacatgtttattaagcagtgagattagttaaaaggctgtataatctaggcaataagagctgaactagtagcagtggaatggtatagtgtaaaaggggtagatttcacagatttgagaagatacttgtgcagtggaattaaacttcaattctctttgtcctcattggtccagaaggtaggagaaatgggagaagagctgggaattggaagtgaaatattactgttatatacctctagaaagtccacattgtttatcggcttatcaaagatttaccatcactatcagaagggtatagctgcctaggacaatttgggatgctaggaattctggatgaaaaaattaagcttttaataaaaagttttataaaataaaccaatttcagtatacttagtggttatccaatttgagtattcataatgtgctagatttaagcaccactgcccacaaattttaacctaggtgacttaataattatccccaaatgtcttccatatgttagattttcacatcccacatagaataagagggtagattttcttcacttttgttatatggcagatacagcagccttaagattacttacgagaagtaagcaagaaagaatgggatctcctctttttttttttttttaattttttgagatggagtcttgctctgttgctcaggctggagtgcagtagtgcgatctcggctcactgcaacctccacctcccaggttccagcgattctcctgcctcagcctcccaagtaacatgttggctaggctgcctcagccgcccaaactcctgacctcaagtgatctgcctgcctgcctcagccgcccaaagtgctgagattagagacctgagccacagtgcccggccagatcctcctcctcctctacttacttactttgttaaatatgctagcctggaaaagtttactttgaatttatgttctaaaaaatttttttaacaaagtaattttaattctgatatttaacttgataggcactctgtgtatccaaatgtaaagacatcatacagaataattctatgccattataaagcttaaacacaactggcgaaaaaaatgcttttccccattttatatcaaaaagagatactttagtttggactcctaaagaatgaaagtactcagaaaagtgtaaggactttgtttttctagaaatattaagcaacataaacactggggacagaactttatgcgtc

SEQ ID NO: 35 - DNA, 1590 bp, Mus musculus
ctgagaaccagacatcaggatggcaggggctctgcttggtgccctgcttctcctgacactctttggcagaagccagggaaagaatgaagagcttagcctgtatcaccatctcttcgacaattatgatccagaatgccggccagttaggagacctgaggacactgtcaccatcaccctcaaggtcaccctaaccaacctcatctcactgaacgagaaagaagaaactctgaccaccagtgtctggattggcattgactggcacgactatcggctcaactacagcaaggacgattttgcaggtgtaggaatcctccgggtcccttcagaacatgtatggctgccagagattgttctagaaaacaatattgatgggcagtttggagtggcctacgacagcaatgttctagtctatgagggaggctatgtgagctggttgcccccagccatctaccgcagcacctgcgcagtggaggtcacctatttcccctttgactggcagaactgctctctcatttttcgctcccagacctacaatgctgaggaggtggagttcatctttgccgtggatgacgacggcaataccatcaacaagattgacattgacacggcagcttttaccgagaatggagaatgggccatagactactgcccaggcatgattcgccgctatgagggaggttccacagaaggtcctggagaaactgacgtcatctatacgctcatcatccgccggaagccgcttttttacgtcattaacatcattgtgccttgcgtgctcatttctggcttggtgctgctcgcttacttcctgcctgcgcaggctggtggccagaaatgcacggtctctatcaacgtcctgctagcccagactgtcttcttgtttctaattgcccagaaaattccagagacttctctgagcgtgccactgctgggcaggtatcttatattcgtcatggtggttgccacgctcattgtcatgaattgcgtcatcgtgctcaacgtatctttgaggacgccaacgactcatgctacatcccctcggctgcgccagattttattagagctgctgccgcgtctcctgggctcgagcccacccccagaggatccccgaactgcctcaccagcgaggcgtgcctcgtctgtgggcattctgctcagagcggaggagctcatcttgaaaaagccgcggagcgaactcgtgtttgagggtcagaggcatcggcacggaacttggaccgcagccctctgccagaacctgggtgctgcagccccagaaatccgctgctgtgtggatgctgtgaactttgtggctgagagcacaagagaccaggaagccactggagaggaactgtccgactgggtgcgtatggggaaggccctggacaatgtctgtttttgggcagctttggtgctcttcagcgttggttctactctcatcttccttgggggttacttcaaccaagttcctgatctcccttacccaccgtgcatccaaccatgagcctgcactggcacccacctctcccccaccccccaagaaagagattttgaaaacaggccgctgacaataaatctggtttgtgaacttgc

SEQ ID NO: 36 - DNA, 2227 bp, Mus musculus
tgtgagcagcaagtagcccttctccctcctgtatcctttctcaatgtagtggccttggatatatcccctttgttaataaagacaattcaaccagcttccaccattttgagatcctactattgttctctctcaatcctggagagatttgagagttgagaatgcagagggtagaggaaaggcattaggctctgtgaagttactgtgataatagagacgaagtaaggtggatgaataggccagggatcagtcctgacacggtaggaccctttgagaatagtttttaccagccccagcagggccaggccagacttctggcttcagtgtttctatatctgggtcttgtaaaaacctcattggctatcaactagataaacattctttaggttagaaggagccaagagcaaaattgaaccaattgcctccaagtgcctgaccaaaccacccacccatcttctacttccctgaggagttggacccacccacatgaccacacaacccctcgggcagttcacaaaccagatttattgtcagcggcctgttttcaaaatctctttcttggggggtgggggagaggtgggtgccagtgcaggctcatggttggatgcacggtgggtaagggagatcaggaacttggttgaagtaacccccaaggaagatgagagtagaaccaacgctgaagagcaccaaagctgcccaaaaacagacattgtccagggccttccccatacgcacccagtcggacagttcctgtgagagagagcttagcgagggaggagcctggagggcggggcatctagcactgctccgcctcaacctcccaacccacctctccagtggcttcctggtctcttgtgctctcagccacaaagttcacagcatccacacagcagcggatttctggggctgcagcacccaggttctggcagagggctgctgctaaggcaacagcaagcgctaggtcattaaaagagcgtcctaacggcgagtgtatgcctttgacccaagagcagtgcttaccggtccaagttccgtgccgatgcctctgaccctcaaacacgagttcgctccgcggctttttcaagatgagctcctccgctctgagcagaatgcccacagacgaggcacgcctcgctggtgaggcagttcggggatcctctgggggtgggctcgagcccaggagacgcggcagcagctctaataaaatctgcagccggggcagagagaggttccaagcccgcttcccacccctgggcagtactttctccaaccagcgcttacctggcgcagccgaggggatgtagcatgagtcgttggcgtcctcaaagatacgttgagcacgatgacgcaattcatgacaatgagcgtggcaaccaccatgacgaatataagatacctgatatacagaagcctgatgtcacagcaccccacaaacaaggcactagctgccctctacctcacaaataccacctcgcacagctggtggcgttacttcttgatcctcctcaacgatgccagtattgtcctggcccttctgcatataccatctgttgcggacatgaaggggattcccagcaatttggacaccctgctgtgggtctaccacttccacagctccaccgaggtgagggtattagaatggcagaatctggagaggtccccagctcttcctgctatggccctttccatgtgatcattccactcactacccttgctcctccaggtggccttacagcctccacttctatcttccctggaacttgctgtggccgcagctcacgaatatctggtgcaaaggttcagagagcttaagtcccaggaccccctggaatccgacaagtcgcccacccagaaggccaccctagggctggtgctaagagaagctgcagccagcatcatgagctttggagccaccttgttagaggtgctgctctgggaggctgagggatgggaataaaagggggagagggctaggccaacaaaagcaaggacctctagcccatatgccccaatgtagatctcggccctgtggctgcagcaggaggtgcagcgactggacggcggcaacgactgcccaggcccagccccagacactggggatcctggtagggcgctggcccgtgtagccctggccgcagggcaggggattcggcaagctggaacggcagctggcgcaagtgcccggtacctgatccagggggcgtggttgtacctgtgtggacgaggtttg

SEQ ID NO: 37 - DNA, 2472 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaaaaatagacaattttaaaaaccaaacagaatgggactgtcttctgcaagcctacctacaaacaggtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgttcgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttaaaaaaaaaaaaaaaaaa

SEQ ID NO: 38 - DNA, 4165 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaagtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgtttgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttagtgataggactctaccttttctcctagaagcagttactgaacatccaggagtacaactccttcccatcattcccatgtggaagggtctctcccatcaaggagaacatgtggcatctctgatcctttacattgagaacatttgttggatatgttcatttattcaatagtcatttattgagcacctactacgtaccttggtactgttcaagctgtgggagatacagcggtagacaaacaatatagagcagaaagttaaatattttatggttcatatgtgaaaaagtaattatgtttataaatagactaactgctggatgttaccaccaagtaagaaagcaacaggtaagataggctttctctctccctataccaagtaatttatacctacacagattgggcaattctagctaatgaaaatatacttaaaagtatttcttaggccgggcatggtggctcacacctgtaatcccagcactttgggaggccgaggcgggcggatcacctgaagtcaggagtttgagaccagcctgaccaacatgatgaaacctcgattctactaaaaatacaaaaattagccaggtgtggtggcatgtgcctgtaatcccagctactcaggaggctgagacaggagaattgcttgaacctgggaagcagacgctgcagtgagctgagattgtgccattgcattccagcctgggcaacaagagcgaaattccgtctcaaaaaaaaaaaaaaaaaaaaaagtattattctccaagaaaaaggtccttaagaaaaaattgagatcaagttgttagatttttaaatactgaagattgcaggcccaattacccatcttacacaaaccataggggttgaagttatcttaatatggcccagccatcactggtaatcaatattcatatcagtgtaagtaaaaagaaatattcactgaacaacgccctccaaactgaaaaagaatgcagtgttctggcatcaggttatagtcactgcatctggttttcatcactacatattctacacacactgggaagctctgacaacttattccctgctattatcaactaaagatcaccctttccactgctgtctctggagcaggagctggcaaactatggcctgctgtctgtttttgtacagttttactgaaacacagccgtgcccatttgtttactcattgtctatggttgctttcatgccctcacagcaaaggcgagtagttgtgatggatcaaatggcccacaaagcctgaaatatttactctttgaccctttacagaaaaaaaccttgttgacccctgctttagagaatgagaagccatgcagggatcagtgatgccagaggaagggaaggaactgcttccagctattgtgacaataataataataataatattgggtctttgactagaacgtgtaacatttccaggtgttctcacttgtgcttcccatgtttatcttacggaaggtcattccatcaagcttatggtcactgtcccttcatggcagttggtcctttcgttctccctttagctctaagagttggggagtacccacaggtgagctgtgatctcagctcagagagagagcatgaggtcttttttaactgtcaggaaacagagctgtgcccaattccactcaacttttggcacaactgttaatctgggccttcacctaccttaaactgagtttctgcaagcatagcattttagacaccctggaataaccttttgggaatgatgccacagaataaagttcactcttaacttttcaa

SEQ ID NO: 39 - DNA, 27 bp, Artificial sequence
  Synthetic oligonucleotide
ggagagaaccacccagcccagaagttc

SEQ ID NO: 40 - DNA, 23 bp, Artificial sequence
  Synthetic oligonucleotide
aggaatggaggcggcccttctgc

SEQ ID NO: 41 - DNA, 23 bp, Artificial sequence
  Synthetic oligonucleotide
cggaggagctcatcttgaaaaag

SEQ ID NO: 42 - DNA, 24 bp, Artificial sequence
  Synthetic oligonucleotide
gatcaggaacttggttgaagtaac

SEQ ID NO: 43 - DNA, 25 bp, Artificial sequence
  Synthetic oligonucleotide
tgtgagcagcaagtaacccttctcc

SEQ ID NO: 44 - DNA, 793 bp, Artificial sequence
  Probe
acagagttgaatgcaagcaatccagaagaagtgttacagctggcagcgcagagaaggaaaaaaaagtttctccaagcaatggcaaaactttacttttaagcagttaaatttttttaacttttattttttaaacaatgggctaaaaataaacagtattaaaaggttaagtttatataatacatatgtacacaattagtggtgttttcttttcagacaaaatactgaaacaaatattagtttaaaaacaaactatacagaagacttcataccgtaacaataaatgtatagtttcttcaaagggagaagagattcacatatctgataacaaaataaactagcaatctagttttctaatctactttatgaggctggattttttttttagaaaagctaatttaaaatatttagaaatagctagcctatgtacagcaagttttcatgtctttttttaataaatagatttctaggagtcagtatatatttaatactcttcttccttaagaaaatagaagtttaggtcaagtgttaagctttatcactttgacactgtccttatctcacaatggaggaatttagaaaggaccttaacagtttcacaaacataaataaagccttagtcacactaaattaaaaaaaaaaattccttagggatatcttagagtagtaaagtgacttcctcatataaatagtttgaaagggtacttaagtttttcacccaaattgtgatatacaaaaaggttattaccaagcaacctacatgtcaagaaagccccagttaggaaggagccacagc

(19) United States
(12) Patent Application Publication
(10) Pub. No.: US 2005/0009771 A1
(43) Pub. Date: Jan. 13, 2005

(54) Methods and systems for identifying naturally occurring antisense transcripts and methods, kits and arrays utilizing same

(76) Inventors: Erez Levanon, Petach Tikva (IL); Jeanne Bernstein, Kfar Yona (IL); Sarah Pollock, Tel Aviv (IL); Alex Diber, Herzlia (IL); Zurit Levine, Herzlia (IL); Sergey Nemzer, Ramat Gan (IL); Vladimir Grebinsky, Highland Park, NJ (US); Hanqing Xie, Lambertville, NJ (US); Brian Meloon, Plainsboro, NJ (US); Andrew Olson, Westfield, NJ (US); Dvir Dahary, Tel Aviv (IL); Yossi Cohen, Woking (GB); Avi Shoshan, Kiryat Gat (IL); Shira Walach, Hod Hasharon (IL); Alon Wasserman, New York, NY (US); Rami Khosravi, Herzlia (IL); Galit Rotman, Herzlia (IL)

Correspondence Address: G.E. EHRLICH (1995) LTD.;c/o ANTHONY CASTORINA, 2001 JEFFERSON DAVIS HIGHWAY, SUITE 207, ARLINGTON, VA, 22202, US

(21) Appl. No.: 10/764,503

(22) Filed: Jan. 27, 2004

Related U.S. Application Data
(63) Continuation-in-part of application No. 10/441,281, filed on May 20, 2003.

(51) Int. Cl. A61K 48/00; C12Q 1/68; G06F 19/00; G01N 33/48; G01N 33/50

(52) U.S. Cl. 514/44; 435/6; 702/20

(57) ABSTRACT

A method of identifying putative naturally occurring antisense transcripts is provided. The method is effected by (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying putative naturally occurring antisense transcripts. Also provided are polynucleotides and polypeptide sequences identified by the above-described methodology.

This is a continuation-in-part of U.S. patent application Ser. No. 10/441,281, filed 20 May, 2003, which claims priority from PCT Patent Application No. IL02/00904, filed Nov. 11, 2002, which claims priority from U.S. patent application Ser. No. 10/201,605, filed Jul. 24, 2002, which is a continuation-in-part of U.S. patent application Ser. No. 09/993,398, filed Nov. 26, 2001, which is a continuation-in-part of U.S. patent application Ser. No. 09/907,923, filed Jul. 18, 2001, which is a continuation-in-part of U.S. patent application Ser. No. 09/785,439, filed Feb. 20, 2001, which is a continuation-in-part of U.S. patent application Ser. No. 09/732,938, filed Dec. 11, 2000. This Application also claims the benefit of priority from U.S. patent application Ser. No. 09/718,407, filed Nov. 24, 2000.

### BACKGROUND AND FIELD OF THE INVENTION

The present invention relates to the field of naturally occurring, antisense transcripts. More particularly, the present invention relates to methods of identifying naturally occurring antisense transcripts, databases storing polynucleotide sequences encoding identified naturally occurring antisense transcripts, oligonucleotides derived therefrom and methods and kits utilizing same.

Naturally occurring antisense RNA transcripts are endogenous transcripts, which exhibit complementarity to sense transcripts of which are typically of a known function. It has been established that these endogenous antisense transcripts play an important role in regulating prokaryotic gene expression and are increasingly implicated as involved in eukaryotic gene regulation.

Cis-encoded antisense transcripts are encoded by the same locus as the sense transcripts and are transcribed from strand of DNA opposite to that encoding the sense transcript; as such, cis encoded antisense transcripts are typically completely complementary with a portion of the sense transcript. Trans-encoded antisense transcripts are by contrast, transcripts, which are encoded on a different locus and as such, may display only partial complementarity with a sense transcript.

Natural antisense RNAs were first described in prokaryote studies, which suggested that such transcripts play a role in gene expression regulation. Prokaryotic antisense transcripts are widely distributed and are involved in the control of numerous biological functions including transposition, plasmid replication, incompatibility and conjugation. In prokaryotes, antisense transcripts are typically involved in down-regulation of sense transcript expression, although involvement in positive regulation was also suggested [reviewed in Wagner E G. and Simons R W. (1994) Annu. Rev. Microbiol. 48:713-742].

The first example of transcription from both strands of eukaryotic DNA was illustrated in human and mouse mitochondrial genes [Anderson S. et al. (1981) Nature 290:457-465 and Bibb M J. et al. (1981) Cell 26:167-180]. Since then, examples of antisense transcripts have been documented in a variety of organisms including viruses, slime molds, insects, amphibians and birds as well as mammals. It is thought that these antisense RNAs are involved in extremely diverse biological functions, such as, hormonal response, control of proliferation, development, structure, viral replication and others. Some antisense RNAs are conserved between species suggesting that these antisense RNAs are not fortuitous but rather play an important role in gene expression regulation [Kidny M S. et al. (1987) Mol. Cell Biol. 7:2857-2862, Nepveu A. and Marcu K B. (1986) EMBO J. 5:2859-2865 and Bentley D L. et al. (1986) Nature 321:702-706].

Antisense transcripts can also encode proteins. Examples for protein encoding antisense transcripts include rev-ErbAx [Lazar M A. (1989) Mol. Cell. Biol. 9:1128-1136], gfg [Kimelman D. et al. (1989) Cell 59:687-696] and n-cym [Armstrong B C. et al. (1992) Cell Growth Differ. 3:385-390]. Such antisense transcripts typically include a distinct open reading frame (ORF) and polyadenylation signal for cytoplasm transportation.

However, it is believed that most antisense transcripts play a role in gene expression regulation. This assumption is mostly based on spatial and/or temporal distributions of sense and antisense transcripts. Indeed, tissue distribution studies suggest that high levels of sense and antisense transcripts rarely occur together, as was exemplified for the dopa decarboxylase transcripts in Drosophila [Spencer C A. et al. (1986) Nature 322:279-281]. Additional studies demonstrated that changes in sense gene expression correlate with presence of antisense RNA. Furthermore, an inverse relationship between levels of accumulation of sense and antisense transcripts such as has been reported for α1 (I) collagen transcripts in chondrocytes under chemotherapy has also been reported [Farrell C M. And Lukens L N. (1995) J. Biol. Chem. 270:3400-3408]. However, it will be appreciated that mutual expression of sense and their corresponding antisense transcripts is also reported and may involve a different mechanism of regulation.

Evidence for involvement of antisense-mediated gene regulation in the development of pathologies has also been presented. For example, endogenous antisense transcripts may be involved in regulation of the expression levels of the tumor suppressor gene WT1 observed in Wilm's tumors [Eccles M R. et al. (1994) Oncogene 9:2059-2063].

Natural antisense regulation of gene expression can be effected via one of several mechanisms.

Nuclear Regulation

Nuclear regulation can be effected via several gene-processing pathways [reviewed in Vanhee-Brosollet C. and Vaquero C. (1998) Gene 211:1-9]

dsRNA-mediated DNA methylation—complementation between endogenous sense transcripts and antisense transcripts of sequences as short as 30 bp may initiate DNA-methylation, a well-established phenomenon in a number of organisms [Sharp A. (2001) Genes Dev. 15:485-490]. Methylation can be directed to different portions of an encoding region of the gene or to the promoter region. DNA methylation results in complete suppression of transcription probably by recruitment of histone deacetylases.

Transcriptional regulation—in which case antisense transcription hampers sense transcription. Such interference may involve the collision of two transcription complexes. Alternatively, interference may result from competition on an essential rate limiting transcription factor resulting in premature termination or in reduced elongation of transcription, the transcripts with the highest rate of transcription being predominant.

Post-transcriptional nuclear regulation—involves antisense intervention of either maturation and/or transport of the sense transcript to the cytoplasm. Alternatively, antisense transcripts displaying similar structural features to sense transcripts can bind proteins expected to interact with their sense counterparts, thereby depriving sense messengers from proteins necessary for their function.

Cytoplasmic Regulation

Messenger stability—double stranded RNA may affect messenger stability via “RNA interference”, which involves short segments of double stranded RNA (dsRNA) homologous in sequence to the silenced gene. These undersized segments, which are generated by a ribonuclease III cleavage of longer dsRNAs, can guide a single stranded target mRNA, via base pairing, to a multisubunit complex which participates in the degradation of the target mRNA. Alternatively, messenger stability may be affected by RNA degradation, which is mediated by double stranded RNA-directed Rnases.

Translation—masking the 3′ untranslated region (UTR) and the polyA tail of the sense transcript is believed to modulate translation efficiency probably via direct or indirect interaction between 3′-proximal elements and upstream sequences or structures [reviewed in Jackson R J. And Standart N. (1990) Cell 62:15-24].

Realizing the fundamental role antisense transcripts play in regulating sense transcription, stability and function, resulted in a number of attempts to systematically identify natural antisense transcripts. Accordingly, differential approaches were taken for exploring non-coding antisense RNA transcripts and antisense transcripts including an ORF. Although the latter carries ORF consensus parameters, uncovering antisense data from general sequence databases has proven to be a complicated task, as many of these sequences include an evolutionary conserved secondary structure rather than a conserved primary sequence, therefore primary sequence alignment methods are often not very effective. Indeed, only a few attempts have been tried to date with only limited success.

Maziel's group [Chen J H. et al. (1990) Comput. Applic. Biosci. 6:7-18 and Le S Y. et al (1990) Human Genome Initiative and DNA Recombination Vol. 1:127-136] has experimented with methods that look for regions of a genome with predicted RNA structures that are significantly more stable thermodynamically than random sequence of the same base composition. Although this approach detected a few highly structured non-coding RNAs, as well as few cis-regulatory structures, it appears that it is of limited use for large-scale applications.

Another approach examined coding dense genomes, having suspicious-looking large regions with little or no coding potential termed “gray holes” [Olivas W M. et al. (1997) Nucleic acids Res. 25:4619-4625]. Fifty nine gray holes were tested in the yeast genome. Northern analysis detected distinct transcripts from 15 of the gray holes. Only one transcript appeared to be a non-coding antisense transcript illustrating the low efficiency of this method.

There is thus a widely recognized need for, and it would be highly advantageous to have, methods of systematically identifying novel naturally occurring antisense molecules and methods of artificially generating and using same for detecting, quantifying and/or regulating sense transcripts, such as for example, mRNA transcripts associated with a pathological state.

### SUMMARY OF THE INVENTION

According to one aspect of the present invention there is provided a method of identifying putative naturally occurring antisense transcripts, the method comprising: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying putative naturally occurring antisense transcripts.

According to another aspect of the present invention there is provided a kit for quantifying at least one mRNA transcript of interest, the kit comprising at least one oligonucleotide being designed and configured so as to be complementary to a sequence region of the mRNA transcript of interest, the sequence region not being complementary with a naturally occurring antisense transcript.

According to yet another aspect of the present invention there is provided a kit for quantifying at least one mRNA transcript of interest, the kit comprising at least one pair of oligonucleotides including a first oligonucleotide capable of binding the at least one mRNA transcript of interest and a second oligonucleotide being capable of binding a naturally occurring antisense transcript complementary to the mRNA of interest.

According to still another aspect of the present invention there is provided a method of designing artificial antisense transcripts, the method comprising: (a) providing a database of naturally occurring antisense transcripts; (b) extracting from the database criteria governing structure and/or function of the naturally occurring antisense transcripts; and (c) designing the artificial antisense transcripts according to the criteria.

According to further features in preferred embodiments of the invention described below the criteria governing structure and/or function of the naturally occurring antisense transcripts are selected from the group consisting of antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

According to an additional aspect of the present invention there is provided a computer readable storage medium comprising a database including a plurality of sequences, wherein each sequence is of a naturally occurring antisense transcript.

According to still further features in the described preferred embodiments the database further includes information pertaining to each sequence of the naturally occurring antisense transcripts, the information is selected from the group consisting of related sense gene, antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

According to still further features in the described preferred embodiments the database further includes information pertaining to generation of the database and potential uses of the database.

According to yet an additional aspect of the present invention there is provided a method of generating a database of naturally occurring antisense transcripts, the method comprising: (a) computationally aligning a first database including sense-oriented polynucleotide sequences with a second database including expressed polynucleotide sequences; (b) identifying expressed polynucleotide sequences from the second database being capable of forming a duplex with at least one sense-oriented polynucleotide sequence of the first database so as to identify putative naturally occurring antisense transcripts; and (c) storing sequence information of the identified naturally occurring antisense transcripts, thereby generating the database of the naturally occurring antisense transcripts.

According to still an additional aspect of the present invention there is provided a system for generating a database of a plurality of putative naturally occurring antisense transcripts, the system comprising a processing unit, the processing unit executing a software application configured for: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database.

According to a further aspect of the present invention there is provided a method of identifying putative naturally occurring antisense transcripts, the method comprising screening a database of expressed polynucleotides sequences according to at least one sequence criterion, the at least one sequence criterion being selected to identify putative naturally occurring antisense transcripts.

According to yet a further aspect of the present invention there is provided A method of quantifying at least one mRNA of interest in a biological sample, the method comprising: (a) contacting the biological sample with at least one oligonucleotide capable of binding with the at least one mRNA of interest, wherein the at least one oligonucleotide is designed and configured so as to be complementary to a sequence region of the mRNA transcript of interest, the sequence region not being complementary with a naturally occurring antisense transcript; and (b) detecting a level of binding between the at least one mRNA of interest and the at least one oligonucleotide to thereby quantify the at least one mRNA of interest in the biological sample.

According to still a further aspect of the present invention there is provided a method of quantifying the expression potential of at least one mRNA of interest in a biological sample, the method comprising: (a) contacting the biological sample with at least one pair of oligonucleotides including a first oligonucleotide capable of binding the at least one mRNA of interest and a second oligonucleotide being capable of binding a naturally occurring antisense transcript complementary to the mRNA of interest; and (b) detecting a level of binding between the at least one mRNA of interest and the first oligonucleotide and a level of binding between the naturally occurring antisense transcript complementary to the mRNA of interest and the second oligonucleotide to thereby quantify the expression potential of the at least one mRNA of interest in the biological sample.

According to other aspect of the present invention there is provided a method of quantifying at least one naturally occurring antisense transcript of interest in a biological sample, the method comprising: (a) contacting the biological sample with at least one oligonucleotide capable of binding with the at least one naturally occurring antisense transcript of interest, wherein the at least one oligonucleotide is designed and configured so as to be complementary to a sequence region of the naturally occurring antisense transcript of interest, the sequence region not being complementary with a naturally occurring mRNA transcript; and (b) detecting a level of binding between the at least one naturally occurring antisense transcript of interest and the at least one oligonucleotide to thereby quantify the at least one naturally occurring antisense transcript of interest in the biological sample.

According to still further features in the described preferred embodiments the first database includes sequences of a type selected from the group consisting of genomic sequences, expressed sequence tags, contigs, intron sequences, complementary DNA (cDNA) sequences, pre-messenger RNA (mRNA) sequences and mRNA sequences.

According to still further features in the described preferred embodiments the second database includes sequences of a type selected from the group consisting of expressed sequence tags, contigs, complementary DNA (cDNA) sequences, pre-messenger RNA (mRNA) sequences and mRNA sequences.

According to still further features in the described preferred embodiments an average sequence length of the expressed polynucleotide sequences of the second database is selected from a range of 0.02 to 0.8 Kb.

According to still further features in the described preferred embodiments the second database is generated by: (i) providing a library of expressed polynucleotides; (ii) obtaining sequence information of the expressed polynucleotides; (iii) computationally selecting at least a portion of the expressed polynucleotides according to at least one sequence criterion; and (iv) storing the sequence information of the at least a portion of the expressed polynucleotides thereby generating the second database.

According to still further features in the described preferred embodiments the at least one sequence criterion for computationally selecting the at least a portion of the expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

According to still further features in the described preferred embodiments the step of testing the putative naturally occurring antisense transcripts for an ability to form the duplex with the at least one sense oriented polynucleotide sequence under physiological conditions.

According to still further features in the described preferred embodiments the method further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

According to still further features in the described preferred embodiments a length of the at least one oligonucleotide is selected from a range of 15-200 nucleotides.

According to still further features in the described preferred embodiments the at least one oligonucleotide is a single stranded oligonucleotide.

According to still further features in the described preferred embodiments the at least one oligonucleotide is a double stranded oligonucleotide.

According to still further features in the described preferred embodiments a guanidine and cytosine content of the at least one oligonucleotide is at least 25%.

According to still further features in the described preferred embodiments the at least one oligonucleotide is labeled.

According to still further features in the described preferred embodiments the at least one oligonucleotide is attached to a solid substrate.

According to still further features in the described preferred embodiments the solid substrate is configured as a microarray and whereas the at least one oligonucleotide includes a plurality of oligonucleotides each attached to the microarray in a regio-specific manner.

According to still further features in the described preferred embodiments a length of each of the first and second oligonucleotides is selected from a range of 15-200 nucleotides.

According to still further features in the described preferred embodiments the first and second oligonucleotides are single stranded oligonucleotides.

According to still further features in the described preferred embodiments the first and second oligonucleotides are double stranded oligonucleotide.

According to still further features in the described preferred embodiments a guanidine and cytosine content of each of the first and second oligonucleotides is at least 25%.

According to still further features in the described preferred embodiments the first and second oligonucleotides are labeled.

According to still further features in the described preferred embodiments the first and second oligonucleotides are attached to a solid substrate.

According to still further features in the described preferred embodiments the solid substrate is configured as a microarray and whereas each of the first and second oligonucleotides includes a plurality of oligonucleotides each attached to the microarray in a regio-specific manner.

According to yet other aspect of the present invention there is provided a method of identifying a novel drug target, the method comprising: (a) determining expression level of at least one naturally occurring antisense transcript of interest in cells characterized by an abnormal phenotype; and (b) comparing the expression level of the at least one naturally occurring antisense transcript of interest in the cells characterized by an abnormal phenotype to an expression level of the at least one naturally occurring antisense transcript of interest in cells characterized by a normal phenotype, to thereby identify the novel drug target.

According to still further features in the described preferred embodiments the abnormal phenotype of the cells is selected from the group consisting of biochemical phenotype, morphological phenotype and nutritional phenotype.

According to still further features in the described preferred embodiments determining expression level of at least one naturally occurring antisense transcript of interest is effected by at least one oligonucleotide designed and configured so as to be complementary to a sequence region of the at least one naturally occurring antisense transcript of interest, the sequence region not being complementary with a naturally occurring mRNA transcript.

According to still other aspect of the present invention there is provided a method of treating or preventing a disease, condition or syndrome associated with an upregulation of a naturally occurring antisense transcript complementary to a naturally occurring mRNA transcript, the method comprising administering a therapeutically effective amount of an agent for regulating expression of the naturally occurring antisense transcript.

According to still further features in the described preferred embodiments the agent for regulating expression of the naturally occurring antisense transcript is at least one oligonucleotide designed and configured so as to hybridize to a sequence region of the at least one naturally occurring antisense transcript.

According to still further features in the described preferred embodiments the at least one oligonucleotide is a ribozyme.

According to still further features in the described preferred embodiments the at least one oligonucleotide is a sense transcript.

According to a supplementary aspect of the present invention there is provided a method of diagnosing a disease, condition or syndrome associated with a substandard expression ratio of an mRNA of interest over a naturally occurring antisense transcript complementary to the mRNA of interest, the method comprising: (a) quantifying expression level of the mRNA of interest and the naturally occurring antisense transcript complementary to the mRNA of interest; (b) calculating the expression ratio of the mRNA of interest over the naturally occurring antisense transcript complementary to the mRNA of interest, thereby diagnosing the disease, condition or syndrome. According to yet a supplementary aspect of the present invention there is provided a method of identifying co-regulated human polynucleotide sequences, the method comprising: (a) computationally identifying non-human polynucleotide sequence pairs, each corresponding to an mRNA sequence and its naturally occurring antisense transcript; (b) computationally identifying for each polynucleotide sequence of the polynucleotide sequence pairs a human orthologue polynucleotide sequence, thereby identifying human polynucleotide sequence pairs; and (c) selecting from the human polynucleotide sequence pairs, specific polynucleotide sequence pairs having oppositely oriented polynucleotide sequences which are localized to a chromosome region, the specific polynucleotide sequence pairs being co-regulated human polynucleotide sequences.

According to still further features in the described preferred embodiments the specific polynucleotide sequence pairs are gapped by a distance not exceeding a predetermined value.

According to still further features in the described preferred embodiments the predetermined value does not exceed 10 Kb.

According to still further features in the described preferred embodiments step (a) is effected by: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying the polynucleotide sequence pairs of mRNA sequences and naturally occurring antisense transcripts complementary to the mRNA sequences.

According to still further features in the described preferred embodiments step (b) is effected by a homology screening software application.

According to still further features in the described preferred embodiments the method further comprising identifying oppositely oriented expressed sequences corresponding to the human co-regulated polynucleotide sequences.

According to still a supplementary aspect of the present invention there is provided A system for generating a database of co-regulated human polynucleotide sequences, the system comprising a processing unit, the processing unit executing a software application configured for: (a) computationally identifying non-human polynucleotide sequence pairs, each corresponding to an mRNA sequence and its naturally occurring antisense transcript; (b) computationally identifying for each polynucleotide sequence of the polynucleotide sequence pairs a human orthologue polynucleotide sequence, thereby identifying human polynucleotide sequence pairs; (c) selecting from the human polynucleotide sequence pairs, specific polynucleotide sequence pairs having oppositely oriented polynucleotide sequences which are localized to a chromosome region, the specific polynucleotide sequence pairs being co-regulated human polynucleotide sequences; and (d) storing the co-regulated human polynucleotide sequences to therevy generate the database of co-regulated human polynucleotide sequences.

According to still further features in the described preferred embodiments the specific polynucleotide sequence pairs are gapped by a distance not exceeding a predetermined value.

According to still further features in the described preferred embodiments the predetermined value does not exceed 10 Kb.

According to still further features in the described preferred embodiments step (a) is effected by: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying the polynucleotide sequence pairs of mRNA sequences and naturally occurring antisense transcripts complementary to the mRNA sequences.

According to still further features in the described preferred embodiments step (b) is effected by a homology screening software application.

According to still further features in the described preferred embodiments the method further comprising identifying oppositely oriented expressed sequences corresponding to the human co-regulated polynucleotide sequences.

According to still a supplementary aspect of the present invention there is provided a computer readable storage medium comprising data stored in a retrievable manner, the data including sequence information of co-regulated human polynucleotide sequences as set forth in files seqs_{—}125 and/or seqs_{—}133 of enclosed CD-1, mouse_seqs, nuc_seqs_{—}136 and/or pep_seqs_{—}136 of enclosed CD-ROM4 and sequence annotations as set forth in the file annotations_{—}136 of enclosed CD-ROM4.

According to still a supplementary aspect of the present invention there is provided a method of modulating an activity or expression of a gene product, the method comprising upregulating or down regulating expression or activity of a naturally occurring antisense transcript of the gene product, thereby modulating the activity or expression of the gene product.

According to still further features in the described preferred embodiments the method further comprising upregulating or down regulating expression or activity of the gene product.

According to still a supplementary aspect of the present invention there is provided an isolated polynucleotide comprising any of the nucleic acid sequences set forth in the file seqs_{—}125 or seqs_{—}133 of the enclosed CD-ROM1; or in the file nuc_seqs_{—}136 of the enclosed CD-ROMs 1-4.

According to a supplementary aspect of the present invention there is provided an isolated polypeptide comprising any of the amino acid sequences set forth in the file pep_seqs_{—}136 of enclosed CD-ROM4.

The present invention successfully addresses the shortcomings of the presently known configurations by providing a novel approach for identifying naturally occurring antisense transcripts, methods of designing artificial antisense transcripts according to information derived therefrom and methods and kits using naturally occurring and synthetic antisense transcripts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates EST alignment along genomic DNA, generated according to the teachings of the present invention. Alignment results identify two strand groups of transcripts i.e., sense transcripts and antisense transcripts with an indicated sequence overlap.

FIG. 2 illustrates a system designed and configured for generating a database of naturally occurring antisense sequences generated according to the teachings of the present invention.

FIG. 3 illustrates a remote configuration of the system described in FIG. 2.

FIGS. 4a-k are sequence alignments of overlapping regions of selected naturally occurring antisense and sense sequence pairs identified according to the teachings of the present invention.

FIGS. 5a-g are sequence alignments of overlapping regions of selected naturally occurring antisense and sense sequence pairs identified according to the teachings of the present invention.

FIG. 6 schematically illustrates two transcription products of 53BP1 gene (red and green) and their corresponding partial complementary antisense transcripts of the 76p gene (blue). Numbers in parenthesis indicate length of sequence complementation. Schematic location of strand-specific RNA probes used for northern blotting of sense (53BP1, Riboprobe#1) and antisense (76p, Riboprobe#2) transcripts is shown.

FIG. 7 is an autoradiogram of a northern blot analysis depicting cellular distribution and expression levels of 53BP1 transcripts. Arrows on the right indicate the molecular weight of the identified 53BP1 transcripts relative to the migration of 28S and 18S ribosomal RNA subunits. |Numbers on the left denote the size of molecular weight markers in Kb.

FIG. 8 is an autoradiogram of a northern blot analysis depicting cellular distribution and expression levels of 76p transcripts. Arrows on the right indicate the molecular weight of the identified 76p transcripts relative to the migration of 28S and 18S ribosomal RNA subunits. |Numbers on the left denote the size of molecular weight markers in Kb.

FIG. 9 is an autoradiogram of a northern blot analysis depicting tissue distribution and expression levels of 76p transcripts. Arrows on the right indicate the molecular weight of the identified 76p transcripts. Numbers on the left denote the migration of molecular weight marker in Kb.

FIG. 10 illustrates the genomic organization of the 53BP1 gene and 76p gene, as elucidated from the RT-PCR analysis presented in the Examples section hereinbelow. Black arrows indicate the location of the primers used for RT-PCR analysis. Asterisks denote stop codons.

FIG. 11 schematically illustrates two transcription products of CIDE-B gene and their corresponding partial complementary antisense transcript of the BLTR2 gene. Schematic location of the strand-specific 430 nucleotide RNA probe used for northern analysis of sense (CIDE-B) and antisense (BLTR2) transcripts is shown. Dashed rectangles indicate the predicted coding sequence of the transcripts.

FIG. 12 is an autoradiogram of a northern blot analysis depicting cellular distribution and expression levels of BLTR2 transcripts. Arrows on the right indicate the molecular weight of the identified BLTR2 transcripts relative to the migration of 28S and 18S ribosomal RNA subunits. Numbers on the left denote the size of molecular weight markers in Kb.

FIG. 13 shows autoradiogram of a northern blot analysis depicting cellular distribution and expression levels of CIDE-B transcripts. Arrows on the right indicate the molecular weight of the identified CIDE-B transcripts relatively to the migration of 28S and 18S ribosomal RNA subunits. Numbers on the left denote the migration size of molecular weight markers in Kb.

FIG. 14 schematically illustrates a transcription product of APAF-1 gene and its corresponding partial complementary antisense transcripts of the EB-1 gene. Schematic location of the strand-specific 366 nucleotide RNA probe used for northern analysis of sense (APAF-1) and antisense (EB-1) transcripts is shown. Asterisks indicate the predicted coding sequence borders of the transcripts.

FIGS. 15a-b are autoradiograms of northern blot analyses depicting cellular distribution and expression levels of EB-1 (FIG. 15a) and APAF-1 transcripts (FIG. 15b). Numbers on the left denote the size of molecular weight marker in Kb.

FIG. 16 schematically illustrates a transcription product of the MINK-2 gene and its corresponding partial complementary antisense transcript of the AchR-ε gene. Schematic location of the strand-specific 280 nucleotide RNA probe used for northern analysis of sense (Mink-2) and antisense (AchR-ε) transcripts is shown.

FIGS. 17a-b are autoradiograms of northern blot analyses depicting cellular distribution and expression levels of AchR-ε antisense transcripts (FIG. 17a) and the sense complementary transcript of Mink-2 (FIG. 17b). Arrows on the right denote the migration of molecular weight markers in Kb.

FIG. 18 schematically illustrates a transcription product of Cyclin-E2 gene and its corresponding partial complementary antisense transcript. Schematic location of strand-specific RNA probes used for northern blotting of sense (Riboprobe#1) and antisense (Riboprobe#2) transcripts is shown.

FIGS. 19a-b are autoradiograms of northern blot analyses depicting cellular distribution and expression levels of Cyclin E2 antisense transcript (FIG. 19a) and the sense complementary transcript (FIG. 19b). Arrows on the left denote the migration of molecular weight markets in Kb.

FIG. 20 illustrates results from RT-PCR analysis of the expression patterns of CIDE-B transcript and its complementary naturally occurring antisense transcript following concentration dependent induction of apoptosis. Lanes: (1) 50 μM etoposide; (2) 100 μM etoposide; (3) 250 μM etoposide; (4) 500 μM etoposide; (5) 10 nM staurosporine; (6) 100 nM staurosporine; (7) 250 nM staurosporine; (8) 1000 nM staurosporine; (9) untreated cells (UT). FIGS. 21a-c are results of RT-PCR analyses depicting expression patterns of AchRε and its naturally occurring antisense transcript following time-dependent induction of differentiation. FIG. 21a illustrates the position of riboprobes used for reverse transcription reaction. FIG. 21b shows the reciprocal expression pattern of sense and antisense transcripts (indicated by arrows). FIG. 21c shows the expression pattern of the antisense transcript alone.

FIGS. 22a-j illustrate results of northern blot analysis of sense/antisense clusters revealing positive signals for sense/antisense genes in the microarray analysis. Diagrams describing genomic organization of the relevant region for each of the sense/antisense clusters are included above the autoradiograms, and regions of overlap (including GenBank accession number) from which the strand-specific riboprobes were derived are included. Sense-antisense pair numbers are as they appear in the microarray (as depicted in Table_S2 on the attached CD-ROM2 and in conversion Table 6). FIG. 22a reveals expression patterns of randomly selected sequence pair number 235, denoted as Rand_{—}235 in Table 6. Similarly, FIG. 22b corresponds to pair number 173, FIG. 22c to pair number 248, FIG. 22d to pair number 6, FIG. 22e to pair number 216, FIG. 22f to pair number 239, FIG. 22g to pair number 202, FIG. 22h to pair number 114, FIG. 22i to pair number 188, and FIG. 22j to pair number 223. Eight pairs (FIGS. 22a-h) evaluated revealed positive signals for both sense and antisense expression, while two (FIGS. 22i-j) revealed a positive signal for only one of the genes, with the counterpart being a known RefSeq mRNA.

FIG. 23 is a Table depicting expression patterns in various cell lines and tissues as probed with a subset of 264 pairs from the putative sense/antisense dataset of the present invention. The pairs are denoted by the pair number and described in Table_S1 of CD-ROM2. “C” and “AC” denote the two counterpart probes. Expression was also verified for positive controls, including the ubiquitously expressed genes gapdh, actin, hsp70 and gnb211 in various concentrations, and 11 previously documented sense/antisense pairs. Expression thresholds were verified and indicated as “+”, if the probe passed the threshold in at least one cell line or tissue or “−”, if the probe did not pass the threshold in all experiments. In cases where both the sense and the antisense oligo passed the expression threshold, the antisense was declared “verified”. In cases where only one of the probes passed the expression threshold, but the other probe was fully contained within a known mRNA deposited in GenBank, the antisense was declared “indirectly verified”. Normalization for microarray signals was conducted as described in the methods section. Rji ratios were obtained for each cell line/tissue assessed. Cases of flagged-out spots for which there was no information were marked “−1.00”. Data represent values of the two reciprocal experiments.

### DESCRIPTION OF THE PREFERRED EMBODIMENTS

The present invention is of methods of identifying naturally occurring antisense transcripts, which can be used in kits and methods for quantifying gene expression levels. Specifically, the antisense molecules and related oligonucleotides generated according to information derived therefrom of the present invention can be used to detect, quantify, or specifically regulate antisense and respective sense transcripts thereby enabling detection and treatment of a wide range of disorders.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

Terminology

As used herein, the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

The term “antisense” refers to a complementary strand of an mRNA transcript e.g., antisense RNA.

The phrase “naturally occurring antisense transcripts” refers to RNA transcripts encoded from an antisense strand of the DNA. These endogenous transcript exhibit at least partial complementarity to mRNA transcripts transcribed from the sense strand of a DNA, also termed sense transcripts. cis-encoded naturally occurring antisense transcripts are transcribed from the same locus as the sense transcripts. trans-encoded antisense transcripts are transcribed from a different locus than the respective sense transcripts.

The phrase “antisense strand” or “anticoding strand” refers to a strand of DNA, which serves as a template for mRNA transcription and as such is complementary to the mRNA transcript formed.

The phrase “sense strand” or “coding strand” refers to the strand of DNA, which is identical to the mRNA transcript formed.

The phrase “complementary DNA” (cDNA) refers to the double stranded or single stranded DNA molecule, which is synthesized from a messenger RNA template.

The phrase “sense oriented polynucleotides” refers to polynucleotide sequences of a complementary or genomic DNA. Such polynucleotide sequences can be from exon regions, in which case they can encode mRNAs or portions thereof, or from intron regions, in which case they typically do not encode mRNA or portions thereof.

The term “contig” refers to a series of overlapping sequences with sufficient identity to create a longer contiguous sequence.

The term “cluster” refers to a plurality of contigs all derived, with a high degree of probability, from a single gene. Clusters are generally formed based upon a specified degree of homology and overlap (e.g., a stringency). The different contigs in a cluster do not typically represent the entire sequence of the gene, rather the gene may comprise one or more unknown intervening sequences between the defined contigs.

The phrase “open reading frame” (ORF) refers to a nucleotide sequence, which could potentially be translated into a polypeptide. Such a stretch of sequence is uninterrupted by a stop codon. An ORF that represents the coding sequence for a full protein begins with an ATG “start” codon and terminates with one of the three “stop” codons. For the purposes of this application, an ORF may be any part of a coding sequence, with or without start and/or stop codons. For an ORF to be considered as a good candidate for coding for a bona fide cellular protein, a minimum size requirement is often set, for example, a stretch of DNA that would code for a protein of 50 amino acids or more. An ORF is not usually considered an equivalent to a gene or locus until a phenotype is associated with a mutation in the ORF, an mRNA transcript for a gene product generated from the ORF's DNA has been detected, and/or the ORF's protein product has been identified.

The term “annotation” refers to a functional or structural description of a sequence, which may include identifying attributes such as locus name, poly(A)/poly(T) tail and/or signal, key words, Medline references and orientation cloning data.

Naturally occurring antisense molecules can play a role in sense transcription stability and function (e.g. translation). To date, most, if not all of the information relating to naturally occurring antisense transcripts was obtained by either low efficiency computational approaches (described hereinabove) or by approaches utilizing RNase protection assays, northern blot analysis, strand-specific RT PCR, subtractive hybridization, differential plaque hybridization, affinity chromatography, electrospray mass spectrometry and the like. These methods, though highly reliable, are extremely laborious, time consuming and are directed at individual target transcripts. As such, current approaches for uncovering antisense transcripts can be used to detect a negligible portion of the number of naturally occurring antisense molecules thought to exist.

As described hereinunder and in the Examples section, which follows, the present invention provides a novel approach for systematically identifying naturally occurring antisense molecules.

Aside from large scale applicability, the present method can be used to identify naturally occurring antisense molecules even in cases where the antisense transcriptional unit is localized to an intron of an expressed gene or to a different locus than the complementary sense encoding gene (e.g., trans-encoded antisense), or in cases where the antisense molecule lacks an open reading frame or appreciable complementarity to known sense molecules. Antisense transcripts uncovered according to the teachings of the present invention can be used for detecting and accurately quantifying respective sense counterparts as well as for sensibly designing artificial antisense molecules suitable for down-regulation of sense counterparts.

Thus, according to one aspect of the present invention there is provided a method of identifying putative naturally occurring antisense transcripts.

The method according to this aspect of the present invention is effected by the following steps.

First, sense-oriented polynucleotide sequences of a first database are computationally aligned with expressed polynucleotide sequences of a second database.

Following computational alignment, expressed polynucleotide sequences are analyzed according to one or more criteria for their ability to hybridize or form a duplex or partial complementation with the sense-oriented polynucleotide sequences (further detailed hereinbelow and in the Examples section which follows).

Expressed polynucleotide sequences which are capable of forming a duplex with sense oriented sequences are considered as putative naturally occurring antisense molecules and as such can be stored in a database which can be generated by a suitable computing platform.

Final confirmation of computationally obtained putative naturally occurring antisense molecules can be effected either computationally or preferably by using suitable laboratorial methodologies, based on nucleotide hybridization including RNase protection assay, subtractive hybridization, differential plaque hybridization, affinity chromatography, electrospray mass spectrometry, northern analysis, RT-PCR and the like (for further details see the Examples section).

Information derived from the sequence, sense position and other structure characteristics of the naturally occurring antisense transcripts identified according to the teachings of the present invention can be used to quantify respective sense transcripts of interest or to generate corresponding artificial antisense polynucleotides, which can be packed in diagnostic or therapeutic kits and implemented in various therapeutic and diagnostic methods.

Expressed polynucleotide sequences used as a potential source for identifying naturally occurring antisense transcripts according to this aspect of the present invention are preferably libraries of expressed messenger RNA [i.e., expressed sequence tags (EST), cDNA clones, contigs, pre-mRNA, etc.] obtained from tissue or cell-line preparations which can include genomic and/or cDNA sequence.

Expressed polynucleotide sequences, according to this aspect of the present invention can be retrieved from pre-existing publicly available databases (i.e., GenBank database maintained by the National Center for Biotechnology Information (NCBI), part of the National Library of Medicine, and the TIGR database maintained by The Institute for Genomic Research) or private databases (i.e., the LifeSeq.™ and PathoSeq.™ databases available from Incyte Pharmaceuticals, Inc. of Palo Alto, Calif.).

Alternatively, the sequence database of the expressed polynucleotide sequences utilized by the present invention can be generated from sequence libraries (e.g., cDNA libraries, EST libraries, mRNA libraries and others). cDNA libraries are suitable sources for expressed sequence information.

Generating a sequence database in such a case is typically effected by tissue or cell sample preparation, RNA isolation, cDNA library construction and sequencing.

It will be appreciated that such cDNA libraries can be constructed from RNA isolated from whole organisms, tissues, tissue sections, or cell populations. Libraries can also be constructed from tissue reflecting a particular pathological or physiological state. Of particular interest are libraries constructed from sources associated with certain disease states, including malignant, neoplastic, hyperplastic tissues and the like.

Once raw sequence data is obtained, sequences are selected and preferably annotated before stored in a database. Selection proceeds according to one or more sequence criterion, which will be further detailed hereinunder. The editing, annotation and selection process is divided into two stages of processing. One stage comprises removal of repetitive, redundant or non-informative and contaminant sequences. The second stage involves selection of suitable candidates of putative naturally occurring antisense sequences.

The following section describes the different selection criteria which can be used for sequence filtering.

Vector contamination—“chops” vector elements and linker motifs used for the process of cloning from desired expressed nucleotide sequences. This selection can be effected by screening manually updated databases of sequences included in commonly used expression or cloning vectors.

Contaminating sequences—includes sequences which are derived from an undesired source. Such sequences can be recognized by their nucleotide distribution and/or by homology searches such as alignment searches using any sequence alignment algorithm such as BLAST (Basic Local Alignment Search Tool, available through www.ncbi.nlm.nih.gov/BLAST) or the Smith-Waterman algorithm. Other contaminating sequences may include sequences exhibiting high occurrence of dinucleotide distribution mostly related to sequencing artifacts and ribosomal RNA sequences.

Repetitive elements and low complexity sequences—eliminates or masks expressed sequences comprising known repetitive elements (ALU, L1 etc.) and low complexity sequences (i.e., a di- or tri-nucleotide repeat). Such elimination is preferably effected by comparison with database of known repetitive elements. It will be appreciated that this type of selection is mostly species specific. Masking of low complexity sequences can be effected by substituting an N (i.e., an inert character) for the actual nucleotide (i.e., G, A, T, or C). Masking of low complexity sequences facilitates further computational analysis and maintains the spacing of the molecule.

Sequence length—preferred expressed sequences are of a length between 20-2000, preferably 20-1000, more preferably 20-500, most preferably 20-300 base pairs.

Sequence annotation—expressed sequences retrieved from external databases, i.e., GenBank, oftentimes include an annotation which indicates direction of the sequencing of the insert clone (i.e., 5′ or 3′ direction). Sequence annotation, though “noisy” by nature due to multiple entries from various sources; artifacts taking place during directional cloning and incidence of palindromic eight-cutter restriction sites located at the end of the sequence, can serve as an important tool for deducing strand identity using dedicated computer software which are further discussed hereinunder

Intron splice site consensus sequence intron splice site sharing—intron sequences nearly always begin with a di-nucleotide sequence of GT (“splice donor”) and end with an AG (“splice acceptor”) preceded by a pyrimidine-rich tract. This consensus sequence is part of the signal for splicing. Intron splice site consensus sequence on the complementary strand (e.g., antisense strand) begins with CT and ends with AC. Thus, combined with genomic data, expressed sequences having a GT. AG can be considered as sense-oriented sequences, while a CT . . . AC pattern is considered as an antisense oriented sequence. This selection criterion is very stringent since only negligible portions of introns have a CT . . . AC pattern. Sequences that share a similar splicing pattern, as deduced by alignment to genomic data, may be considered as having the same sense orientation, also termed herein as “intron sharing”. It will be appreciated by one skilled in the art that using these selection criteria requires a careful and accurate alignment of expressed sequences to genomic sequence.

Poly(A) tails and Poly(T) heads—most eukaryotic mRNA molecules contain a poly-adenylation [poly(A)] tail at their 3′ end. This poly(A) tail is not encoded by DNA. Therefore an expressed sequence which has a poly(A) tail can be considered as sense oriented. Similarly, poly(T) heads, which are not encoded from a genomic sequence indicate that a sequence is of the opposite direction, namely antisense oriented. Notably, genomically encoded Poly(A) tails and poly(T) heads provide no information as to the sequence orientation.

Poly(A) signal—some mature mRNA transcripts contain internal AAUAAA sequence. This internal sequence is part of an endonuclease cleavage signal. Following cleavage by the endonuclease, a poly(A) polymerase adds about 250 A residues to the 3′ end of the transcript. Hence, expressed sequences containing a poly(A) signal can be considered as sense oriented.

Rare restriction site used for cloning—for example, eight cutter endonucleases which cleave 8-mer palindromic sequences and are characterized by a low frequency of cutting often used in genome mapping and EST library preparations (e.g., NotI. Commercially available from Promega: www.promega.com). Therefore, when a cluster of overlapping expressed sequences is characterized by a portion of sequences starting with a digestion site and another portion ending with the same, these sequences may be considered as encoded from the same strand. However, any endonuclease capable of digesting a palindromic sequence (i.e., XhoI, SalI, PacI etc.) may also affect distorted sequence clustering, therefore strand orientation is preferably effected using other parameters as well.

Sequence overlap—sequences that completely overlap are considered to have the same strand orientation.

The above-described parameters are used individually or in combination to analyze the expressed polynucleotide sequences so as to select anti-sense oriented sequences.

Selection can be effected on the basis of a single criterion or several criteria considered individually or in combination.

In cases where several criteria are examined, a scoring system e.g., a scoring matrix, is preferably used.

Since in some cases identifying an intron splicing consensus site may be more important than both sequence annotation and NotI alignment, while in others, detection of poly(A) tails and poly(T) heads might be the most significant criterion, the use of a scoring matrix in which each criterion is weighted enables one to select qualified antisense transcripts.

Such a scoring matrix can list the various expressed polynucleotide sequences across the X-axis of the matrix while each criterion can be listed on the Y-axis of the matrix. Criteria include both a predetermined range of values from which a single value is selected from each sequence, and a weight. Each sequence is scored at each criterion according to its value and the weight of the criterion.

When using such a scoring matrix the scores of each criterion of a specific sequence are summed and the results are analyzed.

Expressed sequences which exhibit a total score greater than a particular stringency threshold are grouped as members of either a sense-oriented sequence set or antisense-oriented sequence set; the higher the score the more stringent the criteria of grouping.

It will be appreciated that the above described analysis can take place prior to computational alignment to sense oriented sequences, i.e., during the process of editing the expressed sequence database which is described hereinabove. Alternatively, selection can take place following computational alignment, thus further facilitating identification of proper duplex formation between the sense oriented polynucleotide sequences and expressed polynucleotide sequences.

Genomic DNA or a portion thereof is preferably used as sense-oriented sequence data according to this aspect of the present invention. It is conceivable that the present invention can determine sense orientation and antisense orientation of a database of expressed sequences simply by computationally aligning the sequences of the expressed database onto the genome, and finding whether two complementary expressed sequences hybridize to the genome (e.g., virtually generate a double stranded portion thereof). Such two overlapping sequences constitute sense and naturally occurring antisense transcripts.

Utilizing genomic DNA as a sense oriented template is preferred for the following reasons: (i) identifying trans-encoded antisense transcripts; (ii) analyzing intron splice consensus site and intron sharing; (iii) omitting genomically encoded poly(A) and poly(T) sequences; and (iv) analyzing sequences encompassing eight-cutter restriction sites.

Computational alignment of expressed polynucleotide sequences to the sense-oriented polynucleotide sequences (e.g., genomic sense sequences) can be effected using any commercially available alignment software, including sequence alignment tools utilizing algorithm such as BLAST (Basic Local Alignment Search Tool, available through www.ncbi.nlm.nih.gov/BLAST) or Smith-Waterman.

Assembly software is preferably used according to this aspect of the present invention. Such software is of high value when complete genomic information is unavailable or when handling large amounts of expressed sequence data. A number of commonly used computer software fragment read assemblers capable of forming clusters of expressed sequences are now available. These packages include but are not limited to, The TIGR Assembler [Sutton G. et al. (1995) Genome Science and Technology 1:9-19], GAP [Bonfield J K. et al. (1995) Nucleic Acids Res. 23:4992-4999], CAP2 [Huang X. et al. (1996) Genomics 33:21-31], The Genome Construction Manager [Laurence C B. Et al. (1994) Genomics 23:192-201], Bio Image Sequence Assembly Manager, SeqMan [Swindell S R. and Plasterer J N. (1997) Methods Mol. Biol. 70:75-89], LEADS and GenCarta (Compugen Ltd. Israel).

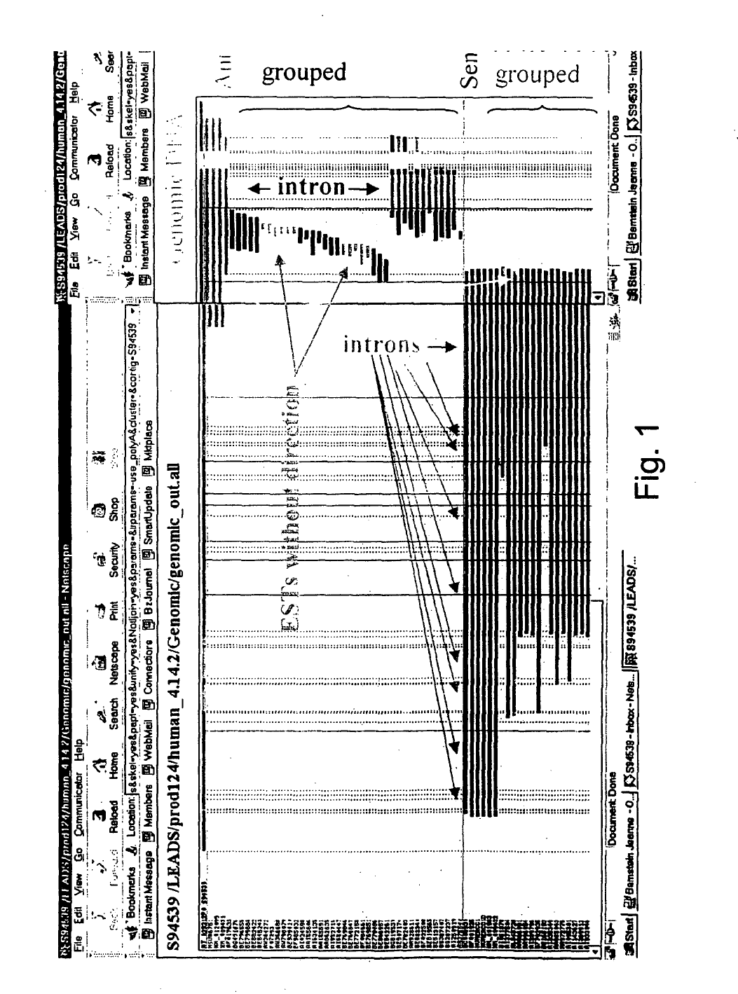

Computer assembly and alignment programs can be modified to incorporate sequence criteria for determining sense or antisense orientation of expressed nucleotide sequences, as described hereinabove. Thereby, avoiding deliberate inversion of sequences during the assembly process, while ignoring the natural orientation of the sequences (i.e., sense or antisense orientation). FIG. 1 illustrates results of expressed sequence assembly against genomic data and final distinction between sense oriented transcripts and antisense oriented transcripts of a single gene.

Following a proper alignment of expressed sequences to sense oriented polynucleotide sequences, duplexes are identified. The term “duplex” is used herein to indicate that a sequence identified according to this aspect of the present invention is complementary to a sense-oriented polynucleotide sequence. Complementation may be to a portion of the sense sequence, i.e., a region thereof, or alternatively, to two or more non-contiguous regions, which may be separated by one or more nucleotides on the sense strand.

The formation of sense-antisense duplexes does not require 100% complementation nor does it require participation of the entire sense/antisense transcript sequence. The sense or antisense transcripts can have a secondary structure (e.g., stem and loop) generated by intra-sequence hybridization which can prevent specific sequence regions in the sense or antisense transcripts from participating in duplex formation. Thus, the antisense of the sequence identified, according to this aspect of the present invention can be complementary to its sense counterparts in several regions, which are not necessarily close to each other when the sense transcript is in linear form.

Although any length of sequence overlap can generate a duplex, overlaps of at least 5, preferably 20, more preferably 30, most preferably 40 bp are considered more indicative of true sense-antisense duplex formation.

In cases where expressed sequence data is unavailable or lacking, identification of co-regulated transcripts i.e., mRNAs and their naturally accurring antisense transcripts, using the above-described methodology can be difficult or impossible.

To this end, the present inventors devised a new set of rules which can be used to identify co-regulated transcripts in cases where expressed sequence data is not available (see Example 10 of the Examples section which follows).

Thus, according to another aspect of the present invention there is provided a method of identifying co-regulated human polynucleotide sequences. The method is effected by first, computationally identifying non-human polynucleotide sequence pairs each corresponding to an mRNA sequence and its naturally occurring antisense transcript; such identification is preferably effected using the above described methodology.

As used herein the phrase “non-human polynucleotide sequences” refers to polynucleotide sequences which are evolutionary related and orthologous to respective human sequences. The non-human polynucleotide sequence pairs of this aspect of the present invention are preferably from mouse origin. Mouse sequence information can be obtained from publicly available databases such as for example the Mouse Genome Resource available at www.ncbi.nlm.nih.gov/genome/guide/mouse.

In the next step of the method, human polynucleotide sequences which are orthologous to the non-human polynucleotide sequences of the pairs are identified thereby generate human polynucleotide sequence pairs. Identification of human orthologs can be effected using specific databases such as HomoloGene which is a resource of curated and calculated orthologs represented by UniGene or by annotation of genomic sequences 9http://www.ncbi.nlm.nih.gov/HomoloGene/).

Once ortholohgous human polynucleotide sequence pairs are obtained, specific polynucleotide sequence pairs which include oppositely oriented polynucleotide sequences and which are preferably gapped by a distance not exceeding a predetermined value (e.g., less than 10 kb when mapped to a chromosomal region) are identified and selected. These specific polynucleotide sequence pairs are considered herein as co-regulated human polynucleotide sequences. Such specific polynucleotide sequence pairs are further validated as described hereinabove.

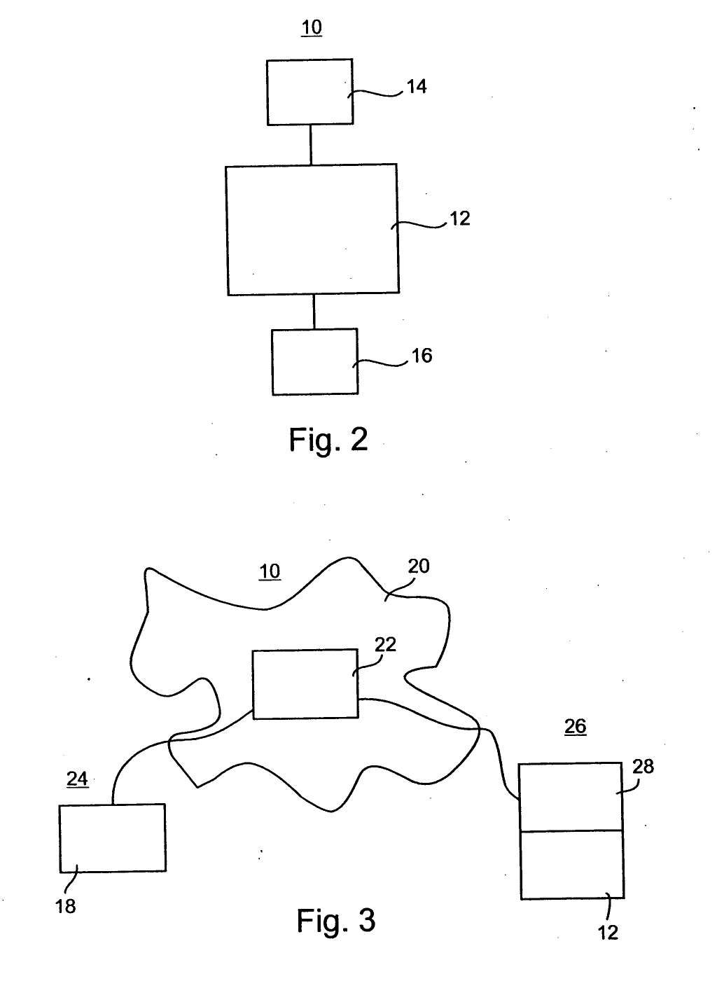

The methods of the present invention are preferably carried out using a dedicated computational system. Thus, according to another aspect of the present invention and as illustrated in FIG. 2, there is provided a system for generating a database of putative naturally occurring antisense sequences which system is referred to hereinunder as system 10.

System 10 includes a processing unit 12, which executes a software application designed and configured for aligning sense oriented polynucleotide sequences with expressed polynucleotide sequences and identifying expressed polynucleotide sequences which are capable of forming a duplex with the sense oriented polynucleotide sequences, thereby recognizing putative naturally occurring antisense transcripts. System 10 may also include a user input interface 14 (e.g., a keyboard and/or a mouse) for inputting database or database related information, and a user output interface 16 (e.g., a monitor) for providing database information to a user.

System 10 preferably stores sequence information of the putative antisense transcripts identified thereby on a computer readable media such as a magnetic, optico-magnetic or optical disk to thereby generate a database of putative antisense transcript sequences. Such a database further includes information pertaining to database generation (e.g., source library), parameters used for selecting polynucleotide sequences, putative uses of the stored sequences, and various other annotations and references which relate to the stored sequences or respective sense transcripts.

System 10 of the present invention may be used by a user to query the stored database of sequences, to retrieve nucleotide sequences stored therein or to generate polynucleotide sequences from user inputted sequences.

System 10 can be any computing platform known in the art including but not limited to, a personal computer, a work station, a mainframe and the like.

The database generated and stored by system 10 can be accessed by an on-site user of system 10, or by a remote user communicating with system 10.

As illustrated in FIG. 3, communication between a remote user 18 and processing unit 12 is preferably effected via a communication network 20. Communication network 20 can be any private or public communication network including, but not limited to, a standard or cellular telephony network, a computer network such as the Internet or intranet, a satellite network or any combination thereof.

As illustrated in FIG. 3, communication network 20 includes one or more communication servers 22 (one shown in FIG. 3) which serves for communicating data pertaining to the polypeptide of interest between remote user 18 and processing unit 12.

It will be appreciated that existing computer networks such as the Internet can provide the infrastructure and technology necessary for supporting data communication between any number of sites 24 and remote analysis sites 26.

For example, using a computer operating a Web browser application and the World Wide Web, any expressed polynucleotide sequence of interest can be “uploaded” by user 18 onto a Web site maintained by a database server 28. Following uploading, database server 28 which serves as processing unit 12 can be instructed by the user to processes the polynucleotide as is described hereinabove.

Following such processing, which can be performed in real time, nucleic acid sequence results can be displayed at the web site maintained by database server 28 and/or communicated back to site 24, via for example, e-mail communication.

Thus, using the Internet, a remote configuration of system 10 can provide polynucleotide sequence analysis services to a plurality of sites 24 (one shown in FIG. 3).

It will be appreciated that this configuration of system 10 of the present invention is especially advantageous in cases where sequence analysis can not be effected on-site. For example, laboratories, which lack the equipment necessary for executing the analysis or lack the necessary skills to operate it.

Novel polynucleotide sequences uncovered using the above-described methodology can be used in various clinical applications (e.g., therapeutic and diagnostic) as is further described hereinbelow.

A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Thus, the present invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

Thus, the present invention also encompasses polypeptides encoded by the polynucleotide sequences of the present invention. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth in the file pep_seqs_{—}136 of the enclosed CD-ROM4. Finally, the present invention also encompasses fragments of the above described polypeptides and polypeptides having mutations, such as deletions, insertions or substitutions of one or more amino acids, either naturally occurring or man induced, either randomly or in a targeted fashion.

Thus, data extracted from the above-described database is of high value for designing oligonucleotides suitable for transcript detection and quantification and for sensibly designing artificial antisense oligonucleotides for down-regulation and elimination of a transcript of interest or changing the balance between sense and complementary antisense transcripts. The possibility of up-regulating a transcript of interest using naturally occurring antisense based-oligonucleotides generated according to the teachings of the present invention is also realized. In addition, data extracted from the database of naturally occurring antisense transcripts may also be used for assessing endogenous double stranded-RNA also termed interfering RNA, which may distort gene-expression due to either RNA-degradation, DNA-methylation, polycomb mediated suppression etc. (for details see the Background section hereinabove).

Antisense technology is based upon the pairing of an artificially designed antisense oligonucleotide, with a target nucleic acid. The use of antisense technology requires a complementarity of the antisense nucleotide sequence to a target zone of an mRNA target sequence that will effect inhibition of gene expression [reviewed in Stein C A. and Cohen J S. (1988) Cancer Res. 48:2659-68]. Based on empiric experience it was shown that the success of antisense technology relies on: (i) cellular uptake; (ii) stability of artificial antisense molecules under physiological conditions (i.e., cellular pH, endonucleases etc.); (iii) complementation between the oligonucleotide and a single stranded target sequence (i.e., tertiary structure of target RNA will not form a good target); (iv) binding specificity of antisense oligonucleotide so as not to compete with other RNA binders (e.g. proteins) to thereby maintain an effective antisense concentration.

Various attempts to employ antisense technology while considering the above discussed limitations included using large amounts of oligonucleotides to overcome cellular uptake and environmental barriers and chemically modified antisense nucleotide compositions, for obtaining higher level of cellular stability. However, even in case where uptake difficulties are traversed, the step of target identification (i.e., RNA-target sequence region) continues to be the major bottleneck for successful implementation of antisense technology.

U.S. Pat. No. 6,183,966 discloses a method and an apparatus for ranking nucleic acid sequences based on stability of nucleic acid oligomer sequence binding interactions to select sequence zones for antisense targeting. This method however systematic, relies on thermodynamic analyses combined with numerous predictions which cannot be considered empirically accurate and reliable.

Thus according to another aspect of the present invention there is provided a method of designing artificial antisense transcripts.

The method according to this aspect of the present invention is effected by the following steps.

First, structural and/or functional parameters pertaining to naturally occurring antisense transcripts are extracted/deduced from a database such as the one described hereinabove. These parameters may be generally deduced from all sequences stored in the database, or extracted from specific antisense sequences or preferably groups of antisense sequences.

Second, artificial antisense molecules of interest are designed according to the extracted parameters.

Such parameters may be divided into three groups, topographical parameters, functional parameters and structural parameters.

Topographical parameters—(i) position of sequence overlap on the sense transcript (i.e., coding region, 5′UTR, 3′UTR); (ii) position of the sequence overlap on the antisense transcript (end overlap, middle overlap, full overlap). (iii) length of overall sequence overlap; (iv) continuity or discontinuity of sequence overlap.

Structural parameters—pertains to both sense and antisense transcripts (i) tertiary structure (i.e., hairpin, helix, stem and loop, pseudoknot, and the like); (ii) single stranded versus double stranded regions; (iii) GC content; (iv) tandem Gs; (v) adenosine/inosine content; (vi) thermodynamic stability of tertiary structures; (vii) duplex melting point; (viii) methylations and other RNA modifications; (ix) RNA-protein interactions; and (x) transcript length.

Functional parameters—(i) alternative splicing; (ii) tissue expression; (iii) pathology specific expression; (iv) antisense promoters; (v) intron content; (vi) open reading frame in antisense transcript.

These parameters can be used individually or in combination, in which case, each parameter is preferably weighted according to its importance. Due to the multi-factorial design of artificial antisense transcripts according to this aspect of the present invention, employing a scoring system (described hereinabove) is preferably used to simplify and increase the accuracy of the process.

Synthetic antisense oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of 10 to about 200 bases preferably 15-150 bases, more preferably 20-100 bases, most preferably 20-50 bases.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistance to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

The present invention also includes antisense molecules, which are chimeric molecules. “Chimeric” antisense molecules”, are oligonucleotides, which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such include RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

Finally, chimeric oligonucleotides of the present invention can comprise a ribozyme sequence. Ribozymes are being increasingly used for the sequence-specific inhibition of gene expression by the cleavage of mRNAs. Several ribozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a ribozyme designed to selectively destroy Hepatitis C Virus (HCV) RNA, (Ribozyme Pharmaceuticals, Incorporated —WEB home page).

It will be appreciated that polynucleotide sequence data (i.e., mRNAs and naturally occurring antisense transcripts thereof, which may be referred to interchangeably) obtained according to the teachings of the present invention may also be used for modulating the expression of a gene of interest by upregulating the expression of its naturally occurring antisense transcript.

Upregulating expression of a naturally occurring antisense transcript of interest may be effected via the administration of at least one of the exogenous polynucleotide sequences of the present invention, ligated into a nucleic acid expression construct designed for expression of coding sequences in eukaryotic cells (e.g., mammalian cells). Accordingly, the exogenous polynucleotide sequence may be a DNA or RNA sequence encoding the naturally occurring antisense transcript of interest.

For therapeutic applications, the nucleic acid construct can be administered to an individual in need therefore by employing any suitable mode of administration described hereinbelow (i.e., in-vivo gene therapy). Alternatively, the nucleic acid construct can be introduced into an isolated cells, of for example, a cell culture, using an appropriate gene delivery vehicle/method (transfection, transduction, homologous recombination, etc.). The genetically modified cells thus generated can then be expanded in culture and returned to the individual (i.e., ex-vivo gene therapy).

To enable cellular expression of the polynucleotides of the present invention, the nucleic acid construct of the present invention further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably also includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

It will be appreciated that when the product of the naturally occurring antisense transcript is a polypeptide which regulates the polypeptide product of the gene of interest (e.g., a phosphatase which regulates a phosphorylated protein), upregulation of the naturally occurring antisense of interest may be effected by administering to the subject a polypeptide agent derived from the product of the naturally occurring antisense of interest. It will be appreciated that since the bioavailability of large polypeptides is relatively small due to high degradation rate and low penetration rate, administration of polypeptides is preferably confined to small peptide fragments (e.g., about 100 amino acids).

The oligonucleotides and polynucleotides generated according to the teachings of the present invention can be used for both diagnostic and therapeutic purposes. For example, oligonucleotides of the present invention can be used to diagnose and treat a variety of diseases or pathological conditions associated with an abnormal expression (i.e., up-regulation or down-regulation) of at least one mRNA molecule of interest, including but not limited to diabetes, autoimmune diseases, Parkinson, Alzheimer' disease, HIV, malaria, cholera, influenza, rabies, diphtheria, breast cancer, colon cancer, cervical cancer, melanoma, lung cancer, ovarian cancer, pancreatic cancer, prostate cancer, lymphomas, leukemias and the like and any other diseases (see Example 8 of the Examples section) which are associated with aberrant expression of multiple mRNAs (i.e., sense and/or antisense) or with unregulated formation of endogenous double stranded RNA complexes.

Present-day mRNA-based diagnostic assays utilize oligonucleotide probes which are complementary to one or more regions of the mRNA to be quantitated. Such probes are designed while considering interspecies sequence variation, sequence length, GC content etc. However design of such prior art probes (i.e., riboprobes or deoxyriboprobes) does not take into consideration the presence of antisense transcripts which can effect probe binding efficiency. Discounting antisense presence can lead to inaccurate diagnosis, which is oftentimes followed by an erroneous treatment protocol.

The present invention provides an mRNA-detection/quantification assay, which is devoid of this limitation.

Thus, according to an additional aspect of the present invention there is provided a method of quantifying at least one mRNA of interest in a biological sample.

As used herein, the phrase “biological sample” refers to any sample derived from biological tissues or fluids, including blood (serum or plasma), sputum, pleural effusions, urine, biopsy specimens, isolated cells and/or cell membrane preparation. Methods of obtaining tissue biopsies and body fluids from mammals are well known in the art.

The method of this aspect of the present invention is effected by contacting mRNA from a cell type or within a cell with one or more oligonucleotides that hybridizes efficiently with a sequence region of an mRNA transcript which is not complementary with a naturally occurring antisense transcript.

In addition to the limitation described above, prior art diagnostic/detection assays also fail to consider the effect of antisense transcription on the protein expression levels of a gene of interest. It stands to reason that presence of antisense transcripts in a biological sample can substantially reduce the resultant protein levels translated from a complementary sense transcript. Consistently, diseases which are associated with endogenous dsRNA complexes, are also very difficult to detect and moreover to treat, due to insufficient sequence data pertaining to duplex forming transcripts.

Thus, for accurate quantification of gene expression, both the sense and antisense levels must be quantified and/or their respective expression ratio must be determined.

By contacting a biological sample with one or more pairs of oligonucleotides, where one oligonucleotide is capable of hybridizing with the mRNA of interest and the second oligonucleotide is capable of hybridizing with a naturally occurring antisense transcript which is complementary with the mRNA of interest such accurate quantification can be effected.

Contacting the oligonucleotides of the present invention with the biological sample is effected by stringent, moderate or mild hybridization (as used in any polynucleotide hybridization assay such as northern blot, dot blot, RNase protection assay, RT-PCR and the like). Wherein stringent hybridization can be effected using a hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; moderate hybridization is effected by a hybridization solution of 6×SSC and 0.1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; whereas mild hybridization is effected by a hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 37° C., final wash solution of 6×SSC and final wash at 22° C.

The oligonucleotides of the present invention can be attached to a solid substrate, which may consist of a particulate solid phase such as nylon filters, glass slides or silicon chips [Schena et al. (1995) Science 270:467-470].

In a particular embodiment, oligonucleotides of the present invention can be attached to a solid substrate, which is designed as a microarray. Microarrays are known in the art and consist of a surface to which probes that correspond in sequence to gene products (e.g., cDNAs, mRNAs, cRNAs, polypeptides, and fragments thereof), can be specifically hybridized or bound at a known position (regiospecificity).

Several methods for attaching the oligonucleotides to a microarray are known in the art including but not limited to glass-printing, described generally by Schena et al., 1995, Science 270:467-47, photolithographic techniques [Fodor et al. (1991) Science 251:767-773], inkjet printing, masking and the like.

In general, quantifying hybridization complexes is well known in the art and may be achieved by any one of several approaches. These approaches are generally based on the detection of a label or marker, such as any radioactive, fluorescent, biological or enzymatic tags or labels of standard use in the art. A label can be applied on either the oligonucleotide probes or nucleic acids derived from the biological sample.

The following illustrates a number of labeling methods suitable for use in the present invention. For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides. It will be appreciated that pairs of fluorophores are chosen when distinction between two emission spectra of two oligonucleotides is desired or optionally, a label other than a fluorescent label is used. For example, a radioactive label, or a pair of radioactive labels with distinct emission spectra, can be used [Zhao et al. (1995) Gene 156:207]. However, because of scattering of radioactive particles, and the consequent requirement for widely spaced binding sites, the use of fluorophores rather than radioisotopes is more preferred.

The intensity of signal produced in any of the detection methods described hereinabove may be analyzed manually or using a software application and hardware suited for such purposes.

In general, mRNA quantification is preferably effected alongside a calibration curve so as to enable accurate mRNA determination. Furthermore, quantifying transcript(s) originating from a biological sample is preferably effected by comparison to a normal sample, which sample is characterized by normal expression pattern of the examined transcript(s).

It will be appreciated that the detection method described above can also be used for quantifying at least one naturally occurring antisense transcript in a biological sample. In such a case, the oligonucleotide used for quantification is designed to hybridize with a sequence region of naturally occurring antisense transcript of interest, which is not complementary with a naturally occurring mRNA transcript.

The diagnostic assays described hereinabove can be used to accurately distinguish between absence, presence and excess expression of any transcripts of interest (e.g., sense, antisense), and to monitor their level during therapeutic intervention. These methods are also capable of diagnosing diseases associated with an improper balance or ratio between sense and antisense expression and diseases associated with endogenous dsRNA.

Further description of oligonucleotide-pair arrays is provided in Example 9 of the Examples section which follows.

As discussed hereinabove oligonucleotides of the present invention can be also used for therapeutic purposes, such as treating diseases or conditions associated with aberrant expression levels of one or more sense and/or antisense transcripts and conditions, which are associated with endogenous dsRNA such as unregulated formation of double-strand RNA (i.e., up/down-regulation).

Accumulative knowledge shows strong correlation between a variety of human diseases and mutations, over-expression and function of the protein building blocks (i.e., protein kinases, phosphatsases) and their effectors and regulators, which constitute numerous intracellular signaling pathways. For instance, inactivation of both copies of ZAP-70 or Jak-3 causes severe combined immunodeficiency and mutation of the X-linked BTK gene results in agammaglobulinemia. Many genetic disorders are also associated with mutations for example, in protein-serine kinases (PSKs) and phosphatases. The Coffin-Lowry syndrome results from inactivation of the X-linked Rsk2 gene, and myotonic dystrophy is due to decreased levels of expression of the myotonic dystrophy PSK. In addition, over-expression of ErbB2 receptor tyrosine kinase is implicated in breast and ovarian carcinoma [reviewed by Hunter T. (2000) Cell 100:113-127].

Given the importance of activated kinases in a variety of disorders such as cancer, it would be anticipated that phosphatases regulation would be found as tumor suppressor genes and as promising drug targets. So far this has not proven to be the case. Furthermore, a number of diseases are associated with insufficient expression of signaling molecules, including non-insulin-dependent diabetes and peripheral neuropathies.

Thus, it is conceivable that identification of naturally occurring antisense transcripts of signaling molecules participating in specified signaling pathways may serve as promising tools for both identification and particularly treatment of a variety of disorders at any gene expression level (i.e., RNA, DNA or protein).

The term “treating” refers to alleviating or diminishing a symptom associated with the disease or the condition. Preferably, treating cures, e.g., substantially eliminates, and/or substantially decreases, the symptoms associated with the diseases or conditions of the present invention.

The treatment method according to the teachings of the present invention includes administering to an individual a therapeutically effective amount of the oligonucleotides, polynucleotides or polypeptides of the present invention. Preferred individual subjects according to the present invention are mammals such as canines, felines, ovines, porcines, equines, bovines, humans and the like.

A therapeutically effective amount implies an amount of agent effective to prevent, alleviate or ameliorate symptoms of disease or prolong the survival of the individual being treated

The agent of the method of the present invention can be administered to an individual per se, or as part of a pharmaceutical composition where it is mixed with a pharmaceutically acceptable carrier.

As used herein a “pharmaceutical composition” refers to a composition of one or more of the agents described hereinabove, or physiologically acceptable salts or prodrugs thereof, with other chemical components. The purpose of a pharmaceutical composition is to facilitate administration of a compound to an organism.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The pharmaceutical compositions of the present invention may employ various penetration enhancers to effect the efficient delivery of nucleic acids, particularly oligonucleotides, to the skin of animals.

Penetration enhancers may be classified as belonging to one of five broad categories, i.e., surfactants, fatty acids, bile salts, chelating agents, and non-chelating non-surfactants [Lee et al., Critical Reviews in Therapeutic Drug Carrier Systems (1991) 92] as disclosed in U.S. Pat. Nos. 6,300,132, 6,271,030, 6,277,633, 6,284,538, 6,287,860, 6,294,382, 6,277,640 and 6,258,601 each of which is herein fully incorporated by reference.

Other substances that enhance uptake of oligonucleotides at the cellular level may also be added to the pharmaceutical compositions of the present invention. For example, cationic lipids, such as lipofectin [U.S. Pat. No. 5,705,188], cationic glycerol derivatives, and polycationic molecules, such as polylysine [PCT Application WO 97/30731], are also known to enhance the cellular uptake of oligonucleotides.

Other reagents may be utilized to enhance the penetration of the administered nucleic acids, including glycols such as ethylene glycol and propylene glycol, pyrrols such as 2-pyrrol, azones, and terpenes such as limonene and menthone.

Certain pharmaceutical compositions of the present invention may also incorporate carrier compounds. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′ isothiocyano-stilbene-2,2′-disulfonic acid [Miyao et al., Antisense Res. Dev., (1995) 5:115-121; Takakura et al., Antisense & Nucl. Acid Drug Dev. (1996) 6:177-183].

In contrast to a carrier compound, an “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical excipients include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration, which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation. Aqueous suspensions may contain substances which increase the viscosity of the suspension including, for example, sodium carboxymethylcellulose, sorbitol and/or dextran. The suspension may also contain stabilizers.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50 found to be effective in in vitro and in vivo animal models. Persons of ordinary skill in the art can easily estimate dosing and repetition rates based on measured residence times and concentrations of the oligonucleotide in bodily fluids or tissues. Following successful treatment, it may be desirable to have the patient undergo maintenance therapy to prevent the recurrence of the disease state, wherein the oligonucleotide is administered in maintenance doses.

The methods of the present invention have evident utility in the diagnosis and treatment of various diseases and conditions. In addition, such methods can also be used in non-clinical applications, such as, for example, differential cloning, detection of rearrangements in DNA sequences as disclosed in U.S. Pat. No. 5,994,320, drug discovery and the like.

The oligonucleotides generated according to the teachings of the present invention can be included in a diagnostic or therapeutic kit. For example, oligonucleotides sets pertaining to specific disease related transcripts can be packaged in a one or more containers with appropriate buffers and preservatives along with suitable instructions for use and used for diagnosis or for directing therapeutic treatment.

Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

Naturally occurring antisense sequences uncovered using the above-described methodology can be annotated using a number of publicly available sources with gene annotations which are well known to those of skill in the art. Examples include, but are not limited to Locus Link and RefSeq: GO annotations, Gencarta (described in Example 10 of the Examples section), GeneCards, GeneLynx, TIGR and the like.

Annotative information obtained using the Gencarta (Compugen, Tel-Aviv, Israel) database is set forth in the file “annotations_{—}136” of the enclosed CD-ROM4.

Elucidating protein function, pattern of expression, therapeutic and diagnostic roles, allows for the design of highly specific and effective clinical tools, for a wide range of diseases as described in the Examples section which follows.

For example, gene products (nucleic acid and/or protein products), which exhibit tumor specific expression (i.e., tumor associated antigens, TAAs) can be utilized for in-vitro generation of antibodies and/or for in-vivo immunization/cancer vaccination, essentially eliciting an immune response against such gene products and cells expressing same (see e.g., U.S. Pat. No. 4,235,877, Vaccine preparation is generally described in, for example, M. F. Powell and M. J. Newman, eds., “Vaccine Design (the subunit and adjuvant approach),” Plenum Press (NY, 1995); Other references describing adjuvants, delivery vehicles and immunization in general include Rolland, Crit. Rev. Therap. Drug Carrier Systems 15:143-198, 1998; Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993; Ulmer et al., Science 259:1745-1749, 1993; Cohen, Science 259:1691-1692, 1993; U.S. Pat. Nos. 4,436,727; 4,877,611; 4,866,034 and 4,912,094; U.S. Pat. Nos. 6,008,200 and 5,856,462; Zitvogel et al., Nature Med. 4:594-600, 19980.

Tumor-specific gene products of the present invention, in particular membrane bound, can be utilized as targeting molecules for binding therapeutic toxins, antibodies and small molecules, to thereby specifically target the tumor cell. Alternatively, neoplastic properties of tumor specific gene products (nucleic acid and/or protein products) of the present invention, may be beneficially used in the promotion of wound healing and neovascularization in ischemic conditions and diabetes.

Secreted variants of known autoantigens associated with a specific autoimmune syndrome, such as for example, those listed in Table 11, below, can be used to treat such syndromes. Typically, autoimmune disorders are characterized by a number of different autoimmune manifestations (e.g., multiple endocrine syndromes). For these reasons secreted variants may be used to treat any combination of autoimmune phenomena of a disease as detailed in Table 11 below. The therapeutic effect of these variants may be a result of (i) competing with autoantigens for binding with autoantibodies; (ii) antigen-specific immunotherapy, essentially suggesting that systemic administration of a protein antigen can inhibit the subsequent generation of the immune response to the same antigen (has been proved in mice models, for Myasthenia Gravis and type I Diabetes).

Biomolecular sequences, which are over-expressed in a pathology can be used as diagnostic markers, such as for cancer. Variants of autoantigens may also be used for diagnosis. The diagnosis of many autoimmune disorders is based on looking for specific autoantibodies to autoantigens known to be associated with an autoimmune condition. Most of the diagnostic techniques are based on having a recombinant form of the autoantigen and using it to look for serum autoantibodies. It is possible that currently considered autoantigens are not “true” autoantigens but rather variants thereof. For example, TPO is a known autoantigen for thyroid autoimmunity. It has been shown that its variant TPOzanelli also takes part in the autoimmune process and can bind the same antibodies as TPO [Biochemistry. 2001 Feb. 27;40(8):2572-9.]. Antibodies formed against the true autoantigen may bind to other variants of the same gene due to sequence overlap but with reduced affinity. Novel splice variant of the genes in Table 11 may be revealed as true autoantigens, therefore their use for detection of autoantibodies is expected to result in a more sensitive and specific test.

Additionally, variants of known drug targets can be used in cases where the known drug has major side effects, the therapeutic efficacy of the known drug is moderate, the drug failed clinical trials due to one of the above. A drug which is specific to a new protein variant of the target or to the target only (without affecting the novel variant) is likely to have less side effects as compared with the original drug, higher efficacy and may treat different indications than the original drug.

For example, COX3, which is a variant of COX1, is known the bind COX inhibitors in different affinity than they bind to COX1. This molecule is also associated with different physiological processes than COX1. Therefore, a compound specific to COX1 or compounds specific to COX3 would have lower side effects (by not affecting the other variant), treat different indications and treat successfully bigger populations.

Apart of clinical applications, the biomolecular sequences of the present invention can find other commercial uses such as in the food, agricultural, electro-mechanical, optical and cosmetic industries [http://www.physics.unc.edu/˜rsuper/XYZweb/XYZchipbiomotors.rs1.doc; http://www.bio.org/er/industrial.asp]. For example, newly uncovered gene products, which can disintegrate connective tissues, can be used as potent anti scarring agents for cosmetic purposes. Other applications include, but are not limited to, the making of gels, emulsions, foams and various specific products, including photographic films, tissue replacers and adhesives, food and animal feed, detergents, textiles, paper and pulp, and chemicals manufacturing (commodity and fine, e.g., bioplastics).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### In-Vitro Expression Substantiation of Computationally Retrieved Naturally Occurring Antisense Transcripts

In-vitro expression assays were conducted in order to validate the existence of naturally occurring antisense sequences identified according to the teachings of the present invention.

Table 1 below lists polynucleotide sequence pairs that were selected for the in-vitro expression validation assays described in examples 1-7.

TABLE 1

| | | | | | | Start of | |
|---|---|---|---|---|---|---|---|
| | | Sense | | Anti-sense | Overlap | overlap | Start of |
| Name of sense | Sense | Length | Antisense | Length | length | sense | overlap |
| antisense pair | transcript | (nt) | transcript | (nt) | (nt) | transcript | anti-sense |
| 53BP1_76P | 53BP1 | 10394 | 76P | 6837 | 3046 | 5463 | 2018 |
| | (SEQ ID NO: 15) | | (SEQ ID NO: 16) | | | | |
| CIDEB_BLTR2 (1) | CIDEB1 | 2289 | BLTR2 | 6530 | 2254 | 17 | 1 |
| | (SEQ ID NO: 19) | | (SEQ ID NO: 21) | | | | |
| CIDEB_BLTR2 (2) | CIDEB2 | 1511 | BLTR2 | 6530 | 1410 | 1 | 1 |
| | (SEQ ID NO: 20) | | | | | | |
| APAF1_EB1 | aAPAF1 | 7042 | EB1a | 1752 | 141 | 6889 | 1612 |
| | (SEQ ID NO: 24) | | (SEQ ID NO: 25) | | | | |
| AChR_MINK2 | AchR | 2457 | MINK2 | 4863 | 236 | 2175 | 4853 |
| | (SEQ ID NO: 29) | | (SEQ ID NO: 30) | | | | |
| M-AchR_Anti-AChR | M-AchR | 1590 | M-Anti-AchR | 2227 | 672 | 934 | 506 |
| | (SEQ ID NO: 35) | | (SEQ ID NO: 36) | | | | |
| CyclinE2_Anti-CyclinE2 | CyclinE2 | 2714 | Anti-CyclinE2 | 5773 | 1855 | 565 | 2006 |
| | (SEQ ID NO: 33) | | (SEQ ID NO: 34) | | | | |

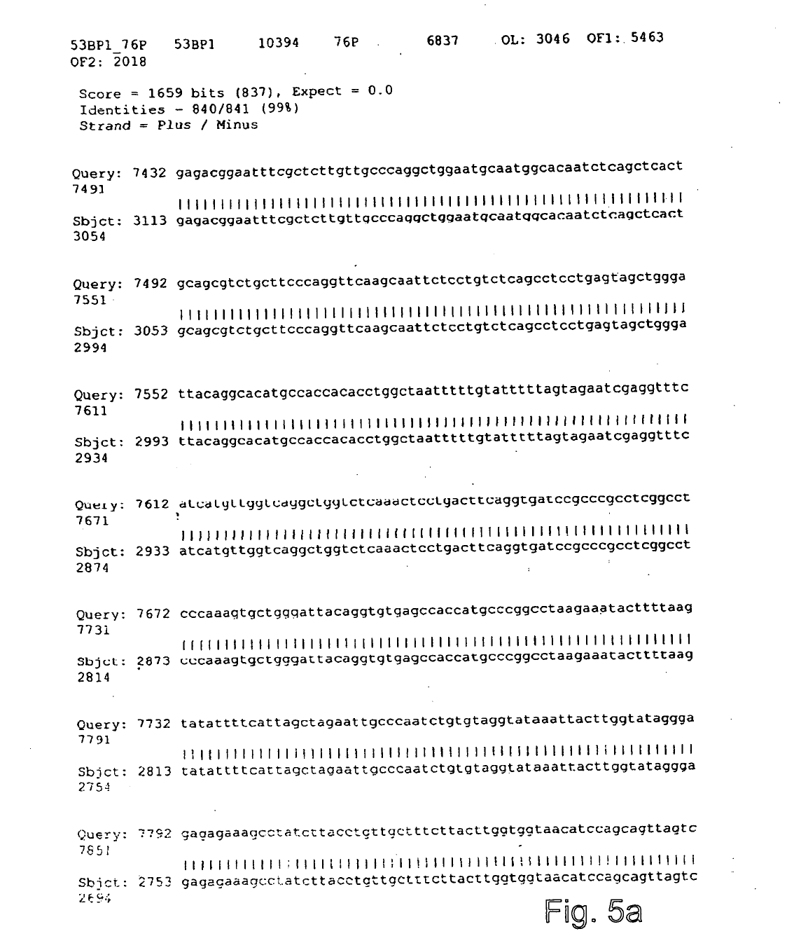

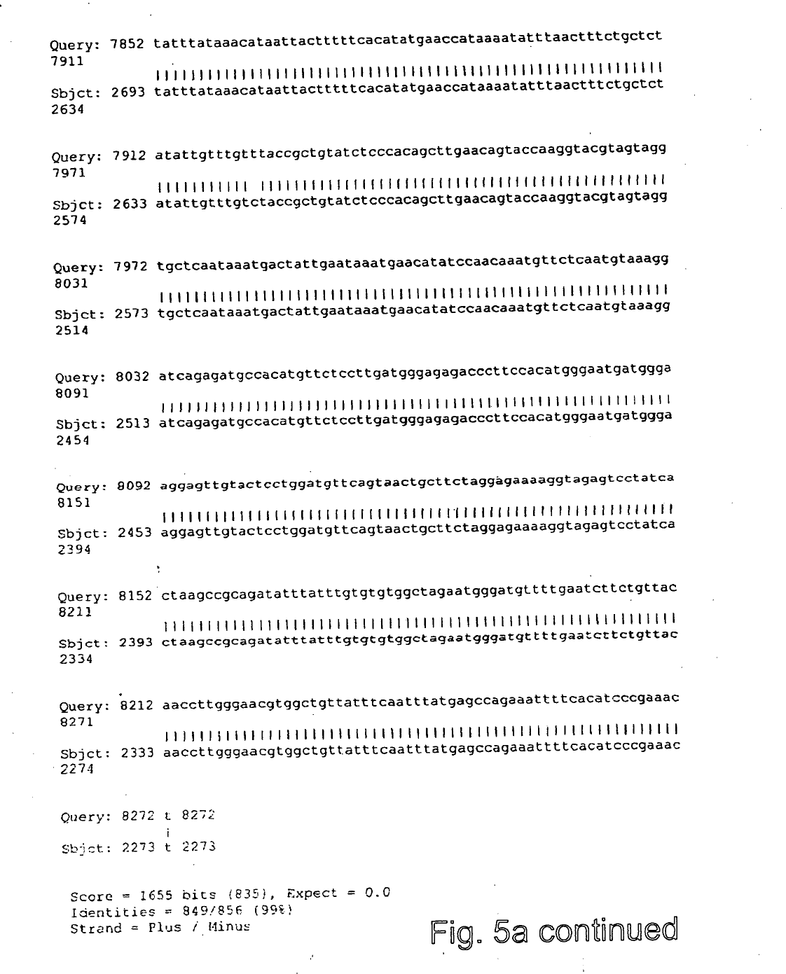

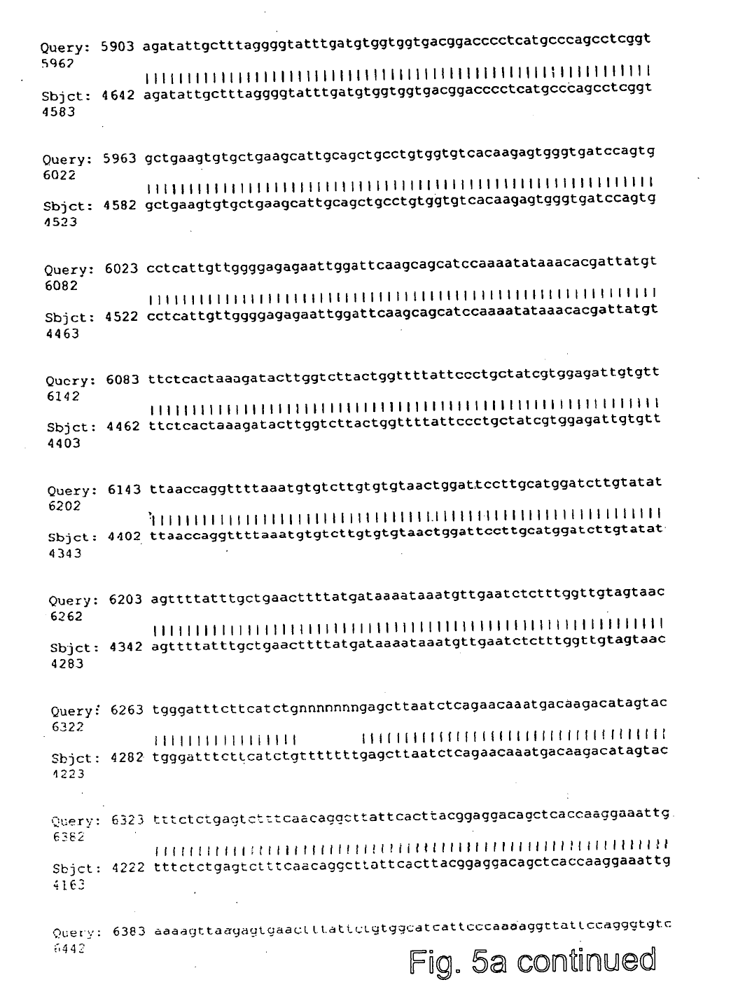

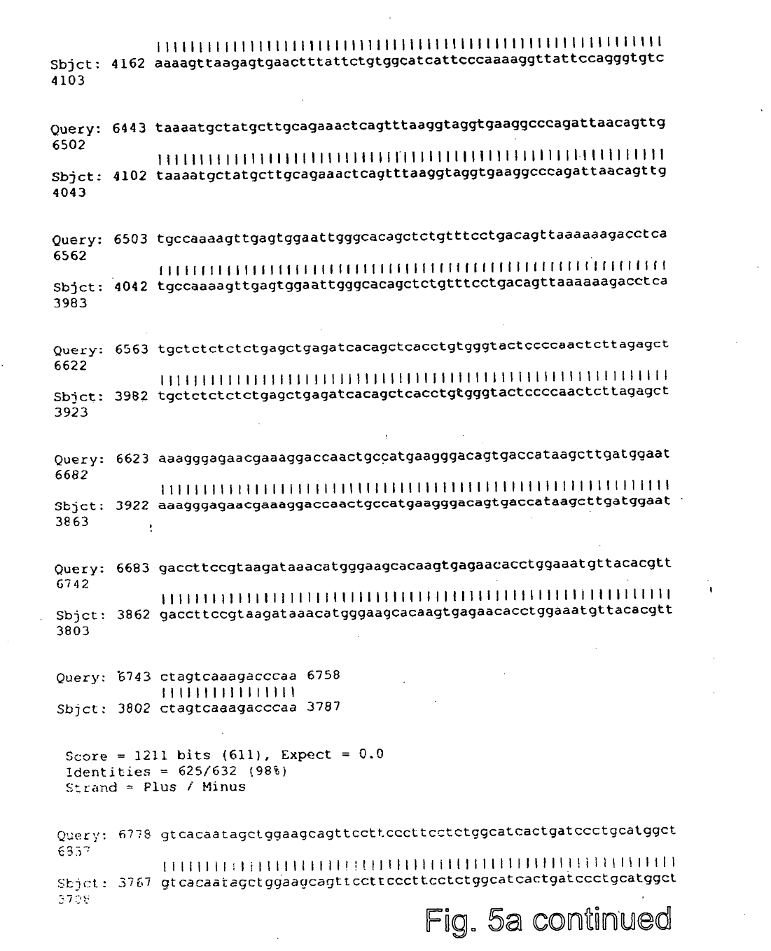

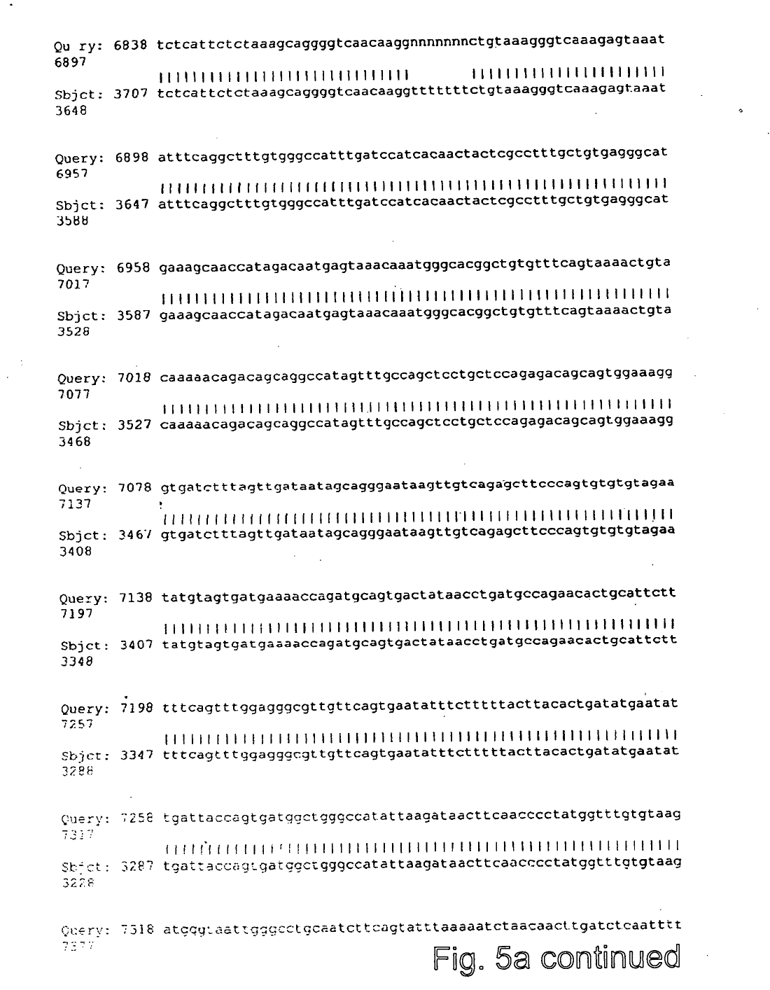

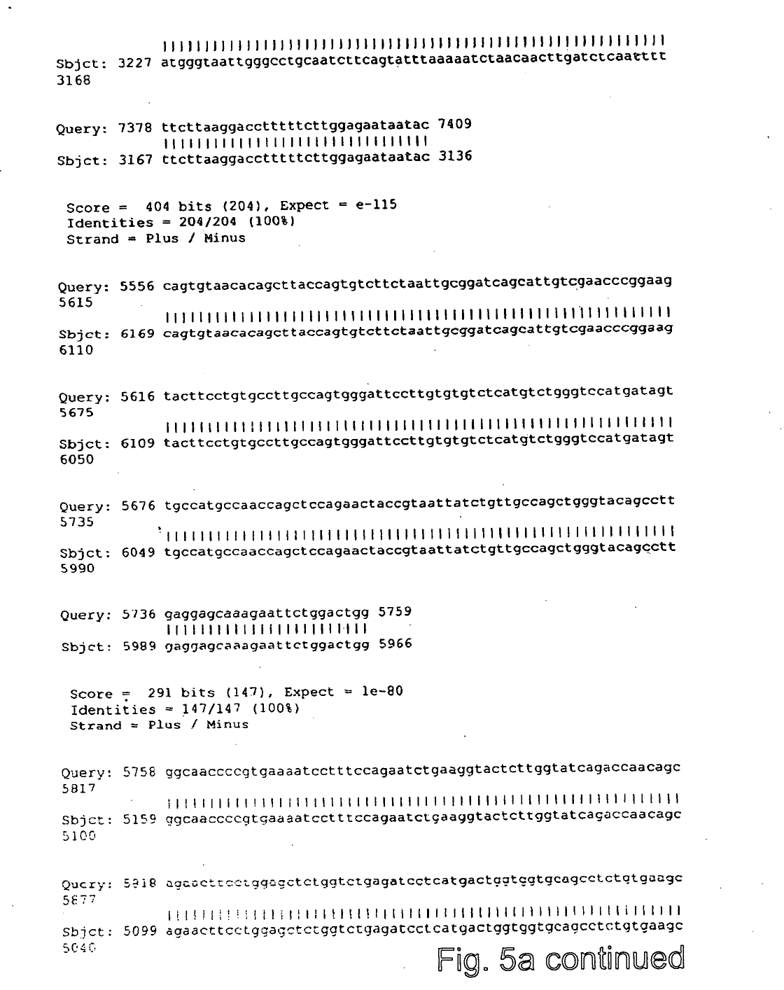

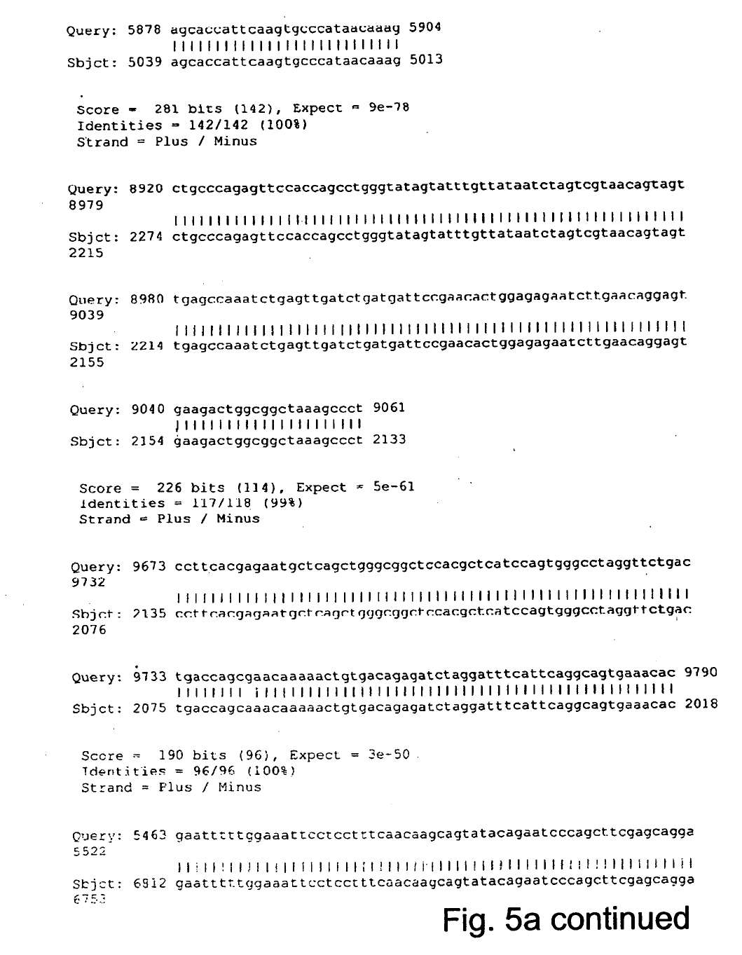

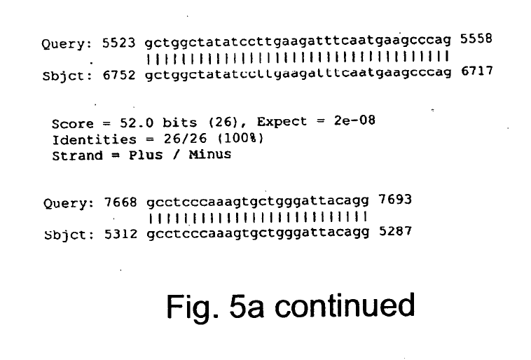

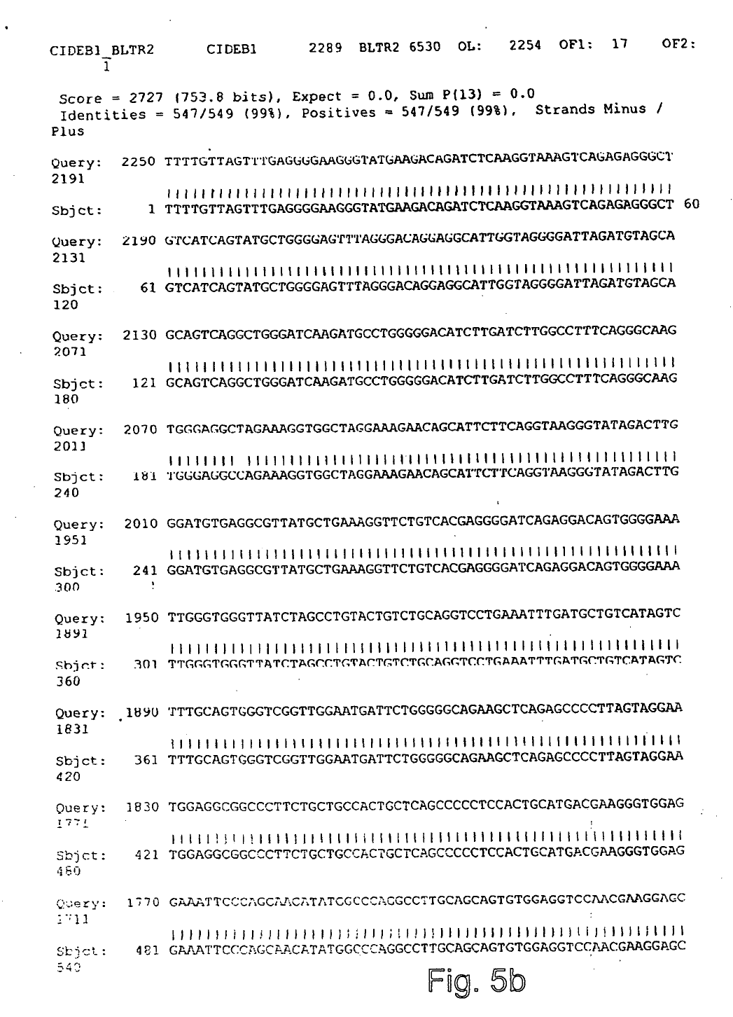

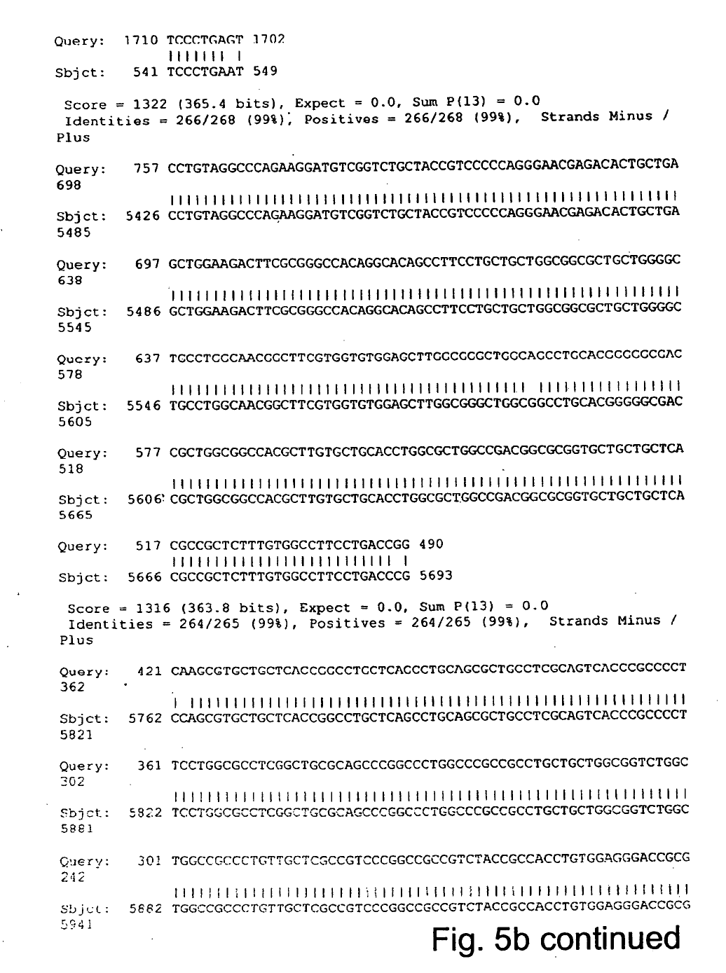

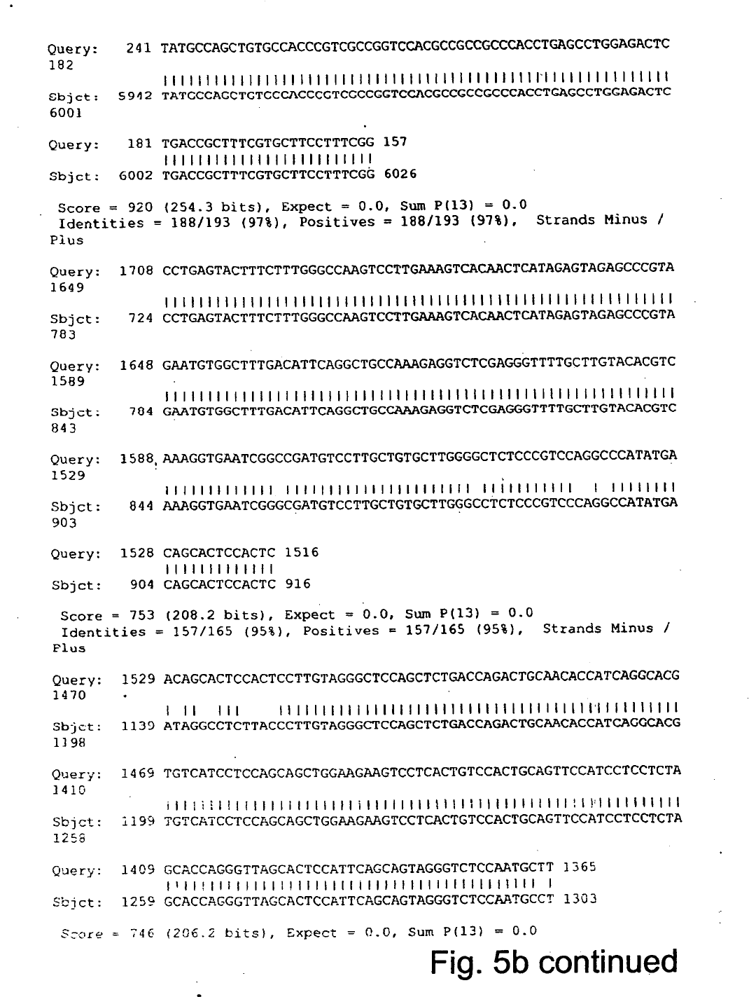

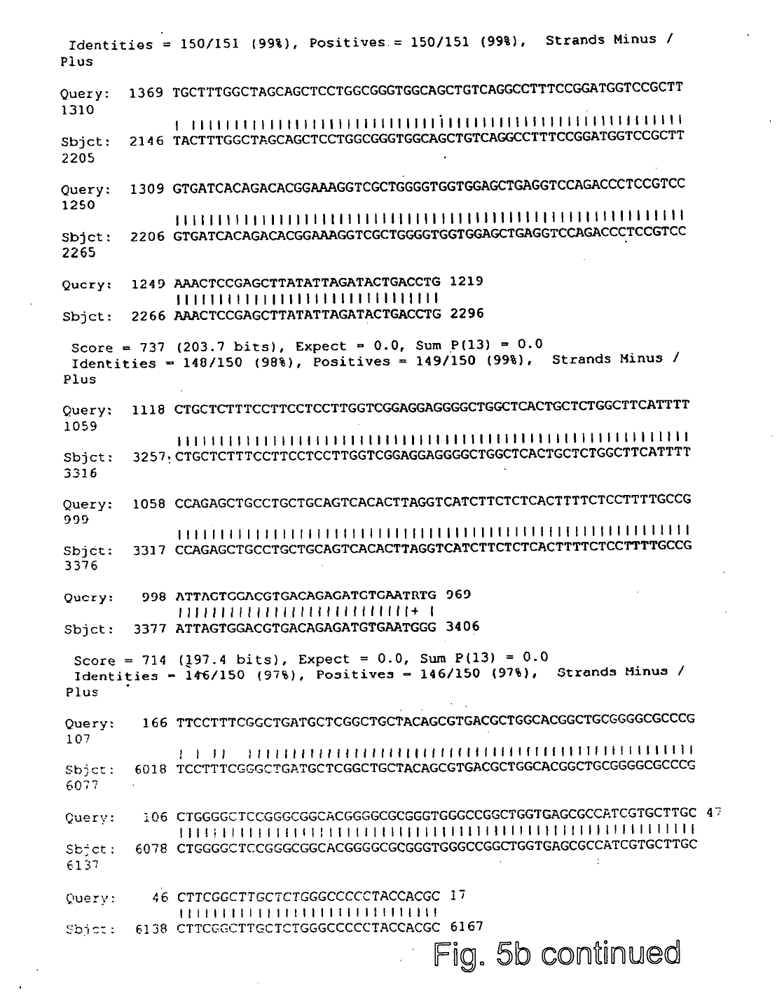

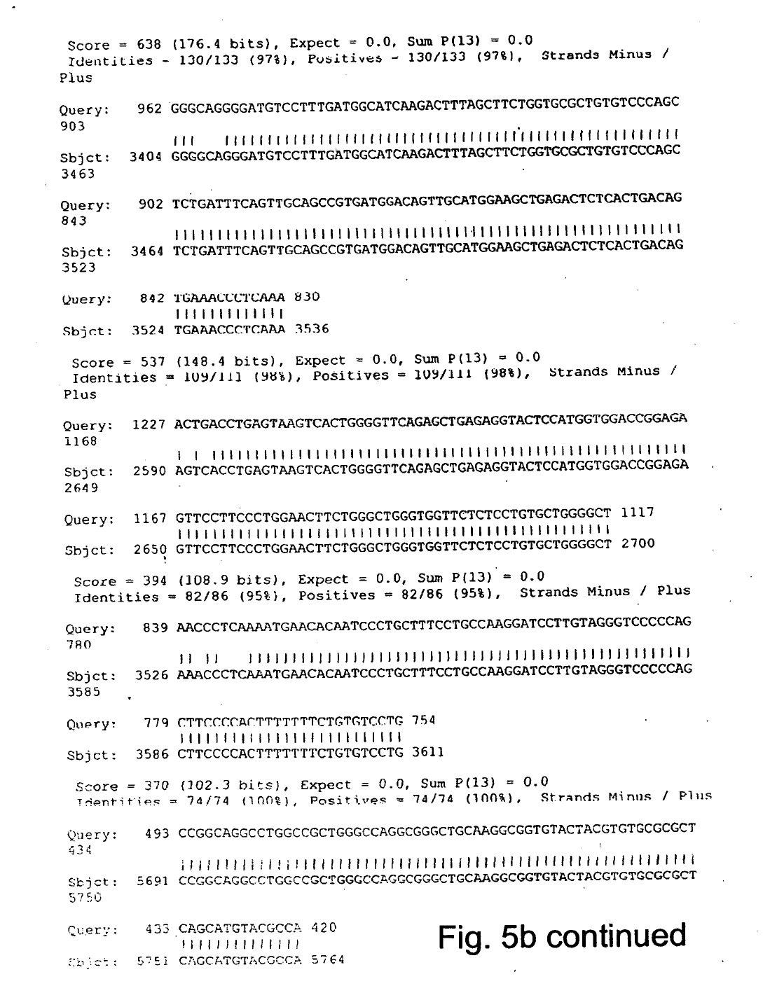

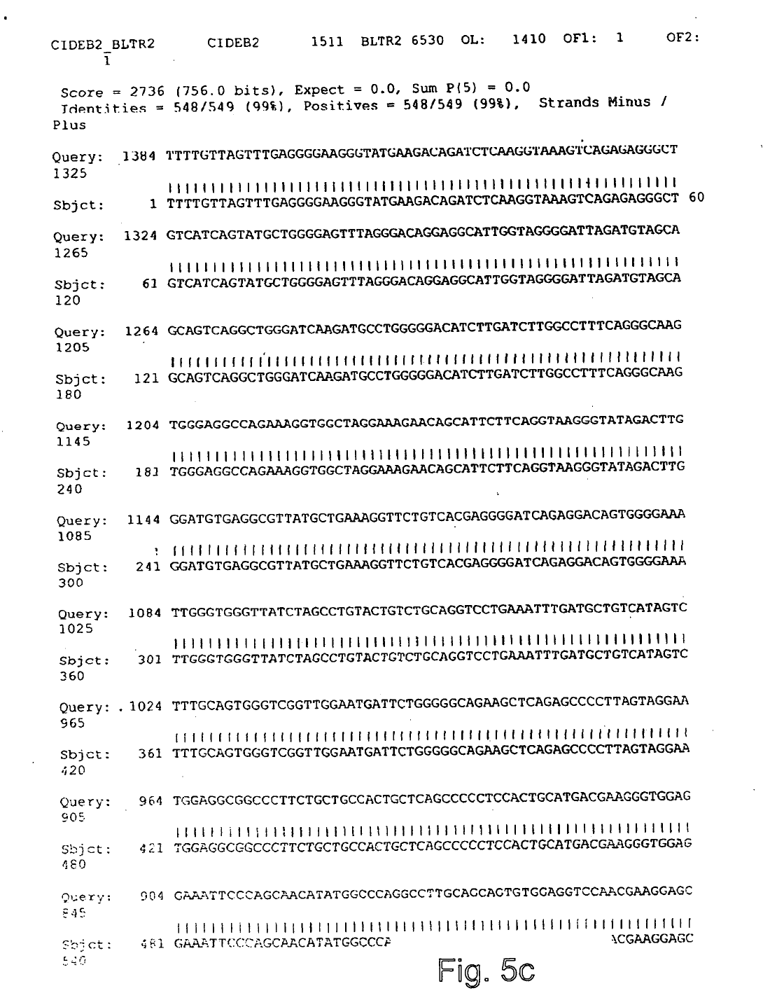

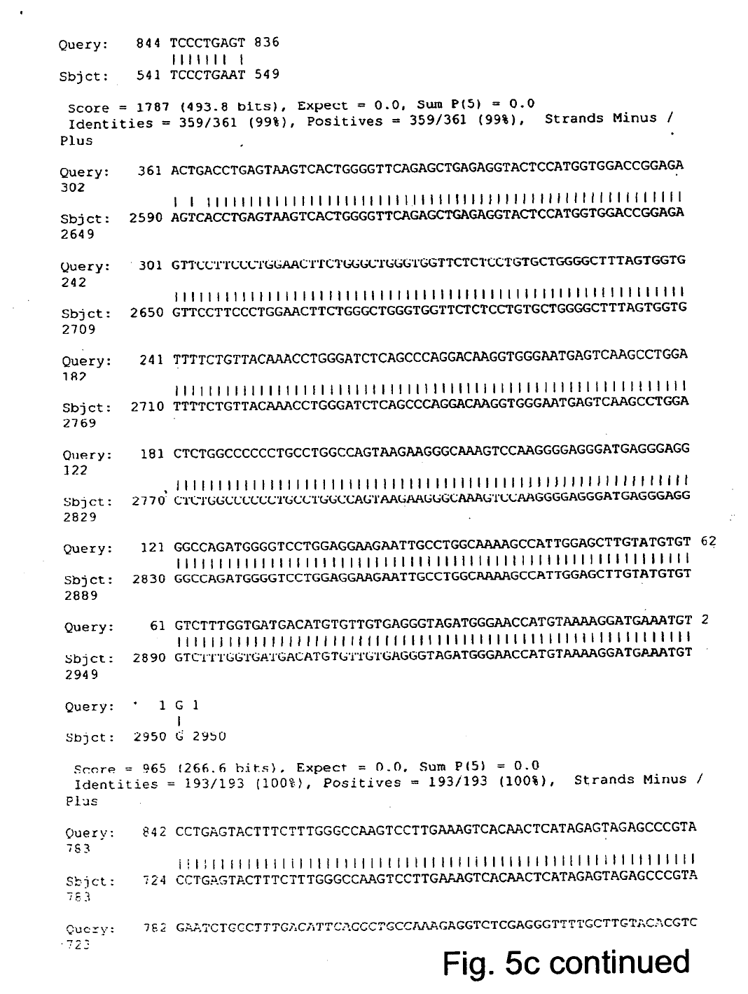

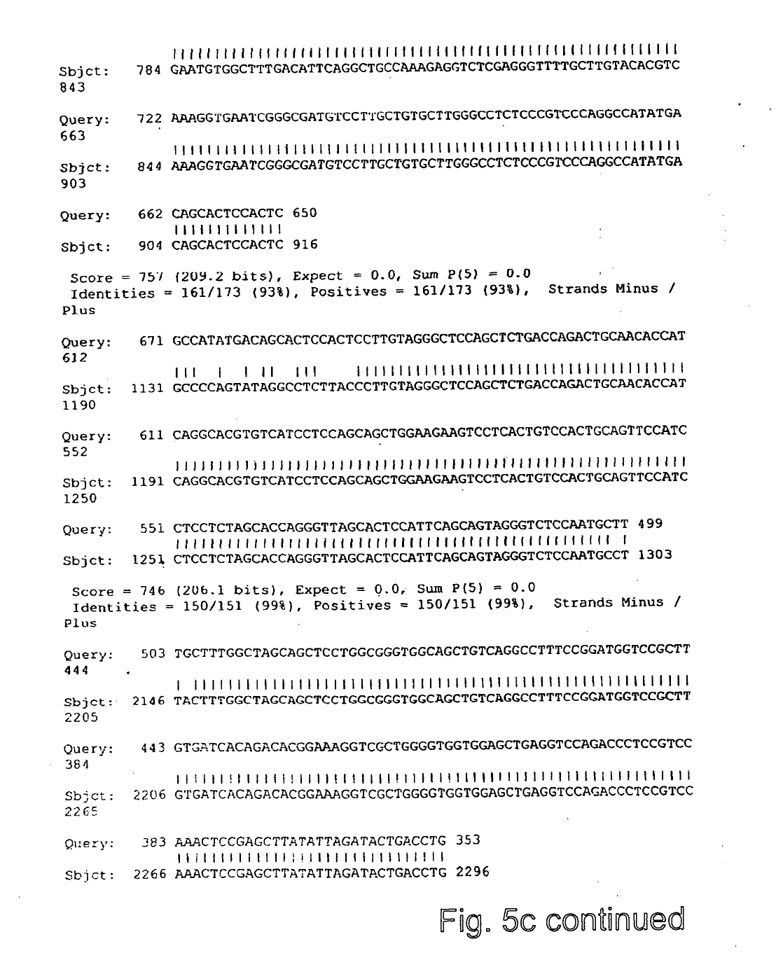

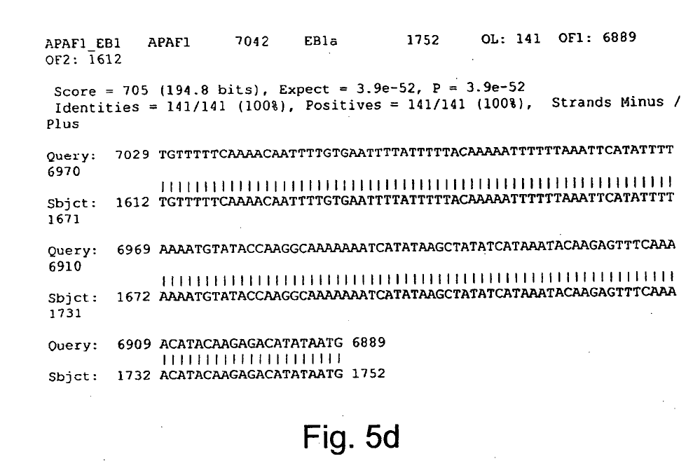

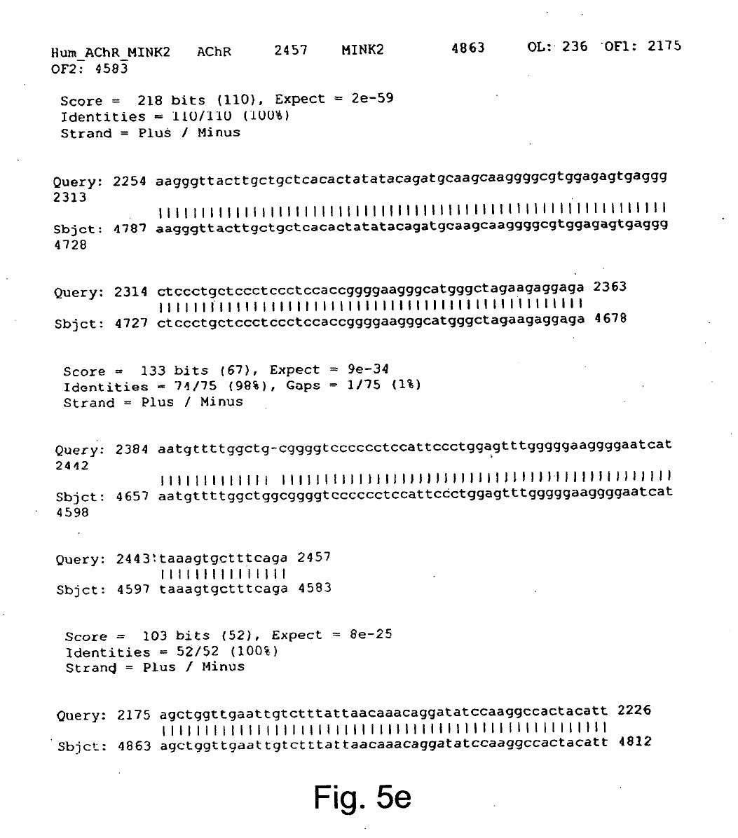

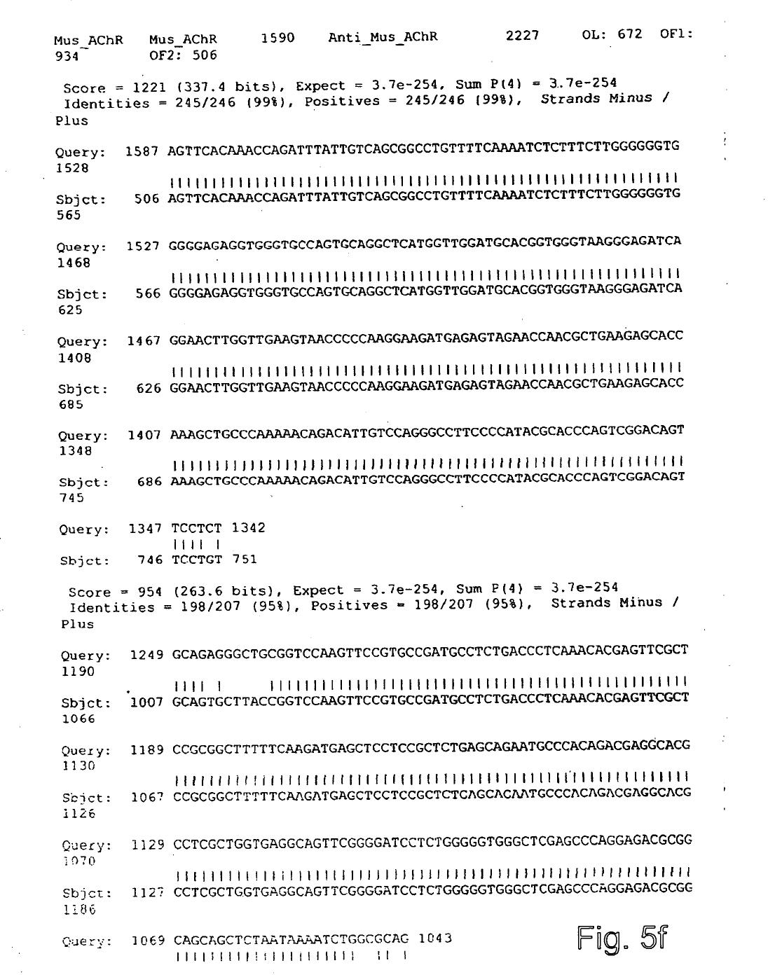

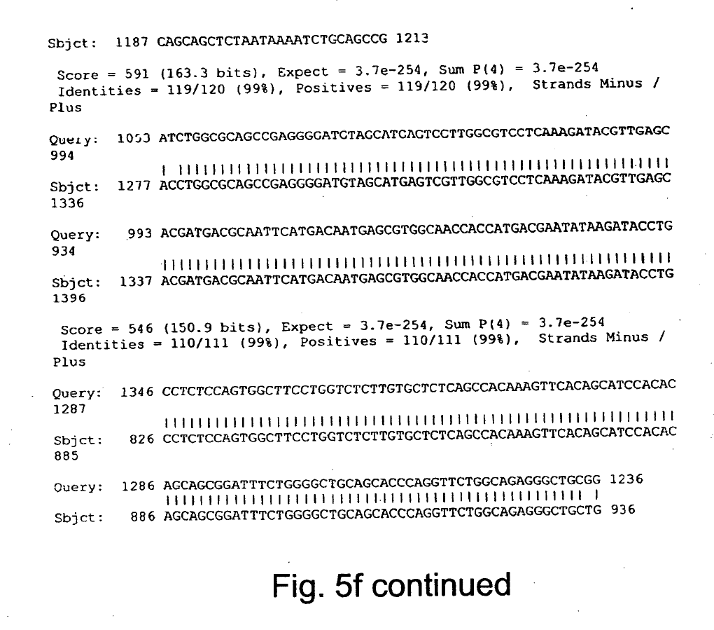

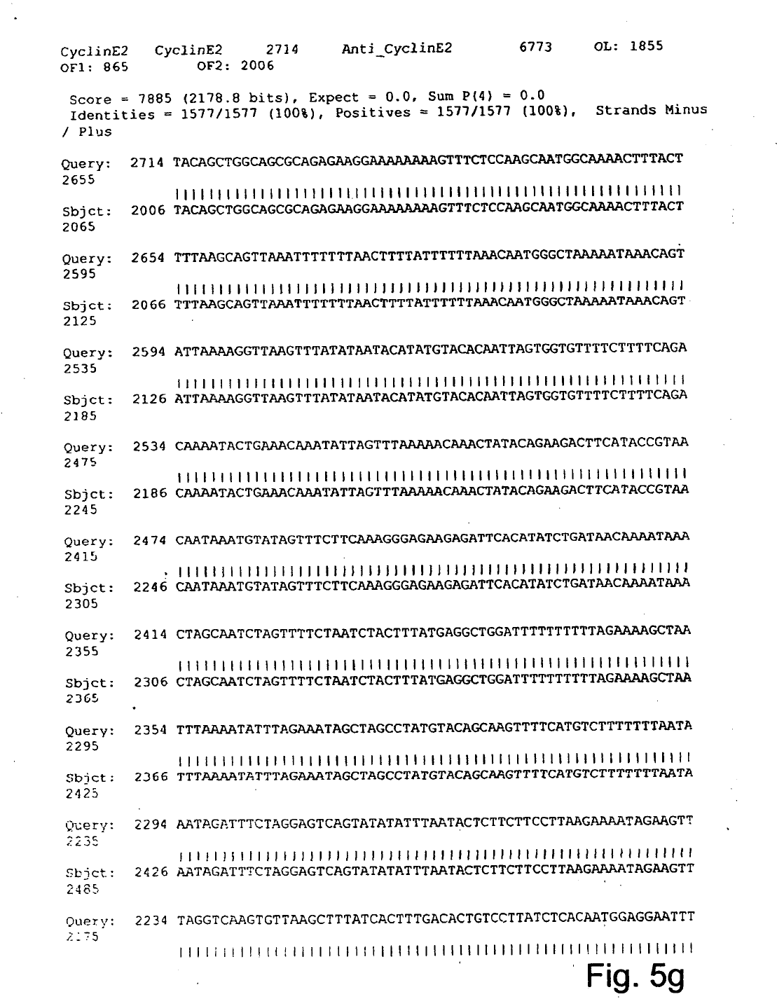

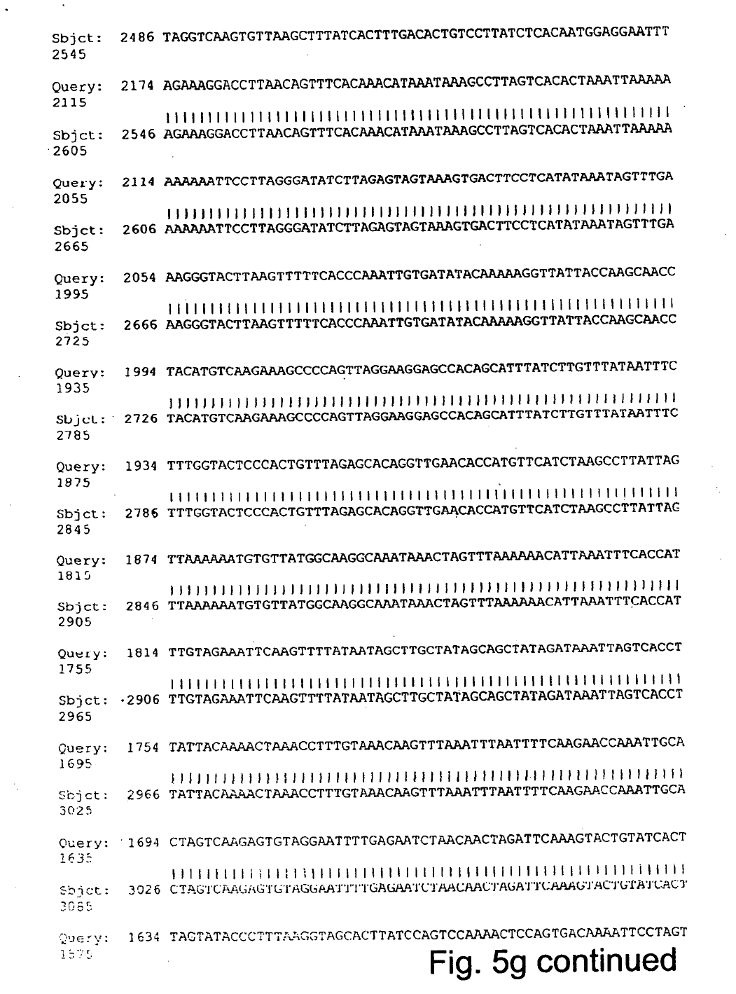

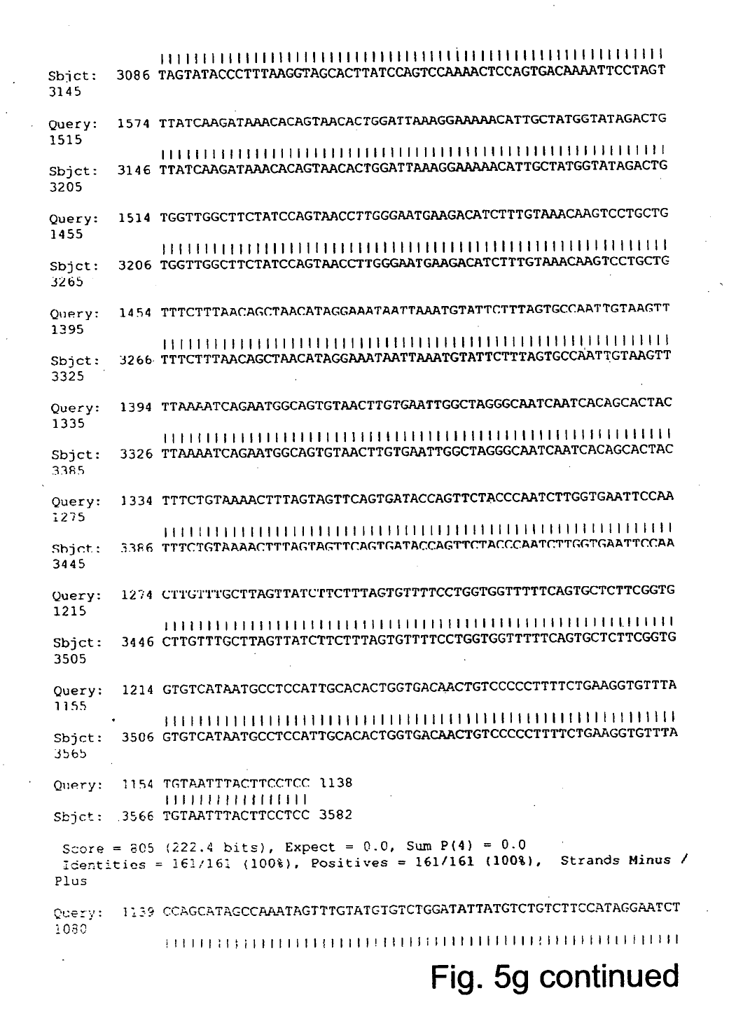

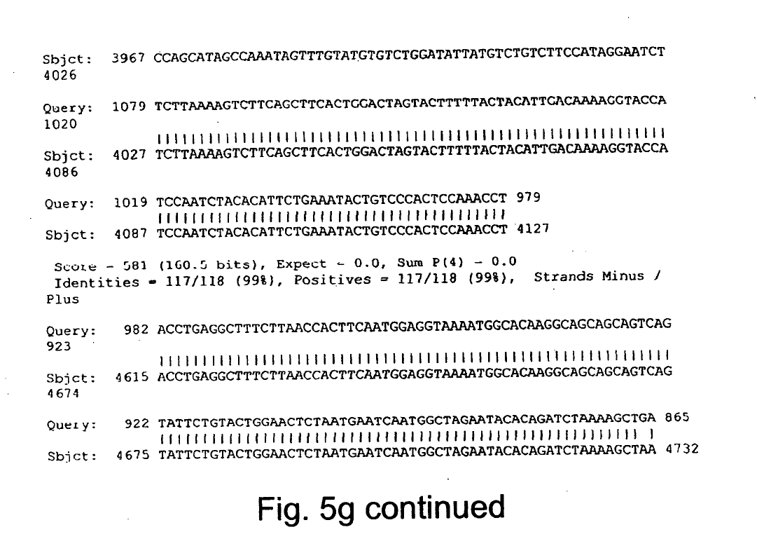

Sequence alignments of overlapping regions of each sense-antisense pair were performed using the BLAST sequence alignment algorithm (Basic Local Alignment Search Tool, available through www.ncbi.nlm.nih.gov/BLAST using the default parameters) and are exhibited in FIG. 5a-g.

A microarray-based analysis was conducted, as well, in order to validate the existence of naturally occurring, antisense sequences identified according to the teachings of the present invention. The results are described in Example 9.

### Materials and Experimental Methods

RNA Probes Generation and Northern Analysis

RNA probes for northern analysis were generated by PCR amplification of a desired DNA fragment and cloning into Zero Blunt TOPO (Invitrogen Corp.) or pSPT18/19 vectors (Roche Ltd.). Alternatively PCR products were ligated into T7 RNA polymerase promoter-containing adaptors using the Lignscribe kit (Ambion Europe Ltd.). Corresponding RNA transcripts were synthesized using T7 RNA polymerase (Roche Ltd.) and labeled with 32P-UTP according to manufacturer's instructions. RNA probes were purified on Mini Quick Spin RNA columns.

Commercial membranes containing Poly(A)-RNA from various human tissues (2 μg RNA per lane) were obtained from Origene (OriGene Technologies Inc.) and Ambion (Ambion Inc.).

Alternatively, 2 μg of poly(A)-RNA prepared from various human cell-lines were electrophoretically separated on 1% agarose gel, and electrotransferred to Nytran SuperCharge membrane (Schleicher & Schuell) and subjected to fixing by UV radiation. Membranes were stained with methylene blue to ensure quantitative RNA transfer. Membranes were then prehybridized in a hybridization solution (UltraHyb solution Ambion Europe Ltd.) for 30 minutes at 68° C. in a rotating hybridization tube.

Hybridization solution was then supplemented with 106 cpm of labeled RNA probe per each ml of hybridization solution. Blots were hybridized for 16 hours at 68° C. in a rotating hybridization tube. Membranes were then washed twice with 2×SSC, 0.1% sodium dodecyl sulfate (SDS) and twice with 0.1% SDS at 68° C. RNA transcripts signals were detected using a phosphoimager (Molecular Dynamics, Sunnyvale Calif.).

Microarray

Oligonucleotide design—oligonucleotide design tools (1) were applied to each pair of sense/antisense genes in order to select two complementary 60-mer oligonucleotides from the region where the two genes overlap. The design criteria included the following: low cross-homology (up to 75%) to other expressed sequences in the human transcriptome; a continuous hit of no more than 17 bp to the sequence of another gene; balanced GC content (30-70%) without significant windows of local imbalance; no more than 2 palindromes with a length of 6 bp; a hit of no more than 15 bp to a repeat, vector or low-complexity region; and no long stretches of identical nucleotides.

Microarray preparation—60-mer oligonucleotides were synthesized by Sigma-Genosys (The Woodlands, Tex.), resuspended at 40 μM in 3×SSC, and spotted in quadruplicates on poly-L-lysine coated glass slides as detailed in the online protocol of the National Human Genome Research Institute (http://www.nhgri.nih.gov/DIR/Microarray/Protocols.pdf). To avoid local differences in the hybridization conditions, the probes selected from the overlapping regions of each sense/antisense pair were spotted in the same block, next to each other.

Human cell lines—The following cell lines utilized were purchased from ATCC (Manassas, Va.): MCF7 (breast adenocarcinoma, Cat. No. HTB-22,), HeLa (cervical adenocarcinoma, Cat. No. CCL-2) HEK-293 (embryonal kidney cells, Cat. No. CRL-1573), Jurkat (acute T-cell leukemia, Cat. No. TIB-152), K-562 (chronic myelogenous leukemia, Cat. No. CCL-243), HepG2 (liver carcinoma, Cat. No. HB-8065), T24 (urinary bladder carcinoma, Cat. No. HTB-4), SK-N-DZ (neuroblastoma, Cat. No. CRL-2149), NK-92 (non-Hodgkin's lymphoma, Cat. No. CRL-2407), MG-63 (osteosarcoma, Cat. No. CRL-1427), DU 145 (prostatic carcinoma, Cat. No. HTB-81), G-361 (melanoma, Cat. No. CRL-1424), PANC-1 (pancreatic carcinoma, Cat. No. CRL-1469), ES-2 (ovary clear cell carcinoma, Cat. No. CRL-1978), Y79 (retinoblastoma, Cat. No. HTB-18), HT-29 (colorectal adenocarcinoma, Cat. No. HTB-38), H1299 (large cell lung carcinoma, Cat. No. CRL-5803), SNU1 (gastric carcinoma, Cat. No. CRL-5971), NL564 (EBV-transformed human lymphoblasts) and MCF10 (benign tumor breast cells).

RNA purification—Total RNA was extracted from the above mentioned human cell lines using TriReagent (Molecular Research Center, Cincinnati, Ohio). Poly(A)+ mRNA was purified using two cycles of the Dynabeads mRNA Purification Kit (Dynal Biotech ASA, Oslo, Norway), as per manufacturer instructions. The removal of traces of ribosomal RNA was confirmed by agarose gel electrophoresis. Poly(A)+ mRNAs from human testis, placenta, lung and brain tissue were purchased from BioChain Institute, Inc. (Hayward, Calif.). mRNAs of all cell lines described above were combined in equal quantities to obtain the reference ‘mRNA pool’.

Preparation of labeled cDNA—For each hybridization, labeled cDNA was synthesized by reverse transcription of 0.5 μg of mRNA, in the presence of 100 pmol of random 9-mers, 1 μg of oligo(dT)20, 1×RT buffer, 10 mM DTT, 3 nmol of Cy5- or Cy3-conjugated dUTP, 0.5 mM of dATP, dGTP and dCTP, and 0.2 mM dTTP, in a final volume of 40 μl (Amersham). The reaction mixture was incubated for 5 minutes at 65° C. and cooled to 42° C. 600 Units of reverse transcriptase (Superscript II, Invitrogen, Carlsbad, Calif.) and 40 U of Rnase inhibitor (RNasin Promega, Madison, Wis.) were added and the reaction was incubated for 30 minutes at 42° C. An additional 200 U of Superscript II were added and the reaction was incubated for another 15 minutes. Remaining RNA was degraded by the addition of 200 mM NaOH and 50 mM EDTA, at 65° C. for 10 minutes. The mixture was neutralized by adding half a volume of 1M Tris-HCl pH 7.5. Hybridizations were performed in duplicate using fluorescent reversal of Cy3- and Cy5-labeled cDNA from test cell mRNAs and pooled mRNAs. Pairs of Cy5/Cy3-labeled cDNA samples were combined, and subsequently purified and concentrated to a final volume of 5-7 μl using a Microcon-30 (Millipore) concentrator.

Hybridization and washing conditions—Microarray slides were prehybridized with 40 μl of 5×SSC, 0.1% SDS and 1% BSA for 30 min at 42° C., washed for 2 minutes with double distilled water, then rinsed with isopropanol, and spun dried at 500 g for 3 minutes. Prior to hybridization, the labeled probe was combined with 10 μg of Cot-1 DNA, 10 μg poly(dA)80, and 4 μg yeast tRNA, in a final volume of 15 μl. The mixture was denatured at 100° C. for 3 minutes and placed on ice. Formamide (final concentration 16%), SSC (to 5× concentration) and 0.1% SDS were added to a final volume of 30 μl. The mixture was placed on the array under a glass cover slip in a tightly sealed hybridization chamber, and immersed in a water bath at 42° C., for 16 hours. Microarray slides were then washed for 4 minutes with 2×SSC, 0.1% SDS; 4 minutes with 1×SSC, 0.01% SDS; 4 minutes with 0.2×SSC and 15 seconds with 0.05×SSC and spun dry by centrifugation for 3 minutes at 500 g.

Image processing—Following hybridization, arrays were scanned using a GenePix 4000B scanner (Axon Instruments, Union City, Calif.). Scanned array images were manually inspected and areas with visible artifacts or deformities were marked. Images were processed using GenePix Pro 3.0 (www.axon.com) software.

Normalization—The intensity for each spot was calculated as its mean intensity minus the median background around the spot. The signal for each oligo was calculated as the average of intensity values of the four redundant spots of each oligo. Normalization of the oligo signals was performed at several levels as is further described below.

Normalization of blocks was carried out in order to normalize the gradient of intensities within each slide. For each block i, an Ai parameter was calculated as the average of intensities of 56 positive control spots (oligonucleotide probes for the ubiquitously expressed housekeeping genes gapdh, actin, hsp70 and gnb211, in various probe concentrations). An average A of all Ai averages was calculated. Based on this, a block normalization factor Bi was calculated for each block, as Bi=A/Ai, and applied to each spot in the block.

Normalization between slides was performed to bring all experiments to the same scale. For each experiment, the average of intensities of the 192 negative control spots on the array was set to be the 0 (zero) of the new scale. For a subset of highly signaling oligos, with intensities between the 70th and the 95th percentiles of the oligo signal distribution of the experiment, the average was arbitrarily set to be 500 in the new scale. The intensity of each oligo signal was accordingly converted to this new scale, to obtain the normalized signal. A ratio between the normalized cell-line signal and the normalized pool signal was calculated for each oligo in each experiment. To avoid misleading ratios coming from signals that were too low, the ratio Rji for oligo j in experiment i was calculated as: Rji=max[100, cell-line-signalji]/max[100, pool-signalji].

To normalize between red/green intensities in reciprocal experiments, the ratio Rjk for oligo j in cell-line k was calculated as the average of calculated ratios Rji between the two reciprocal experiments of the cell-line k. In cases where only one of the two reciprocal experiments showed an elevated or decreased ratio, while in the other the ratio was 1.0, the average Rjk was converted to 1.0.

The actual pool signal for each oligo was calculated to be the average of the normalized oligo signals in the pool channel of all experiments. A virtual pool signal was calculated as the average of the normalized oligo signals in the cell-line channel of all experiments. The virtual pool signals were found to be very close to the actual pool signals, indicating consistency in the analysis.

Threshold determination—To determine an expression threshold above, in which a normalized signal would be considered a ‘positive’ signal indicating expression, the distribution of all 16,512 normalized negative control signals and the standard deviation (neg-std-dev) were calculated. The neg-std-dev obtained was 38. An oligo j was considered ‘present’ in a cell-line k if Rjk×actual-pool-signalj≧4×neg-std-dev.

### Example 1

### Identification of 53BP1 and 76P RNA Transcripts in a Variety of Human Tissues and Cell-Lines

Background:

The tumor suppressor p53 binding protein 1 (SEQ ID NO: 15) is one of the various p53 target proteins. It binds to the DNA-binding domain of p53 and enhances p53-mediated transcriptional activation. 53BP1 is characterized by several structural motifs shared by several proteins involved in DNA repair and/or DNA damage-signaling pathways. 53BP1 becomes hyperphosphorylated and forms discrete nuclear foci in response to DNA damage induced by radiation and chemotherapy. Recent reports suggest that 53BP1 is an ataxia telangiectasia mutated (ATM) substrate that is involved early in the DNA damage-signaling pathways in mammalian cells, attributing a role to 53BP1 in the development of various mammalian pathologies.

Results:

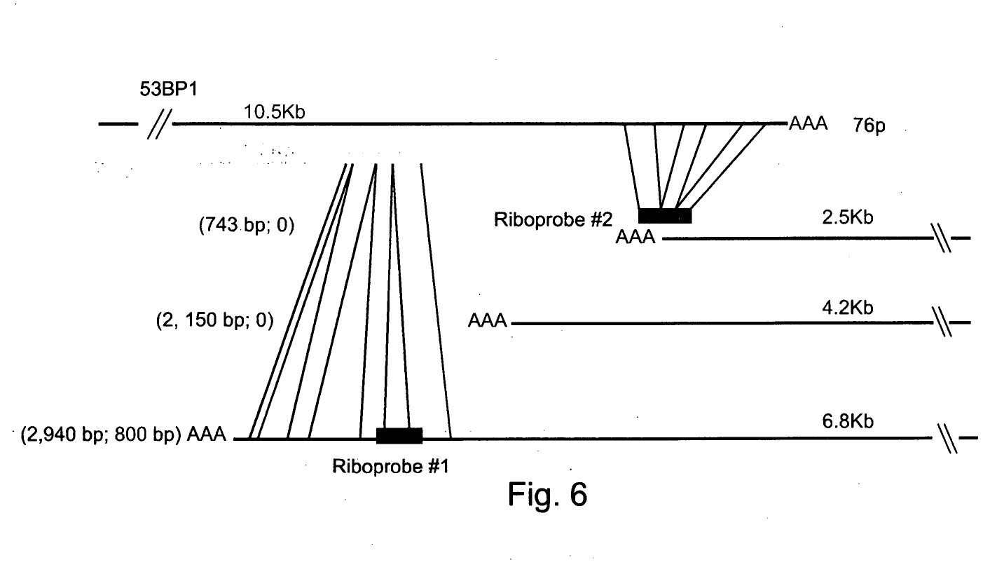

Two 53BP1 RNA sense transcripts with dissimilar 3′ UTRs were previously described [Iwabuchi K. et al. (1994) Proc. Natl. Acad. Sci. USA] and are illustrated in FIG. 6 (red and green). Leads™ assembly program modified to uncover novel antisense transcripts was used to uncover three such transcripts for the 53BP1 gene, which transcripts have different 3′ UTRs (SEQ ID NO: 16, 37 and 38) and encode the 76p gene product (Genbank accession number NM014444, illustrated in blue).

To confirm expression of computationally retrieved antisense transcripts, two RNA-probes were generated. Schematic location of the probes used for sense and antisense validation (Riboprobe#1 and Riboprobe#2, respectively SEQ ID NO: 17 and 18, respectively) is illustrated in FIG. 6. These RNA probes were used to identify the corresponding full-length transcripts.

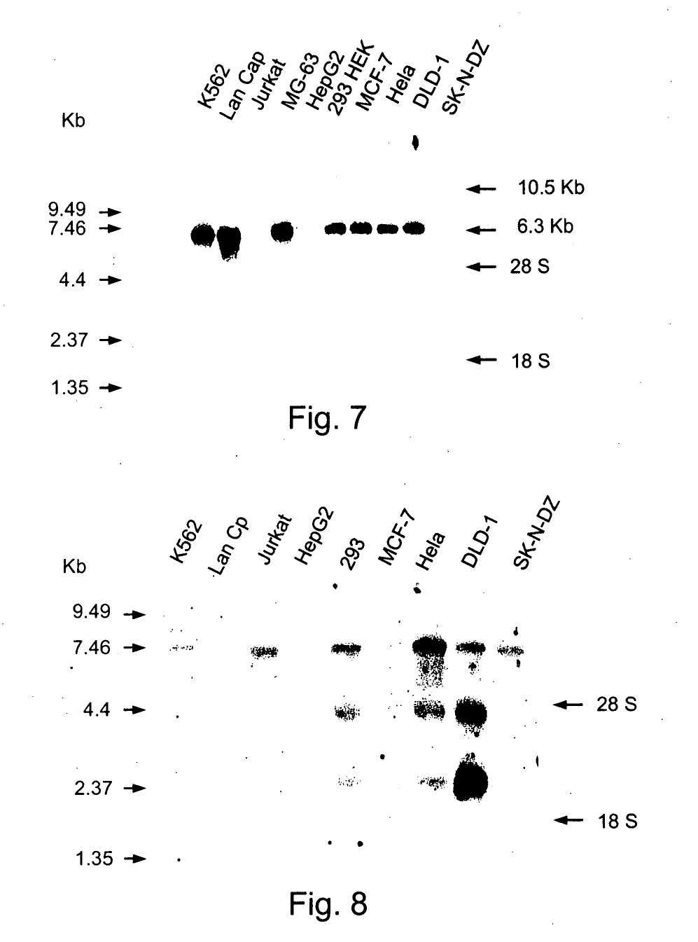

As shown in FIG. 7, Riboprobe#1 detected two transcripts of approximately 6.3 Kb and 10.5 Kb, corresponding to the sense mRNA. The absolute levels of the short messenger were rather homogeneous in all cell-lines examined. The 10.5 Kb variant exhibited a more heterogenic pattern of cellular distribution, and was mostly expressed in K562, MG-63, 293 HEK and Hela cells. In general, the longer sense transcript which is an alternatively polyadenylated variant was markedly lower expressed in the various cell lines examined.

The same membrane was used to perform northern analysis with Riboprobe#2 in order to validate expression of antisense transcripts of 53BP1. Results are shown in FIG. 8. Three variants corresponding to the 76p gene were detected in most of the cell lines: 6.8 Kb, 4.2 Kb and 2.5 Kb. Minor fluctuations of expression were observed and the largest transcript was expressed at significantly higher levels than the smaller transcripts.

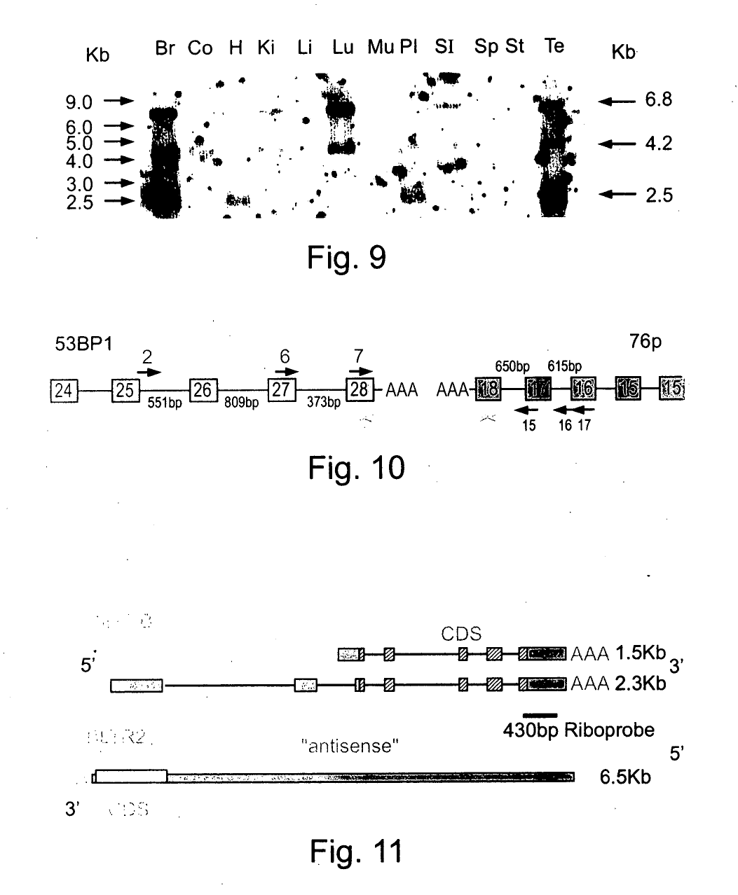

A sense strand probe was used to detect expression of the antisense transcripts in a variety of human tissues (FIG. 9). The three alternatively polyadenylated variants with different 3′ UTRs were expressed in most of the tissues. Total levels of these transcripts varied in the different tissues assayed. For example, highest level of expression for all three transcripts was observed in the brain and testis, while no expression of the 6.8 Kb and 4.2 Kb variants was detected in the spleen. Expression levels of each transcript were summarized in Table 2 below.

TABLE 2

| | Transcript Mol. Weight (Kb) | | |
|---|---|---|---|
| Tissue | 6.8 | 4.2 | 2.5 |
| brain | +++ | ++++ | ++++ |
| colon | + | ++ | + |
| heart | − | + | ++ |
| kidney | ++ | ++ | + |
| Liver | − | − | + |
| lung | ++++ | +++ | + |
| muscle | ++ | + | + |
| placenta | + | ++ | ++ |
| Small intestine. | ++ | ++ | − |
| spleen | − | − | + |
| stomach | − | − | + |
| testis | ++ | ++ | ++++ |

Reverse transcription amplification (RT-PCR) analysis was performed in order to substantiate the northern blot results. Primers were synthesized according to the scheme shown in FIG. 10 (indicated by arrows). The expected amplification products corresponded completely to the observed amplification reaction products, supporting the existence of the various 53BP1 and 76p transcription variants.

### Example 2

### Identification of mRNA and Complementary Transcripts of the Cell Death Inducing DFF45-Like Effector (CIDE)-B

Background:

Cell death inducing DFF45-like effector (CIDE-B) (GenBank Accession numbers AF190901 and AF218586) is a member of a novel family of apoptosis-inducing factors that share homology with the N-terminal region of DFF, the DNA fragmentation factor. Although the molecular mechanism of CIDE-B induced apoptosis in unclear, mitochondrial localization and dimerization, both where shown to be required [Chen Z. et al. (2000) J. Biol. Chem. 275:22619-22622]. Notably, over-expression of CIDE-B in mammalian cells shows strong cell death-inducing activity, suggesting that aberrant expression of this protein may be associated with a number of mammalian pathologies [Inohara N. et al. (1998) EMBO J. 17:2526-2533].

Results:

Two sense transcript of the CIDE-B gene were previously described with different 5′ UTRs [Inohara N. et al. (1998) EMBO J. 17:2526-2533 and Lugovskoy A A. et al. (1999) Cell 99:745-755] (SEQ ID NOs: 19 and 20). Computational analysis recovered a potential elongated BLTR2 transcript (SEQ ID NO: 21), showing full complementary to the CIDE-B mRNA transcripts (FIG. 11).

Northern blot analysis was done in order to determine the distribution of the CIDE-B sense and antisense transcripts in various cell-lines. A 430 base pairs DNA fragment was selected to generate RNA probes for identification of both sense and antisense transcripts (SEQ ID NOs: 22 and 23, respectively).

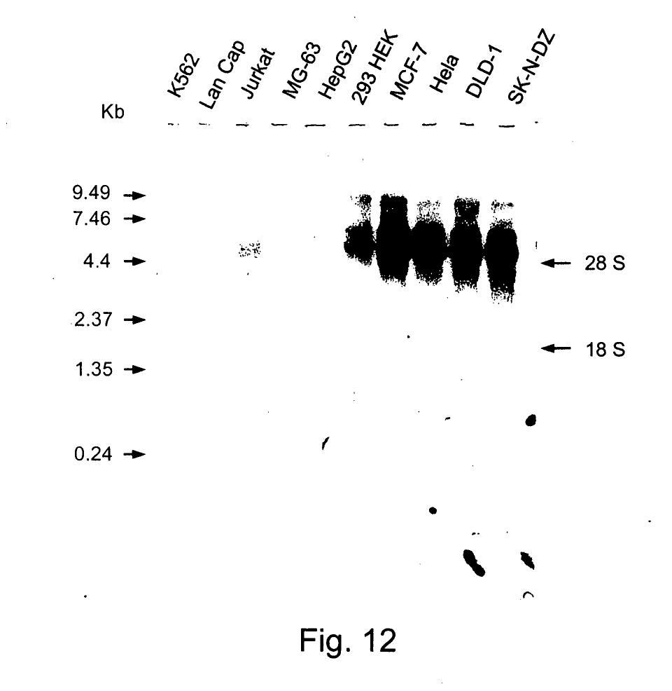

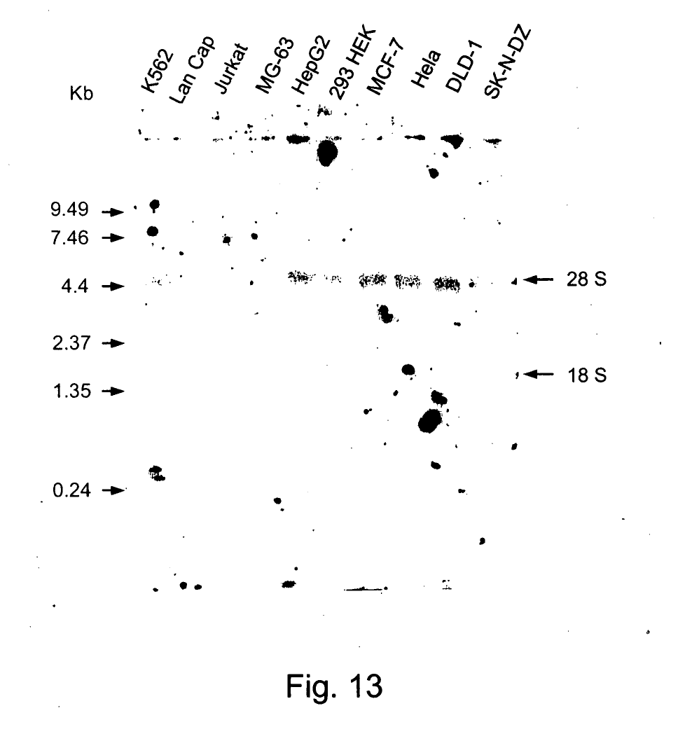

Expression of antisense mRNA transcripts was detected in various cell-lines and especially in the mammary gland adenocarcinome cell line-MCF-7 as a predominant 6.5 Kb transcript, although higher forms were also visualized (FIG. 12). Low hybridization with a CIDE-B probe was detected (FIG. 13).

Conclusion:

BLTR2 was recently identified as a putative seven-transmembrane receptor with a high homology to the Leukotriene B (4) receptor [Tryselius Y. et al. (2000) Biochem. Biophys. Res. Commun. 274:377-82]. Although the mechanism of action of BLTR2 is poorly understood, it is conceivable that BLTR2 mRNA plays a role in the regulation of CIDE-B apoptotic effector and vice versa.

### Example 3

### Identification of mRNA and Complementary Transcripts of the Apoptosis Inducing Factor APAF-1

Background:

A conserved series of events including cellular shrinkage, nuclear condensation, externalization of plasma membrane phosphatidyl serine, and oligonucleosomal DNA fragmentation characterizes apoptotic cell death. Regardless of the circumstance, induction and execution of apoptotic events require activation of caspases, a family of aspartate-specific cysteine proteinases. Caspase activation may be regulated by the mitochondrion and specifically by the apoptosome consisting of an oligomeric complex of apoptotic protease-activating factor-1 (APAF-1), cytochrome C and dATP. The apoptosome recruits and activates caspase-9, which in turn activates the executioner caspases, caspase-3 and -7. The active executioners kill the cell by proteolysis of key cellular substrates [Zou H. et al. (1999) J. Biol. Chem. 274:11549-11556]. Evasion or inactivation of the mitochondrial apoptosis pathway may contribute to oncogenesis by allowing cell proliferation. In this instance, unregulated cell proliferation may occur by inactivation of APAF-1, which has been suggested to occur via genetic loss or inhibition by HSP-70 and HSP-90. Although aberrant expression of APAF-1 was found in a variety of malignancies (including ovarian epithelial cancer), no link was found to accelerated protein degradation.

Results:

One RNA transcript has been previously described for APAF-1 [Zou H. et al. (1999) J. Biol. Chem. 274:11549-11556] (SEQ ID NO: 10) (SEQ ID NO: 24). Computational search for natural antisense transcripts has revealed two complementary transcripts for APAF-1 messenger RNA (SEQ ID NOs: 25 and 26). These antisense transcripts include an open reading frame encoding the EB-1 gene (GenBank accession numbers AF145204; AF164792). The overlap between the APAF-1 messenger RNA and the longer antisense transcript is of at least 300 nucleotides.

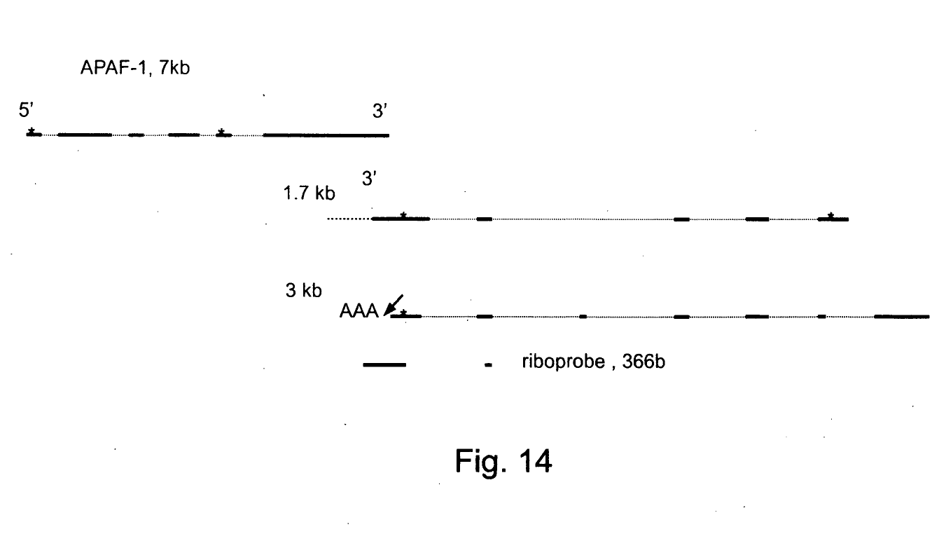

To validate expression of computationally retrieved antisense transcripts for APAF-1, as well as expression of APAF-1 mRNA in the assayed human cell lines, RNA-probes of 366 ribonucleotides were generated (sense and antisense strands, respectively). Schematic location of the probes used for sense and antisense validation (Riboprobe#1 and Riboprobe#2, SEQ ID NOs: 27 and 28, respectively) is illustrated in FIG. 14.

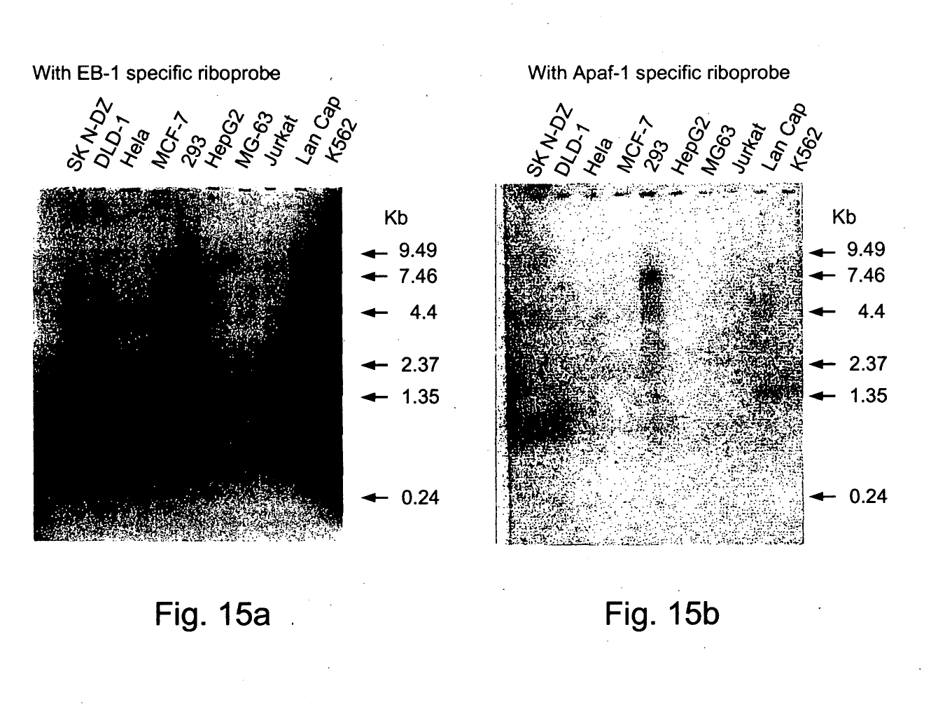

As shown in FIG. 15a, the sense RNA probe directed at visualizing the antisense transcripts, identified a clear band of 3 Kb corresponding to the long computationally retrieved antisense transcript as well as other transcripts sizing from 1 Kb to 8 Kb (FIG. 15a). Transcripts were essentially found in all cell lines but especially in 293 HEK and LN-Cap lines.

Hybridization with an RNA probe directed at visualizing the mRNA transcript of APAF-1 resulted only in a blurred patterns (FIG. 15b). However, a 7 Kb mRNA transcript consistent with APAF-1 mRNA was seen in Ln Cap and 293 HEK cell lines.

Conclusion:

A reciprocal pattern of expression was observed for both APAF-1 and EB-1 transcripts, exhibiting an interesting expressional relationship between the sense and antisense transcripts suggesting antisense-mediated expression regulation.

### Example 4

### mRNA Expression of Muscle Nicotinic Acetyl-Choline Receptor ε Subunit and its Complementary MINK Transcript

Background:

The muscle nicotinic Acetylcholine Receptor ε subunit (AChRε) encodes for one of five subunits of a ligand gated ion channel receptor located at the neuromuscular synapse. AChRε is up-regulated in the postnatal period when it replaces γ subunit of the receptor [Witzamann, V. et al., (1987) FEBS Lett. 223, 104-112]. It is also up-regulated in synapse development, specifically by the trophic factor neuregulin [Martinou J. C. (1991) Pro. Natl. Acad. Sci. USA 88, 7669-7673]. In an attempt to decipher AchRε function and mechanism of regulation, computational screen for AChRε K complementary transcript was carried out.

Results:

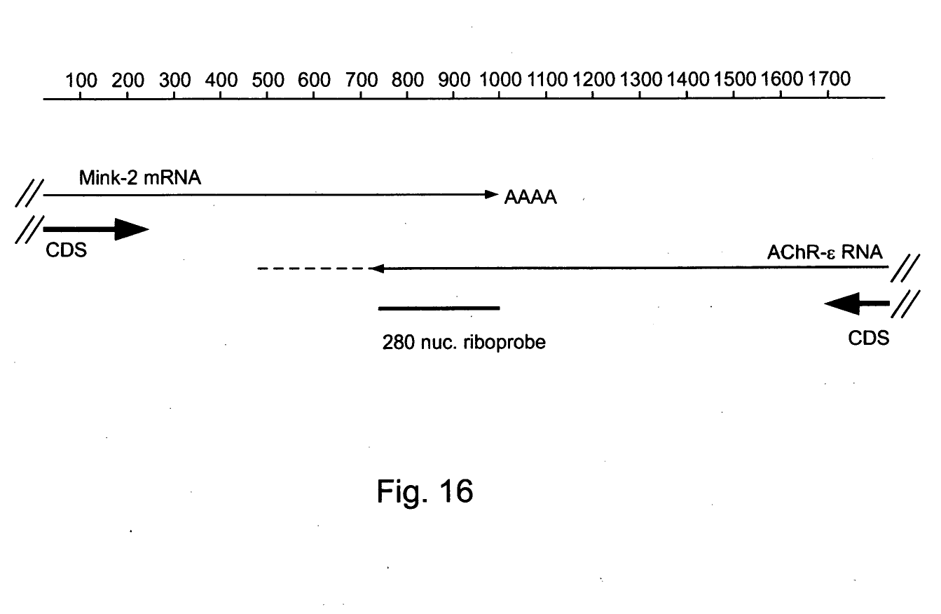

One mRNA transcript of AChRε gene was previously described [Beeson D. Eur. J. Biochem (1993) 215, 229-238] (SEQ ID NO: 29). Computational analysis recovered a complementary transcript belonging to Mink, a new member of the germinal center kinase (GCK) family (SEQ ID NO: 30) [Dan I. FEBS Lett. (2000) 469, 19-23] showing an overlap of at least 280 nucleotides to the AchRε mRNA, as schematically illustrated in FIG. 16.

To validate the overlap of the two genes and to learn about their tissue distribution, northern analysis of a variety of human tissues was performed. Poly(A)—RNA containing membrane was hybridized with a 280 nucleotides RNA probes, corresponding to the overlap region in either antisense or sense orientation (SEQ ID NOs: 31 and 32, respectively).

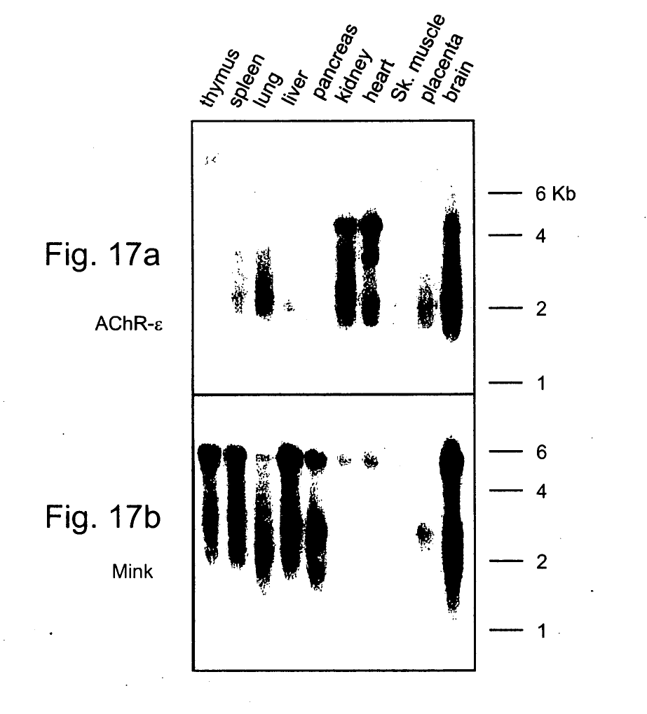

As is evident from FIG. 17a an AChRε transcript was expressed as a predominant 4 Kb band and had the highest expression in the heart, kidney and brain while surprisingly only a limited expression was observed in the skeletal muscle.

Hybridization with a MINK specific RNA probe revealed a major transcript of about 5 Kb, in accordance with previous results [Dan I. FEBS Lett. (2000) 469, 19-23] (FIG. 17b). The mRNA transcript was ubiquitously expressed with strongest expression found in brain, liver, thymus, spleen and pancreas, again in agreement with Dan I. et al.

Conclusion:

The finding that AChRε and Mink genes are antisense each to one another with a significant overlap, and the fact that the two genes are co-expressed in some tissues (eg., brain) suggest the possibility that one of them may regulate the other under certain conditions.

### Example 5

### Expression of Cyclin E2 mRNA and Complementary Transcripts in a Variety of Human Cell-Lines

Background:

The human cyclin E2 gene encodes a 404-amino-acid protein that is most closely related to cyclin E. Cyclin E2 associates with Cdk2 in a functional kinase complex that is inhibited by both p27(Kip1) and p21(Cip1). The catalytic activity associated with cyclin E2 complexes is cell cycle regulated and peaks at the G1/S transition. Overexpression of cyclin E2 in mammalian cells accelerates cell-cycle progression. Unlike cyclin E1, cyclin E2 levels are low to undetectable in nontransformed cells and increase significantly in tumor-derived cells suggesting specific mechanism of regulation.

Results:

One RNA transcript was found for cyclin E2 (SEQ ID NO: 33. Computational search for natural antisense transcripts has revealed one complementary transcript for cyclin E2 messenger RNA (SEQ ID NO: 34). The overlap between the cyclin E2 sense RNA and the antisense transcript is of at least 72 nucleotides.

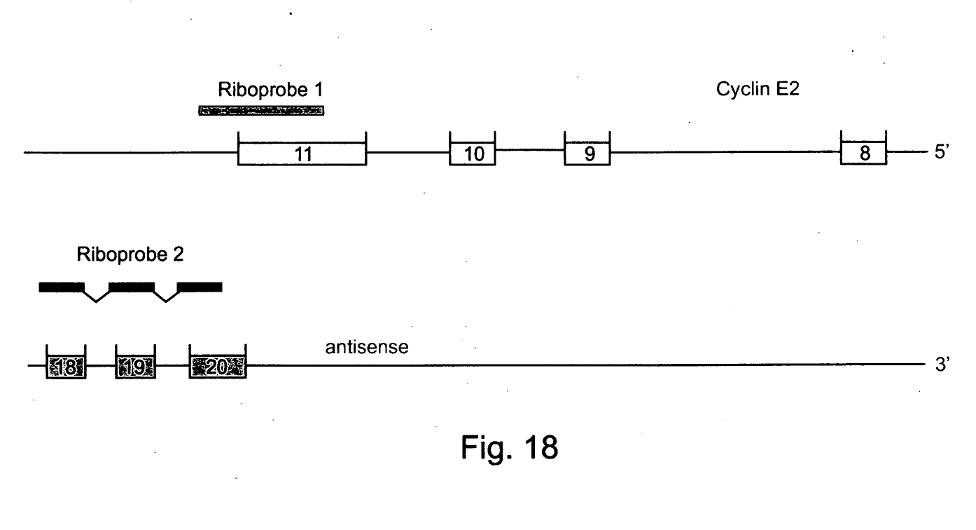

To confirm expression of the computationally retrieved antisense transcript for cyclin E2 as well as of cyclin E2 mRNA in human cell lines, two RNA-probes of 800 ribonucleotides were generated. Schematic location of the probes used for sense and antisense validation (SEQ ID NO: 44, Riboprobe#1 is illustrated in FIG. 18).

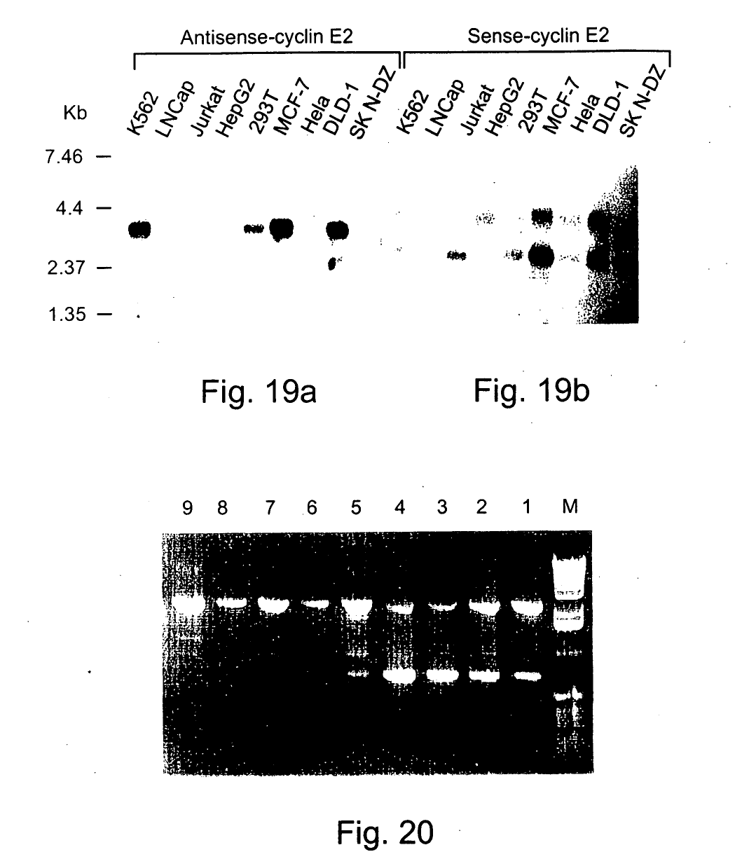

As shown in FIG. 19a, Riboprobe#1 detected two transcripts of approximately 3 Kb and 4.3 Kb. The absolute levels of the transcripts were quite heterogenic in all cell-lines examined. Both transcripts were completely absent from the Ln Cap cell line, while significantly high expression was observed in MCF-7 and DLD-1 lines, especially of the short transcript.

The same membrane was used to perform northern analysis with Riboprobe#2 in order to validate expression of antisense transcripts of cyclin E2. As is evident from FIG. 19b, an antisense transcript 3.8 Kb long was observed in most cells assayed. Significantly high pattern of expression was observed in K562, MCF-7 and DLD-1 cell lines, while only a very moderate level of expression was detected in Ln Cap and HepG2 cell lines.

### Example 6

### Co-Regulated Expression of CIDE-B and its Complementary Transcript upon Induction of Apoptosis

The discovery of a novel naturally occurring antisense transcript to the apoptosis inducing factor, CIDE-B (see Example 2 hereinabove), suggested that the latter may be regulated by its complementary transcript, thereby establishing a novel mechanism of regulation. To address this, differential expression analysis of CIDE-B expression and its endogenous antisense transcript expression was performed following induction of apoptosis.

Materials and Methods

Induction of Apoptosis and Reverse Transcription Analysis—

Monolayers of 293 cells were either left untreated (UT) or incubated with increasing concentrations of etoposide or staurosporine (Sigma IL). Twenty-four hours following addition of the drug, total RNA was extracted as decribed hereinabove. Purified RNA was further treated with DNaseI. A reverse transcription reaction were carried out with equivalent amounts of RNA in a final volume of 20 μl containing 100 pmol of the oligo(dT) primer, 250 ng of total RNA, 0.5 mM each of four deoxynucleoside triphosphates and 5 units of reverse transcriptase. The reaction mixture was incubated at 65° C. for 5 min, 42° C. for 50 min and 70° C. for 15 min. PCR was carried out in a final volume of 25 μl containing 12.5 pmol each of the oligonucleotide primers derived of exons 3 and 7 of CIDE-B (SEQ ID NOs: 39 and 40), 1 μl of RT solution and 1.75 units of Taq polymerase. Amplification was carried out by an initial denaturation step at 94° C. for 5 min followed by 35 cycles of [94° C. for 30 s, 68° C. for 30 s, and 68° C. for 130 min]. At the end of the PCR amplification, products were analyzed on agarose gels stained with ethidium bromide and visualized with UV light.

Results

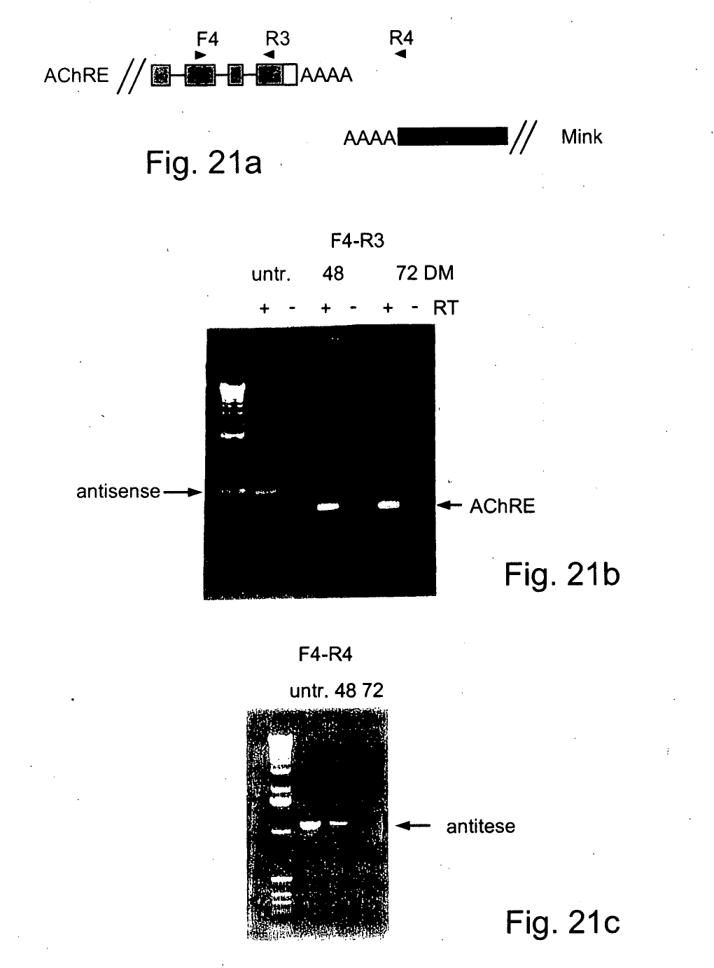

Amplification reaction yielded two major PCR products of 740 bp and 2285 bp (FIG. 20). The small (740 bp) PCR product derived from the sense (CIDE-B) strand, whereas the larger (2285 bp) product represented an intronless antisense transcript. Evidently, an increase of sense transcript, concomitant with a decrease of antisense transcript, was observed following treatment with etoposide (lanes 1-4) as compared to untreated cells (lane 9), while no change was detected following staurosporine treatment (lanes 5-8).

These results suggest that following induction of apoptosis, antisense regulation of CIDE-B is abolished thereby allowing CIDE-B mediated apoptosis to proceed.

### Example 7

### Reciprocal Variation in Sense and Antisense Expression of Mouse Nicotinic Acetylcholine Receptor, Epsilon Subunit During Differentiation

The mouse nicotinic acetylcholine receptor, epsilon (mAchRε) subunit (SEQ ID NO: 35) has a critical function in a variety of differentiation processes. To address a novel concept of antisense regulation of AchRε-mediated differentiation, expression patterns of AchRε and its naturally occurring antisense transcript (SEQ ID NO: 36) were examined following induction of differentiation.

Materials and Methods

Induction of apoptosis and reverse transcription analysis—C2 mouse myoblast cells were incubated with a differentiation medium (Dulbecco's modified Eagle's medium (DMEM) including 10 μg/ml insulin and 10 μg/ml transferring) or control medium (untreated) for 48 and 72 hours. Total RNA was extracted from treated and control cells and reverse-transcribed. PCR was done using F4 and R3 primers, derived from exon numbers 10 and 12 (last exon, SEQ ID NOs: 41 and 42, respectively) of the mouse nicotinic acetylcholine receptor, epsilon subunit (mAChRε) and directed at detecting sense and antisense transcripts (see FIG. 21a).

Results

Amplification reaction showed a gradual increase in AchRε transcript expression, concomitant with the differentiation state of the cells. A second amplification product, which corresponded to an unspliced transcript was seen in untreated cells and disappeared following induction of differentiation. This fragment corresponds to a putative antisense transcript of the AchRε, and may represent an alternative 3′ UTR of the Mink gene, of which the known transcript terminates 400 bp downstream to AchRε (see Example 4). To overcome possible competition between the two transcripts, another PCR reaction was carried out using antisense specific riboprobes F4 and R4 (SEQ ID NO: 43). Reverse transcription products of this amlification reaction showed a single band which corresponded to a naturally occurring antisense transcript of the AchRε. As expected this transcript disappeared following induction of differentiation.

These results imply inverse regulation of the AchRε and its naturally occurring antisense transcript, during muscle cells differentiation from myoblasts to myotubes. Regulation may proceed, possibly through complementation of the sense and antisense transcripts to form dsRNA which can serve as a substrate for double strand RNA processing enzymes such as RNase H.

### Example 8

### A Polynucleotide Database of Sequences Corresponding to the Naturally Occurring Antisense Transcripts Identified by the Present Invention and Their Complementary Sense Sequences

Naturally occurring antisense sequences identified according to the teachings of the present invention and their corresponding sense sequences are provided in the CD-ROM1-4 enclosed herewith (CD content is further described hereinbelow). Generally a “seqs” text file contains the actual polynucleotide sequences; a “table” file contains summarized data pertaining to each sense-antisense sequence pair; an “alignments” file contains sequence alignments of sense and antisense overlapping regions; an “orthology” file contains a table depicting the connection between gene loci which were found to be sense-antisense pairs in the mouse genome and their human orthologous loci.

All analyses (excluding orthology which was performed only on GenBank version 136) were performed on GenBank version 136, 133 and 125, as follows.

Version 136

9 files: table_{—}136, nuc_seqs_{—}136, pep_seqs_{—}136, annotations_{—}136, alignments_{—}136, mouse_table, mouse_seqs, mouse_alignments, orthology.
table_{—}136 is a list of 153; 813 pairs of transcripts representing 6850 pairs of contigs.
Numbering: m_n
m—contigs' pair number.
n—number of transcripts' pair that belongs to a pair of contigs.
(each pair of contigs is represented by one or more pairs of transcripts)
nuc_seqs_{—}136 contains 83,304 sequences of all the transcripts, numbered according to the list in table_{—}136.
pep_seqs_{—}136 contains 45,628 sequences of all the proteins encoded by the transcripts.
alignments_{—}136 contains the alignment of each pair of overlapping transcripts—153,813 alignments.
annotations_{—}136 contains all the annotations for each of the protein coding transcripts as described below.
mouse_table is a list of 17,290 pairs of transcripts representing 444 pairs of contigs.
Numbering: m_n
m—contigs' pair number.
n—number of transcripts' pair that belongs to a pair of contigs.
(each pair of contigs is represented by one or more pairs of transcripts)
mouse_seqs contains 8,653 sequences of all the transcripts, ordered by pairs and numbered according to the list in mouse_table.
Mouse_alignments contains the alignment of each pair of overlapping transcripts—17,290 alignments.
orthology is a table with 444 lines that link between loci in that was found to be an antisense pair in mouse and their human orthologous loci in the following format—
#S_MUS_LOC—sense mouse locus
#S_MUS_CN—sense mouse contig
#AS_MUS_LOC—antisense mouse locus
#AS_MUS_CN—antisense mouse contig
#S_HUM_LOC—sense human locus
#S_HUM_CN—sense human contig
#AS_HUM_LOC—antisense human locus
#AS_HUM_CN—antisense human contig
#RES—result of comparison to human as described below
   Version 133
3 files: table_{—}133, seqs_{—}133, alignments_{—}133.
table is a list of 175,644 pairs of transcripts representing 6230 pairs of contigs.
Numbering: m_n
m—contigs' pair number.
n—number of transcripts' pair that belongs to a pair of contigs.
(each pair of contigs is represented by one or more pairs of transcripts)
seqs contains 99,414 sequences of all the transcripts, ordered by pairs and numbered according to the list in table.
alignments contains the alignment of each pair of overlapping transcripts —175,644 alignments.
   Version 125
3 files: table_{—}125, seqs_{—}125, alignments_{—}125.
table is a list of 223,181 pairs of transcripts representing 4018 pairs of contigs.
Numbering: m_n
m—contigs' pair number.
n—number of transcripts' pair that belongs to a pair of contigs.
(each pair of contigs is represented by one or more pairs of transcripts)
seqs contains 79,884 sequences of all the transcripts, ordered by pairs and numbered according to the list in table.
alignments contains the alignment of each pair of overlapping transcripts —223,181 alignments.

“Table S1” and “Table S2” are further described in Example 9.

Table 3 below exemplifies the format of the Tables provided in CD-ROMs 2, 3 and 4. Each row represents a pair of transcripts. The columns of Table 3 represent (from the left): the serial number of the pair, the name of the first transcript, its length in nucleotides, the name of the second transcript, its length in nucleotides, the number of base pairs that overlap between the two transcripts, offsets of overlap beginning at the first transcript, offsets of overlap beginning at the second transcript.

TABLE 3

| | | | | | | Start of overlap |
|---|---|---|---|---|---|---|
| | | First | | Second | Overlap | in first/ |
| Serial | First | transcript | Second | transcript | length | in second |
| No. | transcript | length (nt) | transcript | length (nt) | (nt) | transcript |
| 570_0 | AV705532_0 | 190 | Z44352_15 | 783 | OL: 52 | OF1: 1 OF2: 1 |
| | (SEQ ID NO: 1) | | (SEQ ID NO: 2) | | | |
| 570_1 | AV705532_0 | 190 | Z44352_14 | 1649 | OL: 52 | OF1: 1 OF2: 1 |
| | | | (SEQ ID NO: 3) | | | |
| 570_2 | AV705532_0 | 190 | Z44352_13 | 1861 | OL: 52 | OF1: 1 OF2: 1 |
| | | | (SEQ ID NO: 4) | | | |
| 571_0 | AW070860_0 | 214 | T81142_7 | 1934 | OL: 54 | OF1: 1 OF2: 1162 |
| | (SEQ ID NO: 5) | | (SEQ ID NO: 6) | | | |
| 571_1 | AW070860_0 | 214 | T81142_6 | 2353 | OL: 54 | OF1: 1 OF2: 1162 |
| | | | (SEQ ID NO: 7) | | | |
| 571_2 | AW070860_0 | 214 | T81142_4 | 2500 | OL: 54 | OF1: 1 OF2: 1264 |
| | | | (SEQ ID NO: 8) | | | |
| 571_3 | AW070860_0 | 214 | T81142_3 | 947 | OL: 54 | OF1: 1 OF2: 171 |
| | | | (SEQ ID NO: 9) | | | |
| 571_4 | AW070860_0 | 214 | T81142_2 | 1366 | OL: 54 | OF1: 1 OF2: 171 |
| | | | (SEQ ID NO: 10) | | | |
| 572_0 | BE046369_0 | 422 | W26553_3 | 1532 | OL: 52 | OF1: 1 OF2: 1532 |
| | (SEQ ID NO: 11) | | (SEQ ID NO: 12) | | | |
| 572_1 | BE046369_0 | 422 | W26553_2 | 1753 | OL: 52 | OF1: 1 OF2: 1753 |
| | | | (SEQ ID NO: 13) | | | |
| 572_2 | BE046369_0 | 422 | W26553_1 | 1832 | OL: 52 | OF1: 1 OF2: 1832 |
| | | | (SEQ ID NO: 14) | | | |

Pairs of transcripts are numbered, (within a contig pair, right to the underscore) that belong to a pair of contigs (numbered left to the underscore).
Transcript names are arbitrary designataions.

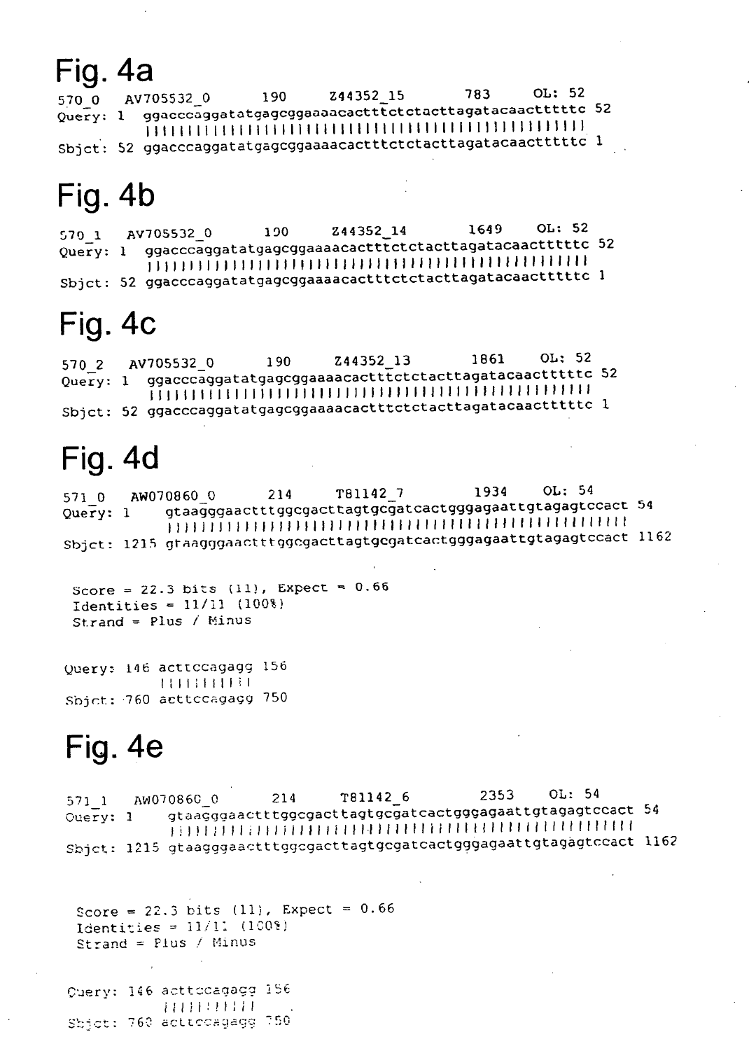

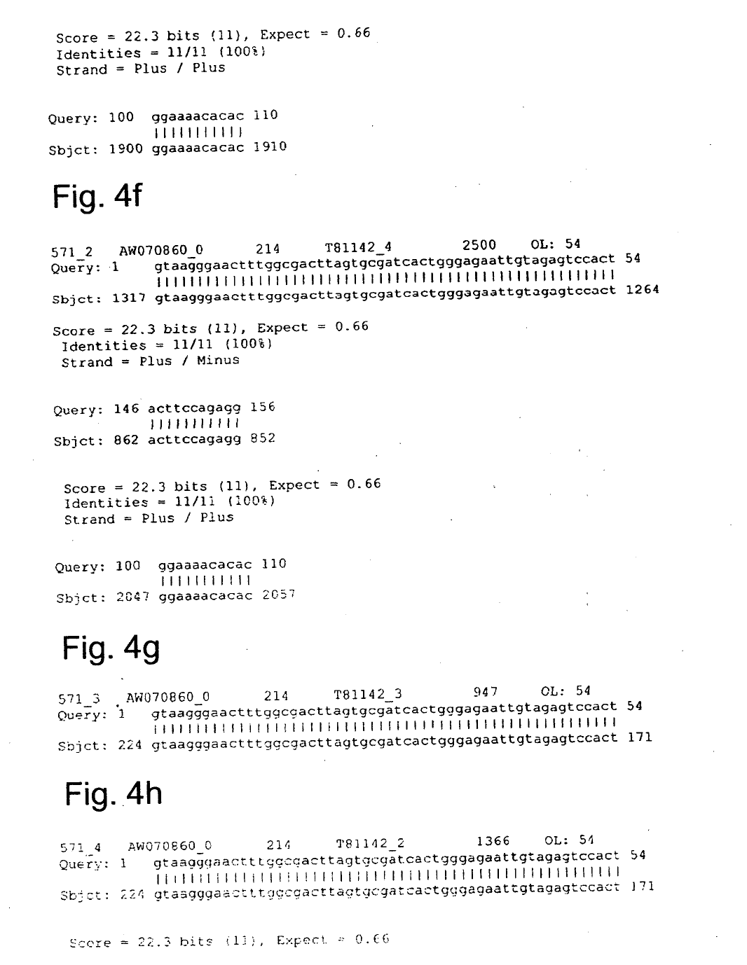

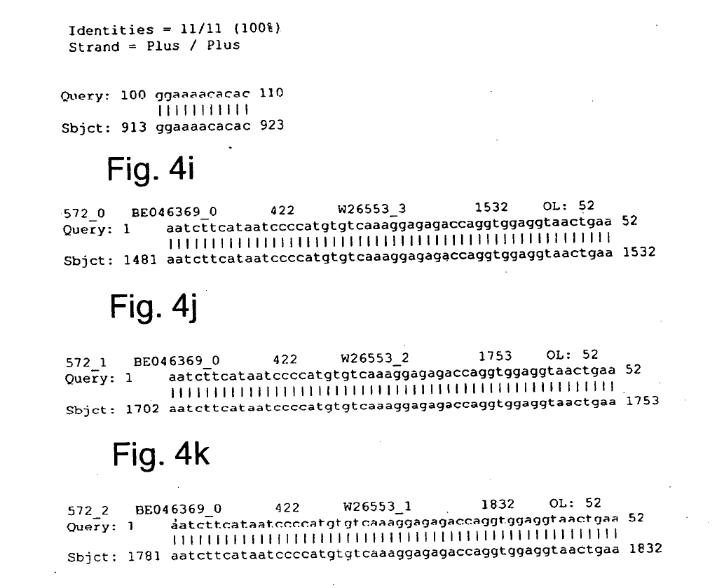

Sequence alignment of the overlapping region in each sense and antisense pair of Table 1 is demonstrated in FIG. 4a-k. Alignments were performed using the BLAST sequence alignment algorithm (Basic Local Alignment Search Tool, available through www.ncbi.nlm.nih.gov/BLAST). Interestingly, alignment profile shows high level of variability with regard to overlap lengths. It is conceivable that short overlaps are due to technical reasons associated with insufficient sequence data.

The putative naturally occurring antisense transcripts identified by the present invention and disclosed in the enclosed CD-ROMs can be used to detect and/or treat a variety of diseases, disorders or conditions, examples of which are listed hereinunder. For example, antisense transcripts or sequence information derived therefrom can be used to construct microarray kits (described in details in the preferred embodiments section) dedicated to diagnosing specific diseases, disorders or conditions.

The following sections list examples of proteins (subsection i), based on their molecular function, which participate in variety of diseases (listed in subsection ii), which diseases can be diagnosed/treated using information derived from naturally occurring antisense transcripts such as those uncovered by the present invention.

The present invention is of biomolecular sequences, which can be classified to functional groups based on known activity of homologous sequences. This functional group classification, allows the identification of diseases and conditions, which may be diagnosed and treated based on the novel sequence information and annotations of the present invention.

This functional group classification includes the following groups:

Proteins Involved in Drug-Drug Interactions:

The phrase “proteins involved in drug-drug interactions” refers to proteins involved in a biological process which mediates the interaction between at least two consumed drugs.

Pharmaceutical compositions including such proteins or protein encoding sequences, antibodies directed against such proteins or polynucleotides capable of altering expression of such proteins, may be used to modulate drug-drug interactions. Antibodies and polynucleotides such as PCR primers and molecular probes designed to identify such proteins or protein encoding sequences may be used for diagnosis of such drug-drug interactions.

Examples of these conditions include, but are not limited to the cytochrom P450 protein family, which is involved in the metabolism of many drugs. Examples of proteins, which are involved in drug-drug interactions are presented in Table 9.

Proteins Involved in the Metabolism of a Pro-Drug to a Drug:

The phrase “proteins involved in the metabolism of a pro-drug to a drug” refers to proteins that activate an inactive pro-drug by chemically chaining it into a biologically active compound. Preferably, the metabolizing enzyme is expressed in the target tissue thus reducing systemic side effects.

Pharmaceutical compositions including such proteins or protein encoding sequences, antibodies directed against such proteins or polynucleotides capable of altering expression of such proteins, may be used to modulate the metabolism of a pro-drug into drug. Antibodies and polynucleotides such as PCR primers and molecular probes designed to identify such proteins or protein encoding sequences may be used for diagnosis of such conditions.

Examples of these proteins include, but are not limited to esterases hydrolyzing the cholesterol lowering drug simvastatin into its hydroxy acid active form.

MDR Proteins:

The phrase “MDR proteins” refers to Multi Drug Resistance proteins that are responsible for the resistance of a cell to a range of drugs, usually by exporting these drugs outside the cell. Preferably, the MDR proteins are ABC binding cassette proteins. Preferably, drug resistance is associated with resistance to chemotherapy.

Pharmaceutical compositions including such proteins or protein encoding sequences, antibodies directed against such proteins or polynucleotides capable of altering expression of such proteins, may be used to treat diseases in which the transport of molecules and macromolecules such as neurotransmitters, hormones, sugar etc. is abnormal leading to various pathologies. Antibodies and polynucleotides such as PCR primers and molecular probes designed to identify such proteins or protein encoding sequences may be used for diagnosis of such diseases.

Examples of these proteins include, but are not limited to the multi-drug resistant transporter MDR1/P-glycoprotein, the gene product of MDR1, which belongs to the ATP-binding cassette (ABC) superfamily of membrane transporters and increases the resistance of malignant cells to therapy by exporting the therapeutic agent out of the cell.

Hydrolases Acting on Amino Acids:

The phrase “hydrolases acting on amino acids” refers to hydrolases acting on a pair of amino acids.

Pharmaceutical compositions including such proteins or protein encoding sequences, antibodies directed against such proteins or polynucleotides capable of altering expression of such proteins, may be used to treat diseases in which the transfer of a glycosyl chemical group from one molecule to another is abnormal thus, a beneficial effect may be achieved by modulation of such reaction. Antibodies and polynucleotides such as PCR primers and molecular probes designed to identify such proteins or protein encoding sequences may be used for diagnosis of such diseases.

Examples of such diseases include, but are not limited to reperfusion of clotted blood vessels by TPA (Tissue Plasminogen Activator) which converts the abundant, but inactive, zymogen plasminogen to plasmin by hydrolyzing a single ARG-VAL bond in plasminogen.

Transaminases:

The term “transaminases” refers to enzymes transferring an amine group from one compound to another.

Pharmaceutical compositions including such proteins or protein encoding sequences, antibodies directed against such proteins or polynucleotides capable of altering expression of such proteins, may be used to treat diseases in which the transfer of an amine group from one molecule to another is abnormal thus, a beneficial effect may be achieved by modulation of such reaction. Antibodies and polynucleotides such as PCR primers and molecular probes designed to identify such proteins or protein encoding sequences may be used for diagnosis of such diseases.

Examples of such transaminases include, but are not limited to two liver enzymes, frequently used as markers for liver function—SGOT (Serum Glutamic-Oxalocetic Transaminase-AST) and SGPT (Serum Glutamic-Pyruvic Transaminase-ALT).

Immunoglobulins:

The term “immunoglobulins” refers to proteins that are involved in the immune and complement systems such as antigens and autoantigens, immunoglobulins, MHC and HLA proteins and their associated proteins.

Pharmaceutical compositions including such proteins or protein encoding sequences, antibodies directed against such proteins or polynucleotides capable of altering expression of such proteins, may be used to treat diseases involving the immune system such as inflammation, autoimmune diseases, infectious diseases, and cancerous processes. Antibodies and polynucleotides such as PCR primers and molecular probes designed to identify such proteins or protein encoding sequences may be used for diagnosis of such diseases.

Examples of such diseases and molecules that may be target for diagnostics include, but are not limited to members of the complement family such as C3 and C4 that their blood level is used for evaluation of autoimmune diseases and allergy state and C1 inhibitor that its absence is associated with angioedema. Thus, new variants of these genes are expected to be markers for similar events. Mutation in variants of the complement family may be associated with other immunological syndromes, such as increased bacterial infection that is associated with mutation in C3. C1 inhibitor was shown to provide safe and effective inhibition of complement activation after reperfused acute myocardial infarction and may reduce myocardial injury [Eur. Heart J. 2002, 23(21): 1670-7], thus, its variant may have the same or improved effect.

Transcription Factor Binding:

The phrase “transcription factor binding” refers to proteins involved in transcription process by binding to nucleic acids, such as transcription factors, RNA and DNA binding proteins, zinc fingers, helicase, isomerase, histones, and nucleases.

Pharmaceutical compositions including such proteins or protein encoding sequences, antibodies directed against such proteins or polynucleotides capable of altering expression of such proteins may be used to treat diseases involving transcription factors binding proteins. Such treatment may be based on transcription factor that can be used to for modulation of gene expression associated with the disease. Antibodies and polynucleotides such as PCR primers and molecular probes designed to identify such proteins or protein encoding sequences may be used for diagnosis of such diseases.

Examples of such diseases include, but are not limited to breast cancer associated with ErbB-2 expression that was shown to be successfully modulated by a transcription factor [Proc. Natl. Acad. Sci. USA. 2000, 97(4): 1495-500]. Examples of novel transcription factors used for therapeutic protein production include, but are not limited to those described for Erythropoietin production [J. Biol. Chem. 2000, 275(43):33850-60; J. Biol. Chem. 2000, 275(43):33850-60] and zinc fingers protein transcription factors (ZFP-TF) variants [J. Biol. Chem. 2000, 275(43):33850-60].

Small GTPase Regulatory/Interacting Proteins:

The phrase “Small GTPase regulatory/interacting proteins” refers to proteins capable of regulating or interacting with GTPase such as RAB escort protein, guanyl-nucleotide exchange factor, guanyl-nucleotide exchange factor adaptor, GDP-dissociation inhibitor, GTPase inhibitor, GTPase activator, guanyl-nucleotide releasing factor, GDP-dissociation stimulator, regulator of G-protein signaling, RAS interactor, RHO interactor, RAB interactor, and RAL interactor.

Pharmaceutical compositions including such proteins or protein encoding sequences, antibodies directed against such proteins or polynucleotides capable of altering expression of such proteins, may be used to treat diseases in which G-proteases mediated signal-transduction is abnormal, either as a cause, or as a result of the disease. Antibodies and polynucleotides such as PCR primers and molecular probes designed to identify such proteins or protein encoding sequences may be used for diagnosis of such diseases.

Examples of such diseases include, but are not limited to diseases related to prenylation. Modulation of prenylation was shown to affect therapy of diseases such as osteoporosis, ischemic heart disease, and inflammatory processes. Small GTPases regulatory/interacting proteins are major component in the prenylation post translation modification, and are required to the normal activity of prenylated proteins. Thus, their variants may be used for therapy of prenylation associated diseases.

Calcium Binding Proteins:

The phrase “calcium binding proteins” refers to proteins involve in calcium binding, preferably, calcium binding proteins, ligand binding or carriers, such as diacylglycerol kinase, Calpain, calcium-dependent protein serine/threonine phosphatase, calcium sensing proteins, calcium storage proteins.

Pharmaceutical compositions including such proteins or protein encoding sequences, antibodies directed against such proteins or polynucleotides capable of altering expression of such proteins, may be used to treat calcium involved diseases. Antibodies and polynucleotides such as PCR primers and molecular probes designed to identify such proteins or protein encoding sequences may be used for diagnosis of such diseases.

Examples of such diseases include, but are not limited to diseases related to hypercalcemia, hypertension, cardiovascular disease, muscle diseases, gastro-intestinal diseases, uterus relaxing, and uterus. An example for therapy use of calcium binding proteins variant may be treatment of emergency cases of hypercalcemia, with secreted variants of calcium storage proteins.

Oxidoreductase:

The term “oxidoreductase” refers to enzymes that catalyze the removal of hydrogen atoms and electrons from the compounds on which they act. Preferably, oxidoreductases acting on the following groups of donors: CH—OH, CH—CH, CH—NH2, CH—NH; oxidoreductases acting on NADH or NADPH, nitrogenous compounds, sulfur group of donors, heme group, hydrogen group, diphenols and related substances as donors; oxidoreductases acting on peroxide as acceptor, superoxide radicals as acceptor, oxidizing metal ions, CH2 groups; oxidoreductases acting on reduced ferredoxin as donor; oxidoreductases acting on reduced flavodoxin as donor; and oxidoreductases acting on the aldehyde or oxo group of donors.

Pharmaceutical compositions including such proteins or protein encoding sequences, antibodies directed against such proteins or polynucleotides capable of altering expression of such proteins, may be used to treat diseases caused by abnormal activity of oxidoreductases. Antibodies and polynucleotides such as PCR primers and molecular probes designed to identify such proteins or protein encoding sequences may be used for diagnosis of such diseases.

Examples of such diseases include, but are not limited to malignant and autoimmune diseases in which the enzyme DHFR (DiHydroFolateReductase) that participates in folate metabolism and essential for de novo glycine and purine synthesis is the target for the widely used drug Methotrexate (MTX).

Receptors:

The term “receptors” refers to protein-binding sites on a cell's surface or interior, that recognize and binds to specific messenger molecule leading to a biological response, such as signal transducers, complement receptors, ligand-dependent nuclear receptors, transmembrane receptors, GPI-anchored membrane-bound receptors, various coreceptors, internalization receptors, receptors to neurotransmitters, hormones and various other effectors and ligands.

Pharmaceutical compositions including such proteins or protein encoding sequences, antibodies directed against such proteins or polynucleotides capable of altering expression of such proteins, may be used to treat diseases caused by abnormal activity of receptors, preferably, receptors to neurotransmitters, hormones and various other effectors and ligands. Antibodies and polynucleotides such as PCR primers and molecular probes designed to identify such proteins or protein encoding sequences may be used for diagnosis of such diseases.

Examples of such diseases include, but are not limited to, chronic myelomonocytic leukemia caused by growth factor β receptor deficiency [Rao D. S., et al., (2001) Mol. Cell Biol., 21(22):7796-806], thrombosis associated with protease-activated receptor deficiency [Sambrano G. R., et al., (2001) Nature, 413(6851):26-7], hypercholesterolemia associated with low density lipoprotein receptor deficiency [Koivisto U. M., et al., (2001) Cell, 105(5):575-85], familial Hibernian fever associated with tumor necrosis factor receptor deficiency [Simon A., et al., (2001) Ned Tijdschr Geneeskd, 145(2):77-8], colitis associated with immunoglobulin E receptor expression [Dombrowicz D., et al., (2001) J. Exp. Med., 193(1):25-34], and alagille syndrome associated with Jagged1 [Stankiewicz P. et al., (2001) Am. J. Med. Genet., 103(2):166-71], breast cancer associated with mutated BRCA2 and androgen. Therapeutic applications of nuclear receptors variants may be based on secreted version of receptors such as the thyroid nuclear receptor that by binding plasma free thyroid hormone to reduce its levels may have a therapeutic effect in cases of thyrotoxicosis. A secreted version of glucocorticoid nuclear receptor, by binding plasma free cortisol, thus, reducing, may have a therapeutic effect in cases of Cushing's disease (a disease associated with high cortisole levels in the plasma).

Another example of a secreted variant of a receptor is a secreted form of the TNF receptor, which is used to treat conditions in which reduction of TNF levels is of benefit including Rheumatoid Arthritis, Juvenile Rheumatoid Arthritis, Psoriatic Arthritis and Ankylosing Spondylitis.

Protein Serine/Threonine Kinases:

The phrase “protein serine/threonine kinases” refers to proteins which phosphorylate serine/threonine residues, mainly involved in signal transduction, such as transmembrane receptor protein serine/threonine kinase, 3-phosphoinositide-dependent protein kinase, DNA-dependent protein kinase, G-protein-coupled receptor phosphorylating protein kinase, SNF1A/AMP-activated protein kinase, casein kinase, calmodulin regulated protein kinase, cyclic-nucleotide dependent protein kinase, cyclin-dependent protein kinase, eukaryotic translation initiation factor 2α kinase, galactosyltransferase-associated kinase, glycogen synthase kinase 3, protein kinase C, receptor signaling protein serine/threonine kinase, ribosomal protein S6 kinase, and IkB kinase.

Pharmaceutical compositions including such proteins or protein encoding sequences, antibodies directed against such proteins or polynucleotides capable of altering expression of such proteins, may be used to treat diseases ameliorated by a modulating kinase activity. Antibodies and polynucleotides such as PCR primers and molecular probes designed to identify such proteins or protein encoding sequences may be used for diagnosis of such diseases.

Examples of such diseases include, but are not limited to schizophrenia. 5-HT(2A) serotonin receptor is the principal molecular target for LSD-like hallucinogens and atypical antipsychotic drugs. It has been shown that a major mechanism for the attenuation of this receptor signaling following agonist activation typically involves the phosphorylation of serine and/or threonine residues by various kinases. Therefore, serine/threonine kinases specific for the 5-HT(2A) serotonin receptor may serve as drug targets for a disease such as schizophrenia. Other diseases that may be treated through serine/thereonine kinases modulation are Peutz-Jeghers syndrome (PJS, a rare autosomal-dominant disorder characterized by hamartomatous polyposis of the gastrointestinal tract and melanin pigmentation of the skin and mucous membranes [Hum. Mutat. 2000, 16(1):23-30], breast cancer [Oncogene. 1999, 18(35):4968-73], Type 2 diabetes insulin resistance [Am. J. Cardiol. 2002, 90(5A):11G-18G], and fanconi anemia [Blood. 2001, 98(13):3650-7].

Channel/Pore Class Transporters:

The phrase “Channel/pore class transporters” refers to proteins that mediate the transport of molecules and macromolecules across membranes, such as α-type channels, porins, and pore-forming toxins.

Pharmaceutical compositions including such proteins or protein encoding sequences, antibodies directed against such proteins or polynucleotides capable of altering expression of such proteins, may be used to treat diseases in which the transport of molecules and macromolecules are abnormal, therefore leading to various pathologies. Antibodies and polynucleotides such as PCR primers and molecular probes designed to identify such proteins or protein encoding sequences may be used for diagnosis of such diseases.

Examples of such diseases include, but are not limited to diseases of the nerves system such as Parkinson, diseases of the hormonal system, diabetes and infectious diseases such as bacterial and fungal infections. For example, α-hemolysin, is a protein product of S. aureus which creates ion conductive pores in the cell membrane, thereby deminishing its integrity.

Hydrolases, Acting on Acid Anhydrides:

The phrase “hydrolases, acting on acid anhydrides” refers to hydrolytic enzymes that are acting on acid anhydrides, such as hydrolases acting on acid anhydrides in phosphorus-containing anhydrides or in sulfonyl-containing anhydrides, hydrolases catalyzing transmembrane movement of substances, and involved in cellular and subcellular movement.

Pharmaceutical compositions including such proteins or protein encoding sequences, antibodies directed against such proteins or polynucleotides capable of altering expression of such proteins may be used to treat diseases in which the hydrolase-related activities are abnormal. Antibodies and polynucleotides such as PCR primers and molecular probes designed to identify such proteins or protein encoding sequences may be used for diagnosis of such diseases.

Examples of such diseases include, but are not limited to glaucoma treated with carbonic anhydrase inhibitors (e.g. Dorzolamide), peptic ulcer disease treated with H(⁺)K(⁺)ATPase inhibitors that were shown to affect disease by blocking gastric carbonic anhydrase (e.g. Omeprazole).

Transferases, Transferring Phosphorus-Containing Groups:

The phrase “transferases, transferring phosphorus-containing groups” refers to enzymes that catalyze the transfer of phosphate from one molecule to another, such as phosphotransferases using the following groups as acceptors: alcohol group, carboxyl group, nitrogenous group, phosphate; phosphotransferases with regeneration of donors catalyzing intramolecular transfers; diphosphotransferases; nucleotidyltransferase; and phosphotransferases for other substituted phosphate groups.

Pharmaceutical compositions including such proteins or protein encoding sequences, antibodies directed against such proteins or polynucleotides capable of altering expression of such proteins may be used to treat diseases in which the transfer of a phosphorous containing functional group to a modulated moiety is abnormal. Antibodies and polynucleotides such as PCR primers and molecular probes designed to identify such proteins or protein encoding sequences may be used for diagnosis of such diseases.

Examples of such diseases include, but are not limited to acute MI [Ann. Emerg. Med. 2003, 42(3):343-50], Cancer [Oral. Dis. 2003, 9(3):119-28; J. Surg. Res. 2003, 113(1):102-8] and Alzheimer's disease [Am. J. Pathol. 2003, 163(3):845-58]. Examples for possible utilities of such transferases for drug improvement include, but are not limited to aminoglycosides treatment (antibiotics) to which resistance is mediated by aminoglycoside phosphotransferases [Front. Biosci. 1999, 1;4:D9-21]. Using aminoglycoside phosphotransferases variants or inhibiting these enzymes may reduce aminoglycosides resistance. Since aminoglycosides can be toxic to some patients, proving the expression of aminoglycoside phosphotransferases in a patient can deter from treating him with aminoglycosides and risking the patient in vain.

Phosphoric Monoester Hydrolases:

The phrase “phosphoric monoester hydrolases” refers to hydrolytic enzymes that are acting on ester bonds, such as nuclease, sulfuric ester hydrolase, carboxylic ester hydrolase, thiolester hydrolase, phosphoric monoester hydrolase, phosphoric diester hydrolase, triphosphoric monoester hydrolase, diphosphoric monoester hydrolase, and phosphoric triester hydrolase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other), is abnormal. Antibodies and polynucleotides such as PCR primers and molecular probes designed to identify such proteins or protein encoding sequences may be used for diagnosis of such diseases.

Examples of such diseases include, but are not limited to diabetes and CNS diseases such as Parkinson and cancer.

Enzyme Inhibitors:

The term “enzyme inhibitors” refers to inhibitors and suppressors of other proteins and enzymes, such as inhibitors of: kinases, phosphatases, chaperones, guanylate cyclase, DNA gyrase, ribonuclease, proteasome inhibitors, diazepam-binding inhibitor, ornithine decarboxylase inhibitor, GTPase inhibitors, dUTP pyrophosphatase inhibitor, phospholipase inhibitor, proteinase inhibitor, protein biosynthesis inhibitors, and α-amylase inhibitors.

Pharmaceutical compositions including such proteins or protein encoding sequences, antibodies directed against such proteins or polynucleotides capable of altering expression of such proteins, may be used to treat diseases in which beneficial effect may be achieved by modulating the activity of inhibitors and suppressors of proteins and enzymes. Antibodies and polynucleotides such as PCR primers and molecular probes designed to identify such proteins or protein encoding sequences may be used for diagnosis of such diseases.

Examples of such diseases include, but are not limited to α-1 antitrypsin (a natural serine proteases, which protects the lung and liver from proteolysis) deficiency associated with emphysema, COPD and liver chirosis. α-1 antitrypsin is also used for diagnostics in cases of unexplained liver and lung disease. A variant of this enzyme may act as protease inhibitor or a diagnostic target for related diseases.

Electron Transporters:

The term “Electron transporters” refers to ligand binding or carrier proteins involved in electron transport such as flavin-containing electron transporter, cytochromes, electron donors, electron acceptors, electron carriers, and cytochrome-c oxidases.

Pharmaceutical compositions including such proteins or protein encoding sequences, antibodies directed against such proteins or polynucleotides capable of altering expression of such proteins, may be used to treat diseases in which beneficial effect may be achieved by modulating the activity of electron transporters. Antibodies and polynucleotides such as PCR primers and molecular probes designed to identify such proteins or protein encoding sequences may be used for diagnosis of such diseases.

Examples of such diseases include, but are not limited to cyanide toxicity, resulting from cyanide binding to ubiquitous metalloenzymes rendering them inactive, and interfering with the electron transport. Novel electron transporters to which cyanide can bind may serve as drug targets for new cyanide antidotes.

Transferases, Transferring Glycosyl Groups:

The phrase “transferases, transferring glycosyl groups” refers to enzymes that catalyze the transfer of a glycosyl chemical group from one molecule to another such as murein lytic endotransglycosylase E, and sialyltransferase.

Pharmaceutical compositions including such proteins or protein encoding sequences, antibodies directed against such proteins or polynucleotides capable of altering expression of such proteins, may be used to treat diseases in which the transfer of a glycosyl chemical group is abnormal. Antibodies and polynucleotides such as PCR primers and molecular probes designed to identify such proteins or protein encoding sequences may be used for diagnosis of such diseases.

Ligases, Forming Carbon-Oxygen Bonds:

The phrase “ligases, forming carbon-oxygen bonds” refers to enzymes that catalyze the linkage between carbon and oxygen such as ligase forming aminoacyl-tRNA and related compounds.

Pharmaceutical compositions including such proteins or protein encoding sequences, antibodies directed against such proteins or polynucleotides capable of altering expression of such proteins, may be used to treat diseases in which the linkage between carbon and oxygen in an energy dependent process is abnormal. Antibodies and polynucleotides such as PCR primers and molecular probes designed to identify such proteins or protein encoding sequences may be used for diagnosis of such diseases.

Ligases:

The term “ligases” refers to enzymes that catalyze the linkage of two molecules, generally utilizing ATP as the energy donor, also called synthetase. Examples for ligases are enzymes such as β-alanyl-dopamine hydrolase, carbon-oxygen bonds forming ligase, carbon-sulfur bonds forming ligase, carbon-nitrogen bonds forming ligase, carbon-carbon bonds forming ligase, and phosphoric ester bonds forming ligase.

Pharmaceutical compositions including such proteins or protein encoding sequences, antibodies directed against such proteins or polynucleotides capable of altering expression of such proteins, may be used to treat diseases in which the joining together of two molecules in an energy dependent process is abnormal. Antibodies and polynucleotides such as PCR primers and molecular probes designed to identify such proteins or protein encoding sequences may be used for diagnosis of such diseases.

Examples of such diseases include, but are not limited to neurological disorders such as Parkinson's disease [Science. 2003, 302(5646):819-22; J. Neurol. 2003, 250 Suppl. 3:III25-III29] or epilepsy [Nat. Genet. 2003, 35(2):125-7], cancerous diseases [Cancer Res. 2003, 63(17):5428-37; Lab. Invest. 2003, 83(9):1255-65], renal diseases [Am. J. Pathol. 2003, 163(4):1645-52], infectious diseases [Arch. Virol. 2003, 148(9):1851-62] and fanconi anemia [Nat. Genet. 2003, 35(2):165-70].

Hydrolases, Acting on Glycosyl Bonds:

The phrase “hydrolases, acting on glycosyl bonds” refers to hydrolytic enzymes that are acting on glycosyl bonds such as hydrolases hydrolyzing N-glycosyl compounds, S-glycosyl compounds, and O-glycosyl compounds.

Pharmaceutical compositions including such proteins or protein encoding sequences, antibodies directed against such proteins or polynucleotides capable of altering expression of such proteins, may be used to treat diseases in which the hydrolase-related activities are abnormal. Antibodies and polynucleotides such as PCR primers and molecular probes designed to identify such proteins or protein encoding sequences may be used for diagnosis of such diseases.

Examples of such diseases include cancerous diseases [J. Natl. Cancer Inst. 2003, 95(17):1263-5; Carcinogenesis. 2003, 24(7):1281-2; author reply 1283] vascular diseases [J. Thorac. Cardiovasc. Surg. 2003, 126(2):344-57], gastrointestinal diseases such as colitis [J. Immunol. 2003, 171(3):1556-63] or liver fibrosis [World J. Gastroenterol. 2002, 8(5):901-7].

Kinases:

The term “kinases” refers to enzymes which phosphorylate serine/threonine or tyrosine residues, mainly involved in signal transduction. Examples for kinases include enzymes such as 2-amino-4-hydroxy-6-hydroxymethyldihydropteridine pyrophosphokinase, NAD(⁺) kinase, acetylglutamate kinase, adenosine kinase, adenylate kinase, adenylsulfate kinase, arginine kinase, aspartate kinase, choline kinase, creatine kinase, cytidylate kinase, deoxyadenosine kinase, deoxycytidine kinase, deoxyguanosine kinase, dephospho-CoA kinase, diacylglycerol kinase, dolichol kinase, ethanolamine kinase, galactokinase, glucokinase, glutamate 5-kinase, glycerol kinase, glycerone kinase, guanylate kinase, hexokinase, homoserine kinase, hydroxyethylthiazole kinase, inositol/phosphatidylinositol kinase, ketohexokinase, mevalonate kinase, nucleoside-diphosphate kinase, pantothenate kinase, phosphoenolpyruvate carboxykinase, phosphoglycerate kinase, phosphomevalonate kinase, protein kinase, pyruvate dehydrogenase (lipoamide) kinase, pyruvate kinase, ribokinase, ribose-phosphate pyrophosphokinase, selenide, water dikinase, shikimate kinase, thiamine pyrophosphokinase, thymidine kinase, thymidylate kinase, uridine kinase, xylulokinase, 1D-myo-inositol-trisphosphate 3-kinase, phosphofructokinase, pyridoxal kinase, sphinganine kinase, riboflavin kinase, 2-dehydro-3-deoxygalactonokinase, 2-dehydro-3-deoxygluconokinase, 4-diphosphocytidyl-2C-methyl-D-erythritol kinase, GTP pyrophosphokinase, L-fuculokinase, L-ribulokinase, L-xylulokinase, isocitrate dehydrogenase (NADP⁺) kinase, acetate kinase, allose kinase, carbamate kinase, cobinamide kinase, diphosphate-purine nucleoside kinase, fructokinase, glycerate kinase, hydroxymethylpyrimidine kinase, hygromycin-B kinase, inosine kinase, kanamycin kinase, phosphomethylpyrimidine kinase, phosphoribulokinase, polyphosphate kinase, propionate kinase, pyruvate, water dikinase, rhamnulokinase, tagatose-6-phosphate kinase, tetraacyldisaccharide 4′-kinase, thiamine-phosphate kinase, undecaprenol kinase, uridylate kinase, N-acylmannosamine kinase, D-erythro-sphingosine kinase.

Pharmaceutical compositions including such proteins or protein encoding sequences, antibodies directed against such proteins or polynucleotides capable of altering expression of such proteins, may be used to treat diseases which may be ameliorated by a modulating kinase activity. Antibodies and polynucleotides such as PCR primers and molecular probes designed to identify such proteins or protein encoding sequences may be used for diagnosis of such diseases.

Examples of such diseases include, but are not limited to, acute lymphoblastic leukemia associated with spleen tyrosine kinase deficiency [Goodman P. A., et al., (2001) Oncogene, 20(30):3969-78], ataxia telangiectasia associated with ATM kinase deficiency [Boultwood J., (2001) J. Clin. Pathol., 54(7):512-6], congenital haemolytic anaemia associated with erythrocyte pyruvate kinase deficiency [Zanella A., et al., (2001) Br. J. Haematol., 113(1):43-8], mevalonic aciduria caused by mevalonate kinase deficiency [Houten S. M., et al., (2001) Eur. J. Hum. Genet., 9(4):253-9], and acute myelogenous leukemia associated with over-expressed death-associated protein kinase [Guzman M. L., et al., (2001) Blood, 97(7):2177-9].

Nucleotide Binding:

The term “nucleotide binding” refers to ligand binding or carrier proteins, involved in physical interaction with a nucleotide, preferably, any compound consisting of a nucleoside that is esterified with [ortho]phosphate or an oligophosphate at any hydroxyl group on the glycose moiety, such as purine nucleotide binding proteins.

Pharmaceutical compositions including such proteins or protein encoding sequences, antibodies directed against such proteins or polynucleotides capable of altering expression of such proteins, may be used to treat diseases that are associated with abnormal nucleotide binding. Antibodies and polynucleotides such as PCR primers and molecular probes designed to identify such proteins or protein encoding sequences may be used for diagnosis of such diseases.

Examples of such diseases include, but are not limited to Gout (a syndrome characterized by high urate level in the blood). Since urate is a breakdown metabolite of purines, reducing purines serum levels could have a therapeutic effect in Gout disease.

Tubulin Binding:

The term “tubulin binding” refers to binding proteins that bind tubulin such as microtubule binding proteins.

Pharmaceutical compositions including such proteins or protein encoding sequences, antibodies directed against such proteins or polynucleotides capable of altering expression of such proteins, may be used to treat diseases which are associated with abnormal tubulin activity or structure. Binding the products of the genes of this family, or antibodies reactive therewith, can modulate a plurality of tubulin activities as well as change microtubulin structure. Antibodies and polynucleotides such as PCR primers and molecular probes designed to identify such proteins or protein encoding sequences may be used for diagnosis of such diseases.

Examples of such diseases include, but are not limited to, Alzheimer's disease associated with t-complex polypeptide 1 deficiency [Schuller E., et al., (2001) Life Sci., 69(3):263-70], neurodegeneration associated with apoE deficiency [Masliah E., et al., (1995) Exp. Neurol., 136(2):107-22], progressive axonopathy associated with disfuctional neurofilaments [Griffiths I. R., et al., (1989) Neuropathol. Appl. Neurobiol., 15(1):63-74], familial frontotemporal dementia associated with tau deficiency [astor P., et al., (2001) Ann. Neurol., 49(2):263-7], and colon cancer suppressed by APC [White R. L., (1997) Pathol. Biol. (Paris), 45(3):240-4]. En example for a drug whose target is tubulin is the anticancer drug—Taxol. Drugs having similar mechanism of action (interfering with tubulin polymerization) may be developed based on tubulin binding proteins.

Receptor Signaling Proteins:

The phrase “receptor signaling proteins” refers to receptor proteins involved in signal transduction such as receptor signaling protein serine/threonine kinase, receptor signaling protein tyrosine kinase, receptor signaling protein tyrosine phosphatase, aryl hydrocarbon receptor nuclear translocator, hematopoeitin/interferon-class (D200-domain) cytokine receptor signal transducer, transmembrane receptor protein tyrosine kinase signaling protein, transmembrane receptor protein serine/threonine kinase signaling protein, receptor signaling protein serine/threonine kinase signaling protein, receptor signaling protein serine/threonine phosphatase signaling protein, small GTPase regulatory/interacting protein, receptor signaling protein tyrosine kinase signaling protein, and receptor signaling protein serine/threonine phosphatase.

Pharmaceutical compositions including such proteins or protein encoding sequences, antibodies directed against such proteins or polynucleotides capable of altering expression of such proteins, may be used to treat diseases in which the signal-transduction is abnormal, either as a cause, or as a result of the disease. Antibodies and polynucleotides such as PCR primers and molecular probes designed to identify such proteins or protein encoding sequences may be used for diagnosis of such diseases.

Examples of such diseases include, but are not limited to, complete hypogonadotropic hypogonadism associated with GnRH receptor deficiency [Kottler M. L., et al., (2000) J. Clin. Endocrinol. Metab., 85(9):3002-8], severe combined immunodeficiency disease associated with IL-7 receptor deficiency [Puel A. and Leonard W. J., (2000) Curr. Opin. Immunol., 12(4):468-7], schizophrenia associated N-methyl-D-aspartate receptor deficiency [Mohn A. R., et al., (1999) Cell, 98(4):427-36], Yesinia-associated arthritis associated with tumor necrosis factor receptor p55 deficiency [Zhao Y. X., et al., (1999) Arthritis Rheum., 42(8):1662-72], and Dwarfism of Sindh caused by growth hormone-releasing hormone receptor deficiency [aheshwari H. G., et al., (1998) J. Clin. Endocrinol. Metab., 83(11):4065-74].

Molecular Function Unknown:

The phrase “molecular function unknown” refers to various proteins with unknown molecular function, such as cell surface antigens.

Pharmaceutical compositions including such proteins or protein encoding sequences, antibodies directed against such proteins or polynucleotides capable of altering expression of such proteins, may be used to treat diseases in which regulation of the recognition, or participation or bind of cell surface antigens to other moieties may have therapeutic effect. Antibodies and polynucleotides such as PCR primers and molecular probes designed to identify such proteins or protein encoding sequences may be used for diagnosis of such diseases.

Examples of such diseases include, but are not limited to, autoimmune diseases, various infectious diseases, cancer diseases which involve non cell surface antigens recognition and activity.

Enzyme Activators:

The term “enzyme activators” refers to enzyme regulators such as activators of: kinases, phosphatases, sphingolipids, chaperones, guanylate cyclase, tryptophan hydroxylase, proteases, phospholipases, caspases, proprotein convertase 2 activator, cyclin-dependent protein kinase 5 activator, superoxide-generating NADPH oxidase activator, sphingomyelin phosphodiesterase activator, monophenol monooxygenase activator, proteasome activator, and GTPase activator.

Pharmaceutical compositions including such proteins or protein encoding sequences, antibodies directed against such proteins or polynucleotides capable of altering expression of such proteins, may be used to treat diseases in which beneficial effect may be achieved by modulating the activity of activators of proteins and enzymes. Antibodies and polynucleotides such as PCR primers and molecular probes designed to identify such proteins or protein encoding sequences may be used for diagnosis of such diseases.

Examples of such diseases include, but are not limited to all complement related diseases, as most complement proteins activate by cleavage other complement proteins.

Transferases, Transferring One-Carbon Groups:

The phrase “transferases, transferring one-carbon groups” refers enzymes that catalyze the transfer of a one-carbon chemical group from one molecule to another such as methyltransferase, amidinotransferase, hydroxymethyl-, formyl- and related transferase, carboxyl- and carbamoyltransferase.

Pharmaceutical compositions including such proteins or protein encoding sequences, antibodies directed against such proteins or polynucleotides capable of altering expression of such proteins, may be used to treat diseases in which the transfer of a one-carbon chemical group from one molecule to another is abnormal so that a beneficial effect may be achieved by modulation of such reaction. Antibodies and polynucleotides such as PCR primers and molecular probes designed to identify such proteins or protein encoding sequences may be used for diagnosis of such diseases.

Transferases:

The term “transferases” refers to enzymes that catalyze the transfer of a chemical group, preferably, a phosphate or amine from one molecule to another. It includes enzymes such as transferases, transferring one-carbon groups, aldehyde or ketonic groups, acyl groups, glycosyl groups, alkyl or aryl (other than methyl) groups, nitrogenous, phosphorus-containing groups, sulfur-containing groups, lipoyltransferase, deoxycytidyl transferases.

Pharmaceutical compositions including such proteins or protein encoding sequences, antibodies directed against such proteins or polynucleotides capable of altering expression of such proteins, may be used to treat diseases in which the transfer of a chemical group from one molecule to another is abnormal. Antibodies and polynucleotides such as PCR primers and molecular probes designed to identify such proteins or protein encoding sequences may be used for diagnosis of such diseases.

Examples of such diseases include, but are not limited to cancerous diseases such as prostate cancer [Urology. 2003, 62(5 Suppl 1):55-62] or lung cancer [Invest. New Drugs. 2003, 21(4):435-43; JAMA. 2003, 22;290(16):2149-58], psychiatric disorders [Am. J. Med. Genet. 2003, 15;123B(1):64-9], colorectal disease such as Crohn's disease [Dis. Colon Rectum. 2003, 46(11):1498-507] or celiac diseases [N Engl. J. Med. 2003, 349(17):1673-4; author reply 1673-4], neurological diseases such as Prkinson's disease [J. Chem Neuroanat. 2003, 26(2):143-51], Alzheimer disease [Hum. Mol. Genet. 2003 21] or Charcot-Marie-Tooth Disease [Mol. Biol. Evol. 2003 31].

Chaperones:

The term “chaperones” refers to functional classes of unrelated families of proteins that assist the correct non-covalent assembly of other polypeptide-containing structures in vivo, but are not components of these assembled structures when they a performing their normal biological function. The group of chaperones include proteins such as ribosomal chaperone, peptidylprolyl isomerase, lectin-binding chaperone, nucleosome assembly chaperone, chaperonin ATPase, cochaperone, heat shock protein, HSP70/HSP90 organizing protein, fimbrial chaperone, metallochaperone, tubulin folding, and HSC70-interacting protein.

Pharmaceutical compositions including such proteins or protein encoding sequences, antibodies directed against such proteins or polynucleotides capable of altering expression of such proteins, may be used to treat diseases which are associated with abnormal protein activity, structure, degradation or accumulation of proteins. Antibodies and polynucleotides such as PCR primers and molecular probes designed to identify such proteins or protein encoding sequences may be used for diagnosis of such diseases.

Examples of such diseases include, but are not limited to neurological syndromes [J. Neuropathol. Exp. Neurol. 2003, 62(7):751-64; Antioxid Redox Signal. 2003, 5(3):337-48; J. Neurochem. 2003, 86(2):394-404], neurological diseases such as Parkinson's disease [Hum. Genet. 2003, 6; Neurol Sci. 2003, 24(3):159-60; J. Neurol. 2003, 250 Suppl. 3:11125-11129] ataxia [J. Hum. Genet. 2003;48(8):415-9] or Alzheimer diseases [J. Mol. Neurosci. 2003, 20(3):283-6; J. Alzheimers Dis. 2003, 5(3):171-7], cancerous diseases [Semin. Oncol. 2003, 30(5):709-16], prostate cancer [Semin. Oncol. 2003, 30(5):709-16] metabolic diseases [J. Neurochem. 2003, 87(1):248-56], infectious diseases, such as prion infection [EMBO J. 2003, 22(20):5435-5445]. Chaperones may be also used for manipulating therapeutic proteins binding to their receptors therefore, improving their therapeutic effect.

Cell Adhesion Molecule:

The phrase “cell adhesion molecule” refers to proteins that serve as adhesion molecules between adjoining cells such as membrane-associated protein with guanylate kinase activity, cell adhesion receptor, neuroligin, calcium-dependent cell adhesion molecule, selectin, calcium-independent cell adhesion molecule, and extracellular matrix protein.

Pharmaceutical compositions including such proteins or protein encoding sequences, antibodies directed against such proteins or polynucleotides capable of altering expression of such proteins, may be used to treat diseases in which adhesion between adjoining cells is involved, typically conditions in which the adhesion is abnormal. Antibodies and polynucleotides such as PCR primers and molecular probes designed to identify such proteins or protein encoding sequences may be used for diagnosis of such diseases.

Examples of such diseases include, but are not limited to cancer in which abnormal adhesion may cause and enhance the process of metastasis and abnormal growth and development of various tissues in which modulation adhesion among adjoining cells can improve the condition. Leucocyte-endothlial interactions characterized by adhesion molecules involved in interactions between cells lead to a tissue injury and ischemia reperfusion disorders in which activated signals generated during ischemia may trigger an exuberant inflammatory response during reperfusion, provoking greater tissue damage than initial ischemic insult [Crit. Care Med. 2002, 30(5 Suppl):S214-9]. The blockade of leucocyte-endothelial adhesive interactions has the potential to reduce vascular and tissue injury. This blockade may be achieved using a soluble variant of the adhesion molecule.

States of septic shock and ARDS involve large recruitment of neutrophil cells to the damaged tissues. Neutrophil cells bind to the endothelial cells in the target tissues through adhesion molecules. Neutrophils possess multiple effector mechanisms that can produce endothelial and lung tissue injury, and interfere with pulmonary gas transfer by disruption of surfactant activity [Eur. J. Surg. 2002, 168(4):204-14]. In such cases, the use of soluble variant of the adhesion molecule may decrease the adhesion of neutrophils to the damaged tissues.

Examples of such diseases include, but are not limited to, Wiskott-Aldrich syndrome associated with WAS deficiency [Westerberg L., et al., (2001) Blood, 98(4):1086-94], asthma associated with intercellular adhesion molecule-1 deficiency [Tang M. L. and Fiscus L. C., (2001) Pulm. Pharmacol. Ther., 14(3):203-10], intra-atrial thrombogenesis associated with increased von Willebrand factor activity [Fukuchi M., et al., (2001) J. Am. Coll. Cardiol., 37(5): 1436-42], junctional epidermolysis bullosa associated with laminin 5-β-3 deficiency [Robbins P. B., et al., (2001) Proc. Natl. Acad. Sci., 98(9):5193-8], and hydrocephalus caused by neural adhesion molecule L1 deficiency [Rolf B., et al., (2001) Brain Res., 891(1-2):247-52].

Motor Proteins:

The term “motor proteins” refers to proteins that generate force or energy by the hydrolysis of ATP and that function in the production of intracellular movement or transportation. Examples of such proteins include microfilament motor, axonemal motor, microtubule motor, and kinetochore motor (dynein, kinesin, or myosin).

Pharmaceutical compositions including such proteins or protein encoding sequences, antibodies directed against such proteins or polynucleotides capable of altering expression of such proteins, may be used to treat diseases in which force or energy generation is impaired. Antibodies and polynucleotides such as PCR primers and molecular probes designed to identify such proteins or protein encoding sequences may be used for diagnosis of such diseases.

Examples of such diseases include, but are not limited to, malignant diseases where microtubules are drug targets for a family of anticancer drugs such as myodystrophies and myopathies [Trends Cell Biol. 2002, 12(12):585-91], neurological disorders [Neuron. 2003, 25;40(1):25-40; Trends Biochem. Sci. 2003, 28(10):558-65; Med. Genet. 2003, 40(9):671-5], and hearing impairment [Trends Biochem. Sci. 2003, 28(10):558-65].

Defense/Immunity Proteins:

The term “defense/immunity proteins” refers to proteins that are involved in the immune and complement systems such as acute-phase response proteins, antimicrobial peptides, antiviral response proteins, blood coagulation factors, complement components, immunoglobulins, major histocompatibility complex antigens and opsonins.

Pharmaceutical compositions including such proteins or protein encoding sequences, antibodies directed against such proteins or polynucleotides capable of altering expression of such proteins, may be used to treat diseases involving the immunological system including inflammation, autoimmune diseases, infectious diseases, as well as cancerous processes or diseases which are manifested by abnormal coagulation processes, which may include abnormal bleeding or excessive coagulation. Antibodies and polynucleotides such as PCR primers and molecular probes designed to identify such proteins or protein encoding sequences may be used for diagnosis of such diseases.

Examples of such diseases include, but are not limited to, late (C5-9) complement component deficiency associated with opsonin receptor allotypes [Fijen C. A., et al., (2000) Clin. Exp. Immunol., 120(2):338-45], combined immunodeficiency associated with defective expression of MHC class II genes [Griscelli C., et al., (1989) Immunodefic. Rev. 1(2):135-53], loss of antiviral activity of CD4 T cells caused by neutralization of endogenous TNFα [Pavic I., et al., (1993) J. Gen. Virol., 74 (Pt 10):2215-23], autoimmune diseases associated with natural resistance-associated macrophage protein deficiency [Evans C. A., et al., (2001) Neurogenetics, 3(2):69-78], Epstein-Barr virus-associated lymphoproliferative disease inhibited by combined GM-CSF and IL-2 therapy [Baiocchi R. A., et al., (2001) J. Clin. Invest., 108(6):887-94], and sepsis in which activated protein C is a therapeutic protein itself.

Intracellular Transporters:

The term “intracellular transporters” refers to proteins that mediate the transport of molecules and macromolecules inside the cell, such as intracellular nucleoside transporter, vacuolar assembly proteins, vesicle transporters, vesicle fusion proteins, type II protein secretors.

Pharmaceutical compositions including such proteins or protein encoding sequences, antibodies directed against such proteins or polynucleotides capable of altering expression of such proteins, may be used to treat diseases in which the transport of molecules and macromolecules is abnormal leading to various pathologies. Antibodies and polynucleotides such as PCR primers and molecular probes designed to identify such proteins or protein encoding sequences may be used for diagnosis of such diseases.

Transporters:

The term “transporters” refers to proteins that mediate the transport of molecules and macromolecules, such as channels, exchangers, and pumps. Transporters include proteins such as: amine/polyamine transporter, lipid transporter, neurotransmitter transporter, organic acid transporter, oxygen transporter, water transporter, carriers, intracellular transports, protein transporters, ion transporters, carbohydrate transporter, polyol transporter, amino acid transporters, vitamin/cofactor transporters, siderophore transporter, drug transporter, channel/pore class transporter, group translocator, auxiliary transport proteins, permeases, murein transporter, organic alcohol transporter, nucleobase, nucleoside, and nucleotide and nucleic acid transporters.

Pharmaceutical compositions including such proteins or protein encoding sequences, antibodies directed against such proteins or polynucleotides capable of altering expression of such proteins, may be used to treat diseases in which the transport of molecules and macromolecules such as neurotransmitters, hormones, sugar etc. is impaired leading to various pathologies. Antibodies and polynucleotides such as PCR primers and molecular probes designed to identify such proteins or protein encoding sequences may be used for diagnosis of such diseases.

Examples of such diseases include, but are not limited to, glycogen storage disease caused by glucose-6-phosphate transporter deficiency [Hiraiwa H., and Chou J. Y. (2001) DNA Cell Biol., 20(8):447-53], tangier disease associated with ATP-binding cassette transporter-i deficiency [McNeish J., et al., (2000) Proc. Natl. Acad. Sci., 97(8):4245-50], systemic primary camitine deficiency associated with organic cation transporter deficiency [Tang N. L., et al., (1999) Hum. Mol. Genet., 8(4):655-60], Wilson disease associated with copper-transporting ATPases deficiency [Payne A. S., et al., (1998) Proc. Natl. Acad. Sci. 95(18):10854-9], and atelosteogenesis associated with diastrophic dysplasia sulphate transporter deficiency [Newbury-Ecob R., (1998) J. Med. Genet., 35(1):49-53], Central Nervous system diseases treated by inhibiting neurotransmitter transporter (e.g. Depression, treated with serotonin transporters inhibitors—Prozac), and Cystic fibrosis mediated by the chloride channel CFTR. Other transporter related diseases are cancer [Oncogene. 2003, 22(38):6005-12] and especially cancer resistant to treatment [Oncologist. 2003, 8(5):411-24; J. Med. Invest. 2003, 50(3-4):126-35], infectious diseases, especially fungal infections [Annu. Rev. Phytopathol. 2003, 41:641-67], neurological diseases, such as Parkinson [FASEB J. 2003, Sep. 4 [Epub ahead of print]], and cardiovascular diseases, including hypercholesterolemia [Am. J. Cardiol. 2003, 92(4B): 10K-16K].

There are about 30 membrane transporter genes linked to a known genetic clinical syndrome. Secreted versions of splice variants of transporters may be therapeutic as the case with soluble receptors. These transporters may have the capability to bind the compound in the serum they would normally bind on the membrane. For example, a secreted form ATP7B, a transporter involved in Wilson's disease, is expected to bind plasma Copper, therefore have a desired therapeutic effect in Wilson's disease.

Lyases:

The term “lyases” refers to enzymes that catalyze the formation of double bonds by removing chemical groups from a substrate without hydrolysis or catalyze the addition of chemical groups to double bonds. It includes enzymes such as carbon-carbon lyase, carbon-oxygen lyase, carbon-nitrogen lyase, carbon-sulfur lyase, carbon-halide lyase, and phosphorus-oxygen lyase.

Pharmaceutical compositions including such proteins or protein encoding sequences, antibodies directed against such proteins or polynucleotides capable of altering expression of such proteins, may be used to treat diseases in which the double bonds formation catalyzed by these enzymes is impaired. Antibodies and polynucleotides such as PCR primers and molecular probes designed to identify such proteins or protein encoding sequences may be used for diagnosis of such diseases.

Examples of such diseases include, but are not limited to, autoimmune diseases [JAMA. 2003, 290(13):1721-8; JAMA. 2003, 290(13):1713-20], diabetes [Diabetes. 2003, 52(9):2274-8], neurological disorders such as epilepsy [J. Neurosci. 2003, 23(24):8471-9], Parkinson [J. Neurosci. 2003, 23(23):8302-9; Lancet. 2003, 362(9385):712] or Creutzfeldt-Jakob disease [Clin. Neurophysiol. 2003, 114(9):1724-8], and cancerous diseases [J. Pathol. 2003, 201(1):37-45; J. Pathol. 2003, 201(1):37-45; Cancer Res. 2003, 63(16):4952-9; Eur. J. Cancer. 2003, 39(13):1899-903].

Actin Binding Proteins:

The phrase “actin binding proteins” refers to proteins binding actin as actin cross-linking, actin bundling, F-actin capping, actin monomer binding, actin lateral binding, actin depolymerizing, actin monomer sequestering, actin filament severing, actin modulating, membrane associated actin binding, actin thin filament length regulation, and actin polymerizing proteins.

Pharmaceutical compositions including such proteins or protein encoding sequences, antibodies directed against such proteins or polynucleotides capable of altering expression of such proteins, may be used to treat diseases in which actin binding is impaired. Antibodies and polynucleotides such as PCR primers and molecular probes designed to identify such proteins or protein encoding sequences may be used for diagnosis of such diseases.

Examples of such diseases include, but are not limited to, neuromuscular diseases such as muscular dystrophy [Neurology. 2003, 61(3):404-6], Cancerous diseases [Urology. 2003, 61(4):845-50; J. Cutan. Pathol. 2002, 29(7):430; Cancer. 2002, 94(6):1777-86; Clin. Cancer Res. 2001, 7(8):2415-24; Breast Cancer Res. Treat. 2001, 65(1): 11-21], renal diseases such as glomerulonephritis [J. Am. Soc. Nephrol. 2002, 13(2):322-31; Eur. J. Immunol. 2001, 31(4):1221-7], and gastrointestinal diseases such as Crohn's disease [J. Cell Physiol. 2000, 182(2):303-9].

Protein Binding Proteins:

The phrase “protein binding proteins” refers to proteins involved in diverse biological functions through binding other proteins. Examples of such biological function include intermediate filament binding, LIM-domain binding, LLR-domain binding, clathrin binding, ARF binding, vinculin binding, KU70 binding, troponin C binding PDZ-domain binding, SH3-domain binding, fibroblast growth factor binding, membrane-associated protein with guanylate kinase activity interacting, Wnt-protein binding, DEAD/H-box RNA helicase binding, β-amyloid binding, myosin binding, TATA-binding protein binding DNA topoisomerase 1 binding, polypeptide hormone binding, RHO binding, FH1-domain binding, syntaxin-1 binding, HSC70-interacting, transcription factor binding, metarhodopsin binding, tubulin binding, JUN kinase binding, RAN protein binding, protein signal sequence binding, importin α export receptor, poly-glutamine tract binding, protein carrier, β-catenin binding, protein C-terminus binding, lipoprotein binding, cytoskeletal protein binding protein, nuclear localization sequence binding, protein phosphatase 1 binding, adenylate cyclase binding, eukaryotic initiation factor 4E binding, calmodulin binding, collagen binding, insulin-like growth factor binding, lamin binding, profilin binding, tropomyosin binding, actin binding, peroxisome targeting sequence binding, SNARE binding, and cyclin binding.

Pharmaceutical compositions including such proteins or protein encoding sequences, antibodies directed against such proteins or polynucleotides capable of altering expression of such proteins, may be used to treat diseases which are associated with impaired protein binding. Antibodies and polynucleotides such as PCR primers and molecular probes designed to identify such proteins or protein encoding sequences may be used for diagnosis of such diseases.

Examples of such diseases include, but are not limited to, neurological and psychiatric diseases [J. Neurosci. 2003, 23(25):8788-99; Neurobiol. Dis. 2003, 14(1):146-56; J. Neurosci. 2003, 23(17):6956-64; Am. J. Pathol. 2003, 163(2):609-19], and cancerous diseases [Cancer Res. 2003, 63(15):4299-304; Semin. Thromb. Hemost. 2003, 29(3):247-58; Proc. Natl. Acad. Sci. USA. 2003, 100(16):9506-1 1].

Ligand Binding or Carrier Proteins:

The phrase “ligand binding or carrier proteins” refers to proteins involved in diverse biological functions such as: pyridoxal phosphate binding, carbohydrate binding, magnesium binding, amino acid binding, cyclosporin A binding, nickel binding, chlorophyll binding, biotin binding, penicillin binding, selenium binding, tocopherol binding, lipid binding, drug binding, oxygen transporter, electron transporter, steroid binding, juvenile hormone binding, retinoid binding, heavy metal binding, calcium binding, protein binding, glycosaminoglycan binding, folate binding, odorant binding, lipopolysaccharide binding and nucleotide binding.

Pharmaceutical compositions including such proteins or protein encoding sequences, antibodies directed against such proteins or polynucleotides capable of altering expression of such proteins, may be used to treat diseases which are associated with impaired function of these proteins. Antibodies and polynucleotides such as PCR primers and molecular probes designed to identify such proteins or protein encoding sequences may be used for diagnosis of such diseases.

Examples of such diseases include, but are not limited to, neurological disorders [J. Med. Genet. 2003, 40(10):733-40; J. Neuropathol. Exp. Neurol. 2003, 62(9):968-75; J. Neurochem. 2003, 87(2):427-36], autoimmune diseases (N. Engl. J. Med. 2003, 349(16):1526-33; JAMA. 2003, 290(13):1721-8]; gastroesophageal reflux disease [Dig. Dis. Sci. 2003, 48(9):1832-8], cardiovascular diseases [J. Vasc. Surg. 2003, 38(4):827-32], cancerous diseases [Oncogene. 2003, 22(43):6699-703; Br. J. Haematol. 2003, 123(2):288-96], respiratory diseases [Circulation. 2003, 108(15):1839-44], and ophtalmic diseases [Ophthalmology. 2003, 110(10):2040-4; Am. J. Ophthalmol. 2003, 136(4):729-32].

ATPases:

The term “ATPases” refers to enzymes that catalyze the hydrolysis of ATP to ADP, releasing energy that is used in the cell. This group include enzymes such as plasma membrane cation-transporting ATPase, ATP-binding cassette (ABC) transporter, magnesium-ATPase, hydrogen-/sodium-translocating ATPase or ATPase translocating any other elements, arsenite-transporting ATPase, protein-transporting ATPase, DNA translocase, P-type ATPase, and hydrolase, acting on acid anhydrides involved in cellular and subcellular movement.

Pharmaceutical compositions including such proteins or protein encoding sequences, antibodies directed against such proteins or polynucleotides capable of altering expression of such proteins, may be used to treat diseases which are associated with impaired conversion of the hydrolysis of ATP to ADP or resulting energy use. Antibodies and polynucleotides such as PCR primers and molecular probes designed to identify such proteins or protein encoding sequences may be used for diagnosis of such diseases.

Examples of such diseases include, but are not limited to, infectious diseases such as helicobacter pylori ulcers [BMC Gastroenterol. 2003, Nov. 6], Neurological, muscular and psychiatric diseases [Int. J. Neurosci. 2003, 13(12):1705-1717; Int. J. Neurosci. 2003, 113(11):1579-1591; Ann. Neurol. 2003, 54(4):494-500], Amyotrophic Lateral Sclerosis [Other Motor Neuron Disord. 2003 4(2):96-9], cardiovascular diseases [J. Nippon. Med. Sch. 2003, 70(5):384-92; Endocrinology. 2003, 144(10):4478-83], metabolic diseases [Mol. Pathol. 2003, 56(5):302-4; Neurosci. Lett. 2003, 350(2):105-8], and peptic ulcer disease treated with inhibitors of the gastric H⁺-K⁺ ATPase (e.g. Omeprazole) responsible for acid secretion in the gastric mucosa.

Carboxylic Ester Hydrolases:

The phrase carboxylic ester hydrolases” refers to hydrolytic enzymes acting on carboxylic ester bonds such as N-acetylglucosaminylphosphatidylinositol deacetylase, 2-acetyl-1-alkylglycerophosphocholine esterase, aminoacyl-tRNA hydrolase, arylesterase, carboxylesterase, cholinesterase, gluconolactonase, sterol esterase, acetylesterase, carboxymethylenebutenolidase, protein-glutamate methylesterase, lipase, and 6-phosphogluconolactonase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other) is abnormal so that a beneficial effect may be achieved by modulation of such reaction. Antibodies and polynucleotides such as PCR primers and molecular probes designed to identify such proteins or protein encoding sequences may be used for diagnosis of such diseases.

Examples of such diseases include, but are not limited to, autoimmune neuromuscular disease Myasthenia Gravis, treated with cholinesterase inhibitors.

Hydrolase, Acting on Ester Bonds:

The phrase “hydrolase, acting on ester bonds” refers to hydrolytic enzymes acting on ester bonds such as nucleases, sulfuric ester hydrolase, carboxylic ester hydrolases, thiolester hydrolase, phosphoric monoester hydrolase, phosphoric diester hydrolase, triphosphoric monoester hydrolase, diphosphoric monoester hydrolase, and phosphoric triester hydrolase.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other), is abnormal. Antibodies and polynucleotides such as PCR primers and molecular probes designed to identify such proteins or protein encoding sequences may be used for diagnosis of such diseases.

Hydrolases:

The term “hydrolases” refers to hydrolytic enzymes such as GPI-anchor transamidase, peptidases, hydrolases, acting on ester bonds, glycosyl bonds, ether bonds, carbon-nitrogen (but not peptide) bonds, acid anhydrides, acid carbon-carbon bonds, acid halide bonds, acid phosphorus-nitrogen bonds, acid sulfur-nitrogen bonds, acid carbon-phosphorus bonds, acid sulfur-sulfur bonds.

Pharmaceutical compositions including such proteins or protein encoding sequences, antibodies directed against such proteins or polynucleotides capable of altering expression of such proteins, may be used to treat diseases in which the hydrolytic cleavage of a covalent bond with accompanying addition of water (—H being added to one product of the cleavage and —OH to the other) is abnormal. Antibodies and polynucleotides such as PCR primers and molecular probes designed to identify such proteins or protein encoding sequences may be used for diagnosis of such diseases.

Examples of such diseases include, but are not limited to, cancerous diseases [Cancer. 2003, 98(9):1842-8; Cancer. 2003, 98(9):1822-9], neurological diseases such as Parkinson diseases [J. Neurol. 2003, 250 Suppl 3:III15-III24; J. Neurol. 2003, 250 Suppl 3:III2-III10], endocrinological diseases such as pancreatitis [Pancreas. 2003, 27(4):291-6] or childhood genetic diseases [Eur. J. Pediatr. 1997, 156(12):935-8], coagulation diseases [BMJ. 2003, 327(7421):974-7], cardiovascular diseases [Ann. Intern. Med. 2003, October 139(8):670-82], autoimmunity diseases [J. Med. Genet. 2003, 40(10):761-6], and metabolic diseases [Am. J. Hum. Genet. 2001, 69(5): 1002-12].

Enzymes:

The term “enzymes” refers to naturally occurring or synthetic macromolecular substance composed mostly of protein, that catalyzes, to various degree of specificity, at least one (bio)chemical reactions at relatively low temperatures. The action of RNA that has catalytic activity (ribozyme) is often also regarded as enzymatic. Nevertheless, enzymes are mainly proteinaceous and are often easily inactivated by heating or by protein-denaturing agents. The substances upon which they act are known as substrates, for which the enzyme possesses a specific binding or active site.

The group of enzymes include various proteins possessing enzymatic activities such as mannosylphosphate transferase, para-hydroxybenzoate:polyprenyltransferase, rieske iron-sulfur protein, imidazoleglycerol-phosphate synthase, sphingosine hydroxylase, tRNA 2′-phosphotransferase, sterol C-24(28) reductase, C-8 sterol isomerase, C-22 sterol desaturase, C-14 sterol reductase, C-3 sterol dehydrogenase (C-4 sterol decarboxylase), 3-keto sterol reductase, C-4 methyl sterol oxidase, dihydronicotinamide riboside quinone reductase, glutamate phosphate reductase, DNA repair enzyme, telomerase, α-ketoacid dehydrogenase, β-alanyl-dopamine synthase, RNA editase, aldo-keto reductase, alkylbase DNA glycosidase, glycogen debranching enzyme, dihydropterin deaminase, dihydropterin oxidase, dimethylnitrosamine demethylase, ecdysteroid UDP-glucosyl/tUDP glucuronosyl transferase, glycine cleavage system, helicase, histone deacetylase, mevaldate reductase, monooxygenase, poly(ADP-ribose) glycohydrolase, pyruvate dehydrogenase, serine esterase, sterol carrier protein X-related thiolase, transposase, tyramine-β hydroxylase, para-aminobenzoic acid (PABA) synthase, glu-tRNA(gln) amidotransferase, molybdopterin cofactor sulfurase, lanosterol 14-α-demethylase, aromatase, 4-hydroxybenzoate octaprenyltransferase, 7,8-dihydro-8-oxoguanine-triphosphatase, CDP-alcohol phosphotransferase, 2,5-diamino-6-(ribosylamino)-4(3H)-pyrimidonone 5′-phosphate deaminase, diphosphoinositol polyphosphate phosphohydrolase, γ-glutamyl carboxylase, small protein conjugating enzyme, small protein activating enzyme, 1-deoxyxylulose-5-phosphate synthase, 2′-phosphotransferase, 2-octoprenyl-3-methyl-6-methoxy-1,4-benzoquinone hydroxylase, 2C-methyl-D-erythritol 2,4-cyclodiphosphate synthase, 3,4 dihydroxy-2-butanone-4-phosphate synthase, 4-amino-4-deoxychorismate lyase, 4-diphosphocytidyl-2C-methyl-D-erythritol synthase, ADP-L-glycero-D-manno-heptose synthase, D-erythro-7,8-dihydroneopterin triphosphate 2′-epimerase, N-ethylmaleimide reductase, O-antigen ligase, O-antigen polymerase, UDP-2,3-diacylglucosamine hydrolase, arsenate reductase, carnitine racemase, cobalamin [5′-phosphate] synthase, cobinamide phosphate guanylyltransferase, enterobactin synthetase, enterochelin esterase, enterochelin synthetase, glycolate oxidase, integrase, lauroyl transferase, peptidoglycan synthetase, phosphopantetheinyltransferase, phosphoglucosamine mutase, phosphoheptose isomerase, quinolinate synthase, siroheme synthase, N-acylmannosamine-6-phosphate 2-epimerase, N-acetyl-anhydromuramoyl-L-alanine amidase, carbon-phosphorous lyase, heme-copper terminal oxidase, disulfide oxidoreductase, phthalate dioxygenase reductase, sphingosine-1-phosphate lyase, molybdopterin oxidoreductase, dehydrogenase, NADPH oxidase, naringenin-chalcone synthase, N-ethylammeline chlorohydrolase, polyketide synthase, aldolase, kinase, phosphatase, CoA-ligase, oxidoreductase, transferase, hydrolase, lyase, isomerase, ligase, ATPase, sulfhydryl oxidase, lipoate-protein ligase, δ-1-pyrroline-5-carboxyate synthetase, lipoic acid synthase, and tRNA dihydrouridine synthase.

Pharmaceutical compositions including such proteins or protein encoding sequences, antibodies directed against such proteins or polynucleotides capable of altering expression of such proteins, may be used to treat diseases which can be ameliorated by modulating the activity of various enzymes which are involved both in enzymatic processes inside cells as well as in cell signaling. Antibodies and polynucleotides such as PCR primers and molecular probes designed to identify such proteins or protein encoding sequences may be used for diagnosis of such diseases.

Cytoskeletal Proteins:

The term “cytoskeletal proteins” refers to proteins involved in the structure formation of the cytoskeleton.

Pharmaceutical compositions including such proteins or protein encoding sequences, antibodies directed against such proteins or polynucleotides capable of altering expression of such proteins, may be used to treat diseases which are caused or due to abnormalities in cytoskeleton, including cancerous cells, and diseased cells such as cells that do not propagate, grow or function normally. Antibodies and polynucleotides such as PCR primers and molecular probes designed to identify such proteins or protein encoding sequences may be used for diagnosis of such diseases.

Examples of such diseases include, but are not limited to, liver diseases such as cholestatic diseases [Lancet. 2003, 362(9390):1112-9], vascular diseases [J. Cell Biol. 2003, 162(6):1111-22], endocrinological diseases [Cancer Res. 2003, 63(16):4836-41], neuromuscular disorders such as muscular dystrophy [Neuromuscul. Disord. 2003, 13(7-8):579-88], or myopathy [Neuromuscul. Disord. 2003, 13(6):456-67] neurological disorders such as Alzheimer's disease [J. Alzheimers Dis. 2003, 5(3):209-28], cardiac disorders [J. Am. Coll. Cardiol. 2003, 42(2):319-27], skin disorders [J. Am. Coll. Cardiol. 2003, 42(2):319-27], and cancer [Proteomics. 2003, 3(6):979-90].

Structural Proteins:

The term “structural proteins” refers to proteins involved in the structure formation of the cell, such as structural proteins of ribosome, cell wall structural proteins, structural proteins of cytoskeleton, extracellular matrix structural proteins, extracellular matrix glycoproteins, amyloid proteins, plasma proteins, structural proteins of eye lens, structural protein of chorion (sensu Insecta), structural protein of cuticle (sensu Insecta), puparial glue protein (sensu Diptera), structural proteins of bone, yolk proteins, structural proteins of muscle, structural protein of vitelline membrane (sensu Insecta), structural proteins of peritrophic membrane (sensu Insecta), and structural proteins of nuclear pores.

Pharmaceutical compositions including such proteins or protein encoding sequences, antibodies directed against such proteins or polynucleotides capable of altering expression of such proteins, may be used to treat diseases which are caused by abnormalities in cytoskeleton, including cancerous cells, and diseased cells such as cells that do not propagate, grow or function normally. Antibodies and polynucleotides such as PCR primers and molecular probes designed to identify such proteins or protein encoding sequences may be used for diagnosis of such diseases.

Examples of such diseases include, but are not limited to, blood vessels diseases such as aneurysms [Cardiovasc. Res. 2003, 60(1):205-13], joint diseases [Rheum. Dis. Clin. North Am. 2003, 29(3):631-45], muscular diseases such as muscular dystrophies [Curr. Opin. Clin. Nutr. Metab. Care. 2003, 6(4):435-9], neuronal diseases such as encephalitis [Neurovirol. 2003, 9(2):274-83], retinitis pigmentosa [Dev. Ophthalmol. 2003, 37:109-25], and infectious diseases [J. Virol. Methods. 2003, 109(1):75-83; FEMS Immunol. Med. Microbiol. 2003, 35(2):125-30; J. Exp. Med. 2003, 197(5):633-42].

Ligands:

The term “ligands” refers to proteins that bind to another chemical entity to form a larger complex, involved in various biological processes, such as signal transduction, metabolism, growth and differentiation, etc. This group of proteins includes opioid peptides, baboon receptor ligand, branchless receptor ligand, breathless receptor ligand, ephrin, frizzled receptor ligand, frizzled-2 receptor ligand, heartless receptor ligand, Notch receptor ligand, patched receptor ligand, punt receptor ligand, Ror receptor ligand, saxophone receptor ligand, SE20 receptor ligand, sevenless receptor ligand, smooth receptor ligand, thickveins receptor ligand, Toll receptor ligand, Torso receptor ligand, death receptor ligand, scavenger receptor ligand, neuroligin, integrin ligand, hormones, pheromones, growth factors, and sulfonylurea receptor ligand.

Pharmaceutical compositions including such proteins or protein encoding sequences, antibodies directed against such proteins or polynucleotides capable of altering expression of such proteins, may be used to treat diseases involved in impaired hormone function or diseases which involve abnormal secretion of proteins which may be due to abnormal presence, absence or impaired normal response to normal levels of secreted proteins. Those secreted proteins include hormones, neurotransmitters, and various other proteins secreted by cells to the extracellular environment. Antibodies and polynucleotides such as PCR primers and molecular probes designed to identify such proteins or protein encoding sequences may be used for diagnosis of such diseases.

Examples of such diseases include, but are not limited to, analgesia inhibited by orphanin FQ/nociceptin [Shane R., et al., (2001) Brain Res., 907(1-2):109-16], stroke protected by estrogen [Alkayed N. J., et al., (2001) J. Neurosci., 21(19):7543-50], atherosclerosis associated with growth hormone deficiency [Elhadd T. A., et al., (2001) J. Clin. Endocrinol. Metab., 86(9):4223-32], diabetes inhibited by α-galactosylceramide [Hong S., et al., (2001) Nat. Med., 7(9): 1052-6], and Huntington's disease associated with huntingtin deficiency [Rao D. S., et al., (2001) Mol. Cell Biol., 21(22):7796-806].

Signal Transducer:

The term “signal transducers” refers to proteins such as activin inhibitors, receptor-associated proteins, α-2 macroglobulin receptors, morphogens, quorum sensing signal generators, quorum sensing response regulators, receptor signaling proteins, ligands, receptors, two-component sensor molecules, and two-component response regulators.

Pharmaceutical compositions including such proteins or protein encoding sequences, antibodies directed against such proteins or polynucleotides capable of altering expression of such proteins, may be used to treat diseases in which the signal-transduction is impaired, either as a cause, or as a result of the disease. Antibodies and polynucleotides such as PCR primers and molecular probes designed to identify such proteins or protein encoding sequences may be used for diagnosis of such diseases.

Examples of such diseases include, but are not limited to, altered sexual dimorphism associated with signal transducer and activator of transcription Sb [Udy G. B., et al., (1997) Proc. Natl. Acad. Sci. USA, 94(14):7239-44], multiple sclerosis associated with sgp130 deficiency [Padberg F., et al., (1999) J. Neuroimmunol., 99(2):218-23], intestinal inflammation associated with elevated signal transducer and activator of transcription 3 activity [Suzuki A., et al., (2001) J Exp Med, 193(4):471-81], carcinoid tumor inhibited by increased signal transducer and activators of transcription 1 and 2 [Zhou Y., et al., (2001) Oncology, 60(4):330-8], and esophageal cancer associated with loss of EGF-STAT1 pathway [Watanabe G., et al., (2001) Cancer J., 7(2): 132-9].

RNA Polymerase II Transcription Factors:

The phrase “RNA polymerase II transcription factors” refers to proteins such as specific and non-specific RNA polymerase II transcription factors, enhancer binding, ligand-regulated transcription factor, and general RNA polymerase II transcription factors.

Pharmaceutical compositions including such proteins or protein encoding sequences, antibodies directed against such proteins or polynucleotides capable of altering expression of such proteins, may be used to treat diseases involving impaired function of RNA polymerase II transcription factors. Antibodies and polynucleotides such as PCR primers and molecular probes designed to identify such proteins or protein encoding sequences may be used for diagnosis of such diseases.

Examples of such diseases include, but are not limited to, cardiac diseases [Cell Cycle. 2003, 2(2):99-104], xeroderma pigmentosum [Bioessays. 2001, 23(8):671-3; Biochim. Biophys. Acta. 1997, 1354(3):241-51], muscular atrophy [J. Cell Biol. 2001, 152(1):75-85], neurological diseases such as Alzheimer's disease [Front Biosci. 2000, 5:D244-57], cancerous diseases such as breast cancer [Biol. Chem. 1999, 380(2):117-28], and autoimmune disorders [Clin. Exp. Immunol. 1997, 109(3):488-94].

RNA Binding Proteins:

The phrase “RNA binding proteins” refers to RNA binding proteins involved in splicing and translation regulation such as tRNA binding proteins, RNA helicases, double-stranded RNA and single-stranded RNA binding proteins, mRNA binding proteins, snRNA cap binding proteins, 5S RNA and 7S RNA binding proteins, poly-pyrimidine tract binding proteins, snRNA binding proteins, and AU-specific RNA binding proteins.

Pharmaceutical compositions including such proteins or protein encoding sequences, antibodies directed against such proteins or polynucleotides capable of altering expression of such proteins, may be used to treat diseases involving transcription and translation factors such as helicases, isomerases, histones and nucleases, diseases where there is impaired transcription, splicing, post-transcriptional processing, translation or stability of the RNA. Antibodies and polynucleotides such as PCR primers and molecular probes designed to identify such proteins or protein encoding sequences may be used for diagnosis of such diseases.

Examples of such diseases include, but are not limited to, cancerous diseases such as lymphomas [Tumori. 2003, 89(3):278-84], prostate cancer [Prostate. 2003, 57(1):80-92] or lung cancer [J. Pathol. 2003, 200(5):640-6], blood diseases, such as fanconi anemia [Curr. Hematol. Rep. 2003, 2(4):335-40], cardiovascular diseases such as atherosclerosis [J. Thromb. Haemost. 2003, 1(7):1381-90] muscle diseases [Trends Cardiovasc. Med. 2003, 13(5):188-95] and brain and neuronal diseases [Trends Cardiovasc. Med. 2003, 13(5):188-95; Neurosci. Lett. 2003, 342(1-2):41-4].

Nucleic Acid Binding Proteins:

The phrase “nucleic acid binding proteins” refers to proteins involved in RNA and DNA synthesis and expression regulation such as transcription factors, RNA and DNA binding proteins, zinc fingers, helicase, isomerase, histones, nucleases, ribonucleoproteins, and transcription and translation factors.

Pharmaceutical compositions including such proteins or protein encoding sequences, antibodies directed against such proteins or polynucleotides capable of altering expression of such proteins, may be used to treat diseases involving DNA or RNA binding proteins such as: helicases, isomerases, histones and nucleases, for example diseases where there is abnormal replication or transcription of DNA and RNA respectively. Antibodies and polynucleotides such as PCR primers and molecular probes designed to identify such proteins or protein encoding sequences may be used for diagnosis of such diseases.

Examples of such diseases include, but are not limited to, neurological diseases such as renitis pigmentoas [Am. J. Ophthalmol. 2003, 136(4):678-87] parkinsonism [Proc. Natl. Acad. Sci. USA. 2003, 100(18):10347-52], Alzheimer [J. Neurosci. 2003, 23(17):6914-27] and canavan diseases [Brain Res Bull. 2003, 61(4):427-35], cancerous diseases such as leukemia [Anticancer Res. 2003, 23(4):3419-26] or lung cancer [J. Pathol. 2003, 200(5):640-6], miopathy [Neuromuscul Disord. 2003, 13(7-8):559-67] and liver diseases [J. Pathol. 2003, 200(5):553-60].

Proteins Involved in Metabolism:

The phrase “proteins involved in metabolism” refers to proteins involved in the totality of the chemical reactions and physical changes that occur in living organisms, comprising anabolism and catabolism; may be qualified to mean the chemical reactions and physical processes undergone by a particular substance, or class of substances, in a living organism. This group includes proteins involved in the reactions of cell growth and maintenance such as: metabolism resulting in cell growth, carbohydrate metabolism, energy pathways, electron transport, nucleobase, nucleoside, nucleotide and nucleic acid metabolism, protein metabolism and modification, amino acid and derivative metabolism, protein targeting, lipid metabolism, aromatic compound metabolism, one-carbon compound metabolism, coenzymes and prosthetic group metabolism, sulfur metabolism, phosphorus metabolism, phosphate metabolism, oxygen and radical metabolism, xenobiotic metabolism, nitrogen metabolism, fat body metabolism (sensu Insecta), protein localization, catabolism, biosynthesis, toxin metabolism, methylglyoxal metabolism, cyanate metabolism, glycolate metabolism, carbon utilization and antibiotic metabolism.

Pharmaceutical compositions including such proteins or protein encoding sequences, antibodies directed against such proteins or polynucleotides capable of altering expression of such proteins, may be used to treat diseases involving cell metabolism. Antibodies and polynucleotides such as PCR primers and molecular probes designed to identify such proteins or protein encoding sequences may be used for diagnosis of such diseases.

Examples of such metabolism-related diseases include, but are not limited to, multisystem mitochondrial disorder caused by mitochondrial DNA cytochrome C oxidase II deficiency [Campos Y., et al., (2001) Ann. Neurol. 50(3):409-13], conduction defects and ventricular dysfunction in the heart associated with heterogeneous connexin43 expression [Gutstein D. E., et al., (2001) Circulation, 104(10):1194-9], atherosclerosis associated with growth suppressor p27 deficiency [Diez-Juan A., and Andres V. (2001) FASEB J., 15(11):1989-95], colitis associated with glutathione peroxidase deficiency [Esworthy R. S., et al., (2001) Am. J. Physiol. Gastrointest. Liver Physiol., 281(3):G848-55], systemic lupus erythematosus associated with deoxyribonuclease I deficiency [Yasutomo K., et al., (2001) Nat. Genet., 28(4):313-4], alcoholic pancreatitis [Pancreas. 2003, 27(4):281-5], amyloidosis and diseases that are related to amyloid metabolism, such as FMF, atherosclerosis, diabetes, and especially diabetes long term consequences, neurological diseases such as Creutzfeldt-Jakob disease, and Parkinson or Rasmussen's encephalitis.

Cell Growth and/or Maintenance Proteins:

The phrase “Cell growth and/or maintenance proteins” refers to proteins involved in any biological process required for cell survival, growth and maintenance, including proteins involved in biological processes such as cell organization and biogenesis, cell growth, cell proliferation, metabolism, cell cycle, budding, cell shape and cell size control, sporulation (sensu Saccharomyces), transport, ion homeostasis, autophagy, cell motility, chemi-mechanical coupling, membrane fusion, cell-cell fusion, and stress response.

Pharmaceutical compositions including such proteins or protein encoding sequences, antibodies directed against such proteins or polynucleotides capable of altering expression of such proteins, may be used to treat or prevent diseases such as cancer, degenerative diseases, for example neurodegenerative diseases or conditions associated with aging, or alternatively, diseases wherein apoptosis which should have taken place, does not take place. Antibodies and polynucleotides such as PCR primers and molecular probes designed to identify such proteins or protein encoding sequences may be used for diagnosis of such diseases, detection of pre-disposition to a disease, and determination of the stage of a disease.

Examples of such diseases include, but are not limited to, ataxia-telangiectasia associated with ataxia-telangiectasia mutated deficiency [Hande et al., (2001) Hum. Mol. Genet., 10(5):519-28], osteoporosis associated with osteonectin deficiency [Delany et al., (2000) J. Clin. Invest., 105(7):915-23], arthritis caused by membrane-bound matrix metalloproteinase deficiency [Holmbeck et al., (1999) Cell, 99(1):81-92], defective stratum corneum and early neonatal death associated with transglutaminase 1 deficiency [Matsuki et al., (1998) Proc. Natl. Acad. Sci. USA, 95(3):1044-9], and Alzheimer's disease associated with estrogen [Simpkins et al., (1997) Am. J. Med., 103(3A):19S-25S].

Chaperones

Information derived from proteins such as ribosomal chaperone, peptidylprolyl isomerase, lectin-binding chaperone, nucleosome assembly chaperone, chaperonin ATPase, cochaperone, heat shock protein, HSP70/HSP90 organizing protein, fimbrial chaperone, metallochaperone, tubulin folding, HSC70-interacting protein can be used to diagnose/treat diseases involving pathological conditions, which are associated with non-normal protein activity or structure. Binding of the products of the proteins of this family, or antibodies reactive therewith, can modulate a plurality of protein activities as well as change protein structure. Alternatively, diseases in which there is abnormal degradation of other proteins, which may cause non-normal accumulation of various proteinaceous products in cells, caused non-normal (prolonged or shortened) activity of proteins, etc.

Example of diseases that involve chaperones are cancerous diseases, such as prostate cancer (Semin Oncol. 2003 October;30(5):709-16.); infectious diseases, such as prion infection (EMBO J. 2003 Oct. 15;22(20):5435-5445.); neurological syndromes (J Neuropathol Exp Neurol. 2003 July;62(7):751-64.; Antioxid Redox Signal. 2003 June;5(3):337-48.; J. Neurochem. 2003 July;86(2):394-404.)

Variants of Proteins Which Accumulate an Element/Compound

Variant proteins which their wild type version naturally binds a certain compound or element inside the cell for storage of accumulation may have terapoetic effect as secreted variants. Ferritin, accumulates iron inside the cells. A secreted variant of this protein is expected to bind plasma iron, reduce its levels and therefore have a desired therapeutic effect in the syndrome of Hemosiderosis characterized by high levels of iron in the blood.

### Diseases that may be Treated/Diagnosed Using the Biomolecular Sequences of the Present Invention

Inflammatory Diseases

Examples of inflammatory diseases include, but are not limited to, chronic inflammatory diseases and acute inflammatory diseases.

Inflammatory Diseases Associated with Hypersensitivity

Examples of hypersensitivity include, but are not limited to, Types I-IV hypersensitivity, immediate hypersensitivity, antibody mediated hypersensitivity, immune complex mediated hypersensitivity, T lymphocyte mediated hypersensitivity and DTH. An example of type I or immediate hypersensitivity is asthma. Examples of type II hypersensitivity include, but are not limited to, rheumatoid diseases, rheumatoid autoimmune diseases, rheumatoid arthritis [Krenn V. et al., Histol Histopathol 2000 July;15 (3):791], spondylitis, ankylosing spondylitis [Jan Voswinkel et al, Arthritis Res 2001; 3 (3): 189], systemic diseases, systemic autoimmune diseases, systemic lupus erythematosus [Erikson J. et al., Immunol Res 1998;17 (1-2):49], sclerosis, systemic sclerosis [Renaudineau Y. et al., Clin Diagn Lab Immunol. 1999 March;6 (2):156; Chan O T. et al., Immunol Rev 1999 June;169:107], glandular diseases, glandular autoimmune diseases, pancreatic autoimmune diseases, diabetes, Type I diabetes [Zimmet P. Diabetes Res Clin Pract 1996 October;34 Suppl:S125], thyroid diseases, autoimmune thyroid diseases, Graves' disease [Orgiazzi J. Endocrinol Metab Clin North Am 2000 June;29 (2):339], thyroiditis, spontaneous autoimmune thyroiditis [Braley-Mullen H. and Yu S, J Immunol 2000 Dec. 15;165 (12):7262], Hashimoto's thyroiditis [Toyoda N. et al., Nippon Rinsho 1999 August;57 (8):1810], myxedema, idiopathic myxedema [Mitsuma T. Nippon Rinsho. 1999 August;57 (8):1759], autoimmune reproductive diseases, ovarian diseases, ovarian autoimmunity [Garza K M. et al., J Reprod Immunol 1998 February;37 (2):87], autoimmune anti-sperm infertility [Diekman A B. et al., Am J Reprod Immunol. 2000 March;43 (3):134], repeated fetal loss [Tincani A. et al., Lupus 1998;7 Suppl 2:S107-9], neurodegenerative diseases, neurological diseases, neurological autoimmune diseases, multiple sclerosis [Cross A H. et al., J Neuroimmunol 2001 Jan. 1;112 (1-2):1], Alzheimer's disease [Oron L. et al., J Neural Transm Suppl. 1997;49:77], myasthenia gravis [Infante A J. And Kraig E, Int Rev Immunol 1999;18 (1-2):83], motor neuropathies [Kornberg A J. J Clin Neurosci. 2000 May;7 (3):191], Guillain-Barre syndrome, neuropathies and autoimmune neuropathies [Kusunoki S. Am J Med Sci. 2000 April;319 (4):234], myasthenic diseases, Lambert-Eaton myasthenic syndrome [Takamori M. Am J Med Sci. 2000 April;319 (4):204], paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies [Antoine J C. and Honnorat J. Rev Neurol (Paris) 2000 January; 156 (1):23], neuropathies, dysimmune neuropathies [Nobile-Orazio E. et al., Electroencephalogr Clin Neurophysiol Suppl 1999;50:419], neuromyotonia, acquired neuromyotonia, arthrogryposis multiplex congenita [Vincent A. et al., Ann N Y Acad. Sci. 1998 May 13;841:482], cardiovascular diseases, cardiovascular autoimmune diseases, atherosclerosis [Matsuura E. et al., Lupus. 1998;7 Suppl 2:S135], myocardial infarction [Vaarala 0. Lupus. 1998;7 Suppl 2:S132], thrombosis [Tincani A. et al., Lupus 1998;7 Suppl 2:S107-9], granulomatosis, Wegener's granulomatosis, arteritis, Takayasu's arteritis and Kawasaki syndrome [Praprotnik S. et al., Wien Klin Wochenschr 2000 Aug. 25; 112 (15-16):660], anti-factor VIII autoimmune disease [Lacroix-Desmazes S. et al., Semin Thromb Hemost.2000;26 (2):157], vasculitises, necrotizing small vessel vasculitises, microscopic polyangiitis, Churg and Strauss syndrome, glomerulonephritis, pauci-immune focal necrotizing glomerulonephritis, crescentic glomerulonephritis [Noel L H. Ann Med Interne (Paris). 2000 May;151 (3):178], antiphospholipid syndrome [Flamholz R. et al., J Clin Apheresis 1999;14 (4): 171], heart failure, agonist-like β-adrenoceptor antibodies in heart failure [Wallukat G. et al., Am J Cardiol. 1999 Jun. 17;83 (12A):75H], thrombocytopenic purpura [Moccia F. Ann Ital Med Int. 1999 April-June;14 (2):114], hemolytic anemia, autoimmune hemolytic anemia [Efremov D G. et al., Leuk Lymphoma 1998 January;28 (3-4):285], gastrointestinal diseases, autoimmune diseases of the gastrointestinal tract, intestinal diseases, chronic inflammatory intestinal disease [Garcia Herola A. et al., Gastroenterol Hepatol. 2000 January;23 (1):16], celiac disease [Landau Y E. and Shoenfeld Y. Harefuah 2000 Jan. 16;138 (2):122], autoimmune diseases of the musculature, myositis, autoimmune myositis, Sjogren's syndrome [Feist E. et al., Int Arch Allergy Immunol 2000 September;123 (1):92], smooth muscle autoimmune disease [Zauli D. et al., Biomed Pharmacother 1999 June;53 (5-6):234], hepatic diseases, hepatic autoimmune diseases, autoimmune hepatitis [Manns M P. J Hepatol 2000 August;33 (2):326] and primary biliary cirrhosis [Strassburg C P. et al., Eur J Gastroenterol Hepatol. 1999 June; 11 (6):595].

Examples of type IV or T cell mediated hypersensitivity, include, but are not limited to, rheumatoid diseases, rheumatoid arthritis [Tisch R, McDevitt H O. Proc Natl Acad Sci USA 1994 Jan. 18;91 (2):437], systemic diseases, systemic autoimmune diseases, systemic lupus erythematosus [Datta S K., Lupus 1998;7 (9):591], glandular diseases, glandular autoimmune diseases, pancreatic diseases, pancreatic autoimmune diseases, Type I diabetes [Castano L. and Eisenbarth G S. Ann. Rev. Immunol. 8:647], thyroid diseases, autoimmune thyroid diseases, Graves' disease [Sakata S. et al., Mol Cell Endocrinol 1993 March;92 (1):77], ovarian diseases [Garza K M. et al., J Reprod Immunol 1998 February;37 (2):87], prostatitis, autoimmune prostatitis [Alexander R B. et al., Urology 1997 December;50 (6):893], polyglandular syndrome, autoimmune polyglandular syndrome, Type I autoimmune polyglandular syndrome [Hara T. et al., Blood. 1991 Mar. 1;77 (5):1127], neurological diseases, autoimmune neurological diseases, multiple sclerosis, neuritis, optic neuritis [Soderstrom M. et al., J Neurol Neurosurg Psychiatry 1994 May;57 (5):544], myasthenia gravis [Oshima M. et al., Eur J Immunol 1990 December;20 (12):2563], stiff-man syndrome [Hiemstra H S. et al., Proc Natl Acad Sci USA 2001 Mar. 27;98 (7):3988], cardiovascular diseases, cardiac autoimmunity in Chagas' disease [Cunha-Neto E. et al., J Clin Invest 1996 Oct. 15;98 (8):1709], autoimmune thrombocytopenic purpura [Semple J W. et al., Blood 1996 May 15;87 (10):4245], anti-helper T lymphocyte autoimmunity [Caporossi A P. et al., Viral Immunol 1998;11 (1):9], hemolytic anemia [Sallah S. et al., Ann Hematol 1997 March;74 (3):139], hepatic diseases, hepatic autoimmune diseases, hepatitis, chronic active hepatitis [Franco A. et al., Clin Immunol Immunopathol 1990 March;54 (3):382], biliary cirrhosis, primary biliary cirrhosis [Jones D E. Clin Sci (Colch) 1996 November;91 (5):551], nephric diseases, nephric autoimmune diseases, nephritis, interstitial nephritis [Kelly C J. J Am Soc Nephrol 1990 August;1 (2):140], connective tissue diseases, ear diseases, autoimmune connective tissue diseases, autoimmune ear disease [Yoo T J. et al., Cell Immunol 1994 August; 157 (1):249], disease of the inner ear [Gloddek B. et al., Ann N Y Acad Sci 1997 Dec. 29;830:266], skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Autoimmune Diseases

Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular and blood diseases include, but are not limited to atherosclerosis [Matsuura E. et al., Lupus. 1998;7 Suppl 2:S135], myocardial infarction [Vaarala O. Lupus. 1998;7 Suppl 2:S132], thrombosis [Tincani A. et al., Lupus 1998;7 Suppl 2:S107-9], Wegener's granulomatosis, Takayasu's arteritis, Kawasaki syndrome [Praprotnik S. et al., Wien Klin Wochenschr 2000 Aug. 25;112 (15-16):660], anti-factor VIII autoimmune disease [Lacroix-Desmazes S. et al., Semin Thromb Hemost.2000;26 (2): 157], necrotizing small vessel vasculitis, microscopic polyangiitis, Churg and Strauss syndrome, pauci-immune focal necrotizing and crescentic glomerulonephritis [Noel L H. Ann Med Interne (Paris). 2000 May;151 (3):178], antiphospholipid syndrome [Flamholz R. et al., J Clin Apheresis 1999;14 (4):171], antibody-induced heart failure [Wallukat G. et al., Am J Cardiol. 1999 Jun. 17;83 (12A):75H], thrombocytopenic purpura [Moccia F. Ann Ital Med Int. 1999 April-June;14 (2):114; Semple J W. et al., Blood 1996 May 15;87 (10):4245], autoimmune hemolytic anemia [Efremov D G. et al., Leuk Lymphoma 1998 January;28 (3-4):285; Sallah S. et al., Ann Hematol 1997 March;74 (3):139], cardiac autoimmunity in Chagas' disease [Cunha-Neto E. et al., J Clin Invest 1996 Oct. 15;98 (8):1709) and anti-helper T lymphocyte autoimmunity [Caporossi A P. et al., Viral Immunol 1998;11 (1):9].

Examples of autoimmune rheumatoid diseases include, but are not limited to rheumatoid arthritis [Krenn V. et al., Histol Histopathol 2000 July;15 (3):791; Tisch R, McDevitt HO. Proc Natl Acad Sci units S A 1994 Jan. 18;91 (2):437) and ankylosing spondylitis [Jan Voswinkel et al., Arthritis Res 2001; 3 (3): 189].

Examples of autoimmune glandular diseases include, but are not limited to, pancreatic disease, Type I diabetes, Type II diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type I diabetes [Castano L. and Eisenbarth G S. Ann. Rev. Immunol. 8:647; Zimmet P. Diabetes Res Clin Pract 1996 October;34 Suppl:S125], autoimmune thyroid diseases, Graves' disease [Orgiazzi J. Endocrinol Metab Clin North Am 2000 June;29 (2):339; Sakata S. et al., Mol Cell Endocrinol 1993 March;92 (1):77], spontaneous autoimmune thyroiditis [Braley-Mullen H. and Yu S, J Immunol 2000 Dec. 15;165 (12):7262], Hashimoto's thyroiditis [Toyoda N. et al., Nippon Rinsho 1999 August;57 (8):1810], idiopathic myxedema [Mitsuma T. Nippon Rinsho. 1999 August;57 (8):1759], ovarian autoimmunity [Garza K M. et al., J Reprod Immunol 1998 February;37 (2):87], autoimmune anti-sperm infertility [Diekman A B. et al., Am J Reprod Immunol. 2000 March;43 (3):134], autoimmune prostatitis [Alexander R B. et al., Urology 1997 December;50 (6):893) and Type I autoimmune polyglandular syndrome [Hara T. et al., Blood. 1991 Mar. 1;77 (5): 1127].

Examples of autoimmune gastrointestinal diseases include, but are not limited to, chronic inflammatory intestinal diseases [Garcia Herola A. et al., Gastroenterol Hepatol. 2000 January;23 (1):16], celiac disease [Landau Y E. and Shoenfeld Y. Harefuah 2000 Jan. 16; 138 (2):122], colitis, ileitis and Crohn's disease and ulcerative colitis.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis [Franco A. et al., Clin Immunol Immunopathol 1990 March;54 (3):382], primary biliary cirrhosis [Jones D E. Clin Sci (Colch) 1996 November;91 (5):551; Strassburg C P. et al., Eur J Gastroenterol Hepatol. 1999 June;11 (6):595) and autoimmune hepatitis [Manns M P. J Hepatol 2000 August;33 (2):326].

Examples of autoimmune neurological diseases include, but are not limited to, multiple sclerosis [Cross A H. et al., J Neuroimmunol 2001 Jan. 1;112 (1-2):1], Alzheimer's disease [Oron L. et al., J Neural Transm Suppl. 1997;49:77], myasthenia gravis [Infante A J. And Kraig E, Int Rev Immunol 1999;18 (1-2):83; Oshima M. et al., Eur J Immunol 1990 December;20 (12):2563], neuropathies, motor neuropathies [Kornberg A J. J Clin Neurosci. 2000 May;7 (3):191], Guillain-Barre syndrome and autoimmune neuropathies [Kusunoki S. Am J Med Sci. 2000 April;319 (4):234], myasthenia, Lambert-Eaton myasthenic syndrome [Takamori M. Am J Med Sci. 2000 April;319 (4):204], paraneoplastic neurological diseases, cerebellar atrophy, paraneoplastic cerebellar atrophy and stiff-man syndrome [Hiemstra H S. et al., Proc Natl Acad Sci units S A 2001 Mar. 27;98 (7):3988], non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies [Antoine J C. and Honnorat J. Rev Neurol (Paris) 2000 January;156 (1):23], dysimmune neuropathies [Nobile-Orazio E. et al., Electroencephalogr Clin Neurophysiol Suppl 1999;50:419], acquired neuromyotonia, arthrogryposis multiplex congenita [Vincent A. et al., Ann NY Acad. Sci. 1998 May 13;841:482], neuritis, optic neuritis [Soderstrom M. et al., J Neurol Neurosurg Psychiatry 1994 May;57 (5):544) multiple sclerosis and neurodegenerative diseases.

Examples of autoimmune muscular diseases include, but are not limited to, myositis, autoimmune myositis and primary Sjogren's syndrome [Feist E. et al., Int Arch Allergy Immunol 2000 September; 123 (1):92) and smooth muscle autoimmune disease [Zauli D. et al., Biomed Pharmacother 1999 June;53 (5-6):234].

Examples of autoimmune nephric diseases include, but are not limited to, nephritis and autoimmune interstitial nephritis [Kelly C J. J Am Soc Nephrol 1990 August; 1 (2): 140], glommerular nephritis.

Examples of autoimmune diseases related to reproduction include, but are not limited to, repeated fetal loss [Tincani A. et al., Lupus 1998;7 Suppl 2:S107-9].

Examples of autoimmune connective tissue diseases include, but are not limited to, ear diseases, autoimmune ear diseases [Yoo T J. et al., Cell Immunol 1994 August; 157 (1):249) and autoimmune diseases of the inner ear [Gloddek B. et al., Ann NY Acad Sci 1997 Dec. 29;830:266].

Examples of autoimmune systemic diseases include, but are not limited to, systemic lupus erythematosus [Erikson J. et al., Immunol Res 1998;17 (1-2):49) and systemic sclerosis [Renaudineau Y. et al., Clin Diagn Lab Immunol. 1999 March;6 (2):156; Chan O T. et al., Immunol Rev 1999 June; 169:107].

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases, and prion diseases.

Graft Rejection Diseases

Examples of diseases associated with transplantation of a graft include, but are not limited to, graft rejection, chronic graft rejection, subacute graft rejection, hyperacute graft rejection, acute graft rejection, and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; malignant lymphoma, such as Birkitt's Non-Hodgkin's; Lymphoctyic leukemia, such as acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate; Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

### Example 9

### Microarray Analysis Based Validation of the Antisense Dataset

A microarray-based analysis using oligonucleotide probes that hybridize to the target in a strand-specific manner, was conducted in order to experimentally validate the predicted antisense/sense pairs of the database. Two complementary 60-mer oligonucleotide probes derived from the predicted overlap region of the sense/antisense pairs, were designed. Single 60-mer oligonucleotides were previously shown to offer reliability and sensitivity for detecting specific transcripts (T. R. Hughes, et al., Nature Biotech. 19, 342 (2001).) Initially only pairs of clusters with an overlap greater than 60 bases (2,464 pairs agree with this restriction) were selected for array construction. The overlap region of each antisense pair was then verified for the presence of 60-mer oligonucleotides that matched a set of standards, such as minimal sequence similarity elsewhere in the human genome, uniform GC-content and Tm, and absence of palindromic sequences, in order to maximize the hybridization specificity. Oligonucleotide probes meeting the criteria set forth were identified for 1,211 sense/antisense pairs and a random sample of 264 pairs, which constitutes roughly one-tenth of the original dataset of 2667 sense/antisense cluster pairs, was selected for analysis by Microarrays (Table_S1 on CD-ROM2, an excerpt of which is shown in Table 5 below). In this sample, the proportion of each of the nine subgroups depicted in Table 4 is similar to that of the original dataset, indicating a good representation of the various subgroups.

TABLE 4

| mRNA/ | No cluster | 1 cluster | 2 clusters | | |
|---|---|---|---|---|---|
| Splicing | w introns | w intron(s) | w intron(s) | Total | |
| No cluster | 48 | 132 | 197 | 377 | (14%) |
| w mRNA | | | | | |
| 1 cluster | 17 | 490 | 1039 | 1546 | (58%) |
| w mRNA | | | | | |
| 2 clusters | 1 | 85 | 658 | 744 | (28%) |
| w mRNA | | | | | |
| Total | 66 | 707 | 1894 | 2667 | (100%) |
| | (2.5%) | (26%) | (71%) | | |

Table represents the proportion of sense/antisense clusters in the dataset of 2667 that contain:
1) a known mRNA and
2) expressed sequences spanning at least one intron, in one of the two clusters, in both clusters or in none of the clusters.

Table 5 below is an excerpt of Table_S1 provided on CD-ROM2; Table 5 exemplifies five of the putative sense/antisense pairs that were selected for microarray analysis. The first column provides the pair number. The next two columns provide the accession numbers of representative expressed sequences from the overlapping region of the sense and the antisense genes, respectively. The two columns identified by the “RNA” header provide the accession numbers of known mRNAs in the sense and antisense clusters (if available), and the last two columns provide the GenBank descriptions of these mRNAs.

TABLE 5

| | sense seq. | antisense | RNA | RNA | description | description |
|---|---|---|---|---|---|---|
| | from over- | seq. from | in | in | of RNA | of RNA |
| Pair | lapping | overlapping | sense | a-sense | in sense | in antisense |
| no. | region | region | cluster | cluster | cluster | cluster |
| 235 | NM_6227 | NM_308 | NM_6227 | NM_308 | Homo sapiens | Homo sapiens |
| | | | | | phospholipid | protective protein for |
| | | | | | transfer protein | beta-galactosidase |
| | | | | | (PLTP), mRNA | (galactosialidosis) |
| | | | | | #DV L26232.1 | (PPGB), mRNA |
| 237 | NM_4703 | NM_2532 | NM_4703 | NM_2532 | Homo sapiens | Homo sapiens |
| | | | | | rabaptin-5 | nucleoporin 88 kD |
| | | | | | (RAB5EP), mRNA | (NUP88) mRNA |
| | | | | | #DV X91141.1 | #DV Y08612.2 |
| 217 | NM_14885 | AV 723808 | NM_14885 | NM_2940 | Homo sapiens | Homo sapiens ATP- |
| | | | | | anaphase-promoting | binding cassette, |
| | | | | | complex 10 | sub-family E |
| | | | | | (APC10) mRNA. | (OABP), member 1 |
| | | | | | #DV AL080090.1 | (ABCE1), mRNA. |
| 209 | BC 8865 | BG 717574 | NM_32231 | NM_3099 | Homo sapiens | Homo sapiens |
| | | | | | hypothetical protein | sorting nexin 1 |
| | | | | | FLJ22875 | (SNX1), mRNA. |
| | | | | | (FLJ22875), mRNA | #DV U53225.1 |
| 196 | BE 885605 | AL 527611 | NM_17832 | NM_3640 | Homo sapiens | Homo sapiens |
| | | | | | hypothetical protein | inhibitor of kappa |
| | | | | | FLJ20457 | light polypeptide gene |
| | | | | | (FLJ20457), mRNA | enhancer in B-cells, |
| | | | | | | kinase complex- |
| | | | | | | associated protein |
| | | | | | | (IKBKAP), mRNA |

Microarrays were constructed by spotting each of the 264 pairs of oligonucleotide probes onto treated glass slides in quadruplicates. The two counterpart oligonucleotide probes of each pair were spotted next to each other to ensure similar hybridization conditions.

As positive controls, each of the blocks contained oligonucleotides spotted at various concentrations for four ubiquitously expressed housekeeping genes: guanine nucleotide binding protein beta polypeptide 2-like 1 (gnb211, HUMMHBA123, NM_{—}006098), heat shock 70 kD protein 10 (hsp70, HSHSC70CDS0, NM_{—}006597), beta actin (actin, ACTB, NM_{—}001101), and glyceraldehyde-3-phosphate dehydrogenase (gapdh, NM_{—}002046).

Two random oligonucleotides were used as negative controls. These computer-generated arbitrary sequences displayed no alignment to human genome sequences but had the same physical characteristics as the other oligonucleotide probes. In addition, 22 probes for 11 previously documented sense/antisense pairs were also analyzed in the Microarrays (entries Pair no. “known l”-“known 11” on Table_S1 of CD-ROM2).

The Microarrays were hybridized with poly(A)+ RNAs obtained from 19 human cell lines representing a variety of tissues and four normal human tissues (see General Materials and Methods section above). Each poly(A)+ RNA was reverse transcribed by priming with oligo(dT) and random nonamers, and engineered to incorporate a fluorescent marker. A pool containing an equal mix of the RNAs from all cell lines was also transcribed and used as a reference target. The resulting fluorescently-labeled cDNAs were combined and hybridized to the oligonucleotide Microarrays.

The experiments were performed in duplicate and utilized a fluorescent reversal of the Cy3- and Cy5-labelled cDNA. Stringent hybridization conditions were utilized in order to minimize the appearance of false positive signals, despite the possibility of compromised detection of low abundance transcripts.

The raw data was normalized at several levels; within each slide, between reciprocal slides, and globally between slides (see General Materials and Methods section above). Non-specific levels of hybridization were estimated from the negative controls. The threshold for significant positive signals resulting from authentic hybridization was set at 4 standard deviations of the mean normalized signals for the negative controls. Processed data was presented as normalized signal intensity and as normalized signal ratios (Table_S2 on CD-ROM2).

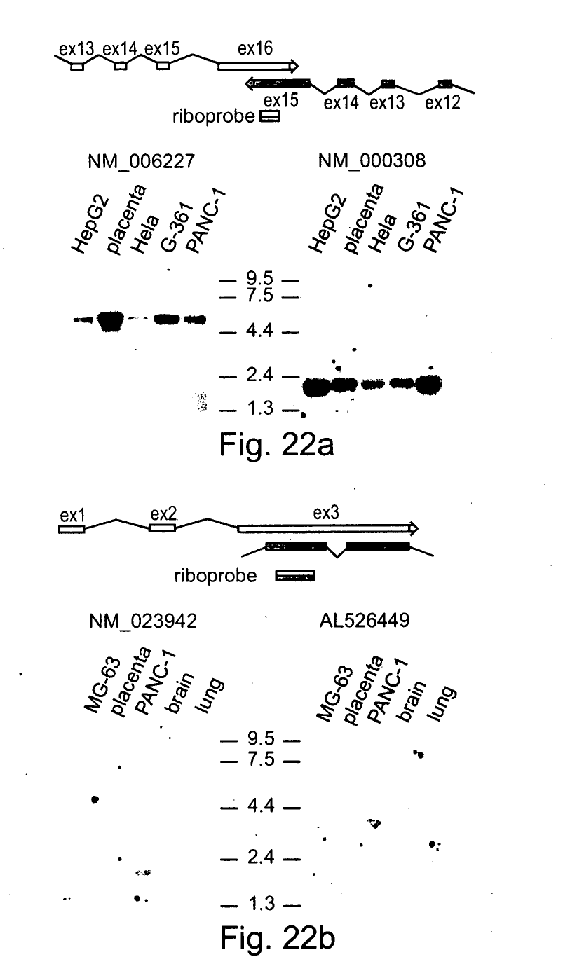

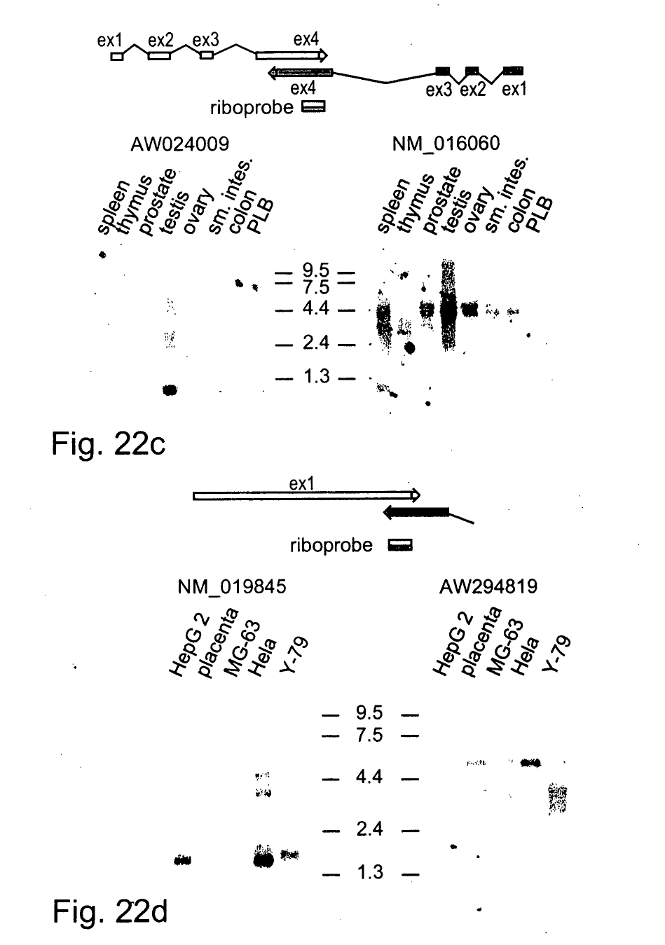

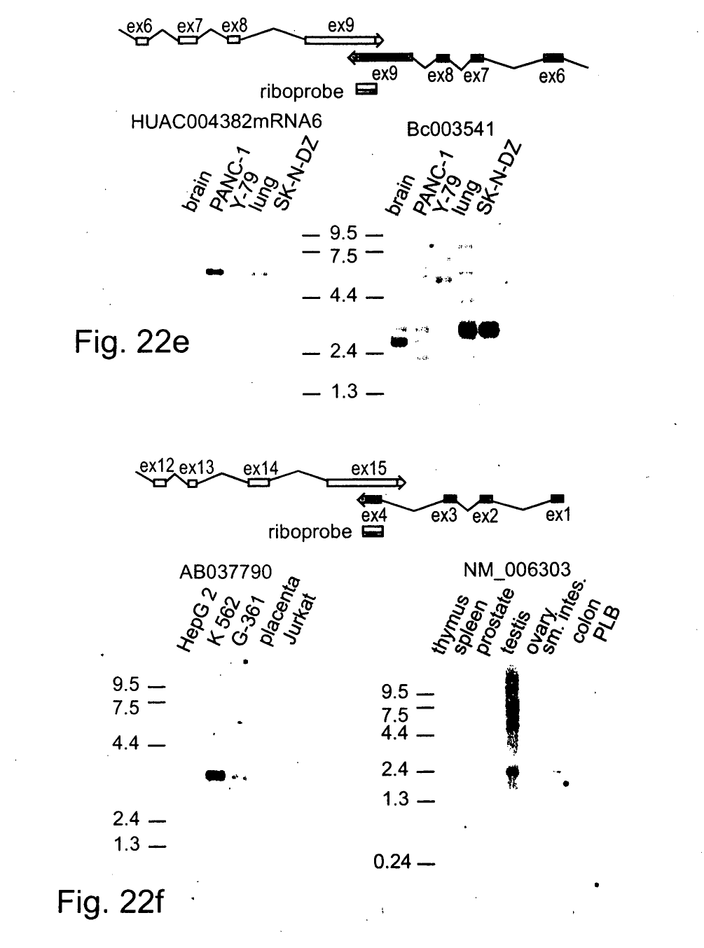

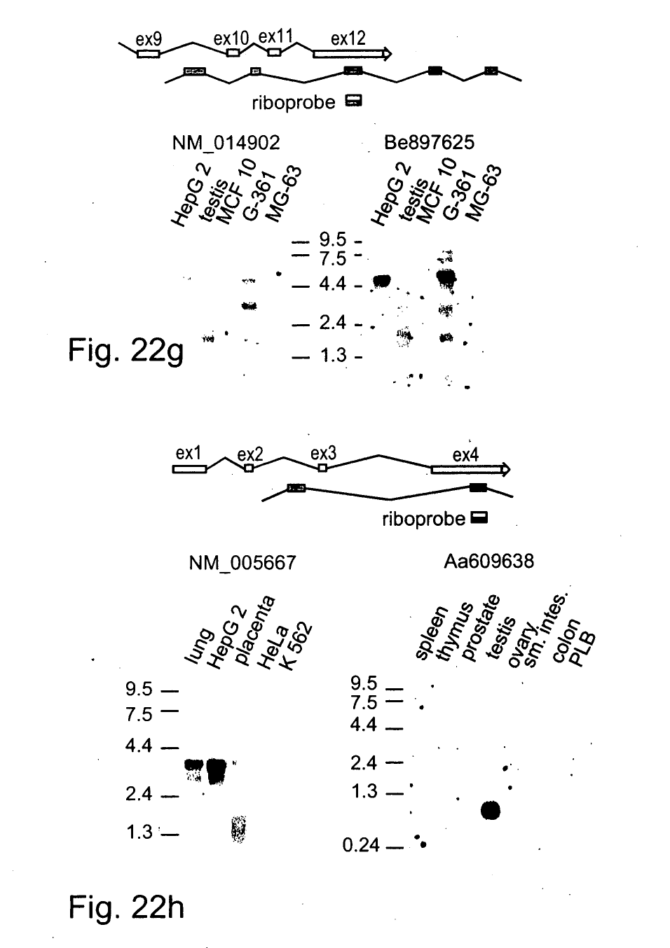

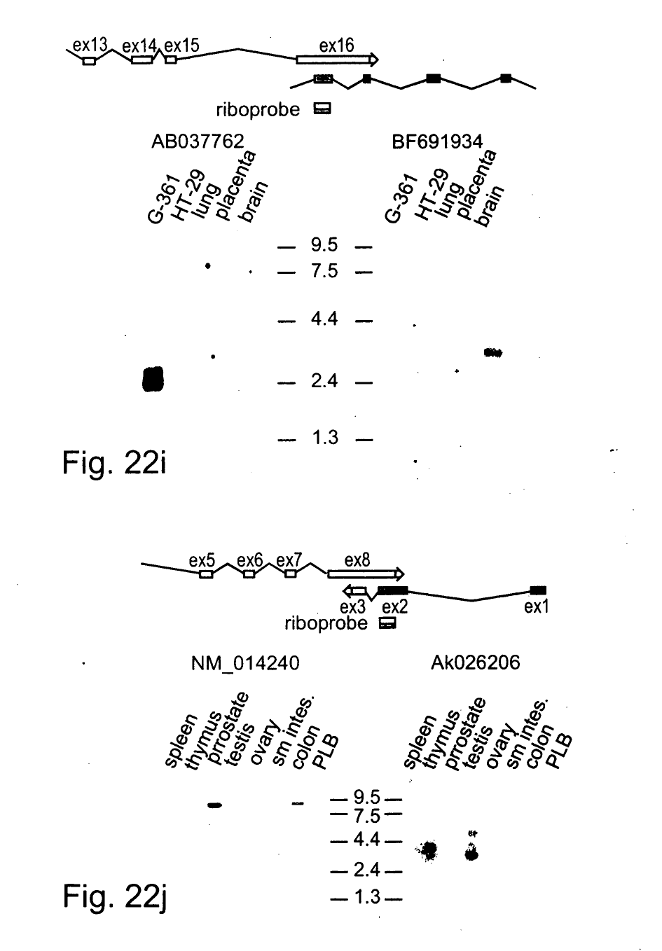

To further substantiate array results, several pairs of oligonucleotides were also utilized in Northern blot analysis. FIGS. 22a-j illustrate results of such northern blot analysis. FIG. 22a reveals expression patterns of randomly selected sequence pair number 235, denoted as Rand_{—}235 in Table 6, below. Similarly, FIG. 22b corresponds to pair number 173, FIG. 22c to pair number 248, FIG. 22d to pair number 6, FIG. 22e to pair number 216, FIG. 22f to pair number 239, FIG. 22g to pair number 202, FIG. 22h to pair number 114, FIG. 22i to pair number 188, and FIG. 22j to pair number 223. Eight pairs (FIGS. 22a-h) evaluated revealed positive signals for both sense and antisense expression, while two (FIGS. 22i-j) revealed a positive signal for only one of the genes, with the counterpart being a known RefSeq mRNA.

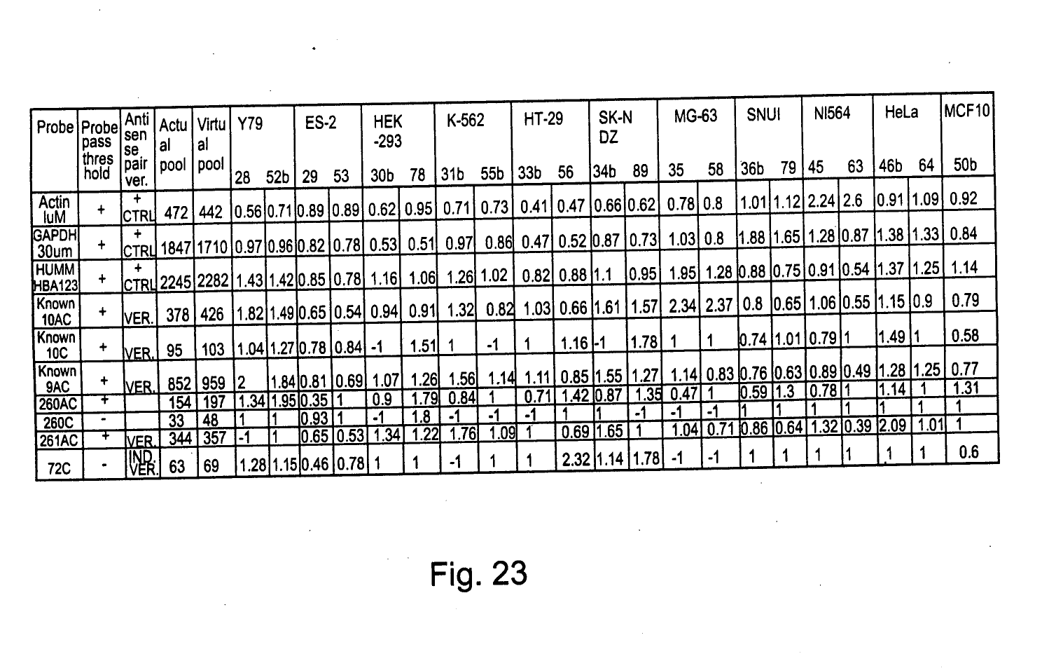

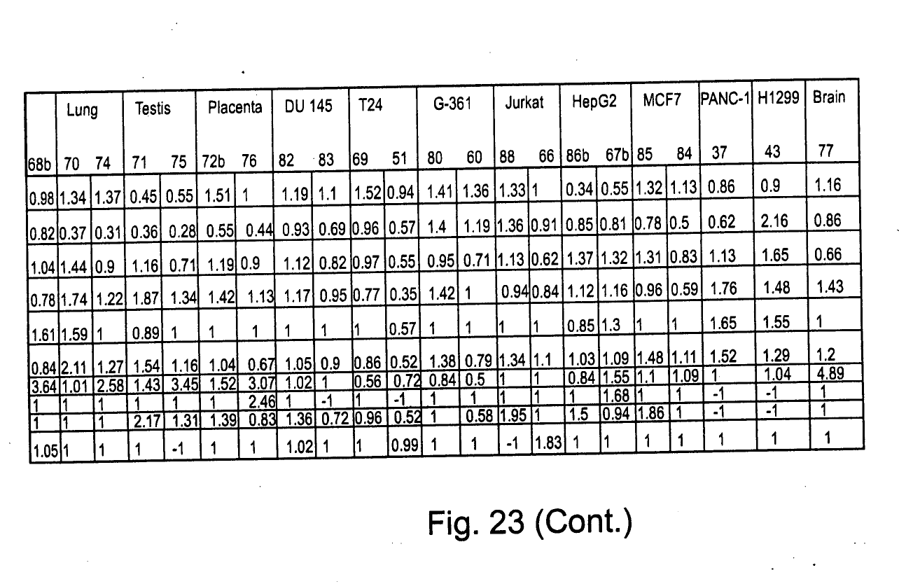

FIG. 23 represents an excerpt of Table_S2 (provided in CD-ROM2) which summarizes the results obtained utilizing the array generated according to the teachings of the present invention. Expression thresholds were verified and indicated and normalization for microarray signals was conducted as described above. Rji ratios were obtained for each cell line/tissue assessed.

Taken cumulatively, the data presented herein revealed positive signals for both sense and antisense transcripts in 65 cluster pairs. In another 47 cases, significant hybridization signals were detected for antisense sequences with known counterpart sense transcripts, i.e. RefSeq mRNAs, which did not give clear hybridization signals on the Microarrays. Thus, 42.5% (112 cases) of the 264 represented on the Microarrays, yielded detectable antisense transcription. The conversion table, assigning the respective serial number as it appears in the “table_{—}125” file of CD-ROM2 and “table_{—}133” file of CD-ROM 3 enclosed herewith, is shown in Table 6 below.

TABLE 6

| Rand_# | Serial No |
|---|---|
| Rand_1 | 2326 |
| Rand_10 | 3647 |
| Rand_100 | 2758 |
| Rand_101 | 1595 |
| Rand_102 | 3686 |
| Rand_103 | 2331 |
| Rand_104 | 3496 |
| Rand_105 | 3134 |
| Rand_106 | 1339 |
| Rand_107 | 908 |
| Rand_108 | 2929 |
| Rand_109 | 2537 |
| Rand_11 | 2806 |
| Rand_110 | 3594 |
| Rand_111 | 2819 |
| Rand_112 | 3019 |
| Rand_113 | 3815 |
| Rand_114 | 2606 |
| Rand_115 | 1662 |
| Rand_116 | 2171 |
| Rand_117 | 2539 |
| Rand_118 | 2802 |
| Rand_119 | 2761 |
| Rand_12 | 1947 |
| Rand_120 | 3228 |
| Rand_121 | 2076 |
| Rand_122 | 1835 |
| Rand_123 | 3029 |
| Rand_124 | 2898 |
| Rand_125 | 1568 |
| Rand_126 | 2456 |
| Rand_127 | 2019 |
| Rand_128 | 2346 |
| Rand_129 | 2460 |
| Rand_13 | 2429 |
| Rand_130 | 3374 |
| Rand_131 | 3292 |
| Rand_132 | 3259 |
| Rand_133 | 3591 |
| Rand_134 | 3340 |
| Rand_135 | 1958 |
| Rand_136 | 2274 |
| Rand_137 | 3527 |
| Rand_138 | 1533 |
| Rand_139 | 2622 |
| Rand_14 | 2058 |
| Rand_140 | 2578 |
| Rand_141 | 3492 |
| Rand_142 | 3928 |
| Rand_143 | 2282 3790 |
| Rand_144 | 2820 |
| Rand_145 | 1329 |
| Rand_146 | 1783 |
| Rand_147 | 1527 |
| Rand_148 | 2662 |
| Rand_149 | 2031 |
| Rand_15 | 2677 |
| Rand_150 | 1303 1659 |
| Rand_151 | 1767 |
| Rand_152 | 3378 |
| Rand_153 | 984 |
| Rand_154 | 3759 |
| Rand_155 | 2046 |
| Rand_156 | 2528 |
| Rand_157 | 283 1798 2048 |
| Rand_158 | 3710 |
| Rand_159 | 3178 |
| Rand_16 | 3336 |
| Rand_160 | 1645 |
| Rand_161 | 2074 3464 |
| Rand_162 | 3436 |
| Rand_163 | 2738 |
| Rand_164 | 2749 |
| Rand_165 | 2206 |
| Rand_166 | 1349 |
| Rand_167 | 2773 |
| Rand_168 | 3305 |
| Rand_169 | 1954 |
| Rand_17 | 3940 |
| Rand_170 | 2813 |
| Rand_171 | 3868 |
| Rand_172 | 762 1424 3942 |
| Rand_173 | 3872 |
| Rand_174 | 3801 |
| Rand_175 | 2547 |
| Rand_176 | 1251 |
| Rand_177 | 1603 |
| Rand_178 | 2769 |
| Rand_179 | 3266 |
| Rand_18 | 3073 |
| Rand_180 | 1794 |
| Rand_181 | 1585 |
| Rand_182 | 3554 |
| Rand_183 | 3377 |
| Rand_184 | 3466 |
| Rand_185 | 3159 |
| Rand_186 | 1413 |
| Rand_187 | 3645 |
| Rand_188 | 3880 |
| Rand_189 | 3009 |
| Rand_19 | 3641 |
| Rand_190 | 2549 |
| Rand_191 | 2874 |
| Rand_192 | 2515 |
| Rand_193 | 3914 |
| Rand_194 | 2751 |
| Rand_195 | 2091 |
| Rand_196 | 1966 |
| Rand_197 | 3778 |
| Rand_198 | 3877 |
| Rand_199 | 2248 |
| Rand_2 | 3172 |
| Rand_20 | 2360 |
| Rand_200 | 2064 |
| Rand_201 | 3597 |
| Rand_202 | 2826 |
| Rand_203 | 2388 |
| Rand_204 | 3889 |
| Rand_205 | 2211 |
| Rand_206 | 3512 |
| Rand_207 | 3452 |
| Rand_208 | 3886 |
| Rand_209 | 1600 |
| Rand_21 | 2952 |
| Rand_210 | 2432 |
| Rand_211 | 1651 3968 |
| Rand_212 | 3074 |
| Rand_213 | 2341 |
| Rand_214 | 1984 |
| Rand_215 | 2803 |
| Rand_216 | 3806 |
| Rand_217 | 2186 |
| Rand_218 | 857 |
| Rand_219 | 1744 |
| Rand_22 | 2285 |
| Rand_220 | 2977 |
| Rand_221 | 3863 |
| Rand_222 | 2846 |
| Rand_223 | 3986 |
| Rand_224 | 579 3688 |
| Rand_225 | 3984 |
| Rand_226 | 2889 |
| Rand_227 | 3869 |
| Rand_228 | 3994 |
| Rand_229 | 3818 |
| Rand_23 | 3890 |
| Rand_230 | 3152 |
| Rand_231 | 3445 |
| Rand_232 | 3663 |
| Rand_233 | 3410 |
| Rand_234 | 1112 |
| Rand_235 | 3918 |
| Rand_236 | 2316 |
| Rand_237 | 3673 |
| Rand_238 | 3990 |
| Rand_239 | 4012 |
| Rand_24 | 3250 |
| Rand_240 | 2932 |
| Rand_241 | 3836 |
| Rand_242 | 3424 |
| Rand_243 | 3982 |
| Rand_244 | 3472 |
| Rand_245 | 2071 |
| Rand_246 | 3904 |
| Rand_247 | 2056 |
| Rand_248 | 3855 |
| Rand_249 | 2980 |
| Rand_25 | 3453 |
| Rand_250 | 3565 |
| Rand_251 | 2459 |
| Rand_252 | 71 3147 |
| Rand_253 | 3967 |
| Rand_254 | 702 2867 3088 |
| Rand_255 | 3156 |
| Rand_256 | 2324 2998 |
| Rand_257 | 2284 |
| Rand_258 | 3807 |
| Rand_259 | 2621 |
| Rand_26 | 4009 |
| Rand_27 | 3393 |
| Rand_28 | 3589 |
| Rand_29 | 1837 |
| Rand_3 | 3046 |
| Rand_30 | 3297 |
| Rand_31 | 3692 |
| Rand_32 | 707 2376 |
| Rand_33 | 2052 |
| Rand_34 | 1904 |
| Rand_35 | 3718 |
| Rand_36 | 3898 |
| Rand_37 | 1821 |
| Rand_38 | 3092 |
| Rand_39 | 3262 |
| Rand_4 | 3558 |
| Rand_40 | 2474 |
| Rand_41 | 3568 |
| Rand_42 | 864 |
| Rand_43 | 1864 |
| Rand_44 | 3045 |
| Rand_45 | 2854 |
| Rand_46 | 3852 |
| Rand_47 | 3096 |
| Rand_48 | 1987 |
| Rand_49 | 2893 |
| Rand_5 | 2060 |
| Rand_50 | 1058 |
| Rand_51 | 3560 |
| Rand_52 | 2604 |
| Rand_53 | 3397 |
| Rand_54 | 2040 |
| Rand_55 | 3784 |
| Rand_56 | 3659 |
| Rand_57 | 2005 2688 |
| Rand_58 | 3187 |
| Rand_59 | 1350 |
| Rand_6 | 2202 |
| Rand_60 | 3183 |
| Rand_61 | 2275 |
| Rand_62 | 3882 |
| Rand_63 | 1044 3899 |
| Rand_64 | 2811 |
| Rand_65 | 3232 |
| Rand_66 | 3242 |
| Rand_67 | 34 112 2727 |
| Rand_68 | 3909 |
| Rand_69 | 4016 |
| Rand_7 | 2337 |
| Rand_70 | 2101 3707 |
| Rand_71 | 3703 |
| Rand_72 | 3477 |
| Rand_73 | 2437 |
| Rand_74 | 3808 |
| Rand_75 | 3905 |
| Rand_76 | 1138 2194 |
| Rand_77 | 819 |
| Rand_78 | 3704 |
| Rand_79 | 2309 |
| Rand_8 | 3441 |
| Rand_80 | 1219 |
| Rand_81 | 1416 |
| Rand_82 | 1543 |
| Rand_83 | 3269 |
| Rand_84 | 532 732 |
| Rand_85 | 2607 |
| Rand_86 | 1867 |
| Rand_87 | 627 3006 |
| Rand_88 | 2068 |
| Rand_89 | 2296 |
| Rand_9 | 3741 |
| Rand_90 | 1076 |
| Rand_91 | 3385 |
| Rand_92 | 2334 |
| Rand_93 | 2833 |
| Rand_94 | 2626 |
| Rand_95 | 3671 |
| Rand_96 | 1923 |
| Rand_97 | 1863 |
| Rand_98 | 3437 |
| Rand_99 | 3469 |
| Rand_260 | 1975 3171 |
| Rand_261 | 4013 |
| Rand_262 | 2418 |
| Rand_263 | 2451 |
| Rand_264 | 3832 |

Rand # = the name of the pair on the chip as it appears in Table_S2 on CD-ROM2, column “Probe”;
Serial No = no of the pair in the Table files on CD-ROMs 2 and 3 (could be more than one in case the antisense event was separated to more than two contigs).

The sensitivity of the experimental approach utilized, i.e. the ability to detect a given transcript, stems from a combination of the stringency used in the microarray analysis and the level of expression and tissue specificity of the RNA. This can be estimated from the positive signals obtained for 65% of the oligos representing known RefSeq mRNAs on the Microarrays. This level of detection is comparable to that obtained in other studies, such as the 58% of known exons verified using microarray analysis (D. D. Shoemaker, et al., Nature 409, 922; 2001).

Thus, the present methodology provides a level of detection for a pair of genes that is 0.65×0.65=0.42, a value supported by the detection of positive signals for both sense and antisense expression in 5 out of 11 (0.45) clusters of previously described sense/antisense pairs (Table_S2 on CD-ROM2).

Of the 264 cluster pairs analyzed in the Microarrays of the present invention, 65 clusters (0.25) showed significant signals for both sense and antisense transcripts, which is 60% of the proposed level of detection for a pair of genes (0.25/0.42). Extrapolating this figure to the predicted antisense dataset of 2667 clusters, predicts at least 1600 sense/antisense transcriptional units in the human genome.

### Example 10

### Identification of Human Complementary Polynucleotide Sequence Pairs of Sense and Antisense Orientations Based on Orthologous Mouse Sequences

Human ESTs and cDNAs were obtained from NCBI GenBank version 136 (www.ncbi.nlm.nih.gov/dbEST) and aligned to the human genome build 32 (April 2003) (www.ncbi.nlm.nih.gov/genome/guide/human), using the LEADS clustering and assembly system (described in Sorek et al. (2002)). Briefly, the software cleans the expressed sequences from vectors and immunoglobulins, and masks them for repeats and low complexity sequences. The software then aligns the expressed sequences to the genome, taking alternative splicing into account, and clusters overlapping expressed sequences into “clusters” that represent genes or partial genes.

Sense/antisense pairs were identified using the same methods described in (Yelin et al. 2003). In brief, these methods screen for LEADS clusters containing sequences that originated from opposite strands of the DNA. The strand of origin of each sequence is determined by examining several sources of information, such as splice junctions, polyA tails and coding sequence annotation.

This entire process was performed with the mouse data: ESTs and cDNAs from NCBI GenBank version 136 (www.ncbi.nlm.nih.gov/dbEST) and build 30 (February 2003) of the mouse genome (www.ncbi.nlm.nih.gov/genome/guide/mouse).

To simplify the orthology definition between human and mouse, only clusters that included at least one mRNA from RefSeq database (www.ncbi.nlm.nih.gov/RefSeq/) were analyzed. This resulted in analysis of about 30% of the clusters in both human and mouse antisense datasets.

To link between the human and the mouse datasets, HomoloGene database of orthologous loci (www.ncbi.nlm.nih.gov/HomoloGene/) was used. Cases in which a locus in the human genome was assigned two or more orthologous loci in the mouse genome, or vice versa, were discarded from the final set of orthologous loci. The final set contained 15,552 pairs of exclusively orthologous loci between human and mouse.

The mouse antisense dataset (755 gene pairs) uncovered in the present study was analyzed for orthologous antisense cases in the human genome. It is estimated that about 80% of the genes in the mouse genome can be assigned a single orthologue in the human genome (Waterston et al. 2002), while for the others, more than one possible orthologue can be identified.

To ensure an orthology relationship for each mouse pair, only cases in which both mouse genes had a single orthologue in the human genome were analyzed.

About 83% of the loci in the mouse antisense dataset had a single human orthologue in the HomoloGene database, and the rest of the loci were eliminated from further analysis. This filter reduced the number of cases that could be analyzed to 526 gene pairs. In order to be further analyzed, both human orthologous loci in each case had to contain a RefSeq mRNA. About 15% of the human loci in the Locus Link database do not contain a RefSeq mRNA, thus, a fraction of the human orthologous loci were not RefSeq-containing, resulting in a second reduction in the number of cases that could be analyzed to 437 gene pairs.

Among the 437 mouse sense/antisense gene pairs, a set of 208 conserved pairs (#RES conserved) was identified, i.e. pairs in which the two genes were found to be antisense to each other in both genomes. The remaining mouse cases and their human orthologues were analyzed as well. These are 229 mouse gene pairs whose human orthologues were not identified as sense/antisense pairs. Two parameters can imply the potential existence of antisense overlap that is not found by Antisensor—
1. the distance on the genome between the candidate loci and their orientation (#RES opposite adjacent);
2. the evidence for antisense overlap for at least one of the loci in the pair (#RES antisense).

Looking at the orthologues of the 229 loci pairs, 172 were found to be adjacent (<10 Kb) and oppositely oriented also in the human genome (#RES opposite adjacent). Furthermore, in 81 of these cases (#RES opposite adjacent antisense), at least one of the genes had ESTs indicating antisense transcription (as identified by the Antisensor), strongly suggesting that there is an overlap also in the human genome between alternative transcripts longer than those deposited in the databases.

### Example 11

### Annotation of Newly Uncovered Naturally Occurring Antisense Transcripts

Newly uncovered naturally occurring transcripts were annotated using the Gencarta (Compugen, Tel-Aviv, Israel) platform. The Gencarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

Brief description of the methodology used to obtain annotative sequence information is summarized infra (for detailed description see U.S. patent application Ser. No. 10/426,002).

The ontological annotation approach—An ontology refers to the body of knowledge in a specific knowledge domain or discipline such as molecular biology, microbiology, immunology, virology, plant sciences, pharmaceutical chemistry, medicine, neurology, endocrinology, genetics, ecology, genomics, proteomics, cheminformatics, pharmacogenomics, bioinformatics, computer sciences, statistics, mathematics, chemistry, physics and artificial intelligence.

An ontology includes domain-specific concepts—referred to, herein, as sub-ontologies. A sub-ontology may be classified into smaller and narrower categories. The ontological annotation approach is effected as follows.

First, biomolecular (i.e., polynucleotide or polypeptide) sequences are computationally clustered according to a progressive homology range, thereby generating a plurality of clusters each being of a predetermined homology of the homology range.

Progressive homology is used to identify meaningful homologies among biomolecular sequences and to thereby assign new ontological annotations to sequences, which share requisite levels of homologies. Essentially, a biomolecular sequence is assigned to a specific cluster if displays a predetermined homology to at least one member of the cluster (i.e., single linkage). A “progressive homology range” refers to a range of homology thresholds, which progress via predetermined increments from a low homology level (e.g. 35%) to a high homology level (e.g. 99%).

Following generation of clusters, one or more ontologies are assigned to each cluster. Ontologies are derived from an annotation preassociated with at least one biomolecular sequence of each cluster; and/or generated by analyzing (e.g., text-mining) at least one biomolecular sequence of each cluster thereby annotating biomolecular sequences.

The hierarchical annotation approach—“Hierarchical annotation” refers to any ontology and subontology, which can be hierarchically ordered, such as, a tissue expression hierarchy, a developmental expression hierarchy, a pathological expression hierarchy, a cellular expression hierarchy, an intracellular expression hierarchy, a taxonomical hierarchy, a functional hierarchy and so forth.

The hierarchical annotation approach is effected as follows.

First, a dendrogram representing the hierarchy of interest is computationally constructed. A “dendrogram” refers to a branching diagram containing multiple nodes and representing a hierarchy of categories based on degree of similarity or number of shared characteristics.

Each of the multiple nodes of the dendrogram is annotated by at least one keyword describing the node, and enabling literature and database text mining, such as by using publicly available text mining software. A list of keywords can be obtained from the GO Consortium (www.geneontlogy.org). However, measures are taken to include as many keywords, and to include keywords which might be out of date. For example, for tissue annotation, a hierarchy is built using all available tissue/libraries sources available in the GenBank, while considering the following parameters: ignoring GenBank synonyms, building anatomical hierarchies, enabling flexible distinction between tissue types (normal versus pathology) and tissue classification levels (organs, systems, cell types, etc.).

In a second step, each of the biomolecular sequences is assigned to at least one specific node of the dendrogram.

The biomolecular sequences can be annotated biomolecular sequences, unannotated biomolecular sequences or partially annotated biomolecular sequences.

Annotated biomolecular sequences can be retrieved from pre-existing annotated databases as described hereinabove.

For example, in GenBank, relevant annotational information is provided in the definition and keyword fields. In this case, classification of the annotated biomolecular sequences to the dendrogram nodes is directly effected. A search for suitable annotated biomolecular sequences is performed using a set of keywords which are designed to classify the biomolecular sequences to the hierarchy (i.e., same keywords that populate the dendrogram)

In cases where the biomolecular sequences are unannotated or partially annotated, extraction of additional annotational information is effected prior to classification to dendrogram nodes. This can be effected by sequence alignment, as described hereinabove. Alternatively, annotational information can be predicted from structural studies. Where needed, nucleic acid sequences can be transformed to amino acid sequences to thereby enable more accurate annotational prediction.

Finally, each of the assigned biomolecular sequences is recursively classified to nodes hierarchically higher than the specific nodes, such that the root node of the dendrogram encompasses the full biomolecular sequence set, which can be classified according to a certain hierarchy, while the offspring of any node represent a partitioning of the parent set.

For example, a biomolecular sequence found to be specifically expressed in “rhabdomyosarcoma”, will be classified also to a higher hierarchy level, which is “sarcoma”, and then to “Mesenchimal cell tumors” and finally to a highest hierarchy level “Tumor”. In another example, a sequence found to be differentially expressed in endometrium cells, will be classified also to a higher hierarchy level, which is “uterus”, and then to “women genital system” and to “genital system” and finally to a highest hierarchy level “genitourinary system”. The retrieval can be performed according to each one of the requested levels.

Annotating gene expression according to relative abundance—Spatial and temporal gene annotations are also assigned by comparing relative abundance in libraries of different origins. This approach can be used to find gene which are differentially expressed in tissues, pathologies and different developmental stages. In principal, the presentation of a contig in at least two tissues of interest is determined and significant over or under representation of the contig in one of the at least two tissues is assessed to identify differential expression. Significant over or under representation is analyzed by statistical pairing.

Annotating spatial and temporal expression can also be effected on splice variants. This is effected as follows. First, a contigue which includes exonal sequence presentation of the at least two splice variants of the gene of interest is obtained. This contigue is assembled from a plurality of expressed sequences;
Then, at least one contigue sequence region unique to a portion (i.e., at least one and not all) of the at least two splice variants of the gene of interest is identified. Identification of such unique sequence region is effected using computer alignment software.

Finally, the number of the plurality of expressed sequences in the tissue having the at least one contigue sequence region is compared with the number of the plurality of expressed sequences not-having the at least one contigue sequence region, to thereby compare the expression level of the at least two splice variants of the gene of interest in the tissue.

Sequence anntotations obtained using the above-described methodologies and other approaches are disclosed in a data table in the file annotations_{—}136 of the enclosed CD-ROM 4.

The data table shows a collection of annotations for biomolecular sequences, which were identified according to the teachings of the present invention using transcript data based on GenBank versions 136.

Each feature in the data table is identified by “#”.

#INDICATION—This field designates the indications (i.e., diseases, disorders, pathological conditions) and therapies that the polypeptide of the present invention can be utilized for. Specifically, an indication lists the disorders or diseases in which the polypeptide of the present invention can be clinically used. A therapy describes a postulated mode of action of the polypeptide for the above-mentioned indication. For example, an indication can be “Cancer, general” while the therapy will be “Anticancer”.

Each protein was assigned a SWISSPROT and/or TremB1 human protein accession as described in section “Assignment of Swissprot/TremB1 accessions to Gencarta contigs” hereinbelow. The information contained in this field is the indication concatenated to the therapies that were accumulated for the SWISSPROT and/or TremB1 human protein from drug databases, such as PharmaProject (PJB Publications Ltd 2003 http://www.pjbpubs.com/cms.asp?pageid=340) and public databases, such as LocusLink (http://www.genelynx.org/cgi-bin/resource?res=locuslink) and Swissprot (http://www.ebi.ac.uk/swissprot/index.html). The field may comprise more than one term wherein a “;” separates each adjacent terms.

Example—#INDICATION Alopecia, general; Antianginal; Anticancer, immunological; Anticancer, other; Atherosclerosis; Buerger's syndrome; Cancer, general; Cancer, head and neck; Cancer, renal; Cardiovascular; Cirrhosis, hepatic; Cognition enhancer; Dermatological; Fibrosis, pulmonary; Gene therapy; Hepatic dysfunction, general; Hepatoprotective; Hypolipaemic/Antiatherosclerosis; Infarction, cerebral; Neuroprotective; Ophthalmological; Peripheral vascular disease; Radio/chemoprotective; Recombinant growth factor; Respiratory; Retinopathy, diabetic; Symptomatic antidiabetic; Urological;

Assignment of Swissprot/TremB1 accessions to Gencarta contigs—Gencarta contigs were assigned a Swissprot/TremB1 human accession as follows. Swissprot/TremB1 data were parsed and for each Swissprot/TremB1 accession (excluding Swissprot/TremB1 that are annotated as partial or fragment proteins) cross-references to EMBL and Genbank were parsed. The alignment quality of the Swissprot/TremB1 protein to their assigned mRNA sequences was checked by frame+p2n alignment analysis. A good alignment was considered as heving the following properties:
For partial mRNAs (those that in the mRNA description have the phrase “partial cds” or annotated as “3′” or “5′”)—an overall identity of 97% and coverage of 80% of the Swissprot/TremB1 protein.
All the rest were considered as full coding mRNAs and for them an overall identity of 97% identity and coverage of the Swissprot/TremB1 protein of over 95%.

The mRNAs were searched in the LEADS database for their corresponding contigs, and the contigs that included these mRNA sequences were assigned the Swissprot/TremB1 accession.

#PHARM—This field indicates possible pharmacological activities of the polypeptide. Each polypeptide was assigned with a SWISSPROT and/or TremB1 human protein accession, as described above. The information contained in this field is the indication concatenated to the therapies that were accumulated for the SWISSPROT and/or TremB1 human protein from drug databases such as PharmaProject (PJB Publications Ltd 2003 http://www.pjbpubs.com/cms.asp?pageid=340) and public databases, such as LocusLink and Swissprot. Note that in some cases this field can include opposite terms in cases where the protein can have contradicting activities—such as:
(i) Stimulant—inhibitor
(ii) Agonist—antagonist
(iii) Activator—inhibitor
(iv) Immunosuppressant—Immunostimulant

In these cases the pharmacology was indicated as “modulator”. For example, if the predicted polypeptide has potential agonistics/antagonistic effects (e.g. Fibroblast growth factor agonist and Fibroblast growth factor antagonist) then the annotation for this code will be “Fibroblast growth factor modulator”

A documentated example for such contradicing activities has been described for the soluble tumor necrosis factor receptors [Mohler et al., J. Immunology 151, 1548-1561]. Essentially, Mohler and co-workers showed that soluble receptor can act both as a carrier of TNF (i.e., agonistic effect) and as an antagonist of TNF activity.

#THERAPEUTIC_PROTEIN—This field predicts a therapeutic role for a protein represented by the contig. A contig was assigned this field if there was information in the drug database or the public databases (e.g., described hereinabove) that this protein, or part thereof, is used or can be used as a drug. This field is accompanied by the swissprot accession of the therapeutic protein which this contig most likely represents. Example: # THERAPEUTIC_PROTEIN UROK_HUMAN

#SEQLIST—This field lists all ESTs and/or mRNA sequences supporting the transcript and the predicted protein derived from Genbank version 136 (Jun. 15, 2003 ftp://ftp.ncbi.nih.gov/genbank/release.notes/gb136.release.notes). These sequences are the sequences which encompass the transcript. For example: BX394917 BX327693 AA894600 AA032291 AK027130 BM665029 BC025257 BE785231 BX371447 BX371446 BG821626 BX394918 BE737007 BE737043 AF213678 AB038318 AB038317 BE315017

GO annotations were predicted as described in “The ontological annotation approach” section hereinabove. Functional annotations of transcripts based on Gene Ontology (GO) are indicated by the following format.
*, ** “#GO_P”, annotations related to Biological Process,
*, ** “#GO_F”, annotations related to Molecular Function, and
*, ** “#GO_C”, annotations related to Cellular Component.

Proloc was used for protein subcellular localization prediction that assigns GO cellular component annotation to the protein. The localization terms were assigned with GO entries.

Cellular localization—ProLoc software, commercially available from Compugen LTD, was used to predict the cellular localization of the proteins. Two main approaches were used: (i) the presence of known extracellular domain/s in a protein; (ii) calculating putative transmembrane segments, if any, in the protein and calculating 2 p-values for the existence of a signal peptide. The latter is done by a searching for a signal peptide at the N-terminal sequence of the protein generating a score. Running the program on real signal peptides and on N-terminal protein sequences that lack a signal peptide resulted in 2 score distributions: the first is the score distribution of the real signal peptides and the second is the score distribution of the N-terminal protein sequences that lack the signal peptide. Given a novel protein product, ProLoc calculates the above-score score and provides the percentage of the scores that are higher than the current score, in the first distribution, as a first p-value (lower p-values mean more reliable signal peptide prediction) and the percentage of the scores that are lower than the current score, in the second distribution, as a second p-value (lower p-values mean more reliable non signal peptide prediction).

Thus, using this algorithm secreted proteins and membrane proteins can be identified, for example. However, proteins which lack signal peptide while are still secreted (such as after lysis of viral infected cells) can be identified such as by homology search to extracellular proteins which were identified as such by ProLoc.

TABLE 7

| | |
|---|---|
| IPR000874 | Bombesin-like peptide |
| IPR001693 | Calcitonin-like |
| IPR001651 | Gastrin/cholecystokinin peptide hormone |
| IPR000532 | Glucagon/GIP/secretin/VIP |
| IPR001545 | Gonadotropin, beta chain |
| IPR004825 | Insulin/IGF/relaxin |
| IPR000663 | Natriuretic peptide |
| IPR001955 | Pancreatic hormone |
| IPR001400 | Somatotropin hormone |
| IPR002040 | Tachykinin/Neurokinin |
| IPR006081 | Alpha defensin |
| IPR001928 | Endothelin-like toxin |
| IPR001415 | Parathyroid hormone |
| IPR001400 | Somatotropin hormone |
| IPR001990 | Chromogranin/secretogranin |
| IPR001819 | Chromogranin A/B |
| IPR002012 | Gonadotropin-releasing hormone |
| IPR001152 | Thymosin beta-4 |
| IPR000187 | Corticotropin-releasing factor, CRF |
| IPR001545 | Gonadotropin, beta chain |
| IPR000476 | Glycoprotein hormones alpha chain |
| IPR000476 | Glycoprotein hormones alpha chain |
| IPR001323 | Erythropoietin/thrombopoeitin |
| IPR001894 | Cathelicidin |
| IPR001894 | Cathelicidin |
| IPR001483 | Urotensin II |
| IPR006024 | Opioid neuropeptide precursor |
| IPR000020 | Anaphylatoxin/fibulin |
| IPR000074 | Apolipoprotein A1/A4/E |
| IPR001073 | Complement C1q protein |
| IPR000117 | Kappa casein |
| IPR001588 | Casein, alpha/beta |
| IPR001855 | Beta defensin |
| IPR001651 | Gastrin/cholecystokinin peptide hormone |
| IPR000867 | Insulin-like growth factor-binding protein, IGFBP |
| IPR001811 | Small chemokine, interleukin-8 like |
| IPR004825 | Insulin/IGF/relaxin |
| IPR002350 | Serine protease inhibitor, Kazal type |
| IPR000001 | Kringle |
| IPR002072 | Nerve growth factor |
| IPR001839 | Transforming growth factor beta (TGFb) |
| IPR001111 | Transforming growth factor beta (TGFb), N-terminal |
| IPR001820 | Tissue inhibitor of metalloproteinase |
| IPR000264 | Serum albumin family |
| IPR005817 | Wnt superfamily |

For each category the following features are optionally addressed:

“#GO_Acc” represents the accession number of the assigned GO entry, corresponding to the following “#GO_Desc” field.

“#GO_Desc” represents the description of the assigned GO entry, corresponding to the mentioned “#GO_Acc” field.

“#CL” represents the confidence level of the GO assignment, when #CL1 is the highest and #CL5 is the lowest possible confidence level. This field appears only when the GO assignment is based on a Swissprot/TremB1 protein accession or Interpro accession and (not on Proloc predictions or viral proteins predictions). Preliminary confidence levels were calculated for all public proteins as follows:
PCL 1: a public protein that has a curated GO annotation,
PCL 2: a public protein that has over 85% identity to a public protein with a curated GO annotation,
PCL 3: a public protein that exhibits 50-85% identity to a public protein with a curated GO annotation,
PCL 4: a public protein that has under 50% identity to a public protein with a curated GO annotation.

For each protein a homology search against all public proteins was done. If the protein has over 95% identity to a public protein with PCL X then the protein gets the same confidence level as the public protein. This confidence level is marked as “#CL X”. If the protein has over 85% identity but not over 95% to a public protein with PCL X than the protein gets a confidence level lower by 1 than the confidence level of the public protein. If the protein has over 70% identity but not over 85% to a public protein with PCL X than the protein gets a confidence level lower by 2 than the confidence level of the public protein. If the protein has over 50% identity but not over 70% to a public protein with PCL X than the protein gets a confidence level lower by 3 than the confidence level of the public protein. If the protein has over 30% identity but not over 50% to a public protein with PCL X than the protein gets a confidence level lower by 4 than the confidence level of the public protein.

A protein may get confidence level of 2 also if it has a true interpro domain that is linked to a GO annotation http://www.geneontology.org/external2go/interpro2go/.

When the confidence level is above “1”, GO annotations of higher levels of the GO hierarchy are assigned (e.g. for “#CL 3” the GO annotations provided, is as appears plus the 2 GO annotations above it in the hierarchy).

“#DB” marks the database on which the GO assignment relies on. The “sp”, as in Example 10a, relates to SwissProt/TremB1 Protein knowledgebase, available from http://www.expasy.ch/sprot/. “InterPro”, as in Example 10c, refers to the InterPro combined database, available from http://www.ebi.ac.uklinterpro/, which contains information regarding protein families, collected from the following databases: SwissProt (http://www.ebi.ac.uk/swissprot/), Prosite (http://www.expasy.ch/prosite/), Pfam (http://www.sanger.ac.uk/Software/Pfam/), Prints (http://www.bioinf.man.ac.uk/dbbrowser/PRINTS/), Prodom (http://prodes.toulouse.inra.fr/prodom/), Smart (http://smart.embl-heidelberg.de/) and Tigrfams (http://www.tigr.org/TIGRFAMs/). “Proloc statistical database”—meaning the statistics Proloc uses for predicting the subcellulat localization of a protein.

“#EN” represents the accession of the entity in the database (#DB), corresponding to the accession of the protein/domain why the GO was predicted. If the GO assignment is based on a protein from the SwissProt/TremB1 Protein database this field will have the locus name of the protein. Examples, “#DB sp #EN NRG2_HUMAN” means that the GO assignment in this case was based on a protein from the SwissProt/Tremb1 database, while the closest homologue (that has a GO assignment) to the assigned protein is depicted in SwissProt entry “NRG2 HUMAN “#DB interpro #EN IPR001609” means that GO assignment in this case was based on InterPro database, and the protein had an Interpro domain, IPR001609, that the assigned GO was based on. In Proloc predictions this field will have a Proloc annotation “#EN Proloc”. In predicitions based on viral proteins this field will have the gi. viral protein accession, “#EN 1491997”.

#GENE_SYMBOL—for each Gencarta contig a HUGO gene symbol was assigned in two ways:
(i) After assigning a Swissprot/TremB1 protein to each contig (see Assignment of Swissprot/TremB1 accessions to Gencarta contigs) all the gene symbols that appear for the Swissprot entry were parsed and added as a Gene symbol annotation to the gene.
(ii) LocusLink information—LocusLink was downloaded from NCBI ftp://ftp.ncbi.nih.gov/refseq/LocusLink/ (files loc2acc, loc2ref, and LL.out_hs). The data was integrated producing a file containing the gene symbol for every sequence. Gencarta contigs were assigned a gene symbol if they contain a sequence from this file that has a gene symbol

Example: #GENE_SYMBOL MMP15

#DIAGNOSTICS— secreted/membranal proteins get an annotation of “can be used as diagnostic markers for” for the list of indications as appearing in the # INDICATION field, described hereinabove. All proteins that were identified as secreted or membranal proteins (as described in the GO field section) will be assigned with this field.

In addition, known Gencarta contigs representing known diagnostic markers (such as listed in Table 8, below) and all transcripts and proteins deriving from this contig will be assigned to this field and will get the above mentioned annotation followed by “as indicated in the Diagnostic markers table”.

TABLE 8

| | | |
|---|---|---|
| Test | Gencarta Contig | Comments |

Enzymes

| | | |
|---|---|---|
| GPT | R35137 (GPT glutamic- | Also called ALT-alanine |
| | pyruvate transaminase | aminotransferase. |
| | (alanine amino- | Standard liver function |
| | transferase)) | test |
| | Z24841 (GPT2 glutamic | |
| | pyruvate transaminase | |
| | (alanine amino- | |
| | transferase) 2) | |
| GOT | M78228 (GOT1 | Also called AST- |
| | glutamic-oxaloacetic | aspartate amino- |
| | transaminase 1, | transferase. |
| | soluble (aspartate | Standard liver |
| | aminotransferase 1)) | function test |
| | M86145 (GOT2 glutamic- | |
| | oxaloacetic trans- | |
| | aminase 2, mito- | |
| | chondrial (aspartate | |
| | aminotransferase 2) | |
| GGT | HUMGGTX (GGT1: gamma- | Liver disease |
| | glutamyltransferase 1) | |
| CPK | T05088 (CKB creatine | Also called CK. |
| | kinase, brain) | Mostly used for |
| | HUMCKMA (CKM creatine | muscle pathologies. |
| | kinase, muscle) | The MB variant is |
| | H20196 (CKMT1 | heart specific and |
| | creatine kinase, | used in the diag- |
| | mitochondrial 1 | nosis of myocardial |
| | (ubiquitous))HUMSMCK | infarction |
| | (CKMT2 creatine | |
| | kinase, mitochondrial | |
| | 2 (sarcomeric)) | |
| CPK-MB | T05088 (CKB creatine | Cardiac problems - |
| | kinase, brain) | hetro-dimer of |
| | HUMCKMA (CKM creatine | CKB and CKM |
| | kinase, muscle) | |
| Alkaline | HSAPHOL- | Bone |
| Phosphatase | ALPL: alkaline | related syndromes |
| | phosphatase, | and liver diseases, |
| | liver/bone/kidney | mostly with biliary |
| | HUMALPHB - ALPI: | involvement |
| | alkaline phosphatase, | |
| | intestinal | |
| | HUMALPP-ALPP: | |
| | alkaline phosphatase, | |
| | placental (Regan | |
| | isozyme) | |
| Amylase | AA367524- (AMY1A: | Blood/Urine. Pancreas |
| | amylase, alpha 1A; | related diseases |
| | salivary) | |
| | T10898- (AMY2B: | |
| | amylase, alpha 2B; | |
| | pancreatic and 2A) | |
| LDH | HSLDHAR (LDHA lactate | Lactate Dehydro- |
| | dehydrogenase A) | genase. Used for |
| | M77886 (LDHB lactate | myocardial in- |
| | dehydrogenase B) | farction diag- |
| | HSU 13680 (LDHC | nosis and neo- |
| | lactate dehydrogenase | plastic syndromes |
| | C) | assessment. |
| | AA398148 (LDHL | |
| | lactate dehydrogenase | |
| | A-like)R09053 (LDHD | |
| | lactate dehydro- | |
| | genase D) | |
| G6PD | S58359 (G6PD glucose- | Glucose 6-phosphate |
| | 6-phosphate dehydro- | dehydrogenase. |
| | genase) | Levels measured when |
| | | deficiency is |
| | | suspected (leading |
| | | to susceptibility |
| | | to hemolysis) |
| Alpha1 | HUMA1ACM (SERPINA3 | Chronic lung |
| antiTrypsin | serine (or cysteine) | diseases |
| | proteinase inhibitor, | |
| | clade A (alpha-1 | |
| | antiproteinase, | |
| | antitrypsin), member | |
| | 3) | |
| | T10891 (AGT angio- | |
| | tensinogen (serine | |
| | (or cysteine) protein- | |
| | ase inhibitor, clade | |
| | A (alpha-1 anti- | |
| | proteinase, anti- | |
| | trypsin), member 8)) | |
| | R83168 (SERPINA6 | |
| | serine (or cysteine) | |
| | proteinase inhibitor, | |
| | clade A (alpha-1 | |
| | antiproteinase, anti- | |
| | trypsin), member 6) | |
| | HUMCINHP (SERPINA5 | |
| | serine (or cysteine) | |
| | proteinase inhibitor, | |
| | clade A (alpha-1 | |
| | antiproteinase, | |
| | antitrypsin), member | |
| | 5) | |
| | HSA1ATCA (SERPINA1 | |
| | serine (or cysteine) | |
| | proteinase inhibitor, | |
| | clade A (alpha-1 | |
| | antiproteinase, anti- | |
| | trypsin), member 1) | |
| | HUMKALLS (SERPINA4 | |
| | serine (or cysteine) | |
| | proteinase inhibitor, | |
| | clade A (alpha-1 | |
| | antiproteinase, anti- | |
| | trypsin), member 4) | |
| | HUMTBG (SERPINA7 | |
| | serine (or cysteine) | |
| | proteinase inhibitor, | |
| | clade A (alpha-1 | |
| | antiproteinase, anti- | |
| | trypsin), member 7) | |
| | T60354 (SERPINA10 | |
| | serine (or cysteine) | |
| | proteinase inhibitor, | |
| | clade A (alpha-1 | |
| | antiproteinase, anti- | |
| | trypsin), member 10) | |
| Renin | HSRENK (REN renin) | Some hypertension |
| | | syndromes |
| Acid | HUMAAPA (ACP1: acid | Used to differentiate |
| Phosphatase | phosphatase 1, | multiple myeloma with |
| | soluble) | other monoclonal |
| | T48863 (ACP2: acid | gammopathies of |
| | phosphatase 2, | uncertain significance |
| | lysosomal) | |
| | HSMRACP5 (ACP5: acid | |
| | phosphatase 5, | |
| | tartrate resistant) | |
| | T85211 (ACP6: | |
| | lysophosphatidic | |
| | acid phosphatase) | |
| | HSPROSAP (ACPP: acid | |
| | phosphatase, | |
| | prostate) AA005037 | |
| | (ACPT: acid | |
| | phosphatase, | |
| | testicular) | |
| Beta | T11069 (GUSB glu- | Used to differentiate |
| glucoronidase | curonidase, beta) | multiple myeloma with |
| | | other monoclonal |
| | | gammopathies of |
| | | uncertain significance |
| Aldolase | HSALDAR (ALDOA aldo- | Glycogen storage |
| | lase A, fructose- | diseases |
| | bisphosphate) | |
| | HSALDOBR (ALDOB | |
| | aldolase B, fructose- | |
| | bisphosphate) | |
| | M62176 (ALDOC | |
| | aldolase C, fructose- | |
| | bisphosphate) | |
| Choline | HUMCHEF (BCHE | Probably used for |
| esterase | butyrylcholinesterase) | organophosphates/ |
| | F00931 (ACHE | “nerve gases” |
| | acetylcholinesterase | intoxications |
| | (YT blood group)) | |
| Pepsinogen | HUMPGCA PGC: pro- | (in the stomach), |
| | gastricsin | high in gastritis, |
| | (pepsinogen C) | low in pernicious |
| | | anemia[ |
| ACE | HSACE (ACE: angio- | Angiotensin- |
| | tensin I converting | converting enzyme. |
| | enzyme (peptidyl- | Sarcoidosis |
| | dipeptidase A) 1) | |
| | AA397955 (ACE2: | |
| | angiotensin I | |
| | converting enzyme | |
| | (peptidyl-dipeptidase | |
| | A) 2) | |

Miscelleneous

| | | |
|---|---|---|
| Prion | HUMPRP0A (PRNP prion | BSE diagnosis |
| Protein | protein (p27-30) | |
| | (Creutzfeld-Jakob | |
| | disease, Gerstmann- | |
| | Strausler-Scheinker | |
| | syndrome, fatal | |
| | familial insomnia)) | |
| | W73057 (PRND | |
| | prion protein | |
| | 2 (dublet)) | |
| Myelin basic | M78010 (MBP myelin | In CSF. In Multiple |
| protein | basic protein) | sclerosis |
| | R13982 (MOBP myelin- | |
| | associated oligo- | |
| | dendrocyte basic | |
| | protein) | |
| Albumin | HSALB1 (ALB albumin) | Mostly liver function |
| | | and failure of intes- |
| | | tine absorption |
| Prealbumin | HSALB1 (ALB albumin) | early diagnosis of mal- |
| | | absorption |
| Ferritin | HUMFERLS (FTL | Iron deficiency anemia |
| | ferritin, light | |
| | polypeptide) | |
| | HUMFERHA (FTH1 | |
| | ferritin, heavy | |
| | polypeptide 1) | |
| Transferrin | S95936 (TF trans- | Iron deficiency anemia |
| | ferrin) | |
| Haptoglobin | HUMHPA1B (HP hapto- | Used in anemia states |
| | globin) | and neo-plastic |
| | | syndromes |
| CRP | HSCREACT (CRP C-re- | C reactive protein. |
| | active protein, | Associated with active |
| | pentraxin-related) | inflammation |
| AFP | D11581 (AFP alpha- | Alpha Feto Protein. |
| | fetoprotein) | Used in pregnancy |
| | | for abnormalities |
| | | screening and as |
| | | a cancer marker. |
| C3 | T40158 (C3 complement | Various auto-immune |
| | component 3) | and allergy syndromes |
| C4 | HSCOC4 (C4A comple- | Various auto-immune |
| | ment component 4A; | and allergy syn- |
| | C4B complement | dromes |
| | component 4B) | |
| Ceruloplasmin | HSCP2 (CP cerulo- | Wilson's disease |
| | plasmin (ferroxidase)) | (liver disease) |
| Myoglobin | T11628 (MB myoglobin) | Rhabdomyolysis, Myo- |
| | | cardial infarction |
| FABP | S67314 (FABP3: fatty | myoglobin and Fatty |
| | acid binding protein | Acid Binding |
| | 3, muscle and heart) | |
| | D111754 (FABP1 liver- | |
| | L-FABP-fatty acid | |
| | binding protein 1) | |
| | AW605378 (FABP2: | |
| | fatty acid binding | |
| | protein 2, intestinal) | |
| | HUMALBP (FABP4: fatty | |
| | acid binding protein | |
| | 4, adipocyte) | |
| | T06152 (FABP5: fatty | |
| | acid binding protein | |
| | 5 (psoriasis-associ- | |
| | ated) | |
| | HSI15PGN1 (FABP6: | |
| | fatty acid binding | |
| | protein 6, ileal | |
| | (gastrotropin) | |
| | R60348 (FABP7: fatty | |
| | acid binding protein | |
| | 7, brain) | |
| Troponin I | HUMTROPNIN (TNNI2 | Acute myocardial |
| | troponin I, skeletal, | infarction |
| | fast) | |
| | Z25083 (TNNI1 | |
| | troponin I, skeletal, | |
| | slow) | |
| | HUMTROPIA (TNNI3 | |
| | troponin I, cardiac) | |
| Beta-2- | HSB2MMU (B2M beta-2- | |
| microglobulin | microglobulin) | |
| Macroglobin | M62177 (A2M: alpha-2- | Elevated in in- |
| | macroglobulin) | flammation |
| Alpha-1 | T72188 (A1BG: alpha- | Elevated in in- |
| glycoprotein | 1-B glycoprotein) | flammation and |
| | | tumors. |
| Apo A-I | HUMAPOAIP (APOA1: | Risk for coronary |
| | apolipoprotein A-I) | artery disease |
| Apo B-100 | HSAPOBR2 (APOB: | Atherosclerotic |
| | apolipoprotein B | heart disease |
| | (including Ag(x) | |
| | antigen)) | |
| Apo E | T61627 (APOE: apoli- | diagnosis of Type III |
| | poprotein E) | hyperlipoproteinemia, |
| | | evaluate a possible |
| | | genetic component to |
| | | atherosclerosis, or |
| | | to help confirm a |
| | | diagnosis of late |
| | | onset AD |
| CF gene | HUMCFTRM (CFTR: | Cystic fibrosis |
| | cystic fibrosis | disease (a DNA test - |
| | transmembrane | blood sample) |
| | conductance regulator, | |
| | ATP-binding cassette | |
| | (sub-family C, | |
| | member 7)) | |
| PSEN1 gene | T89701 (PSEN1: pre- | Early onset of |
| | senilin 1 (Alzheimer | familial AD (a DNA |
| | disease 3)) | test - blood sample) |

Hormones

| | | |
|---|---|---|
| Erythropoietin | HSERPR (EPO erythro- | Hardly used for diag- |
| | poietin) | nosis. Used as treat- |
| | | ment |
| GH | HSGROW1 (GH1 growth | Growth Hormone. Endo- |
| | hormone 1) | crine syndromes |
| | HUMCS2 (GH2 growth | |
| | hormone 2) | |
| TSH | AV745295 (TSHB thy- | Part of thyroid |
| | roid stimulating | functions tests |
| | hormone, beta) | |
| BetaHCG | R27266 (CGB5 chor- | Pregnancy, malignant |
| | ionic gonadotropin, | syndromes in men and |
| | beta polypeptide 5) | women |
| LH | HUMCGBB50 (LHB lu- | Part of standard |
| | teinizing hormone | hormonal profile |
| | beta polypeptide) | for fertility, gyneco- |
| | | logical syndromes |
| | | and endocrine |
| | | syndromes |
| FSH | AV754057 (FSHB | Part of standard |
| | follicle stimulating | hormonal profile |
| | hormone, beta poly- | for fertility, gyneco- |
| | peptide | logical syndromes |
| | | and endocrine |
| | | syndromes |
| TBG | S40807 (TG thyro- | Thyroxin binding |
| | globulin) | globulin. Thyroid |
| | | syndromes |
| Prolactin | HSLACT (PRL | Various endocrine |
| | prolactin) | syndromes |
| Thyroglobulin | S40807 (TG thyro- | Follow up of thyroid |
| | globulin) | cancer patients |
| PTH | HSTHYR (PTH para- | Parathyroid Hormone. |
| | thyroid hormone) | Syndromes of calcium |
| | | management |
| Insulin/Pre | HSPPI (INS insulin) | Diabetes |
| Insulin | | |
| Gastrin | HSGAST (GAS gastrin) | Peptic ulcers |
| Oxytocin | HUMOTCB (OXT oxy- | Endocrine syndromes |
| | tocin, prepro-(neuro- | related to lactation |
| | physin I)) | |
| AVP | HUMVPC (AVP arginine | Arginine Vasopressin. |
| | vasopressin (neuro- | Endocrine syndromes |
| | physin II, anti | related to the osmotic |
| | diuretic hormone, | pressure of body fluids |
| | diabetes insipidus, | |
| | neurohypophyseal)) | |
| ACTH | HUMPOMCMTC (POMC: | Secreted from the ant- |
| | proopiomelanocortin | erior pituitary gland. |
| | (adrenocorticotropin/ | Regulation of cortisol. |
| | beta-lipotropin/ | Abnormalities are in- |
| | alpha-melanocyte | dicative of Cushing's |
| | stimulating hormone/ | disease, addison's |
| | beta-melanocyte | disease and adrenal |
| | stimulating hormone/ | tumors |
| | beta-endorphin)) | |
| BNP | HUMNATPEP (NPPB: | Heart failure |
| | natriuretic peptide | |
| | precursor B) | |

Blood Clotting

| | | |
|---|---|---|
| Protein C | S50739 (PROC protein | Inherited Clotting |
| | C (inactivator of co- | disorders |
| | agulation factors | |
| | Va and VIIIa)) | |
| Protein S | HSSPROTR (PROS1 | Inherited Clotting |
| | protein S (alpha)) | disorders |
| Fibrinogen | D11940 (FGA: fibrino- | Clotting disorders |
| | gen, A alpha poly- | |
| | peptide) | |
| | HUMFBRB (FGB: | |
| | fibrinogen, B | |
| | beta polypeptide) | |
| | T24021 (FGG: fibrino- | |
| | gen, gamma polypep- | |
| | tide) | |
| Factors 2, 5, 7, | HUMPTHROM | Inherited Clotting |
| 9, 10, 11, 12, | (F2 coagulation | disorders |
| 13 | factor II (thrombin)) | |
| | HUMTFPC (F3 coagula- | |
| | tion factor III | |
| | (thromboplastin, | |
| | tissue factor)) | |
| | HUMF5A (F5 coagula- | |
| | tion factor V | |
| | (proaccelerin, | |
| | labile factor)) | |
| | M78203 (F7 coagula- | |
| | tion factor VII (serum | |
| | prothrombin conver- | |
| | sion accelerator)) | |
| | HUMF8C (F8 coagula- | |
| | tion factor VIII, | |
| | procoagulant com- | |
| | ponent (hemophilia | |
| | A)) | |
| | HUMCFIX (F9 coagula- | |
| | tion factor IX (plasma | |
| | thromboplastic com- | |
| | ponent, Christmas | |
| | disease, hemophilia | |
| | B)) | |
| | HUMCFX (F10: coagu- | |
| | lation factor X) | |
| | HUMFXI (F11 coagu- | |
| | lation factor XI | |
| | (plasma thromboplas- | |
| | tin antecedent)) | |
| | HUMCFXIIA (F12 coagu- | |
| | lation factor XII | |
| | (Hageman factor)) | |
| | HUMFXIIIA (F13A1 co- | |
| | agulation factor XIII, | |
| | A1 polypeptide) | |
| | R28976 (F13B coagu- | |
| | lation factor XIII, | |
| | B polypeptide) | |
| vWF | HUMVWF (VWF von | Von Willebrand factor. |
| | Willebrand factor) | Inherited Clotting |
| | | disorders |
| Antithrombin | T62060 (SERPINC1 | Inherited Clotting |
| III | serine (or cysteine) | disorders |
| | proteinase inhibitor, | |
| | clade C (antithrom- | |
| | bin), member 1) | |

Cancer Markers

| | | |
|---|---|---|
| AFP | D11581 (AFP alpha- | Pregnancy, testicular |
| | fetoprotein) | cancer and hepato- |
| | | cellular cancer |
| CA125 | HSIAI3B (M17S2 mem- | Ovarian cancer |
| | brane component, | |
| | chromosome 17, | |
| | surface marker 2 | |
| | (ovarian carcinoma | |
| | antigen CA125)) | |
| CA-15-3 | HSMUC1A (MUC1 mucin | Breast cancer |
| | 1, transmembrane) | |
| CA-19-9 | HSAFUTF (FUT3: fuco- | Gastrointestinal |
| | syltransferase 3 | cancer, pancreatic |
| | (galactoside 3(4)- | cancer |
| | L-fucosyltransferase, | |
| | Lewis blood group in- | |
| | cluded)) | |
| CEA | T10888 HUMCEA | Carcinoembryonic |
| | (CEACAM3 carcino- | Antigen. Colorectal |
| | embryonic antigen- | cancer |
| | related cell adhesion | |
| | molecule 3) | |
| PSA | HSCDN9 (KLK3: kalli- | |
| | krein 3, (prostate | |
| | specific antigen)) | |
| PSMA | HUMPSM (FOLH1: folate | |
| | hydrolase (prostate- | |
| | specific membrane | |
| | antigen) 1) | |
| TPA, TATI, | HSPSTI (SPINK1: | Ovarian cancer |
| OVX1, LASA, | serine protease | |
| CA54/81 | inhibitor, Kazal | |
| | type 1) | |
| BRCA 1 | H90415 (BRCA1: breast | |
| | cancer 1, early | |
| | onset) | |
| BRCA 2 | H47777 (BRCA2: breast | Breast cancer |
| | cancer 2, early | (ovarian cancer?) |
| | onset) | |
| HER2/Neu | S57296 (ERBB2: v-erb- | Breast cancer |
| | b2 erythroblastic | |
| | leukemia viral | |
| | oncogene homolog 2, | |
| | neuro/glioblastoma | |
| | derived oncogene | |
| | homolog (avian)) | |
| Estrogen | HSERG5UTA (ESR1: | Breast cancer |
| receptor | estrogen receptor 1) | |
| | HSRNAERB (ESR2: | |
| | estrogen receptor 2 | |
| | (ER beta)) | |
| Progesterone | T09102 (PGRMC1: pro- | Breast cancer |
| receptor | gesterone receptor | |
| | membrane component 1) | |
| | Z32891 (PGRMC2: pro- | |
| | gesterone receptor | |
| | membrane component 2) | |

Note:
(i) Small portion of these “markers” are also drug targets, whether already for approved drugs (such as alpha1 antiTrypsin) or under development (e.g., GOT).
(ii) Some of these “markers” are also used as therapeutic proteins (e.g., Erythropoietin).
(iii) All markers are found in the blood/serum unless otherwise specified.

#DRUG_DRUG_INTERACTION: refers to proteins involved in a biological process which mediates the interaction between at least two consumed drugs. Novel splice variants of known proteins involved in interaction between drugs may be used, for example, to modulate such drug-drug interactions. Examples of proteins involved in drug-drug interactions are presented in Table 9 together with the corresponding internal gene contig name, enabling to allocate the new sloce variants within the data files in the attached CD-ROM 4.

TABLE 9

| | Gene | |
|---|---|---|
| Contig | Symbol | Description |
| HUMANTLA | SLC3A2 | 4f2 cell-surface antigen |
| | | heavy chain |
| Z43093 | HTR6 | 5-hydroxytryptamine 6 receptor |
| HSXLALDA | ABCD1 | Adrenoleukodystrophy protein |
| R35137 | GPT | Alanine aminotransferase |
| D11683 | ALDH1 | Aldehyde dehydrogenase, |
| | | cytosolic |
| T53833 | AOX1 | Aldehyde oxidase |
| HUMD4G08M3 | ORM1 | Alpha-1-acid glycoprotein 1 |
| HUMD4G08M3 | ORM2 | Alpha-1-acid glycoprotein 2 |
| HUMABPA | ABP1 | Amiloride-sensitive amine |
| | | oxidase [copper- |
| | | containing] |
| S62734 | MAOB | Amine oxidase [flavin- |
| | | containing] b |
| AA526963 | SLC6A14 | Amino acid transporter b0+ |
| HSAE2 | SLC4A2 | Anion exchange protein 2 |
| M78110 | SLC4A3 | Anion exchange protein 3 |
| M78052 | ABCB2 | Antigen peptide transporter 1 |
| HUMMHCIIAB | ABCB3 | Antigen peptide transporter 2 |
| F02693 | APOD | Apolipoprotein d |
| M62234 | ASNA1 | Arsenical pump-driving ATPase |
| HUMNORTR | NAT1 | Arylamine n-acetyltransferase |
| | | 1 |
| T67129 | NAT1 | Arylamine n-acetyltransferase |
| | | 1 |
| AI262683 | NAT2 | Arylamine n-acetyltransferase |
| | | 2 |
| Z39550 | ABCB9 | ATP-binding cassette protein |
| | | abcb9 |
| Z44377 | ABCA1 | ATP-binding cassette, sub- |
| | | family a, member 1 |
| M78056 | ABCA2 | ATP-binding cassette, sub- |
| | | family a, member 2 |
| T05334 | ABCA3 | ATP-binding cassette, sub- |
| | | family a, member 3 |
| T79973 | ABCB6 | ATP-binding cassette, sub- |
| | | family b, member 6, mito- |
| | | chondrial |
| T78010 | ABCB7 | ATP-binding cassette, sub- |
| | | family b, member 7, mito- |
| | | chondrial |
| R89046 | ABCB8 | ATP-binding cassette, sub- |
| | | family b, member 8, mito- |
| | | chondrial |
| H64439 | ABCD2 | ATP-binding cassette, sub- |
| | | family d, member 2 |
| M85760 | ABCD3 | ATP-binding cassette, sub- |
| | | family d, member 3 |
| Z21904 | ABCD4 | ATP-binding cassette, sub- |
| | | family d, member 4 |
| Z39977 | ABCG1 | ATP-binding cassette, sub- |
| | | family g, member 1 |
| Z45628 | ABCG2 | ATP-binding cassette, sub- |
| | | family g, member 2 |
| T80665 | SLC7A9 | B(0, +)-type amino acid |
| | | transporter 1 |
| AF091582 | ABCB11 | Bile salt export pump |
| Z38696 | BLMH | Bleomycin hydrolase |
| T08127 | BNPI | Brain-specific na-dependent |
| | | inorganic phosphate cotrans- |
| | | porter |
| F00545 | SLC12A2 | Bumetanide-sensitive sodium- |
| | | (potassium)-chloride cotrans- |
| | | porter 2 |
| HSU07969 | CDH17 | Cadherin-17 |
| T10238 | SLC25A12 | Calcium-binding mitochondrial |
| | | carrier protein aralar1 |
| Z40674 | SLC25A13 | Calcium-binding mitochondrial |
| | | carrier protein aralar2 |
| T61818 | ABCC2 | Canalicular multispecific |
| | | organic anion transporter 1 |
| T39953 | ABCC3 | Canalicular multispecific |
| | | organic anion transporter 2 |
| HUMCRE | CBR1 | Carbonyl reductase [nadph] |
| | | 1 |
| AA320697 | CBR3 | Carbonyl reductase [nadph] |
| | | 3 |
| F03362 | COMT | Catechol o-methyltransferase, |
| | | membrane-bound form |
| T11004 | COMT | Catechol o-methyltransferase, |
| | | membrane-bound form |
| T39368 | SLC7A4 | Cationic amino acid trans- |
| | | porter-4 |
| S74445 | RBP5 | Cellular retinol-binding |
| | | protein iii |
| T55952 | RBP5 | Cellular retinol-binding |
| | | protein iii |
| HSU39905 | SLC18A1 | Chromaffin granule amine |
| | | transporter |
| R52371 | SLC35A1 | Cmp-sialic acid transporter |
| D20754 | CNT3 | Concentrative nucleoside |
| | | transporter 3 |
| HSMNKMBP | ATP7A | Copper-transporting ATPase 1 |
| HUMWND | ATP7B | Copper-transporting ATPase 2 |
| HUMCFTRM | ABCC7 | Cystic fibrosis transmembrane |
| | | conductance regulator |
| F10774 | SLC7A11 | Cystine/glutamate transporter |
| HUMCYPADA | CYP11B1 | Cytochrome P450 11B1, mitochon- |
| | | drial |
| HUMARM | CYP19 | Cytochrome P450 19 |
| HUMCYP145 | CYP1A1 | Cytochrome P450 1A1 |
| R21282 | CYP26 | Cytochrome P450 26 |
| AF209774 | CYP2A13 | Cytochrome P450 2A13 |
| HSC45B2C | CYP2A6 | Cytochrome P450 2A6 |
| HSC45B2C | CYP2A7 | Cytochrome P450 2A7 |
| HSP452B6 | CYP2B6 | Cytochrome P450 2B6 |
| HUM2C18 | CYP2C18 | Cytochrome P450 2C18 |
| HSCP450 | CYP2C19 | Cytochrome P450 2C19 |
| HUM2C18 | CYP2C19 | Cytochrome P450 2C19 |
| HUMCYPAX | CYP2C8 | Cytochrome P450 2C8 |
| HSCP450 | CYP2C9 | Cytochrome P450 2C9 |
| HSP450 | CYP2D6 | Cytochrome P450 2D6 |
| M77918 | CYP2E1 | Cytochrome P450 2E1 |
| HUMCYPIIF | CYP2F1 | Cytochrome P450 2F1 |
| H09076 | CYP2J2 | Cytochrome P450 2J2 |
| R07010 | CYP39A1 | Cytochrome P450 39A1 |
| HUMCYPHLP | CYP3A3 | Cytochrome P450 3A3 |
| HUMCYPHLP | CYP3A4 | Cytochrome P450 3A4 |
| AA416822 | CYP3A43 | Cytochrome P450 3A43 |
| HUMCYP3A | CYP3A5 | Cytochrome P450 3A5 |
| T82801 | CYP3A7 | Cytochrome P450 3A7 |
| HSCYP4AA | CYP4A11 | Cytochrome P450 4A11 |
| S67580 | CYP4A11 | Cytochrome P450 4A11 |
| HUMCP45IV | CYP4B1 | Cytochrome P450 4B1 |
| T98002 | CYP4F12 | Cytochrome P450 4F12 |
| AA377259 | CYP4F2 | Cytochrome P450 4F2 |
| AI400898 | CYP4F8 | Cytochrome P450 4F8 |
| HSU09178 | DPYD | Dihydropyrimidine dehydro- |
| | | genase [nadp+] |
| W03174 | DPYD | Dihydropyrimidine dehydro- |
| | | genase [nadp+] |
| HUMFMO1 | FMO1 | Dimethylaniline monooxygenase |
| | | [n-oxide forming] 1 |
| HSFLMON2R | FMO2 | Dimethylaniline monooxygenase |
| | | [n-oxide forming] 2 |
| T64494 | FMO2 | Dimethylaniline monooxygenase |
| | | [n-oxide forming] 2 |
| T40157 | FMO3 | Dimethylaniline monooxygenase |
| | | [n-oxide forming] 3 |
| HSFLMON2R | FMO4 | Dimethylaniline monooxygenase |
| | | [n-oxide forming] 4 |
| D12220 | FMO5 | Dimethylaniline monooxygenase |
| | | [n-oxide forming] 5 |
| H25503 | HET | Efflux transporter like |
| | | protein |
| T12485 | HET | Efflux transporter like |
| | | protein |
| M78151 | EPHX1 | Epoxide hydrolase 1 |
| T66884 | SLC29A1 | Equilibrative nucleoside trans- |
| | | porter 1 |
| HSHNP36 | SLC29A2 | Equilibrative nucleoside trans- |
| | | porter 2 |
| T08444 | SLC1A3 | Excitatory amino acid trans- |
| | | porter 1 |
| HSU01824 | SLC1A2 | Excitatory amino acid trans- |
| | | porter 2 |
| HSU03506 | SLC1A1 | Excitatory amino acid trans- |
| | | porter 3 |
| F07883 | SLC1A6 | Excitatory amino acid trans- |
| | | porter 4 |
| N39099 | SLC1A7 | Excitatory amino acid trans- |
| | | porter 5 |
| F00548 | SLC2A9 | Facilitative glucose trans- |
| | | porter family member glut9 |
| T95337 | SLC27A1 | Fatty acid transport protein |
| Z44099 | SLC27A1 | Fatty acid transport protein |
| HUMALBP | FABP4 | Fatty acid-binding protein, |
| | | adipocyte |
| S67314 | FABP3 | Fatty acid-binding protein, |
| | | heart |
| AW605378 | FABP2 | Fatty acid-binding protein, |
| | | intestinal |
| L25227 | SLC19A1 | Folate transporter 1 |
| HS115PGN1 | FABP6 | Gastrotropin |
| Z40427 | G6PT1 | Glucose 5-phosphate transporter |
| D11793 | SLC2A1 | Glucose transporter type 1, |
| | | erythrocyte/brain |
| N27535 | SLC2A10 | Glucose transporter type 10 |
| T52633 | SLC2A11 | Glucose transporter type 11 |
| HUMLGTPA | SLC2A2 | Glucose transporter type 2, |
| | | liver |
| HUMLGTPA | SLC2A2 | Glucose transporter type 2, |
| | | liver |
| T07239 | SLC2A3 | Glucose transporter type 3, |
| | | brain |
| HUMIRGT | SLC2A4 | Glucose transporter type 4, |
| | | insulin-responsive. |
| M62105 | SLC2A5 | Glucose transporter type 5, |
| | | small intestine |
| T59518 | SLC2A8 | Glucose transporter type 8 |
| HUMLGTH1 | GSTA1 | Glutathione s-transferase a1 |
| HUMLGTH1 | GSTA2 | Glutathione s-transferase a2 |
| T98291 | GSTA3 | Glutathione s-transferase a3-3 |
| Z21581 | GSTA4 | Glutathione s-transferase a4-4 |
| HSGST4 | GSTM1 | Glutathione s-transferase mu 1 |
| D31291 | GSTM2 | Glutathione s-transferase mu 2 |
| HSGST4 | GSTM2 | Glutathione s-transferase mu 2 |
| T08311 | GSTM3 | Glutathione s-transferase mu 3 |
| HUMGSTM4B | GSTM4 | Glutathione s-transferase mu 4 |
| HUMGSTM5 | GSTM5 | Glutathione s-transferase mu 5 |
| T05391 | GSTP1 | Glutathione s-transferase p |
| AA346312 | GSTT1 | Glutathione s-transferase theta |
| | | 1 |
| R08187 | GSTT2 | Glutathione s-transferase theta |
| | | 2 |
| Z25318 | GSTK1 | Glutathione s-transferase, mito- |
| | | chondrial |
| H03163 | SLC37A1 | Glycerol-3-phosphate transporter |
| AA363955 | SLC5A7 | High affinity choline transporter |
| HSRRMRNA | SLC7A1 | High-affinity cationic amino |
| | | acid transporter-1 |
| R22196 | SLC31A1 | High-affinity copper uptake |
| | | protein 1 |
| AA918012 | SLC10A2 | Ileal sodium/bile acid trans- |
| | | porter |
| F00840 | SLC7A5 | Large neutral amino acid trans- |
| | | porter small subunit 1 |
| M79133 | SLC7A5 | Large neutral amino acid trans- |
| | | porter small subunit 1 |
| Z38621 | SLC7A8 | Large neutral amino acids trans- |
| | | porter small subunit 2 |
| HUMCARAA | CES1 | Liver carboxylesterase |
| S52379 | CES1 | Liver carboxylesterase |
| T55488 | SLC21A6 | Liver-specific organic anion |
| | | transporter |
| W78748 | SLC5A4 | Low affinity sodium-glucose co- |
| | | transporter |
| T54842 | SLC7A2 | Low-affinity cationic amino acid |
| | | transporter-2 |
| T87799 | ABCA7 | Macrophage abc transporter |
| Z17844 | LRP | Major vault protein |
| Z24885 | GSTZ1 | Maleylacetoacetate isomerase |
| T39939 | MT1A | Metallothionein-IA |
| R99207 | MT1B | Metallothionein-IB |
| T39939 | MT1E | Metallothionein-IE |
| D11725 | MT1F | Metallothionein-IF |
| S68949 | MT1G | Metallothionein-IG |
| S68954 | MT1G | Metallothionein-IG |
| HSFMET | MT1H | Metallothionein-IH |
| S52379 | MT2A | Metallothionein-II |
| M78846 | MT3 | Metallothionein-III |
| AA570216 | MT1K | Metallothionein-IK |
| S68954 | MT1K | Metallothionein-IK |
| D11725 | MT1L | Metallothionein-IL |
| HSPP15 | MT1L | Metallothionein-IL |
| HSPP15 | MT1R | Metallothionein-IR |
| NM032935 | MT4 | Metallothionein-IV |
| HUMGST | MGST1 | Microsomal glutathione s-trans- |
| | | ferase 1 |
| H59104 | MGST2 | Microsomal glutathione s-trans- |
| | | ferase 2 |
| T47062 | MGST3 | Microsomal glutathione s-trans- |
| | | ferase 3 |
| SSMPCP | SLC25A3 | Mitochondrial phosphate carrier |
| | | protein |
| R14814 | SULT1A3 | Monoamine-sulfating phenol sulfo- |
| | | transferase |
| HUMARYTRAB | SULT1A3 | Monoamine-sulfating phenol sulfo- |
| | | transferase |
| M62141 | SLC16A1 | Monocarboxylate transporter 1 |
| H90048 | SLC16A6 | Monocarboxylate transporter 2 |
| F02520 | SLC16A2 | Monocarboxylate transporter 3 |
| AI005004 | SLC16A8 | Monocarboxylate transporter 4 |
| T59354 | SLC16A3 | Monocarboxylate transporter 5 |
| R22416 | SLC16A4 | Monocarboxylate transporter 6 |
| T78890 | SLC16A5 | Monocarboxylate transporter 7 |
| F01173 | SLC16A7 | Monocarboxylate transporter 8 |
| Z41819 | ABCB1 | Multidrug resistance protein 1 |
| AL041030 | ABCB4 | Multidrug resistance protein 3 |
| SATHRMRP | ABCC1 | Multidrug resistance-associated |
| | | protein 1 |
| R00050 | ABCC4 | Multidrug resistance-associated |
| | | protein 4 |
| M78673 | ABCC5 | Multidrug resistance-associated |
| | | protein 5 |
| R99091 | ABCC6 | Multidrug resistance-associated |
| | | protein 6 |
| T69749 | ABCC6 | Multidrug resistance-associated |
| | | protein 6 |
| D11495 | DIA4 | Nad(p)h dehydrogenase |
| | | [quinone] 1 |
| HUMNRAMP | SLC11A1 | Natural resistance-associated |
| | | macrophage protein 1 |
| Z38360 | SLC11A2 | Natural resistance-associated |
| | | macrophage protein 2 |
| HUMASCT1A | SLC1A4 | Neutral amino acid transporter |
| | | a |
| AW237674 | SLC1A5 | Neutral amino acid transporter |
| | | b(0) |
| M78631 | SLC3A1 | Neutral and basic amino acid |
| | | transport protein rbat |
| HSU08021 | NNMT | Nicotinamide n-methyltransferase |
| T87759 | SLC22A4 | Novel organic cation transporter |
| | | 1 |
| Z41935 | SLC15A2 | Oligopeptide transporter, kidney |
| | | isoform |
| HSU21936 | SLC15A1 | Oligopeptide transporter, small |
| | | intestine isoform |
| M62053 | OAT1 | Organic anion transporter 1 |
| H18607 | OAT3 | Organic anion transporter 3 |
| R16970 | OAT4 | Organic anion transporter 4 |
| T39111 | SLC21A9 | Organic anion transporter b |
| Z41576 | SLC21A11 | Organic anion transporter oATP-d |
| T23657 | SLC21A12 | Organic anion transporter oATP-e |
| Z21041 | SLC21A14 | Organic anion transporting poly- |
| | | peptide 14 |
| H75435 | SLC21A8 | Organic anion transporting poly- |
| | | peptide 8 |
| HSU77086 | SLC22A1 | Organic cation transporter 1 |
| HSOCTK | SLC22A2 | Organic cation transporter 2 |
| R00207 | SLC22A3 | Organic cation transporter 3 |
| H30224 | ORCTL4 | Organic cation transporter |
| | | like 4 |
| H25503 | ORCTL2 | Organic cation transporter- |
| | | like 2 |
| Z38659 | SLC22A5 | Organic cation/carnitine trans- |
| | | porter 2 |
| AB010438 | ORCTL3 | Organic-cation transporter |
| | | like 3 |
| T95621 | ORNT1 | Ornithine transporter |
| AA398593 | ORNT2 | Ornithine transporter 2 |
| R79412 | NTT5 | Orphan sodium- and chloride- |
| | | dependent neurotransmitter |
| | | transporter ntt5 |
| H82347 | NTT73 | Orphan sodium- and chloride- |
| | | dependent neurotransmitter |
| | | transporter ntt73 |
| Z43484 | NTT73 | Orphan sodium- and chloride- |
| | | dependent neurotransmitter |
| | | transporter ntt73 |
| Z44749 | SLC25A17 | Peroxisomal membrane protein |
| | | pmp34 |
| HUMARYLSUL | SULT1A1 | Phenol-sulfating phenol sulfo- |
| | | transferase 1 |
| HUMARYLSUL | SULT1A2 | Phenol-sulfating phenol sulfo- |
| | | transferase 2 |
| D12243 | RBP4 | Plasma retinol-binding protein |
| HUMATPAD | ATP12A | Potassium-transporting ATPase |
| | | alpha chain 2 |
| Z40030 | ATP8A1 | Potential phospholipid-trans- |
| | | porting ATPase ia |
| Z40188 | FIC1 | Potential phospholipid-trans- |
| | | porting ATPase ic |
| T86800 | SLC31A2 | Probable low-affinity copper |
| | | uptake protein 2 |
| Z41717 | PTGIS | Prostacyclin synthase |
| S78220 | PTGS1 | Prostaglandin g/h synthase 1 |
| HUMENDOSYN | PTGS2 | Prostaglandin g/h synthase 2 |
| T85296 | SLC21A2 | Prostaglandin transporter |
| M62053 | SLC22A6 | Renal organic anion transport |
| | | protein 1 |
| HSU26209 | SLC13A2 | Renal sodium/dicarboxylate |
| | | cotransporter |
| Z40774 | SLC13A2 | Renal sodium/dicarboxylate |
| | | cotransporter |
| HSNAPI1 | SLC17A1 | Renal sodium-dependent phosphate |
| | | transport protein 1 |
| HUMNAPI3X | SLC34A1 | Renal sodium-dependent phosphate |
| | | transport protein 2 |
| H85361 | ABCA4 | Retinal-specific ATP-binding |
| | | cassette transporter |
| S74445 | CRABP1 | Retinoic acid-binding protein i, |
| | | cellular |
| HUMCRABP | CRABP2 | Retinoic acid-binding protein ii, |
| | | cellular |
| HUMCRBP | RBP1 | Retinol-binding protein i, |
| | | cellular |
| S57153 | RBP1 | Retinol-binding protein i, |
| | | cellular |
| T07054 | RBP2 | Retinol-binding protein ii, |
| | | cellular |
| T63266 | RBP2 | Retinol-binding protein ii, |
| | | cellular |
| HUMBGT1R | SLC6A12 | Sodium- and chloride-dependent |
| | | betaine transporter |
| HUMCRTR | SLC6A8 | Sodium- and chloride-dependent |
| | | creatine transporter 1 |
| R20043 | SLC6A13 | Sodium- and chloride-dependent |
| | | gaba transporter 2 |
| S70609 | SLC6A9 | Sodium- and chloride-dependent |
| | | glycine transporter 1 |
| AA625644 | SLC6A5 | Sodium- and chloride-dependent |
| | | glycine transporter 2 |
| M78677 | SLC6A6 | Sodium- and chloride-dependent |
| | | taurine transporter |
| T10761 | SLC4A4 | Sodium bicarbonate cotransporter |
| | | nbc1 |
| AA452802 | NBC4 | Sodium bicarbonate cotransporter |
| | | nbc4a |
| HUMCNC | SLC8A1 | Sodium/calcium exchanger 1 |
| R20720 | SLC8A2 | Sodium/calcium exchanger 2 |
| T07666 | SLC8A3 | Sodium/calcium exchanger 3 |
| T07666 | SLC8A3 | Sodium/glucose cotransporter 1 |
| HUMSGLCT | SLC5A2 | Sodium/glucose cotransporter 2 |
| S83549 | SLC9A2 | Sodium/hydrogen exchanger 2 |
| HSU66088 | SLC5A5 | Sodium/iodide cotransporter |
| HSU62966 | SLC28A1 | Sodium/nucleoside cotransporter |
| | | 1 |
| AA358822 | SLC28A2 | Sodium/nucleoside cotransporter |
| | | 2 |
| HUMNTCP | SLC10A1 | Sodium/taurocholate cotrans- |
| | | porting polypeptide |
| HSGAT1MR | SLC6A1 | Sodium-and chloride-dependent |
| | | gaba transporter 1 |
| F05686 | SLC6A11 | Sodium-and chloride-dependent |
| | | gaba transporter 3 |
| AA604857 | SVCT1 | Sodium-denpendent vitamin c |
| | | transporter 1 |
| T27309 | SVCT2 | Sodium-denpendent vitamin c |
| | | transporter 2 |
| S44626 | SLC6A3 | Sodium-dependent dopamine |
| | | transporter |
| Z39412 | NADC3 | Sodium-dependent high-affinity |
| | | dicarboxylate transporter |
| T77525 | SLC5A6 | Sodium-dependent multivitamin |
| | | transporter |
| HUMNORTR | SLC6A2 | Sodium-dependent noradrenaline |
| | | transporter |
| HSZ83953 | SLC17A3 | Sodium-dependent phosphate |
| | | transport protein 3 |
| R06460 | SLC17A3 | Sodium-dependent phosphate |
| | | transport protein 3 |
| HSZ83953 | SLC17A4 | Sodium-dependent phosphate |
| | | transport protein 4 |
| HSY10506 | SLC17A4 | Sodium-dependent phosphate |
| | | transport protein 4 |
| H40741 | SLC6A7 | Sodium-dependent proline trans- |
| | | porter |
| HSSERT | SLC6A4 | Sodium-dependent serotonin |
| | | transporter |
| T64950 | SLC21A3 | Sodium-independent organic anion |
| | | transporter |
| M79233 | EPHX2 | Soluble epoxide hydrolase |
| Z39813 | SLC25A18 | Solute carrier |
| HUMSTAR | STAR | Steroidogenic acute regulatory |
| | | protein |
| Z20453 | STAR | Steroidogenic acute regulatory |
| | | protein |
| R69741 | SLC26A2 | Sulfate transporter |
| T08860 | ABCC8 | Sulfonylurea receptor 1 |
| R73927 | ABCC9 | Sulfonylurea receptor 2 |
| T84623 | SULT1C1 | Sulfotransferase 1C1 |
| R58632 | SULT1C2 | Sulfotransferase 1C2 |
| T95810 | SLC18A2 | Synaptic vesicle amine trans- |
| | | porter |
| AF080246 | TRAG3 | Taxol resistant associated |
| | | protein 3 |
| R20880 | SLC19A2 | Thiamine transporter 1 |
| HSU44128 | SLC12A3 | Thiazide-sensitive sodium- |
| | | chloride cotransporter |
| S62904 | TPMT | Thiopurine s-methyltransferase |
| HSPBX2 | G17 | Transporter protein |
| T62038 | G17 | Transporter protein |
| R53836 | SLC35A3 | UDP n-acetylglucosamine trans- |
| | | porter |
| T60594 | SLC35A2 | UDP-galactose translocator |
| HUMUGT1FA | UGT1 | UDP-glucuronosyltransferase 1-1, |
| | | microsomal |
| HUMUGT1FA | UGT1A10 | UDP-glucuronosyltransferase 1A10 |
| HUMUGT1FA | UGT1A7 | UDP-glucuronosyltransferase 1A7 |
| HUMUGT1FA | UGT1A8 | UDP-glucuronosyltransferase 1A8 |
| HUMUGT1FA | UGT1A9 | UDP-glucuronosyltransferase 1A9 |
| HSUGT2BIO | UGT2B10 | UDP-glucuronosyltransferase 2B10, |
| | | microsomal |
| HSUDPGT | UGT2B11 | UDP-glucuronosyltransferase 2B11, |
| | | microsomal |
| N70316 | UGT2B11 | UDP-glucuronosyltransferase 2B11, |
| | | microsomal |
| HSU08854 | UGT2B15 | UDP-glucuronosyltransferase 2B15, |
| | | microsomal |
| T24450 | UGT2B17 | UDP-glucuronosyltransferase 2B17, |
| | | microsomal |
| HSUDPGT | UGT2B4 | UDP-glucuronosyltransferase 2B4, |
| | | microsomal |
| HUMUDPGTA | UGT2B7 | UDP-glucuronosyltransferase 2B7, |
| | | microsomal |
| AI002801 | SLC14A1 | Urea transporter, erythrocyte |
| Z19313 | SLC14A1 | Urea transporter, erythrocyte |
| AI002801 | SLC14A2 | Urea transporter, kidney |
| HSU09210 | SLC18A3 | Vesicular acetylcholine trans- |
| | | porter |
| HUMKCHB | KCNA4 | Voltage-gated potassium channel |
| | | protein kv 1.4 |
| R09608 | XDH | Xanthine dehydrogenase/oxidase |
| T64266 | SLC7A7 | Y + 1 amino acid transporter 1 |
| T10628 | SLC30A1 | Zinc transporter 1 |
| AA322641 | SLC30A4 | Zinc transporter 4 |

#DISEASE_RELATED_CLINICAL_PHENOTYPE—This field denotes the possibility of using biomolecular sequences of the present invention for the diagnosis and/or treatment of genetic diseases such as listed in the following URL: http://www.geneclinics.org/servlet/access?id=8888891 &key=X9D790O5re1Az&db=genetests&res=&fcn=b&grp=g&genesearch=true&testtype=both&ls=l&type=e&qry=&submit=Search and in Table 10, below. This list includes genetic diseases and genes which may be used for the detection and/or treatment thereof. As such, newly uncovered variants of these genes may be used for improved diagnosis and/or treatment when used singly or in combination with the previously described genes.

TABLE 10

| Gencarta | Gene | |
|---|---|---|
| Contig | Symbol | Disease |
| HSCFTRMA | CFTR | Congenital Bilateral Absence of the Vas Deferens; Cystic Fibrosis |
| HUMCFTRM | CFTR | Congenital Bilateral Absence of the Vas Deferens; Cystic Fibrosis |
| HUMFGFR3 | FGFR3 | Achondroplasia; Crouzon Syndrome with Acanthosis Nigricans; FGFR- |
| | | Related Craniosynostosis Syndromes; Hypochondroplasia; Muenke |
| | | Syndrome; Severe Achondroplasia with Developmental Delay and |
| | | Acanthosis Nigricans (SADDAN); Thanatophoric Dysplasia |
| T07012 | FGD1 | Aarskog Syndrome |
| HSCA1III | COL3A1 | Ehlers-Danlos Syndrome, Vascular Type |
| HUMCOL2A1B | COL2A1 | Achondrogenesis Type 2; Kniest Dysplasia; Spondyloepimetaphyseal |
| | | Dysplasia, Strudwick Type; Spondyloepiphyseal Dysplasia, Congenita; |
| | | Stickler Syndrome; Stickler Syndrome Type I |
| R68817 | APRT | Adenine Phosphoribosyltransferase Deficiency |
| HUMAMPD1 | AMPD1 | Adenosine Monophosphate Deaminase 1 |
| M62124 | PXR1 | Zellweger Syndrome Spectrum |
| HSXLALDA | ABCD1 | Adrenoleukodystrophy, X-Linked |
| T28718 | BTK | X-Linked Agammaglobulinemia |
| R91110 | IL2RG | X-Linked Severe Combined Immunodeficiency |
| HUMPEDG | OCA2 | Oculocutaneous Albinism Type 2 |
| HSU01873 | TYR | Oculocutaneous Albinism Type 1 |
| HSOA1MRNA | OA1 | Ocular Albinism, X-Linked |
| R14843 | TYRP1 | Oculocutaneous Albinism Type 3 (TRP1 Related) |
| HSALDAR | ALDOA | Aldolase A Deficiency |
| T40633 | HBA1 | Alpha-Thalassemia |
| T40633 | HBA2 | Alpha-Thalassemia; Hemoglobin Constant Spring |
| HSU09820 | ATRX | Alpha-Thalassemia X-Linked Mental Retardation Syndrome |
| HUMCOL4A5 | COL4A5 | Alport Syndrome; Alport Syndrome, X-Linked |
| T61627 | APOE | Apolipoprotein E Genotyping; Familial Combined Hyperlipidemia; |
| | | Hyperlipoproteinemia Type III |
| T89701 | PSEN1 | Alzheimer Disease Type 3; Early-Onset Familial Alzheimer Disease |
| R05822 | PSEN2 | Alzheimer Disease Type 4; Early-Onset Familial Alzheimer Disease |
| HSTTRM | TTR | Transthyretin Amyloidosis |
| T23978 | SOD1 | Amyotrophic Lateral Sclerosis |
| HUMANDREC | AR | Androgen Insensitivity Syndrome; Spinal and Bulbar Muscular Atrophy |
| Z19491 | UBE3A | Angelman Syndrome |
| HUMPAX6AN | PAX6 | Aniridia; Anophthalmia; Isolated Aniridia; Peters Anomaly; Peters Anomaly |
| | | with Cataract; Wilms Tumor-Aniridia-Genital Anomalies-Retardation Syndrome |
| HUMKGFRA | FGFR2 | Apert Syndrome; Beare-Stevenson Syndrome; Crouzon Syndrome; FGFR- |
| | | Related Craniosynostosis Syndromes; Jackson-Weiss Syndrome; Pfeiffer |
| | | Syndrome Type 1, 2, and 3 |
| HSU03272 | FBN2 | Congenital Contractural Arachnodactyly |
| Z19459 | AMCD1 | Arthrogryposis Multiplex Congenita, Distal, Type I |
| T88756 | ATM | Ataxia-Telangiectasia |
| H30056 | BBS1 | Bardet-Biedl Syndrome |
| Z25009 | BBS2 | Bardet-Biedl Syndrome |
| T64876 | BBS4 | Bardet-Biedl Syndrome |
| N27125 | PTCH | Nevoid Basal Cell Carcinoma Syndrome |
| N25339 | VMD2 | Best Vitelliform Macular Dystrophy |
| N71795 | VMD2 | Best Vitelliform Macular Dystrophy |
| HUMHBB3E | HBB | Beta-Thalassemia; Hemoglobin E; Hemoglobin S Beta-Thalassemia; |
| | | Hemoglobin SC; Hemoglobin SD; Hemoglobin SO; Hemoglobin SS; Sickle |
| | | Cell Disease |
| H53763 | BLM | Bloom Syndrome |
| N22283 | EYA1 | Branchiootorenal Syndrome |
| H90415 | BRCA1 | BRCA1 and BRCA2 Hereditary Breast/Ovarian Cancer; BRCA1 Hereditary |
| | | Breast/Ovarian Cancer |
| H47777 | BRCA2 | BRCA1 and BRCA2 Hereditary Breast/Ovarian Cancer; BRCA2 Hereditary |
| | | Breast/Ovarian Cancer |
| Z33575 | SOX9 | Campomelic Dysplasia |
| S67156 | ASPA | Canavan Disease |
| T52465 | CPS1 | Carbamoylphosphate Synthetase I Deficiency |
| HSVD3HYD | CYP27A1 | Cerebrotendinous Xanthomatosis |
| S66705 | MPZ | Charcot-Marie-Tooth Neuropathy Type 1; Charcot-Marie-Tooth Neuropathy |
| | | Type 1B; Congenital Hypomyelination |
| HSGAS3MR | PMP22 | Charcot-Marie-Tooth Neuropathy Type 1; Charcot-Marie-Tooth Neuropathy |
| | | Type 1 A; Charcot-Marie-Tooth Neuropathy Type 1E; Hereditary Neuropathy |
| | | with Liability to Pressure Palsies |
| T93208 | PMP22 | Charcot-Marie-Tooth Neuropathy Type 1; Charcot-Marie-Tooth Neuropathy |
| | | Type 1A; Charcot-Marie-Tooth Neuropathy Type 1E; Hereditary Neuropathy |
| | | with Liability to Pressure Palsies |
| HSGAPJR | GJB1 | Charcot-Marie-Tooth Neuropathy Type X |
| HSXCGD | CYBB | Chronic Granulomatous Disease |
| S67289 | CYBB | Chronic Granulomatous Disease |
| HSASD | ASS | Citrullinemia |
| HUMPAX2A | PAX2 | Anophthalmia; Renal-Coloboma Syndrome |
| HUMP45C21 | CYP21A2 | 21-Hydroxylase Deficiency |
| S74720 | NROB1 | Complex Glycerol Kinase Deficiency; Dosage-Sensitive Sex Reversal; |
| | | Isolated X-Linked Adrenal Hypoplasia Congenita; X-Linked Adrenal |
| | | Hypoplasia Congenita |
| HSKERTRNS | TGM1 | Autosomal Recessive Congenital Ichthyosis |
| BF928311 | CPO | Hereditary Coproporphyria |
| HSCPPOX | CPO | Hereditary Coproporphyria |
| HUMTGFBIG | TGFBI | Avellino Corneal Dystrophy; Granular Corneal Dystrophy; Lattice Corneal |
| | | Dystrophy Type I |
| R08437 | MSX2 | Craniosynostosis Type II; Parietal Foramina 1 |
| HUMPRP0A | PRNP | Prion Diseases |
| T08652 | DRPLA | DRPLA |
| Z46151 | DRPLA | DRPLA |
| HSWT1 | WT1 | Denys-Drash Syndrome; Wilms Tumor; Wilms Tumor-Aniridia-Genital |
| | | Anomalies-Retardation Syndrome; WT1-Related Disorders |
| T52050 | WT1 | Denys-Drash Syndrome; Wilms Tumor; Wilms Tumor-Aniridia-Genital |
| | | Anomalies-Retardation Syndrome; WT1-Related Disorders |
| M78080 | ATP2A2 | Darier Disease |
| Z30219 | DCR | Down Syndrome Critical Region |
| T11279 | DKC1 | Dyskeratosis Congenita |
| T08131 | DYT1 | Early-Onset Primary Dystonia (DYT1) |
| T50729 | ED1 | Hypohidrotic Ectodermal Dysplasia; Hypohidrotic Ectodermal Dysplasia, X- |
| | | Linked |
| HUMPA1V | COL5A1 | Ehlers-Danlos Syndrome, Classic Type |
| HUMLYSYL | PLOD | Ehlers-Danlos Syndrome, Kyphoscoliotic Form |
| HSCOLIA | COL1A2 | Ehlers-Danlos Syndrome, Arthrochalasia Type; Osteogenesis Imperfecta |
| HUMCG1PA1 | COL1A1 | Ehlers-Danlos Syndrome, Arthrochalasia Type; Osteogenesis Imperfecta |
| Z30171 | TAZ | 3-Methylglutaconic Aciduria Type 2; Cardiomyopathy; Dilated |
| | | Cardiomyopathy; Endocardial Fibroelastosis; Familial Isolated |
| | | Noncompaction of Left Ventrical Myocardium |
| Z39302 | TAZ | 3-Methylglutaconic Aciduria Type 2; Cardiomyopathy; Dilated |
| | | Cardiomyopathy; Endocardial Fibroelastosis; Familial Isolated |
| | | Noncompaction of Left Ventrical Myocardium |
| HUMKERK5A | KRT5 | Epidermolysis Bullosa Simplex |
| R72295 | KRT14 | Epidermolysis Bullosa Simplex |
| HUMKTEP2A | KRT1 | Epidermolytic Hyperkeratosis; Nonepidermolytic Palmoplantar |
| | | Hyperkeratosis |
| HUMK10A | KRT10 | Epidermolytic Hyperkeratosis |
| M78482 | CHS1 | Chediak-Higashi Syndrome |
| HSTCD1 | CHM | Choroideremia |
| HSAGALAR | GLA | Fabry Disease |
| T79651 | GLA | Fabry Disease |
| HUMF5A | F5 | Factor V Leiden Thrombophilia; Factor V R2 Mutation Thrombophilia |
| HUMFXI | F11 | Factor XI Deficiency |
| M79108 | APC | Colon Cancer (APC I1307K related); Familial Adenomatous Polyposis |
| T10619 | IKBKAP | Familial Dysautonomia |
| HUMFMR1 | FMR1 | Fragile X Syndrome |
| M78417 | FMR2 | FRAXE Syndrome |
| R06415 | FRDA | Friedreich Ataxia |
| HSALDOBR | ALDOB | Hereditary Fructose Intolerance |
| HUMALFUC | FUCA1 | Fucosidosis |
| M85904 | FH | Fumarate Hydratase Deficiency |
| H85361 | ABCA4 | Age-Related Macular Degeneration; Retinitis Pigmentosa, Autosomal |
| | | Recessive; Stargardt Disease 1 |
| R31596 | GALK1 | Galactokinase Deficiency |
| T53762 | GALT | Galactosemia |
| HUMGCB | GBA | Gaucher Disease |
| T48672 | GBA | Gaucher Disease |
| HSGCRAR | NR3C1 | Glucocorticoid Resistance |
| S58359 | G6PD | Glucose-6-Phosphate Dehydrogenase Deficiency |
| HSGKTS1 | GK | Glycerol Kinase Deficiency |
| HSRNAGLK | GK | Glycerol Kinase Deficiency |
| U01120 | G6PC | Glycogen Storage Disease Type Ia |
| HUMGAAA | GAA | Glycogen Storage Disease Type II |
| F00985 | AGL | Glycogen Storage Disease Type III |
| HUMHGBE | GBE1 | Glycogen Storage Disease Type IV |
| HSPHOSR1 | PYGM | Glycogen Storage Disease Type V |
| D12179 | PYGL | Glycogen Storage Disease Type VI |
| HSHMPFK | PFKM | Glycogen Storage Disease Type VII |
| HUMGLI3A | GLI3 | GLI3-Related Disorders; Greig Cephalopolysyndactyly Syndrome; Pallister- |
| | | Hall Syndrome |
| F09335 | ATP2C1 | Hailey-Hailey Disease |
| M62210 | CCM1 | Angiokeratoma Corporis Diffusum with Arteriovenous Fistulas; Familial |
| | | Cerebral Cavernous Malformation |
| T59431 | HFE | HFE-Associated Hereditary Hemochromatosis |
| HSALK1A | ACVRL1 | Hereditary Hemorrhagic Telangiectasia |
| HUMENDO | ENG | Hereditary Hemorrhagic Telangiectasia |
| HUMF8C | F8 | Hemophilia A |
| HUMFVIII | F8 | Hemophilia A |
| HUMCFIX | F9 | Hemophilia B |
| HSU03911 | MSH2 | Hereditary Non-Polyposis Colon Cancer |
| Z24775 | MLH1 | Hereditary Non-Polyposis Colon Cancer |
| HSRETTT | RET | Hirschsprung Disease; Multiple Endocrine Neoplasia Type 2 |
| HUMSHH | SHH | Holoprosencephaly 3 |
| N81026 | TBX5 | Holt-Oram Syndrome |
| M78262 | CBS | Homocystinuria |
| T06035 | IDS | Mucopolysaccharidosis Type II |
| T03828 | HD | Huntington Disease |
| H27612 | IDUA | Mucopolysaccharidosis Type I |
| M62205 | GFAP | Alexander Disease |
| HUMCD40L | TNFSF5 | Hyper IgM Syndrome, X-Linked |
| HUMPTHROM | F2 | Prothrombin G20210A Thrombophilia |
| T61466 | MTHFR | MTHFR Deficiency; MTHFR Thermolabile Variant |
| HUMSKM1A | SCN4A | Hyperkalemic Periodic Paralysis Type 1; Hypokalemic Periodic Paralysis; |
| | | Hypokalemic Periodic Paralysis Type 2; Myotonia Congenita, Dominant; |
| | | Paramyotonia Congenita |
| HSU09784 | CACNA1S | Hypokalemic Periodic Paralysis; Hypokalemic Periodic Paralysis Type 1; |
| | | Malignant Hyperthermia Susceptibility |
| HUMLPLAA | LPL | Familial Lipoprotein Lipase Deficiency |
| HUMPEX | PHEX | Hypophosphatemic Rickets, X-Linked Dominant |
| M78626 | STS | Ichthyosis, X-Linked |
| R56102 | IKBKG | Incontinentia Pigmenti |
| Z39843 | IVD | Isovaleric Acidemia |
| S60085S1 | KAL1 | Kallmann Syndrome, X-Linked |
| T55061 | KEL | Kell Antigen Genotyping |
| HUMGALC | GALC | Krabbe Disease |
| HUMZFPSREB | ZNF9 | Myotonic Dystrophy Type 2 |
| Z19342 | KIF1B | Charcot-Marie-Tooth Neuropathy Type 2 |
| T11351 | NPC2 | Niemann-Pick Disease Type C |
| Z39096 | NDRG1 | Charcot-Marie-Tooth Neuropathy Type 4 |
| AA984421 | PRX | Charcot-Marie-Tooth Neuropathy Type 4; Charcot-Marie-Tooth Neuropathy |
| | | Type 4F |
| HUMRETGC | GUCY2D | Leber Congenital Amaurosis |
| HSU18991 | RPE65 | Leber Congenital Amaurosis; Retinitis Pigmentosa, Autosomal Recessive |
| C16899 | MTND6 | Leber Hereditary Optic Neuropathy; Mitochondrial Disorders; Mitochondrial |
| | | DNA-Associated Leigh Syndrome and NARP |
| AA069417 | MTND4 | Leber Hereditary Optic Neuropathy; Mitochondrial Disorders; Mitochondrial |
| | | DNA-Associated Leigh Syndrome and NARP |
| HUMCYP3A | MTND4 | Leber Hereditary Optic Neuropathy; Mitochondrial Disorders; Mitochondrial |
| | | DNA-Associated Leigh Syndrome and NARP |
| HSCPHC22 | MTND1 | Leber Hereditary Optic Neuropathy; Mitochondrial Disorders; Mitochondrial |
| | | DNA-Associated Leigh Syndrome and NARP |
| HUMHPRT | HPRT1 | Lesch-Nyhan Syndrome |
| HUMLHHCGR | LHCGR | Leydig Cell Hypoplasia/Agenesis; Male-Limited Precocious Puberty |
| HSP53 | TP53 | Li-Fraumeni Syndrome |
| Z19198 | HADHB | Long Chain 3-Hydroxyacyl-CoA Dehydrogenase Deficiency |
| M79018 | HADHA | Long Chain 3-Hydroxyacyl-CoA Dehydrogenase Deficiency |
| R72332 | HADHA | Long Chain 3-Hydroxyacyl-CoA Dehydrogenase Deficiency |
| W93500 | KCNQ1 | Atrial Fibrillation; Jervell and Lange-Nielsen Syndrome; LQT 1;Romano- |
| | | Ward Syndrome |
| S62085 | OCRL | Lowe Syndrome |
| T48981 | FBN1 | Marfan Syndrome |
| HUMASFB | ARSB | Mucopolysaccharidosis Type VI |
| M62202 | GNAS | Albright Hereditary Osteodystrophy; McCune-Albright Syndrome; Osseus |
| | | Heteroplasia, Progressive |
| N46342 | SACS | ARSACS |
| T81605 | FANCD2 | Fanconi Anemia |
| H47777 | FANCD1 | Fanconi Anemia |
| T23877 | ACADM | Medium Chain Acyl-Coenzyme A Dehydrogenase Deficiency |
| AA906866 | PARK2 | Parkin Type of Juvenile Parkinson Disease |
| BE140729 | GJB4 | Erythrokeratodermia Variabilis |
| HSU26727 | CDKN2A | Familial Malignant Melanoma |
| T47218 | SPINK5 | Netherton Syndrome |
| HSMNKMBP | ATP7A | ATP7A-Related Copper Transport Disorders |
| R37821 | SHFM4 | Ectrodactyly |
| Z38987 | GSN | Amyloidosis V |
| HSARYA | ARSA | Chromosome 22q13.3 Deletion Syndrome; Metachromatic Leukodystrophy |
| S68531 | COL10A1 | Metaphyseal Chondrodysplasia, Schmid Type |
| T59742 | CACNA1A | Episodic Ataxia Type 2; Familial Hemiplegic Migraine; Spinocerebellar |
| | | Ataxia Type 6 |
| HSCP2 | HPS3 | Hermansky-Pudlak Syndrome; Hermansky-Pudlak Syndrome 3 |
| R21301 | HPS3 | Hermansky-Pudlak Syndrome; Hermansky-Pudlak Syndrome 3 |
| HUMBGALRP | GLB1 | GM1 Gangliosidosis; Mucopolysaccharidosis Type IVB |
| HSU12507 | KCNJ2 | Andersen Syndrome |
| R28488 | MEN1 | Multiple Endocrine Neoplasia Type 1 |
| HUMCOMP | COMP | COMP-Related Multiple Epiphyseal Dysplasia; Multiple Epiphyseal |
| | | Dysplasia, Dominant; Pseudoachondroplasia |
| H30258 | COL9A2 | Multiple Epiphyseal Dysplasia, Dominant |
| T48133 | EXT1 | Hereditary Multiple Exostoses; Multiple Exostoses, Type I |
| T06129 | EXT2 | Hereditary Multiple Exostoses; Multiple Exostoses, Type II |
| T05624 | LAMA2 | Congenital Muscular Dystrophy with Merosin Deficiency |
| HSDYSTIA | DMD | Duchenne/Becker Muscular Dystrophy; Dystrophinopathies; X-Linked |
| | | Dilated Cardiomyopathy |
| HSSTA | EMD | Emery-Dreifuss Muscular Dystrophy, X-Linked |
| HSU20165 | BMPR2 | Primary Pulmonary Hypertension |
| M79239 | CAPN3 | Calpainopathy; Limb-Girdle Muscular Dystrophies, Autosomal Recessive |
| HSU34976 | SGCG | Gamma-Sarcoglycanopathy; Limb-Girdle Muscular Dystrophies, Autosomal |
| | | Recessive; Sarcoglycanopathies |
| HUMADHA | SGCA | Alpha-Sarcoglycanopathy; Limb-Girdle Muscular Dystrophies, Autosomal |
| | | Recessive; Sarcoglycanopathies |
| AI340083 | SGCA | Alpha-Sarcoglycanopathy; Limb-Girdle Muscular Dystrophies, Autosomal |
| | | Recessive; Sarcoglycanopathies |
| Z25374 | SGCB | Beta-Sarcoglycanopathy; Limb-Girdle Muscular Dystrophies, Autosomal |
| | | Recessive; Sarcoglycanopathies |
| N29439 | SGCD | Delta-Sarcoglycanopathy; Dilated Cardiomyopathy; Limb-Girdle Muscular |
| | | Dystrophies, Autosomal Recessive; Sarcoglycanopathies |
| N56180 | CASQ2 | Catecholaminergic Ventricular Tachycardia, Autosomal Recessive |
| T23560 | CHRNB2 | Nocturnal Frontal Lobe Epilepsy, Autosomal Dominant |
| HSCHRNA44 | CHRNA4 | Nocturnal Frontal Lobe Epilepsy, Autosomal Dominant |
| M78654 | CHRNA4 | Nocturnal Frontal Lobe Epilepsy, Autosomal Dominant |
| T86329 | CDH23 | Usher Syndrome Type 1 |
| D11677 | PABPN1 | Oculopharyngeal Muscular Dystrophy |
| AW449267 | PCDH15 | Usher Syndrome Type 1 |
| HUMCLC | CLCN1 | Myotonia Congenita, Dominant; Myotonia Congenita, Recessive |
| S86455 | DMPK | Myotonic Dystrophy Type 1 |
| T70260 | MTM1 | Myotubular Myopathy, X-Linked |
| T12579 | LMX1B | Nail-Patella Syndrome |
| HSTRKT1 | TPM3 | Nemaline Myopathy |
| HUMTROPCK | TPM3 | Nemaline Myopathy |
| Z19248 | NEB | Nemaline Myopathy |
| AF030626 | AVPR2 | Nephrogenic Diabetes Insipidus; Nephrogenic Diabetes Insipidus, X-Linked |
| AA780862 | NPHS1 | Congenital Finnish Nephrosis |
| T08860 | ABCC8 | ABCC8-Related Hyperinsulinism; Familial Hyperinsulinism |
| AA679741 | KCNJ11 | Familial Hyperinsulinism; KCNJ11-Related Hyperinsulinism |
| M77935 | NF1 | Neurofibromatosis 1 |
| HSMEORPRA | NF2 | Neurofibromatosis 2 |
| T08995 | CLN3 | CLN3-Related Neuronal Ceroid-Lipofuscinosis; Neuronal Ceroid- |
| | | Lipofuscinoses |
| T72120 | CLN2 | CLN2-Related Neuronal Ceroid-Lipofuscinosis; Neuronal Ceroid- |
| | | Lipofuscinoses |
| T41059 | GRHPR | Hyperoxaluria, Primary, Type 2 |
| HUMGCRFC | FCGR3A | Neutrophil Antigen Genotyping |
| R21657 | NPC1 | Niemann-Pick Disease Type C; Niemann-Pick Disease Type C1 |
| M77961 | SMPD1 | Niemann-Pick Disease Due to Sphingomyelinase Deficiency |
| T87256 | SUOX | Sulfocysteinuria |
| D79813 | SOST | SOST-Related Sclerosing Bone Dysplasias |
| T94707 | MATN3 | Multiple Epiphyseal Dysplasia, Dominant |
| HSCOL9AL | COL9A1 | Multiple Epiphyseal Dysplasia, Dominant |
| S69208 | TNNT1 | Nemaline Myopathy |
| Z19459 | TPM2 | Nemaline Myopathy |
| D11793 | SLC2A1 | Glucose Transporter Type 1 Deficiency Syndrome |
| HSCHRX | NDP | Norrie Disease |
| T62791 | OPA1 | Optic Atrophy 1 |
| Z24812 | OFD1 | Oral-Facial-Digital Syndrome Type I |
| HUMOTC | OTC | Ornithine Transcarbamylase Deficiency |
| R66505 | MKKS | Bardet-Biedl Syndrome; McKusick-Kaufman Syndrome |
| Z19438 | CHAC | Choreoacanthocytosis |
| HUMRDSA | RDS | Patterned Dystrophy of Retinal Pigment Epithelium; Retinitis Pigmentosa, |
| | | Autosomal Dominant |
| Z30072 | PLP1 | Hereditary Spastic Paraplegia, X-Linked; PLP-Related Disorders |
| HSFGR1IG | FGFR1 | FGFR-Related Craniosynostosis Syndromes; Pfeiffer Syndrome Type 1, 2, |
| | | and 3 |
| HUMPHH | PAH | Phenylalanine Hydroxylase Deficiency |
| HSKITCR | KIT | Gastrointestinal Stromal Tumor; Piebaldism |
| HSGROW1 | GH1 | Pituitary Dwarfism I |
| F00079 | GHR | Pituitary Dwarfism II |
| HSPIT1 | POU1F1 | Pituitary-Specific Transcription Factor Defects (PIT1) |
| T58874 | SDHD | Familial Nonchromaffin Paragangliomas |
| HUMINTB3 | ITGB3 | Integrin, Beta 3; Platelet Antigen Genotyping |
| T09245 | PKD1 | Polycystic Kidney Disease 1, Autosomal Dominant; Polycystic Kidney |
| | | Disease, Autosomal Dominant |
| T55657 | PKD2 | Polycystic Kidney Disease 2, Autosomal Dominant; Polycystic Kidney |
| | | Disease, Autosomal Dominant |
| T77325 | PKD2 | Polycystic Kidney Disease 2, Autosomal Dominant; Polycystic Kidney |
| | | Disease, Autosomal Dominant |
| W27963 | PKD2 | Polycystic Kidney Disease 2, Autosomal Dominant; Polycystic Kidney |
| | | Disease, Autosomal Dominant |
| R05352 | PKHD1 | Polycystic Kidney Disease, Autosomal Recessive |
| M77871 | PCLD | Polycystic Liver Disease |
| M78097 | UROD | Porphyria Cutanea Tarda |
| HUMPBG | HMBS | Acute Intermittent Porphyria |
| HUMRODSA | UROS | Congenital Erythropoietic Porphyria |
| T10891 | AGT | Angiotensinogen |
| T67463 | CTSK | Pycnodysostosis |
| M77954 | PDHA1 | Pyruvate Dehydrogenase Deficiency, X-linked |
| Z19400 | PHYH | Refsum Disease, Adult |
| R07476 | PEX1 | Zellweger Syndrome Spectrum |
| Z24965 | RCA1 | Renal Cell Carcinoma |
| H37900 | RHO | Retinitis Pigmentosa, Autosomal Dominant; Retinitis Pigmentosa, |
| | | Autosomal Recessive |
| T24020 | RB1 | Retinoblastoma |
| Z44098 | RS1 | X-Linked Juvenile Retinoschisis |
| H84683 | RS1 | X-Linked Juvenile Retinoschisis |
| HSRH30A | RHCE | Rh C Genotyping; Rh E Genotyping |
| S57971 | RHCE | Rh C Genotyping; Rh E Genotyping |
| AI282496 | RHCE | Rh C Genotyping; Rh E Genotyping |
| T11224 | RHCE | Rh C Genotyping; Rh E Genotyping |
| R60192 | PEX7 | Refsum Disease, Adult; Rhizomelic Chondrodysplasia Punctata Type 1 |
| HUMMLC1AA | MLC1 | Megalencephalic Leukoencephalopathy with Subcortical Cysts |
| M79106 | MLC1 | Megalencephalic Leukoencephalopathy with Subcortical Cysts |
| T64905 | PITX2 | Anophthalmia; Peters Anomaly; Rieger Syndrome |
| Z41163 | CREBBP | Rubinstein-Taybi Syndrome |
| HSBHLH | TWIST1 | Saethre-Chotzen Syndrome |
| F00367 | EIF2B1 | Childhood Ataxia with Central Nervous System Hypomyelination/Vanishing |
| | | White Matter |
| Z20030 | EIF2B2 | Childhood Ataxia with Central Nervous System Hypomyelination/Vanishing |
| | | White Matter |
| Z41323 | EIF2B3 | Childhood Ataxia with Central Nervous System Hypomyelination/Vanishing |
| | | White Matter |
| Z17882 | EIF2B4 | Childhood Ataxia with Central Nervous System Hypomyelination/ Vanishing |
| | | White Matter |
| R13846 | EIF2B5 | Childhood Ataxia with Central Nervous System Hypomyelination/ Vanishing |
| | | White Matter; Cree Leukoencephalopathy |
| T03917 | HEXB | Sandhoff Disease |
| HUMSRYA | SRY | XX Male Syndrome; XY Gonadal Dysgenesis |
| HUMSCAD | ACADS | Short Chain Acyl-CoA Dehydrogenase Deficiency |
| HSALAS2R | ALAS2 | Sideroblastic Anemia, X-Linked |
| T47846 | GPC3 | Simpson-Golabi-Behmel Syndrome |
| T11069 | GUSB | Mucopolysaccharidosis Type VII |
| T08813 | SPG3A | Hereditary Spastic Paraplegia, Dominant; SPG 3 |
| Z21409 | SPG3A | Hereditary Spastic Paraplegia, Dominant; SPG 3 |
| M77964 | SPG4 | Hereditary Spastic Paraplegia, Dominant; SPG 4 |
| N36808 | SMN1 | Spinal Muscular Atrophy |
| Z38265 | SMN1 | Spinal Muscular Atrophy |
| T06490 | SCA1 | Spinocerebellar Ataxia Type 1 |
| T55469 | SCA2 | Spinocerebellar Ataxia Type 2 |
| Z41764 | SCA2 | Spinocerebellar Ataxia Type 2 |
| T61453 | MJD | Spinocerebellar Ataxia Type 3 |
| HUMELASF | ELN | Cutis Laxa, Autosomal Dominant; Supravalvular Aortic Stenosis |
| T05970 | HEXA | Hexosaminidase A Deficiency |
| M79184 | THRB | Thyroid Hormone Resistance |
| Z20729 | TCOF1 | Treacher Collins Syndrome |
| R48739 | TRPS1 | Trichorhinophalangeal Syndrome Type I |
| T77655 | TSC1 | Tuberous Sclerosis 1; Tuberous Sclerosis Complex |
| M78940 | TSC2 | Tuberous Sclerosis 2; Tuberous Sclerosis Complex |
| HSFAA | FAH | Tyrosinemia Type I |
| T39510 | TBX3 | Ulnar-Mammary Syndrome |
| HUMM7AA | MYO7A | Usher Syndrome Type 1 |
| W22160 | USH1C | Usher Syndrome Type 1 |
| T08506 | ACADVL | Very Long Chain Acyl-CoA Dehydrogenase Deficiency |
| HUMHIPLIND | VHL | Von Hippel-Lindau Syndrome |
| HUMVWF | VWF | Von Willebrand Disease |
| HSU02368 | PAX3 | Waardenburg Syndrome Type I |
| N64051 | WRN | Werner Syndrome |
| HUMWND | ATP7B | Wilson Disease |
| T40645 | WAS | WAS-Related Disorders |
| HSLAL | LIPA | Wolman Disease |
| HSASL1 | ASL | Argininosuccinicaciduria |
| HSAGAGENE | AGA | Aspartylglycosaminuria |
| T88756 | ATD | Asphyxiating Thoracic Dystrophy |
| Z19164 | ASAH | Farber Disease |
| HUMALD | FBP1 | Fructose 1,6 Bisphosphatase Deficiency |
| HSLDHAR | LDHA | Lactate Dehydrogenase Deficiency |
| M77886 | LDHB | Lactate Dehydrogenase Deficiency |
| HSU13680 | LDHC | Lactate Dehydrogenase Deficiency |
| Z46189 | MAN2B1 | Alpha-Mannosidosis |
| M79249 | MANBA | Beta-Mannosidosis |
| H26723 | GALNS | Mucopolysaccharidosis Type IVA |
| H23053 | SLC26A4 | DFNB 4; Enlarged Vestibular Aqueduct Syndrome; Nonsyndromic Hearing |
| | | Loss and Deafness, Autosomal Recessive; Pendred Syndrome |
| HSPGK1 | PGK1 | Phosphoglycerate Kinase Deficiency |
| HSU08818 | MET | Papillary Renal Carcinoma |
| M79231 | PRCC | Papillary Renal Carcinoma |
| T08200 | GNS | Mucopolysaccharidosis Type IIID |
| HUMNAGB | NAGA | Schindler Disease |
| T08881 | NEU1 | Mucolipidosis I |
| R81783 | SLC17A5 | Free Sialic Acid Storage Disorders |
| HUMAUTONH | MTATP6 | Mitochondrial Disorders; Mitochondrial DNA-Associated Leigh Syndrome |
| | | and NARP |
| F09306 | SCA7 | Spinocerebellar Ataxia Type 7 |
| AF248482 | DAZ | Y Chromosome Infertility |
| HSU21663 | DAZ | Y Chromosome Infertility |
| T47024 | JAG1 | Alagille Syndrome |
| HSRYRRM1 | RBMY1A1 | Y Chromosome Infertility |
| HSRYRRM2 | RBMY1A1 | Y Chromosome Infertility |
| HSVD3R | VDR | Osteoporosis; Rickets-Alopecia Syndrome |
| T40157 | FMO3 | Trimethylaminuria |
| HUMPHOSLIP | PPGB | Galactosialidosis |
| HUMPPR | PPGB | Galactosialidosis |
| H22222 | FANCC | Fanconi Anemia |
| D12009 | RPS6KA3 | Coffin-Lowry Syndrome |
| M78282 | PTEN | PTEN Hamartoma Tumor Syndrome (PHTS) |
| M78802 | FY | Duffy Antigen Genotyping |
| HSU04270 | KCNH2 | LQT 2; Romano-Ward Syndrome |
| T19733 | SCN5A | Brugada Syndrome; LQT 3; Romano-Ward Syndrome |
| HSTFIIDX | TBP | Spinocerebellar Ataxia Type 17 |
| HUMKCHA | KCNA1 | Episodic Ataxia Type 1 |
| HSU78110 | NRTN | Hirschsprung Disease |
| HSET3AA | EDN3 | Hirschsprung Disease |
| Z17351 | ECE1 | Hirschsprung Disease |
| T47284 | DHCR7 | Smith-Lemli-Opitz Syndrome |
| HUMXIHB | HBZ | Alpha-Thalassemia |
| HSCP2 | CP | Aceruloplasminemia |
| N25320 | CLN6 | CLN6-Related Neuronal Ceroid-Lipofuscinosis; Neuronal Ceroid- |
| | | Lipofuscinoses |
| T11340 | NBS1 | Nijmegen Breakage Syndrome |
| Z40114 | NBS1 | Nijmegen Breakage Syndrome |
| HSU03688 | CYP1B1 | Glaucoma, Recessive (Congenital); Peters Anomaly |
| D62980 | MYOC | Glaucoma, Dominant (Juvenile Onset) |
| T98453 | NAGLU | Mucopolysaccharidosis Type IIIB |
| AA779817 | RUNX2 | Cleidocranial Dysplasia |
| HUMCBFA | RUNX2 | Cleidocranial Dysplasia |
| HSMARENO | MEFV | Familial Mediterranean Fever |
| F02180 | PHKB | Phosphorylase Kinase Deficiency of Liver and Muscle |
| D11905 | HPS1 | Hermansky-Pudlak Syndrome; Hermansky-Pudlak Syndrome 1 |
| R95987 | CRX | Retinitis Pigmentosa, Autosomal Dominant |
| T05762 | EVC | Ellis-van Creveld Syndrome |
| T12126 | FLNA | Frontometaphyseal Dysplasia; Melnick-Needles Syndrome; Otopalatodigital |
| | | Syndrome; Periventricular Heterotopia, X-Linked |
| T60913 | EBP | Chondrodysplasia Punctata, X-Linked Dominant |
| HSHNF4 | HNF4A | Maturity-Onset Diabetes of the Young Type I |
| HUMBGLUKIN | GCK | Familial Hyperinsulinism; GCK-Related Hyperinsulinism; Maturity-Onset |
| | | Diabetes of the Young Type II |
| M62026 | GCK | Familial Hyperinsulinism; GCK-Related Hyperinsulinism; Maturity-Onset |
| | | Diabetes of the Young Type II |
| R94860 | CIAS1 | Chronic Infantile Neurological Cutaneous and Articular Syndrome; Familial |
| | | Cold Urticaria; Muckle-Wells Syndrome |
| T08221 | SMARCAL1 | Schimke Immunoosseous Dysplasia |
| T95621 | SLC25A15 | Hyperornithinemia-Hyperammonemia-Homocitrullinuria Syndrome |
| HUMOATC | OAT | Ornithine Aminotransferase Deficiency |
| R08989 | MLYCD | Malonyl-CoA Decarboxylase Deficiency |
| N35888 | PMM2 | Congenital Disorders of Glycosylation |
| HSRPMI | MPI | Congenital Disorders of Glycosylation |
| HSSRECV6 | MGAT2 | Congenital Disorders of Glycosylation |
| T91755 | MGAT2 | Congenital Disorders of Glycosylation |
| HSCPTI | CPT1A | Carnitine Palmitoyltransferase IA (liver) Deficiency |
| D12096 | CPT2 | Carnitine Palmitoyltransferase II Deficiency |
| HSA1ATCA | SERPINA1 | Alpha-1-Antitrypsin Deficiency |
| N36808 | SMN2 | Spinal Muscular Atrophy |
| Z38265 | SMN2 | Spinal Muscular Atrophy |
| HUMACADL | ACADL | Long Chain Acyl-CoA Dehydrogenase Deficiency |
| Z25247 | CACT | Carnitine-Acylcarnitine Translocase Deficiency |
| HUMETFA | ETFA | Glutaricacidemia Type 2 |
| HSETFBS | ETFB | Glutaricacidemia Type 2 |
| S69232 | ETFDH | Glutaricacidemia Type 2 |
| T09377 | MEB | Muscle-Eye-Brain Disease |
| Z40427 | G6PT1 | Glycogen Storage Disease Type Ib |
| AI002801 | SLC14A1 | Kidd Genotyping |
| Z19313 | SLC14A1 | Kidd Genotyping |
| HUMPGAMM | PGAM2 | Phosphoglycerate Mutase Deficiency |
| H86930 | MPP4 | Retinitis Pigmentosa, Autosomal Recessive |
| HSU14910 | RGR | Retinitis Pigmentosa, Autosomal Recessive |
| AA775466 | CARD15 | Crohn Disease |
| AA306952 | GAN | Giant Axonal Neuropathy |
| T99245 | CLCN5 | Dent Disease |
| T23537 | NR3C2 | Pseudohypoaldosteronism Type 1, Dominant |
| HSLASNA | SCNN1A | Pseudohypoaldosteronism Type 1, Recessive |
| H26938 | SCNN1B | Pseudoaldosteronism; Pseudohypoaldosteronism Type 1, Recessive |
| HUMGAMM | SCNN1G | Pseudoaldosteronism; Pseudohypoaldosteronism Type 1, Recessive |
| HSP450AL | CYP11B2 | Familial Hyperaldosteronism Type 1; Familial Hypoaldosteronism Type 2 |
| HUMCYPADA | CYP11B1 | Familial Hyperaldosteronism Type 1 |
| AF017089 | COL11A1 | Stickler Syndrome; Stickler Syndrome Type II |
| HUMCA1XIA | COL11A1 | Stickler Syndrome; Stickler Syndrome Type II |
| HUMA2XICOL | COL11A2 | Stickler Syndrome |
| S61523 | PIGA | Paroxysmal Nocturnal Hemoglobinuria |
| T58881 | PHKA2 | Glycogen Storage Disease Type IX |
| Z39614 | DHAPAT | Rhizomelic Chondrodysplasia Punctata Type 2 |
| N89899 | SH2D1A | Lymphoproliferative Disease, X-Linked |
| HUMUGT1FA | UGT1A1 | Gilbert Syndrome |
| HUMNC1A | COL7A1 | Epidermolysis Bullosa Dystrophica, Bart Type; Epidermolysis Bullosa |
| | | Dystrophica, Cockayne-Touraine Type; Epidermolysis Bullosa |
| | | Dystrophica, Hallopeau- Siemens Type; Epidermolysis Bullosa |
| | | Dystrophica, Pasini Type; Epidermolysis Bullosa, Pretibial |
| T49684 | ITGB4 | Epidermolysis Bullosa Letalis with Pyloric Atresia |
| S66196 | ITGA6 | Epidermolysis Bullosa Letalis with Pyloric Atresia |
| T10988 | LAMC2 | Epidermolysis Bullosa Junctional, Herlitz-Pearson Type |
| HUMLAMAA | LAMA3 | Epidermolysis Bullosa Junctional, Herlitz-Pearson Type |
| Z24848 | LAMA3 | Epidermolysis Bullosa Junctional, Herlitz-Pearson Type |
| TI0484 | LAMB3 | Epidermolysis Bullosa Junctional, Disentis Type; Epidermolysis Bullosa |
| | | Junctional, Herlitz- Pearson Type |
| HUMBP180AA | COL17A1 | Epidermolysis Bullosa Junctional, Disentis Type |
| M78889 | PLEC1 | Epidermolysis Bullosa with Muscular Dystrophy |
| Z38659 | SLC22A5 | Carnitine Deficiency, Systemic |
| T85099 | CTNS | Cystinosis |
| W27253 | CNGA3 | Achromatopsia; Achromatopsia 2 |
| HSU66088 | SLC5A5 | Thyroid Hormonogenesis Defect I |
| HUMTEKRPTK | TEK | Venous Malformation, Multiple Cutaneous and Mucosal |
| R69741 | SLC26A2 | Achondrogenesis Type 1B; Atelosteogenesis Type 2; Diastrophic Dysplasia; |
| | | Multiple Epiphyseal Dysplasia, Recessive |
| R70146 | PEX10 | Zellweger Syndrome Spectrum |
| S55790 | COL4A3 | Alport Syndrome; Alport Syndrome, Autosomal Recessive |
| HSCOL4A4 | COL4A4 | Alport Syndrome; Alport Syndrome, Autosomal Recessive |
| T10559 | SHFM3 | Ectrodactyly |
| T99040 | FANCA | Fanconi Anemia |
| H47777 | FANCB | Fanconi Anemia |
| AA542822 | FANCE | Fanconi Anemia |
| HUMPSPB | PSAP | Metachromatic Leukodystrophy |
| HUMSAPA1 | PSAP | Metachromatic Leukodystrophy |
| S69686 | PSAP | Metachromatic Leukodystrophy |
| AA252786 | NCF1 | Chronic Granulomatous Disease |
| HUMNCF1A | NCF1 | Chronic Granulomatous Disease |
| HSTGFB1 | TGFB1 | Camurati-Engelmann Disease |
| R24242 | CYBA | Chronic Granulomatous Disease |
| HUMNOXF | NCF2 | Chronic Granulomatous Disease |
| S41458 | PDE6B | Retinitis Pigmentosa, Autosomal Recessive |
| AA002150 | PDE6B | Retinitis Pigmentosa, Autosomal Recessive |
| R21727 | DYSF | Dysferlinopathy; Limb-Girdle Muscular Dystrophies, Autosomal Recessive |
| AF055580 | USH2A | Usher Syndrome Type 2; Usher Syndrome Type 2A |
| N36632 | MITF | Waardenburg Syndrome Type II; Waardenburg Syndrome Type IIA |
| M78027 | MYH9 | DFNA 17; Epstein Syndrome; Fechtner Syndrome; May-Hegglin Anomaly; |
| | | Sebastian Syndrome |
| Z40194 | HPS4 | Hermansky-Pudlak Syndrome |
| AA333774 | GP1BA | Platelet Antigen Genotyping |
| M79110 | GP1BB | Platelet Antigen Genotyping |
| HUMGPIIBA | ITGA2B | Platelet Antigen Genotyping |
| T29174 | ITGA2 | Glycoprotein 1a Deficiency; Platelet Antigen Genotyping |
| HSGST4 | GSTM1 | Lung Cancer |
| AA773443 | CHEK2 | Li-Fraumeni Syndrome |
| T78869 | CHEK2 | Li-Fraumeni Syndrome |
| T03839 | SH3BP2 | Cherubism |
| T67412 | IRF6 | IRF6-Related Disorders |
| AB037973 | FGF23 | Hypophosphatemic Rickets, Dominant |
| T60199 | FBLN5 | Cutis Laxa, Autosomal Recessive |
| T03890 | ARX | ARX-Related Disorders |
| M79175 | NSD1 | Sotos Syndrome |
| T07860 | NSD1 | Sotos Syndrome |
| M79181 | COH1 | Cohen Syndrome |
| MIHS75KDA | NDUFS1 | Leigh Syndrome (nuclear DNA mutation); Mitochondrial Respiratory Chain |
| | | Complex I Deficiency |
| T09312 | NDUFV1 | Leigh Syndrome (nuclear DNA mutation); Mitochondrial Respiratory Chain |
| | | Complex I Deficiency |
| AA399371 | SALL4 | Acrorenoocular Syndrome; Okihiro Syndrome |
| HUMA8SEQ | TIMP3 | Pseudoinflammatory Fundus Dystrophy |
| Z40623 | GDAP1 | Charcot-Marie-Tooth Neuropathy Type 4; Charcot-Marie-Tooth Neuropathy |
| | | Type 4A |
| AA128030 | FOXL2 | Blepharophimosis, Epicanthus Inversus, Ptosis |
| HUMCRTR | SLC6A8 | Creatine Deficiency Syndrome, X-Linked |
| T08882 | JPH3 | Huntington Disease-Like 2 |
| T07283 | SNRPN | Autistic Disorder; Pervasive Developmental Disorders |
| Z38837 | SPR | Sepiapterin Reductase Deficiency (SR) |
| HUMANTIR | AGTR1 | Angiotensin II Receptor, Type 1 |
| T46961 | SEPN1 | Congenital Muscular Dystrophy with Early Spine Rigidity; Multiminicore |
| | | Disease |
| Z43954 | TRIM32 | Limb-Girdle Muscular Dystrophies, Autosomal Recessive |
| Z19219 | TTID | Limb-Girdle Muscular Dystrophies, Autosomal Dominant |
| HSECADH | CDH1 | Hereditary Diffuse Gastric Cancer |
| Z41199 | WFS1 | Nonsyndromic Low-Frequency Sensorineural Hearing Loss; Wolfram |
| | | Syndrome |
| HUMLORAA | LOR | Progressive Symmetric Erythrokeratoderma |
| Z38324 | HR | Alopecia Universalis; Papular Atrichia |
| T09039 | RYR1 | Central Core Disease of Muscle; Malignant Hyperthermia Susceptibility; |
| | | Multiminicore Disease |
| T10442 | GALE | Galactose Epimerase Deficiency |
| D82541 | PDB2 | Paget Disease of Bone |
| HSU20759 | CASR | Autosomal Dominant Hypocalcemia; Familial Hypocalciuric Hypercalcemia, |
| | | Type I; Familial Isolated Hypopara-thyroidism; Neonatal Severe Primary |
| | | Hyperparathyroidism |
| AA071082 | SALL1 | Townes-Brocks Syndrome |
| T81692 | EDAR | Hypohidrotic Ectodermal Dysplasia; Hypohidrotic Ectodermal Dysplasia, |
| | | Autosomal |
| HUMHPA1B | HP | Anhaptoglobinemia |
| HSU01922 | TIMM8A | Deafness-Dystonia-Optic Neuronopathy Syndrome |
| HUMHSDI | HSD3B2 | Prostate Cancer |
| HSU05659 | HSD17B3 | Prostate Cancer |
| Z38915 | NPHP4 | Nephronophthisis 4; Senior-Loken Syndrome |
| HSC1INHR | SERPING1 | Hereditary Angioneurotic Edema |
| D62739 | BBS7 | Bardet-Biedl Syndrome |
| T64266 | SLC7A7 | Lysinuric Protein Intolerance |
| S52028 | CTH | Cystathioninuria |
| Z30254 | EFEMP1 | Doyne Honeycomb Retinal Dystrophy; Patterned Dystrophy of Retinal |
| | | Pigment Epithelium |
| D59254 | ELOVL4 | Stargardt Disease 3 |
| S43856 | GCH1 | Dopa-Responsive Dystonia; GTP Cyclohydrolase 1-Deficient DRD; GTP |
| | | Cyclohydrolase-1 Deficiency (GTPCH) |
| M78468 | PAFAH1B1 | 17-Linked Lissencephaly |
| M78473 | PAFAH1B1 | 17-Linked Lissencephaly |
| S51033 | MID1 | Opitz Syndrome, X-Linked |
| Z40343 | MID1 | Opitz Syndrome, X-Linked |
| HUM6PTHS | PTS | Pyruvoyltetrahydropterin Synthase Deficiency |
| M62103 | CIRH1A | North American Indian Childhood Cirrhosis |
| HSDHPR | QDPR | Dihydropteridine Reductase Deficiency (DHPR) |
| T23665 | FKRP | Congenital Muscular Dystrophy Type 1C; Limb-Girdle Muscular |
| | | Dystrophies, Autosomal Recessive |
| T60498 | LRPPRC | Leigh Syndrome, French-Canadian Type |
| BG772870 | LRPPRC | Leigh Syndrome, French-Canadian Type |
| HSACHRA | CHRNA1 | Congenital Myasthenic Syndromes |
| HSACHRB | CHRNB1 | Congenital Myasthenic Syndromes |
| HSACHRG | CHRND | Congenital Myasthenic Syndromes |
| HSACETR | CHRNE | Congenital Myasthenic Syndromes |
| HSACRAP | RAPSN | Congenital Myasthenic Syndromes |
| M78334 | COLQ | Congenital Myasthenic Syndromes |
| S56138 | CHAT | Congenital Myasthenic Syndromes |
| D11584 | SDHC | Familial Nonchromaffin Paragangliomas |
| HSPSTI | SPINK1 | Hereditary Pancreatitis |
| HSSPROTR | PROS1 | Protein S Heerlen Variant |
| HUMLAP | ITGB2 | Leukocyte Adhesion Deficiency, Type 1 |
| T12572 | ADAMTS13 | Familial Thrombotic Thrombocytopenia Purpura |
| HUMCOMIIP | SDHB | Carotid Body Tumors and Multiple Extraadrenal Pheochromocytomas |
| NM005912 | MC4R | Obesity |
| HUMPAX8A | PAX8 | Congenital Hypothyroidism |
| AA037119 | FOXE1 | Bamforth-Lazarus Syndrome; Congenital Hypothyroidism |
| AV754057 | FSHB | Isolated Follicle Stimulating Hormone Deficiency |
| HUMHOMEOA | PCBD | Pterin-4a Carbinolamine Dehydratase Deficiency (PCD) |
| HSTHR | TH | Dopa-Responsive Dystonia; Tyrosine Hydroxylase-Deficient DRD |
| AA219596 | ZIC3 | Heterotaxy Syndrome |
| HSU20324 | CSRP3 | Dilated Cardiomyopathy |
| HUMPHLAM | PLN | Dilated Cardiomyopathy |
| F10219 | ALMS1 | Alstrom Syndrome |
| T06612 | VCL | Dilated Cardiomyopathy |
| AF388366 | USH3A | Usher Syndrome Type 3 |
| Z40797 | SGCE | Myoclonus-Dystonia |
| T08448 | RAB7 | Charcot-Marie-Tooth Neuropathy Type 2 |
| D12383 | GARS | Charcot-Marie-Tooth Neuropathy Type 2 |
| Z36734 | HRPT2 | HRPT2-Related Disorders |
| H19914 | EDARADD | Hypohidrotic Ectodermal Dysplasia; Hypohidrotic Ectodermal Dysplasia, |
| | | Autosomal |
| T08852 | PPT1 | Neuronal Ceroid-Lipofuscinoses; PPT1-Related Neuronal Ceroid- |
| | | Lipofuscinosis |
| HUMDRA | SLC26A3 | Familial Chloride Diarrhea |
| R16324 | AGPAT2 | Berardinelli-Seip Congenital Lipodystrophy |
| Z41967 | BSCL2 | Berardinelli-Seip Congenital Lipodystrophy |
| W28410 | OPN1MW | Blue-Mono-Cone-Monochromatic Type Colorblindness |
| T27896 | OPN1LW | Blue-Mono-Cone-Monochromatic Type Colorblindness |
| AI469991 | PHOX2A | Congenital Fibrosis of Extraocular Muscles |
| HSFSTHR | FSHR | Premature Ovarian Failure, Autosomal Recessive |
| HSLPH | LCT | Hypolactasia, Adult Type |
| Z41000 | BCS1L | Gracile Syndrome; Mitochondrial Respiratory Chain Complex III Deficiency |
| HSCGJP | GJA1 | Oculodentodigital Dysplasia |
| HSPERFP1 | PRF1 | Familial Hemophagocytic Lymphohistiocytosis 2 |
| M78112 | GLUD1 | Familial Hyperinsulinism; GLUD1-Related Hyperinsulinism |
| Z39336 | GLUD1 | Familial Hyperinsulinism; GLUD1-Related Hyperinsulinism |
| W79230 | RAX | Anophthalmia |
| AF041339 | PITX3 | Anophthalmia |
| AA151708 | HESX1 | Anophthalmia |
| HSSOXB | SOX3 | Anophthalmia; Mental Retardation, X-Linked, with Growth Hormone |
| | | Deficiency |
| HUMHMGBOX | SOX2 | Anophthalmia |
| HSGM2APA | GM2A | GM2 Activator Deficiency |
| Z19280 | GLC1E | Glaucoma, Dominant (Adult Onset) |
| T20165 | PHF6 | Borjeson-Forssman-Lehmann Syndrome |
| Z40394 | CMT4B2 | Charcot-Marie-Tooth Neuropathy Type 4 |
| HUMIHH | IHH | Brachydactyly Type A1 |
| HUMCDPK | CDK4 | Familial Malignant Melanoma |
| T39355 | SBDS | Shwachman-Diamond Syndrome |
| HSHMPLK | MPL | Amegakaryocytic Thrombocytopenia, Congenital |
| Z38860 | TRIM37 | Mulibrey Nanism |
| M62027 | DTNA | Familial Isolated Noncompaction of Left Ventrical Myocardium |
| Z39175 | DDB2 | Xeroderma Pigmentosum |
| T09329 | MUTYH | MYH-Associated Polyposis |
| HUMAPA | APP | Alzheimer Disease Type 1; Early-Onset Familial Alzheimer Disease |
| M79090 | GSS | 5-Oxoprolinuria |
| Z26981 | OXCT | 3-Oxoacid CoA Transferase |
| D12046 | PMS1 | Hereditary Non-Polyposis Colon Cancer |
| T08186 | PMS2 | Hereditary Non-Polyposis Colon Cancer |
| R20984 | MSH6 | Hereditary Non-Polyposis Colon Cancer |
| T60457 | NDUFS4 | Leigh Syndrome (nuclear DNA mutation); Mitochondrial Respiratory Chain |
| | | Complex I Deficiency |
| D30864 | NDUFS8 | Leigh Syndrome (nuclear DNA mutation) |
| M78107 | SDHA | Leigh Syndrome (nuclear DNA mutation) |
| R15290 | NDUFS7 | Leigh Syndrome (nuclear DNA mutation) |
| HUMPCBA | PC | Pyruvate Carboxylase Deficiency |
| R11095 | AASS | Hyperlysinemia |
| T23789 | PEX3 | Zellweger Syndrome Spectrum |
| T09086 | STK11 | Peutz-Jeghers Syndrome |
| T87335 | HAL | Histidinemia |
| Z19082 | ALDH4A1 | Hyperprolinemia, Type II |
| Z25227 | MADH4 | Juvenile Polyposis Syndrome |
| M78130 | XPB | Xeroderma Pigmentosum |
| T08987 | XPD | Xeroderma Pigmentosum |
| D81449 | XPF | Xeroderma Pigmentosum |
| HSXPGAA | XPG | Xeroderma Pigmentosum |
| HSAUHMR | AUH | 3-Methylglutaconic Aciduria Type 1 |
| T19530 | MMAB | Methylmalonicaciduria |
| Z40169 | MMAA | Methylmalonicaciduria |
| T93695 | BCAT1 | Hyperleucine-Isoleucinemia |
| Z41266 | BCAT2 | Hyperleucine-Isoleucinemia |
| HSU03506 | SLC1A1 | Dicarboxylicaminoaciduria |
| R88591 | PRODH | Hyperprolinemia, Type I |
| T05380 | EPM2A | Progressive Myoclonus Epilepsy, Lafora Type |
| T27227 | FANCF | Fanconi Anemia |
| H49070 | FANCF | Fanconi Anemia |
| Z41736 | FANCG | Fanconi Anemia |
| R66178 | ED4 | Ectodermal Dysplasia, Margarita Island Type |
| L25197 | KCNE1 | Jervell and Lange-Nielsen Syndrome; LQT 5; Romano-Ward Syndrome |
| HUMUMOD | UMOD | Familial Nephropathy with Gout; Medullary Cystic Kidney Disease 2 |
| HSU66583 | CRYGD | Cataract, Crystalline Aculeiform |
| HSPHR | PTHR1 | Chondrodysplasia, Blomstrand Type |
| T97980 | MTRR | Homocystinuria-Megaloblastic Anemia |
| S60710 | ADSL | Adenylosuccinase deficiency |
| Z38216 | SLC25A19 | Amish Lethal Microcephaly |
| T35049 | SLC25A19 | Amish Lethal Microcephaly |
| T11501 | DBH | Dopamine Beta-Hydroxylase Deficiency |
| H11439 | NLGN3 | Autistic Disorder; Pervasive Developmental Disorders |
| R12551 | NLGN4 | Autistic Disorder; Pervasive Developmental Disorders |
| M78212 | ATP1A2 | Familial Hemiplegic Migraine |
| T96957 | SPCH1 | Severe Speech Delay |
| AI266171 | PHOX2B | Congenital Central Hypoventilation Syndrome |
| BG723199 | DSG4 | Localized Autosomal Recessive Hypotrichosis |
| T46918 | HSD11B2 | Apparent Mineralocorticoid Excess Syndrome |
| HUMFERLS | FTL | Hyperferritinemia Cataract Syndrome |
| HUMCKRASA | KRAS2 | Familial Pancreatic Cancer |
| S39383 | PTPN11 | LEOPARD Syndrome; Noonan Syndrome |
| HUMSTAR | STAR | Cholesterol Desmolase Deficiency |
| Z20453 | STAR | Cholesterol Desmolase Deficiency |
| HUMVPC | AVP | Neurohypophyseal Diabetes Insipidus |
| M62144 | MECP2 | Rett Syndrome |
| HSCA2VR | COL5A2 | Ehlers-Danlos Syndrome, Classic Type |
| HUMGENX | TNXB | Ehlers-Danlos-like Syndrome Due to Tenascin-X Deficiency |
| R02385 | TNXB | Ehlers-Danlos-like Syndrome Due to Tenascin-X Deficiency |
| T39901 | LITAF | Charcot-Marie-Tooth Neuropathy Type 1 |
| AA621310 | FOXE3 | Anophthalmia |
| H18132 | CFC1 | Heterotaxy Syndrome |
| R36719 | EBAF | Heterotaxy Syndrome |
| HSACTIIRE | ACVR2B | Heterotaxy Syndrome |
| T52017 | CRELD1 | Heterotaxy Syndrome |
| D11851 | LMNA | Dilated Cardiomyopathy; Emery-Dreifuss Muscular Dystrophy, Autosomal |
| | | Dominant; Familial Partial Lipodystrophy, Dunnigan Type; Hutchinson- |
| | | Gilford Progeria Syndrome; Limb-Girdle Muscular Dystrophies, Autosomal |
| | | Dominant; Mandibuloacral Dysplasia |
| D12062 | DSP | Cardiomyopathy, Dilated, with Woolly Hair and Keratoderma; Keratosis |
| | | Palmoplantaris Striata |
| H99382 | MSH3 | Hereditary Non-Polyposis Colon Cancer |
| AW205295 | NOG | Multiple Synostoses Syndrome |
| AA135181 | GJB3 | Erythrokeratodermia Variabilis |
| F10278 | PEO1 | Mitochondrial DNA Deletion Syndromes |
| M62022 | MASS1 | Febrile Seizures |
| HUMQBPCA | UQCRB | Mitochondrial Respiratory Chain Complex III Deficiency |
| HUMEGR2A | EGR2 | Charcot-Marie-Tooth Neuropathy Type 1; Charcot-Marie-Tooth Neuropathy |
| | | Type 1D; Charcot-Marie-Tooth Neuropathy Type 4; Charcot-Marie-Tooth |
| | | Neuropathy Type 4E |
| HSFLT4X | FLT4 | Milroy Congenital Lymphedema |
| Z24968 | PEX26 | Zellweger Syndrome Spectrum |
| AA338362 | ANKH | Craniometaphyseal Dysplasia, Dominant |
| HUMRPS24A | RPS19 | Diamond-Blackfan Anemia |
| T11633 | RPS19 | Diamond-Blackfan Anemia |
| HSACMHCP | MYH7 | Dilated Cardiomyopathy; Familial Hypertrophic Cardiomyopathy |
| Z25920 | TNNT2 | Dilated Cardiomyopathy; Familial Hypertrophic Cardiomyopathy |
| HUMTRO | TPM1 | Dilated Cardiomyopathy; Familial Hypertrophic Cardiomyopathy |
| Z18303 | MYBPC3 | Dilated Cardiomyopathy; Familial Hypertrophic Cardiomyopathy |
| HSU09466 | COX10 | Leigh Syndrome (nuclear DNA mutation) |
| S72487 | ECGF1 | Mitochondrial Neurogastrointestinal Encephalopathy Syndrome |
| M62196 | KIF5A | Hereditary Spastic Paraplegia, Dominant |
| T07578 | KIF5A | Hereditary Spastic Paraplegia, Dominant |
| D11648 | HSPD1 | Hereditary Spastic Paraplegia, Dominant |
| T47330 | SOX18 | Hypotrichosis-Lymphedema-Telangiectasia Syndrome |
| AA448334 | CAV3 | Caveolinopathy; Limb-Girdle Muscular Dystrophies, Autosomal Dominant |
| AW071529 | ALX4 | Parietal Foramina 2 |
| M61973 | CD2AP | Focal Segmental Glomerulosclerosis |
| W21801 | NR2E3 | Enhanced S-Cone Syndrome |
| Z20305 | TREM2 | PLOSL |
| T05421 | ANK2 | LQT 4; Romano-Ward Syndrome |
| HUMROR2A | ROR2 | ROR2-Related Disorders |
| Z25920 | CMD1D | Dilated Cardiomyopathy |
| AA887962 | HLXB9 | Currarino Syndrome |
| R00281 | ALDH5A1 | Succinic Semialdehyde Dehydrogenase Deficiency |
| HSPCCAR | PCCA | Propionic Acidemia |
| N43992 | DLL3 | Spondylocostal Dysostosis, Autosomal Recessive; Syndactyly, Type IV |
| Z39790 | MUT | Methylmalonicaciduria |
| HUMARGL | ARG1 | Argininemia |
| M78631 | SLC3A1 | Cystinuria |
| T80665 | SLC7A9 | Cystinuria |
| T27286 | HGD | Alkaptonuria |
| HUMBCKDH | BCKDHA | Maple Syrup Urine Disease |
| HUMBCKDHA | BCKDHB | Maple Syrup Urine Disease |
| HSTRANSP | DBT | Maple Syrup Urine Disease |
| Z44722 | HLCS | Holocarboxylase Synthetase Deficiency |
| Z38396 | BTD | Biotinidase Deficiency |
| T48178 | POMT1 | Walker-Warburg Syndrome |
| T28737 | GJB2 | DFNA 3 Nonsyndromic Hearing Loss and Deafness; DFNB 1 Nonsyndromic |
| | | Hearing Loss and Deafness; GJB2-Related DFNA 3 Nonsyndromic Hearing |
| | | Loss and Deafness; GJB2-Related DFNB 1 Nonsyndromic Hearing Loss and |
| | | Deafness; Nonsyndromic Hearing Loss and Deafness, Autosomal Dominant; |
| | | Nonsyndromic Hearing Loss and Deafness, Autosomal Recessive; |
| | | Vohwinkel Syndrome |
| T05861 | COCH | DFNA 9 (COCH); Nonsyndromic Hearing Loss and Deafness, Autosomal Dominant |
| HSBRN4 | POU3F4 | DFN3 |
| HSU21938 | TTPA | Ataxia with Vitamin E Deficiency (AVED) |
| T93783 | KIAA1985 | Charcot-Marie-Tooth Neuropathy Type 4 |
| BE735997 | SANS | Usher Syndrome Type 1 |
| AA548783 | HOXD13 | Syndactyly, Type II |
| R33750 | HOXA13 | Hand-Foot-Uterus Syndrome |
| HUMPP | GLDC | GLDC-Related Glycine Encephalopathy; Glycine Encephalopathy |
| F04230 | AMT | AMT-Related Glycine Encephalopathy; Glycine Encephalopathy |
| T54795 | DECR | 2,4-Dienoyl-CoA Reductase Deficiency |
| R07295 | ACAT1 | Ketothiolase Deficiency |
| S70578 | ACAT1 | Ketothiolase Deficiency |
| HUMMEVKIN | MVK | Hyper IgD Syndrome; Mevalonicaciduria |
| T11245 | HMGCL | 3-Hydroxy-3-Methylglutaryl-Coenzyme A Lyase Deficiency |
| Z41427 | GCDH | Glutaricacidemia Type 1 |
| HSSHOXA | SHOX | Langer Mesomelic Dwarfism; Leri-Weill Dyschondrosteosis; Short Stature |
| HUMDOPADC | DDC | Aromatic L-Amino Acid Decarboxylase Deficiency |
| HSCOL3A4 | COL6A3 | Limb-Girdle Muscular Dystrophies, Autosomal Dominant |
| HSCOL1A4 | COL6A1 | Limb-Girdle Muscular Dystrophies, Autosomal Dominant |
| HSCOL2C2 | COL6A2 | Limb-Girdle Muscular Dystrophies, Autosomal Dominant |
| H16770 | RECQL4 | Rothmund-Thomson Syndrome |
| H11473 | SGSH | Mucopolysaccharidosis Type IIIA |
| H67137 | MCCC1 | 3-Methylcrotonyl-CoA Carboxylase Deficiency |
| R88931 | MCCC2 | 3-Methylcrotonyl-CoA Carboxylase Deficiency |
| Z24865 | TCAP | Dilated Cardiomyopathy; Limb-Girdle Muscular Dystrophies, Autosomal |
| | | Recessive |
| M86030 | DCX | DCX-Related Malformations |
| HUMACTASK | ACTA1 | Nemaline Myopathy |
| HSDGIGLY | DSG1 | Keratosis Palmoplantaris Striata |
| HSRETSA | SAG | Retinitis Pigmentosa, Autosomal Recessive |
| HSAPHOL | ALPL | Hypophosphatasia |
| N73784 | XPA | Xeroderma Pigmentosum |
| T28958 | XPC | Xeroderma Pigmentosum |
| N69543 | POLH | Xeroderma Pigmentosum |
| T54103 | POLH | Xeroderma Pigmentosum |
| H56484 | CKN1 | Cockayne Syndrome |
| Z38185 | ERCC6 | Cockayne Syndrome |
| F07041 | PI12 | Familial Encephalopathy with Neuroserpin Inclusion Bodies |
| AA633404 | KCNE2 | LQT 6; Romano-Ward Syndrome |
| AF302095 | KCNE2 | LQT 6; Romano-Ward Syndrome |
| HSTITINC2 | CMD1G | Dilated Cardiomyopathy |
| N99II5 | NPHP1 | Nephronophthisis 1; Senior-Loken Syndrome |
| HUMELANAA | ELA2 | ELA2-Related Neutropenia |
| S67325 | PCCB | Propionic Acidemia |
| HSGA7331 | M1S1 | Corneal Dystrophy, Gelatinous Drop-Like |
| HSACE | ACE | Angiotensin I Converting Enzyme 1 |
| S49816 | TSHR | Congenital Hypothyroidism; Familial Non-Autoimmune Hyperthyroidism |
| Z30221 | VMGLOM | Multiple Glomus Tumors |
| H88042 | COL9A3 | Multiple Epiphyseal Dysplasia, Dominant |
| M78119 | ADA | Adenosine Deaminase Deficiency |
| T55785 | GAMT | Guanidinoacetate Methyltransferase Deficiency |
| HUMCST4BA | CSTB | Myoclonic Epilepsy of Unverricht and Lundborg |
| S73196 | AQP2 | Nephrogenic Diabetes Insipidus; Nephrogenic Diabetes Insipidus, Autosomal |
| HSU76388 | NR5A1 | XY Sex Reversal with Adrenal Failure |
| HSCPHC22 | MTRNR1 | MTRNR1-Related Hearing Loss and Deafness |
| H21596 | PPARG | Diabetes Mellitus with Acanthosis Nigricans and Hypertension |
| D56550 | FOXC1 | Anophthalmia; Rieger Syndrome |
| M78868 | AP3B1 | Hermansky-Pudlak Syndrome |
| T47068 | NOTCH3 | CADASIL |
| HSHMF1C | TCF1 | Maturity-Onset Diabetes of the Young Type III |
| AA223508 | TCF1 | Maturity-Onset Diabetes of the Young Type III |
| AF049893 | IPF1 | Maturity-Onset Diabetes of the Young Type IV |
| HSU30329 | IPF1 | Maturity-Onset Diabetes of the Young Type IV |
| HSVHNF1 | TCF2 | Maturity-Onset Diabetes of the Young Type V |
| HUMLDLRFMT | LDLR | Familial Hypercholesterolemia |
| HSAPOBR2 | APOB | Familial Hypercholesterolemia Type B |
| T78010 | ABCB7 | Sideroblastic Anemia and Ataxia |
| AF076215 | PROP1 | PROP1-Related Combined Pituitary Hormone Deficiency |
| S99468 | ALAD | Acute Hepatic Porphyria |
| T61818 | ABCC2 | Dubin-Johnson Syndrome |
| HUMLCAT | LCAT | Lecithin Cholesterol Acyltransferase Deficiency |
| Z38510 | HADHSC | Short Chain 3-Hydroxyacyl-CoA Dehydrogenase Deficiency, Liver |
| AF041240 | PPOX | Variegate Porphyria |
| T77011 | PPOX | Variegate Porphyria |
| Z40014 | ALDH10 | Sjogren-Larsson Syndrome |
| S79867 | KRT16 | Nonepidermolytic Palmoplantar Hyperkeratosis; Pachyonychia Congenita |
| HUMKER56K | KRT6A | Pachyonychia Congenita |
| HSKERELP | KRT17 | Pachyonychia Congenita; Steatocystoma Multiplex |
| R11850 | KRT6B | Pachyonychia Congenita |
| S69510 | KRT9 | Epidermolytic Palmoplantar Keratoderma |
| HSCYTK | KRT13 | White Sponge Nevus of Cannon |
| T92918 | KRT4 | White Sponge Nevus of Cannon |
| S54769 | SPG7 | Hereditary Spastic Paraplegia, Recessive; SPG 7 |
| T50707 | FECH | Erythropoietic Protoporphyria |
| HUMPOMM | PXMP3 | Zellweger Syndrome Spectrum |
| R05392 | PEX6 | Zellweger Syndrome Spectrum |
| Z38759 | PEX12 | Zellweger Syndrome Spectrum |
| R14480 | PEX16 | Zellweger Syndrome Spectrum |
| R10031 | PEX13 | Zellweger Syndrome Spectrum |
| R13532 | PXF | Zellweger Syndrome Spectrum |
| Z30136 | AGPS | Rhizomelic Chondrodysplasia Punctata Type 3 |
| HSU07866 | ACOX | Pseudoneonatal Adrenoleukodystrophy |
| N63143 | ALG6 | Congenital Disorders of Glycosylation |
| HSTNFR1A | TNFRSF1A | Familial Hibernian Fever |
| AA018811 | RP1 | Retinitis Pigmentosa, Autosomal Dominant |
| HSG11 | RP1 | Retinitis Pigmentosa, Autosomal Dominant |
| T07942 | RP1 | Retinitis Pigmentosa, Autosomal Dominant |
| H28658 | PRPF31 | Retinitis Pigmentosa, Autosomal Dominant |
| T07062 | PRPF8 | Retinitis Pigmentosa, Autosomal Dominant |
| T05573 | RP18 | Retinitis Pigmentosa, Autosomal Dominant |
| HUMNRLGP | NRL | Retinitis Pigmentosa, Autosomal Dominant |
| T87786 | CRB1 | Retinitis Pigmentosa, Autosomal Recessive |
| H92408 | TULP1 | Retinitis Pigmentosa, Autosomal Recessive |
| S42457 | CNGA1 | Retinitis Pigmentosa, Autosomal Recessive |
| H30568 | PDE6A | Retinitis Pigmentosa, Autosomal Recessive |
| M78192 | RLBP1 | Retinitis Pigmentosa, Autosomal Recessive; Retinitis Pigmentosa, |
| | | Autosomal Recessive, Bothnia Type |
| T10761 | SLC4A4 | Proximal Renal Tubular Acidosis with Ocular Abnormalities |
| N64339 | GJB6 | DFNA 3 Nonsyndromic Hearing Loss and Deafness; DFNB 1 Nonsyndromic |
| | | Hearing Loss and Deafness; GJB6-Related DFNB 1 Nonsyndromic Hearing |
| | | Loss and Deafness; GJB6- Related DFNA 3 Nonsyndromic Hearing Loss and |
| | | Deafness; Hidrotic Ectodermal Dysplasia 2; Nonsyndromic Hearing Loss and |
| | | Deafness, Autosomal Dominant; Nonsyndromic Hearing Loss and |
| | | Deafness, Autosomal Recessive |
| T67968 | MAT1A | Isolated Persistent Hypermethioninemia |
| HUMUMPS | UMPS | Oroticaciduria |
| HSPNP | NP | Purine Nucleoside Phosphorylase Deficiency |
| AB006682 | AIRE | Autoimmune Polyendocrinopathy Syndrome Type 1 |
| BE871354 | JUP | Naxos Disease |
| T08214 | JUP | Naxos Disease |
| F00120 | DES | Dilated Cardiomyopathy |
| R28506 | MOCS1 | Molybdenum Cofactor Deficiency |
| T70309 | MOCS2 | Molybdenum Cofactor Deficiency |
| T08212 | SNCA | Parkinson Disease |
| R99091 | ABCC6 | Pseudoxanthoma Elasticum |
| T69749 | ABCC6 | Pseudoxanthoma Elasticum |
| AA207040 | PRG4 | Arthropathy Camptodactyly Syndrome |
| T57014 | PRG4 | Arthropathy Camptodactyly Syndrome |
| F07016 | OPPG | Osteoporosis Pseudoglioma Syndrome |
| H27782 | SCO2 | Fatal Infantile Cardioencephalopathy due to COX Deficiency |
| S54705S1 | PRKAR1A | Carney Complex |
| Z25903 | SCA10 | Spinocerebellar Ataxia Type 10 |
| AA592984 | WISP3 | Progressive Pseudorheumatoid Arthropathy of Childhood |
| Z39666 | MCOLN1 | Mucolipidosis IV |
| HSEMX2 | EMX2 | Familial Schizencephaly |
| HUMSP18A | SFTPB | Pulmonary Surfactant Protein B Deficiency |
| Z40188 | ATP8B1 | Benign Recurrent Intrahepatic Cholestasis; Progressive Familial Intrahepatic |
| | | Cholestasis; Progressive Familial Intrahepatic Cholestasis 1 |
| U46845 | CYP27B1 | Pseudovitamin D Deficiency Rickets |
| Z21585 | MAPT | Frontotemporal Dementia with Parkinsonism-17 |
| HSPPD | HPD | Tyrosinemia Type III |
| HUMUGT1FA | UGT1A | Crigler-Najjar Syndrome |
| R20880 | SLC19A2 | Thiamine-Responsive Megaloblastic Anemia Syndrome |
| H42203 | TFAP2B | Char Syndrome |
| Z30126 | RYR2 | Catecholaminergic Ventricular Tachycardia, Autosomal Dominant |
| HSSPYRAT | AGXT | Hyperoxaluria, Primary, Type 1 |
| T80758 | SEDL | Spondyloepiphyseal Dysplasia Tarda, X-Linked |
| T89449 | SEDL | Spondyloepiphyseal Dysplasia Tarda, X-Linked |
| AA373083 | FOXC2 | Lymphedema with Distichiasis |
| HUMPROP2AB | SCA12 | Spinocerebellar Ataxia Type 12 |
| Z30145 | ACTC | Dilated Cardiomyopathy |
| HS1900 | GDNF | Hirschsprung Disease |
| M62223 | NEFL | Charcot-Marie-Tooth Neuropathy Type 1F/2E; Charcot-Marie-Tooth |
| | | Neuropathy Type 2; Charcot-Marie-Tooth Neuropathy Type 2E/1F |
| T10920 | SERPINE1 | Plasminogen Activator Inhibitor I |
| HSNCAML1 | L1CAM | Hereditary Spastic Paraplegia, X-Linked; L1 Syndrome |
| T11074 | L1CAM | Hereditary Spastic Paraplegia, X-Linked; L1 Syndrome |
| HUMHPROT | GCSH | Glycine Encephalopathy |
| HSTATR | TAT | Tyrosinemia Type II |
| Z19514 | CPT1B | Carnitine Palmitoyltransferase IB (muscle) Deficiency |
| BE149388 | CPT1B | Carnitine Palmitoyltransferase IB (muscle) Deficiency |
| HSALK3A | BMPR1A | Juvenile Polyposis Syndrome |
| T78581 | CLN5 | CLN5-Related Neuronal Ceroid-Lipofuscinosis; Neuronal Ceroid- |
| | | Lipofuscinoses |
| N32269 | CLN8 | CLN8-Related Neuronal Ceroid-Lipofuscinosis; Neuronal Ceroid- |
| | | Lipofuscinoses |
| HSU44128 | SLC12A3 | Gitelman Syndrome |
| AI590292 | NPHS2 | Focal Segmental Glomerulosclerosis; Steroid-Resistant Nephrotic Syndrome |
| M62209 | ACTN4 | Focal Segmental Glomerulosclerosis |
| H53423 | CNGB3 | Achromatopsia; Achromatopsia 3 |
| HSEPAR | HCI | Hemangioma, Hereditary |
| R14741 | ZIC2 | Holoprosencephaly 5 |
| H84264 | SIX3 | Anophthalmia; Holoprosencephaly 2 |
| T10497 | TGIF | Holoprosencephaly 4 |
| Z30052 | USP9Y | Y Chromosome Infertility |
| N85185 | DBY | Y Chromosome Infertility |
| T11164 | SPTLC1 | Hereditary Sensory Neuropathy Type I |
| T68440 | GNE | GNE-Related Myopathies; Sialuria, French Type |
| HSPROPERD | PFC | Properdin Deficiency, X-Linked |
| T46865 | SURF1 | Leigh Syndrome (nuclear DNA mutation) |
| AI015025 | VAX1 | Anophthalmia |
| BM727523 | VAX1 | Anophthalmia |
| AA310724 | SIX6 | Anophthalmia |
| R37821 | TP63 | TP63-Related Disorders |
| AF091582 | ABCB11 | Progressive Familial Intrahepatic Cholestasis |
| HUMHOX7 | MSX1 | Hypodontia, Autosomal Dominant; Tooth-and-Nail Syndrome |
| R15034 | CACNB4 | Episodic Ataxia Type 2 |
| T52100 | TYROBP | PLOSL |
| F09012 | MTMR2 | Charcot-Marie-Tooth Neuropathy Type 4 |
| T08510 | APTX | Ataxia with Oculomotor Apraxia; Ataxia with Oculomotor Apraxia 1 |
| HUMHAAC | HF1 | Hemolytic-Uremic Syndrome |
| C16899 | MTND5 | Leber Hereditary Optic Neuropathy; Mitochondrial DNA-Associated Leigh |
| | | Syndrome and NARP |

#AUTOANTIGEN_IN_AUTOIMMUNE_DISEASE—Secreted splice variants of know-n autoantigens associated with a specific autoimmune syndrome, as for example, these listed in table 11, can be used to treat the syndrome. The proposed therapeutic mechanism is that the secreted splice variant would bind the auto-antibodies which formed against the autoantigen, therefore reduce their circulating levels, that would lead to less binding of the autoantigen by auto antibodies and as a consequence diminish the autoimmune clinical symptoms.

Examples of proteins which are involved in autoimmune diseases are presented in Table 11 together with the corresponding internal gene contig name, enabling to allocate the new sloce variants within the data files in the attached CD-ROM 4.

TABLE 11

| Contig | Disease | Description |
|---|---|---|
| HUMROSSA | Sjogren's syndrome | 52 kDa Ro protein |
| HUMI69KAA | Insulin dependent diabetes | 69 kDa islet cell autoantigen |
| | Mellitus | |
| S55790 | Goodpasture's syndrome | alpha 3 chain of collagen IV |
| HSACHRA | Myasthenia Gravis | Alpha chain of nicotinic Acetyl Choline receptor |
| Z21711 | Rheumatoid Arthritis | Annexin A11 |
| Z21711 | Sjogren's syndrome | Annexin A11 |
| Z21711 | SLE | Annexin A11 |
| S38729 | SLE | ATP-dependent DNA helicase II, 70 kDa subunit |
| M77907 | SLE | ATP-dependent DNA helicase II, 80 kDa subunit |
| T08224 | scleroderma | Autoantigen p27 |
| T08224 | Sjogren's syndrome | Autoantigen p27 |
| M85815 | Pemphigus | bullous pemphigoid antigen 1 |
| HUMROSSAA | SLE | calreticulin |
| HUMCENPRO | General autoimmune | Centromere autoantigen C |
| | response | |
| HSU14518 | General autoimmune | Centromere protein A |
| | response | |
| M62116 | dermatomyositis | Chromodomain helicase-DNA-binding protein 3 |
| T05980 | dermatomyositis | Chromodomain helicase-DNA-binding protein 4 |
| H18687 | Autoimmune demyelinating | claudin 11 |
| | disease | |
| M79258 | dermatomyositis | Dermatomyositis associated with cancer putative |
| | | autoantigen-1 |
| HSDGIGLY | Pemphigus foliaceus | Desmoglein 1 |
| HUMPVA | Pemphigus vulgaris | Desmoglein 3 |
| BG723199 | Pemphigus vulgaris | desmoglein 4 |
| M77924 | Primary billiary cirrhosis | Dihydrolipoamide acetyltransferase component of |
| | | pyruvate dehydrogenase complex, mitochondrial |
| D11598 | Polymyositis | Exosome complex exonuclease RRP45 |
| D11598 | scleroderma | Exosome complex exonuclease RRP45 |
| HUMACTINBI | Grave's disease | Filamin B |
| Z17837 | Rheumatoid Arthritis | follistatin-like 1 |
| HUMGAD | Insulin dependent diabetes | glutamate decarboxylase 1 (GAD 1) |
| | Mellitus | |
| HSGLAD2A | Insulin dependent diabetes | glutamate decarboxylase 2 (GAD 2) |
| | Mellitus | |
| D12383 | dermatomyositis | glycyl-tRNA synthetase |
| D12383 | Polymyositis | glycyl-tRNA synthetase |
| Z40013 | Sjogren's syndrome | Golgi autoantigen, golgin subfamily A member 1 |
| N28220 | Rheumatoid Arthritis | Golgi autoantigen, golgin subfamily B member 1 |
| N28220 | Sjogren's syndrome | Golgi autoantigen, golgin subfamily B member 1 |
| HUMMSCA | Grave's disease | Grave's disease carrier protein |
| HUMGRAVIN | Myasthenia Gravis | gravin |
| HUMRNPSMBA | SLE | Homo sapiens small nuclear ribonucleoprotein |
| | | polypeptides B and B1 |
| HUMINSR | Insulin resistant diabetes | insulin receptor |
| | Mellitus | |
| HSRNAIFMH | Pernicious Anemia | intrinsic factor |
| D12018 | dermatomyositis | isoleucine-tRNA synthetase |
| D12018 | Polymyositis | isoleucine-tRNA synthetase |
| T97710 | Pemphigus | ladinin 1 |
| HSAUTAN64 | Autoimmune thyroid disease | Leiomodin 1 |
| HSLAANT | SLE | Lupus La protein |
| HUM60RO | SLE | Lupus Ro Protein |
| F02808 | dermatomyositis | lysyl-tRNA synthetase |
| F02808 | Polymyositis | lysyl-tRNA synthetase |
| F01282 | General autoimmune | Major centromere autoantigen B |
| | response | |
| M78010 | multiple sclerosis | myelin basic protein |
| R89508 | Autoimmune demyelinating | Myelin oligodendrocyte glycoprotein (MOG) |
| | disease | |
| HUMHSTNBP | Autoimmune infertility | Nuclear autoantigenic sperm protein |
| S80305 | Antiphospholipid syndrome | Phospholipid beta 2 glycoprotein 1 complex |
| D11598 | Polymyositis | polymyositis/scleroderma autoantigen 1 |
| D11598 | scleroderma | polymyositis/scleroderma autoantigen 1 |
| HUMAUA | Polymyositis | Polymyositis/scleroderma autoantigen 2 |
| HUMAUA | scleroderma | Polymyositis/scleroderma autoantigen 2 |
| HUMMCH | Vitiligo | Pro-melanin-concentrating hormone |
| T05361 | Insulin dependent diabetes | protein tyrosine phosphatase |
| | Mellitus | |
| HSP3MY | Wegener's granulomatosis | Proteinase 3 (ANCA - antineutrophil cytoplasmic |
| | | antibody) |
| F02560 | Insulin dependent diabetes | Protein-tyrosine phosphatase-like N [Precursor] |
| | Mellitus | |
| T05361 | Insulin dependent diabetes | Receptor-type protein-tyrosine phosphatase N2 |
| | Mellitus | |
| HUM60RO | Sjogren's syndrome | Sjogren syndrome antigen A2 |
| H81770 | Sjogren's syndrome | Sjogren's syndrome nuclear autoantigen 1 |
| HUMSNRNPD | SLE | Small nuclear ribonucleoprotein Sm D1 |
| HUMMSCA | Grave's disease | solute carrier family 25 |
| Z17347 | Insulin dependent diabetes | SOX-13 protein |
| | Mellitus | |
| N79953 | Autoimmune infertility | Sperm surface protein Sp17 |
| T08224 | scleroderma | SSSCA1 |
| T08224 | Sjogren's syndrome | SSSCA1 |
| R54783 | interstitial cystitis | synaptonemal complex protein SC65 (SC65) |
| S40807 | Hashimoto's thyroditis | thyroglobulin |
| S38729 | Autoimmune thyroid disease | thyroid autoantigen 70 kDa |
| HUMTPOA | Hashimoto's thyroditis | Thyroid peroxidase |
| HUMBF7A | Celiac disease | transglutaminase 2 |
| S49816 | Grave's disease | TSH receptor |

Differentially Expressed Biomolecular Sequences—Field Description

#TS—This field denotes tissue-specific genes which gene products are upregulated in at least one tissue. Such gene products might be used as tissue or pathological markers. Therapeutic uses of such gene products vary and may include, for example, anti-cancer vaccination and drug-targeting. Other exemplary uses are described hereinabove. It will be appreciated that avary differentially expressed gene product can be assigned to higher hierarchies of classification. Thus, for example, a prostate cancer specific gene product may be used as a diagnostic marker for this cancer, but may be also used as epithelial cancer marker and as a general cancer marker. See for example, Table 12, below.

TABLE 12

| Tissue-tumor searched | Cancer sub-type | Cancer type | Cancer - general |
|---|---|---|---|
| All tumor types | | | All tumor types |
| prostate-tumor | prostate-tumor | All epithelial tumors | All tumor types |
| lung-tumor | lung-tumor | All epithelial tumors | All tumor types |
| head and neck-tumor | head and neck-tumor | All epithelial tumors | All tumor types |
| stomach-tumor | stomach-tumor | All epithelial tumors | All tumor types |
| colon-tumor | colon-tumor | All epithelial tumors | All tumor types |
| mammary-tumor | mammary-tumor | All epithelial tumors | All tumor types |
| kidney-tumor | kidney-tumor | All epithelial tumors | All tumor types |
| ovary-tumor | ovary-tumor | All epithelial tumors | All tumor types |
| uterus/cervix-tumor | uterus/cervix-tumor | All epithelial tumors | All tumor types |
| thyroid-tumor | thyroid-tumor | All epithelial tumors | All tumor types |
| adrenal-tumor | adrenal-tumor | All epithelial tumors | All tumor types |
| pancreas-tumor | pancreas-tumor | All epithelial tumors | All tumor types |
| liver-tumor | liver-tumor | All epithelial tumors | All tumor types |
| skin-tumor | skin-tumor | All epithelial tumors | All tumor types |
| brain-tumor | brain-tumor | | All tumor types |
| eye-tumor | eye-tumor | | All tumor types |
| bone-tumor | bone-tumor | Sarcoma | All tumor types |
| bone marrow-tumor | bone marrow-tumor | Blood cancer | All tumor types |
| blood-cancer | blood-cancer | Blood cacner | All tumor types |
| T-cells-tumor | T-cells-tumor | Blood cancer | All tumor types |
| lymph nodes-tumor | lymph nodes-tumor | Blood cancer | All tumor types |
| muscle-tumor | muscle-tumor | Sarcoma | All tumor types |
| testis-tumor | testis-tumor | | All tumor types |

The annotation format of differentially expressed gene products is as follows.

#TS tissue-name—where the “tissue name” field specifies the list of tissues for which tissue-specific genes/variants were searched, as follows: amniotic+placenta; Blood; Bone; Bone marrow; Brain; Cervix+uterus; Colon; Endocrine, adrenal gland; Endocrine, pancreas; Endocrine, parathyroid+thyroid; Gastrointestinal tract; Genitourinary; Head and neck; Immune, T-cells; Kidney; Liver; Lung; Lymph node; Mammary gland; Muscle; Ovary; Prostate; Skin; Thymus.

#TAA—This field denotes genes or transcript sequences over-expressed in cancer. The annotation format is as follows.

#TAA tissue-name—where the “tissue name” field specifies the list of tissues for which tissue-tumor specific genes/variants were searched, as follows: All tumor types; All epithelial tumors; prostate-tumor; lung-tumor; head and neck-tumor; stomach-tumor; colon-tumor; mammary-tumor; kidney-tumor; ovary-tumor; uterus/cervix-tumor; thyroid-tumor; adrenal-tumor; pancreas-tumor; liver-tumor; skin-tumor; brain-tumor; bone-tumor; bone marrow-tumor; blood-cancer; T-cells-tumor; lymph nodes-tumor; muscle-tumor.

#TAAT—This field denotes splice variants over expressed in cancer. The annotation format is as follows.

#TAAT tissue-name start nucleotide-end nucleotide—, where the “start nucleotide-end nucleotide”field denotes the start and end nucleotides are the location on the transcript of the unique exon/s of this transcript which are over expressed in cancer.

The following are examples of annotational data, described hereinabove, for differentially expressed biomolecular sequences uncovered using the methodology of the present invention.

>125 T12234_S7 (124 T12234_S5) #PHARM B cell inhibitor #PHARM B cell stimulant #INDICATION Allergy, general; Anaemia, general; Anti-inflammatory; Antiallergic, non-asthma; Antianaemic; Antiarthritic, immunological; Antiarthritic, other; Antiasthma; Anticancer, immunological; Anticancer, other; Antidiabetic; Arthritis, rheumatoid; Asthma; Cancer, basal cell; Cancer, breast; Cancer, colorectal; Cancer, leukaemia, general; Cancer, lung, non-small cell; Cancer, lymphoma, B-cell; Cancer, lymphoma, general; Cancer, lymphoma, non-Hodgkin's; Cancer, melanoma; Cancer, myeloma; Cancer, prostate; Cancer, renal; Cancer, sarcoma, Kaposi's; Cancer, stomach; Chemotherapy-induced injury, bone marrow, general; Chemotherapy-induced injury, general; Cytokine; Diabetes, Type I; Diagnosis, cancer; Gene therapy; Haematological; Immunoconjugate, other; Immunodeficiency, IgA deficiency; Immunodeficiency, IgG deficiency; Immunomodulator, anti-infective; Immunostimulant, anti-AIDS; Immunostimulant, other; Immunosuppressant; Infection, HIV/AIDS; Infection, cytomegalovirus; Infection, hepatitis-B virus; Infection, hepatitis-B virus prophylaxis; Infection, hepatitis-C virus; Infection, influenza virus; Infection, respiratory tract, lower; Inflammation, general; Lupus erythematosus, systemic; Lupus nephritis; Menstruation disorders; Monoclonal antibody, chimaeric; Monoclonal antibody, human; Monoclonal antibody, other; Non-antisense oligonucleotides; Prophylactic vaccine; Radio/chemoprotective; Recombinant growth factor; Recombinant interleukin; Recombinant vaccine; Releasing hormones; Renal failure; Reproductive/gonadal, general; Stomatological; Transplant rejection, general; Urological; Vaccine adjunct; #TS amniotic+placenta #SEQLIST CB959801 CB993198 BG723218 CB988266 CB990001 CB960437 CB960673 AY152547 HSU88047 NM005224 BM560075 BG480550 BG481613 BG336181 BC033163 BM914890 BM915483 BG774041 BE407615 BE278788 BU553664 AL528528 BE281155 BG335245 AW502116 AW502448 AW502360 T12234 BG336194 BG336792 BG471353 BE251115 BM728646 BF988865 BG480658 BF752956 BI055866 BX349962 AW874049 BX327713 AW361327 AW604456 AA705382 AI394608 R36384 AW009403 CA424222 BU953740 BC007077 AA371391 AI635170 BU616621 BE018489 CA420992 BX344903 AL563180 BI090573 BX282372 AA232770 AI343403 BE350191 AA219626AI128378

>89 AA176616_TO (88 AA176616_P2) #TS brain #SEQLIST AA176616 AL706148 AF188700 BC032777 AL710268 AL706541 NM021638 AI878896 AL708077 AL044957 BI561136 BG818703 AL597876 BF931341

>121 AA542845_T6 #TAA all tumor types #SEQLIST BM821505 BM820228 BM833450 BM822871 BM450551 BM822584 BG685476 BG759086 BF975093 BG758047 BG684967 BE879584 BG613292 BF670091 BM741097 BI226181 BC032142 CD248060 BG033600 BU935172 BG616080 BF238873 BG496847 AY028916 BE513408 NM032117 BX118316 AW803742 CA430591 BU622320 AW173084 BG027970 CB053175 BG109991 BQ876910 BU533354 CB053174 BQ888320 BF513683 AA782986 BG678591 BG213307 BE775171 AA971073 BG187870 BG201266 BG211199 BG190562 BG188927 BU953916 AW972924 AA542845 BG031442

>1780 D12188_T22 (1779 D12188_P10) #TAA stomach-tumor #SEQLIST BI667214 AA069168 CB120972 AA146921 BF339541 BE697327 AA018956 BI868974 AW977547 AW016369 BF994680 BF994678 AA768226 AA482525 AA417892 AV747968 AV749122 BI018849 BF327760 AA815174 T11015 CB121829 CB265681 CB114032 T10894 R07220 AU099455 BE940424 AA034472 AA085190 CB122775 CD110517 AW812500 BF445602 BM835953 AL702485 CB137205 AA317134 BM698061 AV686120 BM844438 BF963067 R84427 BQ347914 CB132190 BE812639H53309H54062 CB322047 BX420238 AW752802 BG008882 AW752803 AL712969 AW752822 AW838203 BM844307 AW403110 BQ694780 BM843951 BQ272011 W56384 CB119170 BQ291729 AA037057 AA063367 AA021068 BM468187 WO5307 BU561523 AV689084 CB122111 AW674114 AA058777 CB115968 BQ340054 R18396 CB119210 AA975948 AA374973 BG898631 BM888115 BM462720 BG704216 CB114864 BE894309 AA348659 BM847309 AL559362 CB114023 BM843812 CA391445 BQ227099 BM747740 CB115337 R86059 AW838393 BE000940 AW376878 BG940230 BG988188H44528H44511 BI056192 R83531H44513 R73359 AA551357H44512 BQ271689 AW973514 AA994108 BU948701 BG940229 AI280227 AA534047 AA953711 AA094698 BF832976 BF856679 BM843946 D79108 AV708137 AV703503 CB045840 CB115801 CB110101 AA307112 AA309647 BM819549 BF115653 AA019960 BM761384 CB119259 CB178328 BM788339 BI915305 AI125690 W56155 CB140821 CB123983 CB114859 CB 149671 CB122938 CB122913 BG898806 CA406239 BM542792 Z21191 AW068861 CB122934 CB144641 BU599940 BF665043 CA395566 BF945470 BM791398 CB134041 BQ231812 BM456716 BU 164262 BQ777351 BE894021 BM791005 AU137511 BQ953788 BM843126 BM452319 BE540905 CA773780 BI551564 CB216095 CB215747 AW239473 BE269198 BQ214343 BM791465 AU135994 AA303881 BF082675 AA877149 BF893173 BE068965 BQ331544 CB119266 BM772290 CA406825 CB158897 CB122643 BM760734 BM765063 BF082716 BG949629 BI549175 BI010948 BI016251 BF893182 BF773210 BF768828 BI015143 BI013525 W05482 BE892227 CA442266 BE886787 BM999021 AA363541 AL036270 CB110183 BG773048 AU137419 BI092416 CB988632H16540 R16060 BF852596 BQ108743 CB242845 AV708995 CD251708 BI029212 BI030865 BI030862 BG723362 BG107552 BG772916 AW800206 F06911 BU189109 BU177966 AA216699 BI468513 CB993967 BF341343 BG171853 BE888095 BE890937 BF967377 BM707195 BI091903 N94298 BI090331 AA325593 BG171642 AA037516 BE565830 CB119330 BM752427 BE562276 BQ424269 BQ437514 BU186557 AA322781 BG390997 BG114948 BQ310814 BM837070 BQ720930 BE547324 R58206 BE897153 BG388576 AF498929 BG899293 BQ681067 CB128905 AU132656 BG698150 BE773333 BG705788 BQ433491 BF540961 BQ377040 BI764787 BF692590 BQ424046 BE885985 BQ308854 BU195290 AW956847 BE935829 AW954378 CD105507 BU162355 BI912425 BI599480 BQ308017 AA393842 AA868907 AV728310 BI760445 AV661126 NM004161 AV727669 HUMRAB1A AW627895 BE786127 BG250484 AI208230 BQ437146 BG534065 AV661125 AA282775 BG250152 AA525489 BG281078 BF970841 BQ223273 BF530743 BI858729 BM452068 BQ921303 R31123 BM450994 BF821830 BF822942 CD556388 CD519333 BU170353 BX345433 BU170821 BM756987 BX460643 AA165326 AV717718 BM786746 BF691745 BI601531 CB164305 BM800733 BI598835 BM476507 BM922791 BF029031 BF247598 W00963 T29874 BE958017 BX345434 BF211990 R14095 AV708027 CB121142 BQ314772 BM919860 N28650 BG573345 AW850068 AW849755 BG743352 CA771560 BG500384 BI495590 BG168366 BI496921 BM829716 C03749 CA942358 BX426888 CB108527 BG619962 AV702665 BX448589 BM452262 BM542833 AA609771 BF673431 AF170935 AA447942 BX463467 BF890884 BF932035 AW605322 CB131651 BF792766 BE568870 BM784959 BG547236 CD108335 BM767367 BG111725 BG562818 BF090111 BE000976 AW888620 BM450140 BI087362 AW955054 BG538626 BF037863 BG563261 BM904432 CD245285 BU193816 AL539022 CB161342 AA229813 R25145 AL530265 W04313 BX440905H04049 BM694415 BG776554 BE617480 BM686049 BG676937 BG432954 BE786784 AW389890 BG779464 BU945327 AA393153 AA112860 R31365 BI913132 CA867672 CB161701 BX452629 AI342700 BM706159 AA962389 CB164662 BG032817 BX332699 BM702777 BQ276789 BM747028 BE818819 AA604440 BG622470 BU927812 AW949877 AL580999 BE771083 AV702319 BE617921 BF967807 CA389222 BX345431 BM826571 BI092003H01861 BE771069 BI913092 BF447660 BI869965 BX332698 D51100 AA825801 BU567689 BX411609 BG617277 BM783973 AA903879 BE771068 BX345432 AA229649 R88420 AI299811 N51901 AA115325 AI422754 AA857140 BG178268 AI285303 AA782737 BF215497 BM983826 BQ003293 CA443454 BQ276678 R16059 BM983670 N94989 BQ788033 AA047226 CB178572 BG434409 BE972858 AU185510 AA448877 HSM800023 AV645424 N73941 CB116472 AU156411 AU154149 BM973320 CB114088 CB122944 CB119169 AA702144 BC000905 N36763 CB119152 AA283077 CB116486 CB118471 AI056955 CB119061 BE465097 AI636837 AV645778 CB118460 AA043751 AA058471 AI858694H03362 CB122915 CB114037 CB110114N75497 CB110081 CB113929 CB122736 CB113962 CB119817 AI872853 CB121359 CB118415 N34579 BQ448090 CB115729 AI026998 AA018921 AW169620 BU677700 AA019266 AW002352 BU622272 N70762 CA311086 BU736924 AW663003 BM667225 BM971301 BE714687 AI434392 BM991470 BG223478 BU688425 AW136631 AA020983 AA019890 N66759 AW104753 R31083 AI860577 AI889183 AW575163 BM999282 AI628146 BQ772048 AI350328 AA746643 BU626516 BU680296 BM984215 BQ014597 BU608906 BI468512 CB306393 N22842 BM984471 AW069359 CA503384 AI754132 AW673786 AA435590 BX424956 AI828874 AA844547 C75589 AI287282 AA035154 CB118341 AW473264 AI343795 BF372829 AI191816 C75414 BG231998 CA867063 BU069071 AI066620 C75465 AA805211 C75659 AI097435 C75516 W60992 BM969765H88552 AW166902 R25146 AW471315 AI884351 AI127749 C75610 BQ000946 AI143341 AA855141 R42459 AI148222 AI952757 AA860442 AI800097 AW150848 AI191331 AI684028 N69689 CB107598 AA601550 AI089357 CB113484 AI097427 AA037361 N74146 D58246 AA776990 BG939358 AA165327 AW972204 AA778332 AI799192 AW236263 N70637 AI245751 D12188 CB994890 BE879644 BF440024 BE962443 AI094813 AA769867 AI720190 AA553840 N70238 AA983962 AA033620 AA216604 BF029770 AA069169 BQ776896 F03178 BG257928 AA962096 AA600022 BQ010358 CD239850 BI495589 AI886405 BG059991 BU726083 CA441504 AA551680 CA446990 CB219015 CA422823 BF382544 BG059705 AA586815 BM975245 AI096519 CA425640H01862 AW190066 BG236221 AI025608 AA507519 AA398553 BE568059 AA918487 C75502 AI680344 BQ776581 BF433185 CA771253 C75521 AW969792 C75459 AI335718 AA484873 BF238483 AU146032 AW086107 BE139600 BE646347 AA076117 BM472577 BG938435 BI086445 BX413207 CD514144 BX452630 AA253286 AA456890 Z32881 BM766511 BI917513 W74145 W74146 W74151 BM472811 BF029576 N45488 BG498271 CB157466 BG498187 W30880 AA400752 BE874417 BX448588 W30883 BM689897 BF667421 BF692063 BF028711 BE564328 BM827080 BE566877 BE564359 BE564278 BG538932 AA493231 BI090805 BG492697 AA418454 BF246949 AI697924 BX417813 AA628947 AL530264 AW970415 BI764324 BF433701 BE670383 AI765971 AI805951 AI690022 AI291415 AW188359 AA908254 BE464880 AI694931 BM795518 AI188743 BF224091 BE503079 BE669944 AI302751 AI693340 C01263 AI871744 AW263291 AI373523 AW235080 BF590042 BF593086 AI633918 AI962999 AW078858 AW262562 AI377218 AI804431 AK055927 AI656152 AI683808 BG150110 AI394179 BQ017287 CA418030 AW300526 AI797649 BU753351 AI933975 AI685760 AI283710 AI221410 AI623655 AI146623 AA535127 AI950013 AA418384 AI768809 AW771276 AI245073 AA400670 AA506113 CD369826 BQ030029 AW236683 AI913948 AI500621 BU620635 AI085359 AW571693 BE673936 AW299978 R39965 CA429063 AW069008 AW194519 AI378576 BU619001 AI288901 BU634305 BM968348 AI204696 AI276084 BE671896 AI096452 BM661969 AI290774 AA514463 BM981294 AA906864 AW196314 AA457046 BF878685H00768H00677 BX112077 BQ023552 BF431990 AI223034 AW631338 AI216459 #DN IPR003577 Ras small GTPase, Ras type #DN IPR002041 GTP-binding nuclear protein Ran #DN IPR003578 Ras small GTPase, Rho type #DN IPR001806 Ras GTPase superfamily #DN IPR006688 ADP-ribosylation factor #DN IPR003579 Ras small GTPase, Rab type

>44100 D63246_Ti (44099 D63246_P2) #TAAT all tumor types 1-447, #SEQLIST AI459211 AI298516 BQ336762 AI218063 D63246 BM983853 BG200539 BQ186241 BQ184762 BE549966 AW087501 AW589555 BF061478 BU603861 BU536429 BU954011 BG198439 BQ267681 AA346773 AA642108 AA807781 AI632300 AI633800 AI479561 AA405485 AI419510 AW016718 BU678979 BM311591 BM692249 BM673518 AA652250 CA771710 AI492091 BM310984 AI494386 CA950854 BM311000 AW961666 AA346774 CA772543 CA951103 CA848186 BM126029 BI837048 BI834774 BI559674 AA327608 BG705044 BG703547 BF967333 BG168937 BC015348 BX119411 AA405635 BG722153 NM152773 BC021177 BM548106 AI380016 AI990640 BX098544 AA917719 CA308507 BU633848 CA430273 AI002739 BG490753 CD368238 BE897067 AA380953 BC013113 NM138461 BM550337 BI860838 BQ678650 CA489370 BM808243 BM810125 BG027765

>20301 D45585_TO (20300 D45585_P1) #TAA brain-tumor #TAAT all tumor types 5350-5769 #SEQLIST AA078583 BF852870 N42349 BX100987 N30436 AA078590 BF325559 BF358933 BG979863 BE254942 BF817778 AW504141 BM458377 BM011407 BU501666 BG398407 BG759894 AL134029 BE408840 BF026970 AA077540 CA309755 BE890305 AI085174 BF372046 AW815926 AW815924 AU124991 AK022628 BI224200 BG272215 AI002796 AA077835 BG950470 CD171714 BG575647 BF871631 BM462627 BF811628 BM467542 BF933509 BF838980 CB854836 CB854837 BF515576 BG675707 BC039159 BM479268 BG111365 BQ017628 BE547671 BM716560 BM711371 BI094547 AA463437 BE881465 BI036534 R72665 BU619478 BU682838 BG117492 BQ001621 AW007319 AA663735 CA444773 CA444806 AI459241 AA987211 BE222061 AW341312 AU148750 AI914217 AI1683508 BF001419 BM055310 AW058367 BE674110 AI309597 AI356881 BM055031 AI540797 AA938193 AA632081 AI357119 BF059293 BE503366 T96349 AU121951 AI356665 BE646431 AI913226 AA760871 AI128965 AW193657 AW050889 D45585 C20562

>93H63975_TO (92H63975_P1) #TS lung #TAA all tumor types #SEQLIST BF832090 BM917407 BF087575 BG008463 BC017022 NM152426 BE888971 BX340829 BF841711 BE827866 AL598990 BF879160 AI621256 CB215343 BX368513 BX326934 BE885482 N79740 BX279693H63975 BX116531 AI022304 WO7257

>137 AA985547_TO #TS kidney #SEQLIST AI681733 AI733428 AA985547 CB132776 AI791772 CB959047 BM467433 AI791738 BG249301 BE162114

>2298 AA337524_TO (2297 AA337524_P1) #TS ovary #TS cervix+uterus #TAA all tumor types #SEQLIST AI889508 BX093157 AI820938 AA482061 AA828779 AI829497 AA337524

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### CD-ROM Content

The following CD-ROMs are attached herewith:

Information provided as: File name/byte size/date of creation/operating system/machine format

CD-ROM1:

1. seqs_{—}125/335,513 Kbytes/Nov. 15, 2001/Microsoft Windows Internet Explorer/PC.
2. seqs_{—}133/253,406 Kbytes/Apr. 8, 2003/Microsoft Windows Internet Explorer/PC.
   CD-ROM2:
1. alignments_{—}125/391,693 Kbytes/Nov. 15, 2001/Microsoft Windows Internet Explorer/PC.
2. table_{—}125/13,926 Kbytes/Nov. 15, 2001/Microsoft Windows Internet Explorer/PC.
3. Table_S1.txt/41 Kbytes/Jul. 31, 2003/Microsoft Windows Microsoft Excel Worksheet/PC.
4. Table_S2.txt/135 Kbytes/Jul. 31, 2003/Microsoft Windows Microsoft Excel Worksheet/PC.
   CD-ROM3:
1. alignments_{—}133/454,180 Kbytes/Apr. 8, 2003/Microsoft Windows Internet Explorer/PC.
2. table_{—}133/10,741 Kbytes/Apr. 8, 2003/Microsoft Windows Internet Explorer/PC.
   CD-ROM4:
1. alignments_{—}136/19,190 Kbytes/Jan. 11, 2004/Microsoft Windows Internet Explorer/PC.
2. mouse_alignments/44,096 Kbytes/Jan. 11, 2004/Microsoft Windows Internet Explorer/PC.
3. mouse_seqs/23,009 Kbytes/Jan. 11, 2004/Microsoft Windows Internet Explorer/PC.
4. mouse_table/1,052 Kbytes/Jan. 11, 2004/Microsoft Windows Internet Explorer/PC.
5. nuc_seqs_{—}136/223,641 Kbytes/Jan. 11, 2004/Microsoft Windows Internet Explorer/PC.
6. orthology/76 Kbytes/Jan. 11, 2004/Microsoft Windows Internet Explorer/PC.
7. pep_seqs_{—}136/20,088 Kbytes/Jan. 11, 2004/Microsoft Windows Internet Explorer/PC.
8. table_{—}136/9,357 Kbytes/Jan. 11, 2004/Microsoft Windows Internet Explorer/PC.
9. annotations_{—}136/125,716 Kbytes/Jan. 11, 2004/Microsoft Windows Internet Explorer/PC.
10. Antisense.txt/1 Kbytes/Jan. 11, 2004/Microsoft Windows Internet Explorer/PC.

1. A method of identifying putative naturally occurring antisense transcripts, the method comprising:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database, thereby identifying putative naturally occurring antisense transcripts.

2. The method of claim 1, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

3. The method of claim 1, wherein said second database includes sequences of a type selected from the group consisting of expressed sequence tags, contigs, complementary DNA (cDNA) sequences, pre-messenger RNA (mRNA) sequences and mRNA sequences.

4. The method of claim 1, wherein an average sequence length of said expressed polynucleotide sequences of said second database is selected from a range of 0.02 to 0.8 Kb.

5. The method of claim 1, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

6. The method of claim 5, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

7. The method of claim 1 further comprising the step of testing the putative naturally occurring antisense transcripts for an ability to form said duplex with said at least one sense oriented polynucleotide sequence under physiological conditions.

8. The method of claim 1 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

9. A kit for quantifying at least one mRNA transcript of interest, the kit comprising at least one oligonucleotide being designed and configured so as to be complementary to a sequence region of the mRNA transcript of interest, said sequence region not being complementary with a naturally occurring antisense transcript.

10. The kit of claim 9, wherein a length of said at least one oligonucleotide is selected from a range of 15-200 nucleotides.

11. The kit of claim 9, wherein said at least one oligonucleotide is a single stranded oligonucleotide.

12. The kit of claim 9, wherein said at least one oligonucleotide is a double stranded oligonucleotide.

13. The kit of claim 9, wherein a guanidine and cytosine content of said at least one oligonucleotide is at least 25%.

14. The kit of claim 9, wherein said at least one oligonucleotide is labeled.

15. The kit of claim 9, wherein said at least one oligonucleotide is attached to a solid substrate.

16. The kit of claim 15, wherein said solid substrate is configured as a microarray and whereas said at least one oligonucleotide includes a plurality of oligonucleotides each attached to said microarray in a regio-specific manner.

17. A kit for quantifying at least one mRNA transcript of interest, the kit comprising at least one pair of oligonucleotides including a first oligonucleotide capable of binding the at least one mRNA transcript of interest and a second oligonucleotide being capable of binding a naturally occurring antisense transcript complementary to the mRNA of interest.

18. The kit of claim 17, wherein a length of each of said first and second oligonucleotides is selected from a range of 15-200 nucleotides

19. The kit of claim 17, wherein said first and second oligonucleotides are single stranded oligonucleotides.

20. The kit of claim 17, wherein said first and second oligonucleotides are double stranded oligonucleotide.

21. The kit of claim 17, wherein a guanidine and cytosine content of each of said first and second oligonucleotides is at least 25%.

22. The kit of claim 17, wherein said first and second oligonucleotides are labeled.

23. The kit of claim 17, wherein said first and second oligonucleotides are attached to a solid substrate.

24. The kit of claim 23, wherein said solid substrate is configured as a microarray and whereas each of said first and second oligonucleotides includes a plurality of oligonucleotides each attached to said microarray in a regio-specific manner.

25. A kit for quantifying at least one naturally occurring antisense transcript of interest, the kit comprising at least one oligonucleotide being designed and configured so as to be complementary to a sequence region of the at least one naturally occurring antisense transcript of interest, said sequence region not being complementary with a naturally occurring mRNA transcript.

26. The kit of claim 25, wherein a length of said at least one oligonucleotide is selected from a range of 15-200 nucleotides.

27. The kit of claim 25, wherein said at least one oligonucleotide is a single stranded oligonucleotide.

28. The kit of claim 25, wherein said at least one oligonucleotide is a double stranded oligonucleotide.

29. The kit of claim 25, wherein a guanidine and cytosine content of said at least one oligonucleotide is at least 25%.

30. The kit of claim 25, wherein said at least one oligonucleotide is labeled.

31. The kit of claim 25, wherein said at least one oligonucleotide is attached to a solid substrate.

32. The kit of claim 31, wherein said solid substrate is configured as a microarray and whereas said at least one oligonucleotide includes a plurality of oligonucleotides each attached to said microarray in a regio-specific manner.

33. A method of designing artificial antisense transcripts, the method comprising:
(a) providing a database of naturally occurring antisense transcripts;
(b) extracting from said database criteria governing structure and/or function of said naturally occurring antisense transcripts; and
(c) designing the artificial antisense transcripts according to said criteria.

34. The method of claim 33, wherein said criteria governing structure and/or function of said naturally occurring antisense transcripts are selected from the group consisting of antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

35. The method of claim 33, wherein said step of providing said database of naturally occurring antisense transcripts is effected by:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database,
(c) storing a sequence of said expressed polynucleotide sequences identified in step (b), thereby providing said database of said naturally occurring antisense transcripts.

36. The method of claim 35, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

37. The method of claim 35, wherein said second database includes sequences of a type selected from the group consisting of expressed sequence tags, contigs, complementary DNA (cDNA) sequences, pre-messenger RNA (mRNA) sequences and mRNA sequences.

38. The method of claim 35, wherein an average sequence length of said expressed polynucleotide sequences of said second database is selected from a range of 0.02 to 0.8 Kb.

39. The method of claim 35, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

40. The method of claim 39, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

41. The method of claim 35, further comprising the step of testing said putative naturally occurring antisense transcripts for an ability to form said duplex with said at least one sense oriented polynucleotide sequence under physiological conditions.

42. The method of claim 35 further comprising the step of computationally testing said putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

43. A computer readable storage medium comprising a database including a plurality of sequences, wherein each sequence is of a naturally occurring antisense transcript.

44. The computer readable storage medium of claim 43, wherein said database further includes information pertaining to each sequence of said naturally occurring antisense transcripts, said information is selected from the group consisting of related sense gene, antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

45. The computer readable storage medium of claim 43, wherein said database further includes information pertaining to generation of said database and potential uses of said database.

46. A method of generating a database of naturally occurring antisense transcripts, the method comprising:
(a) computationally aligning a first database including sense-oriented polynucleotide sequences with a second database including expressed polynucleotide sequences;
(b) identifying expressed polynucleotide sequences from said second database being capable of forming a duplex with at least one sense-oriented polynucleotide sequence of said first database so as to identify putative naturally occurring antisense transcripts; and
(c) storing sequence information of said identified naturally occurring antisense transcripts, thereby generating the database of the naturally occurring antisense transcripts.

47. The method of claim 46, wherein the database is set forth in the file seqs_{—}125 and/or seqs_{—}133 of the enclosed CD-ROM1, alignments_{—}125, table 125, Table_S1 and/or Table_S2 of the enclosed CD-ROM2, alignments_{—}133 and/or table_{—}133 of the enclosed CD-ROM3, mouse_alignments, mouse_seqs, mouse_table, nuc_seqs_{—}136, orthology, pep_seqs_{—}136, table_{—}136, annotations_{—}136 and/or antisense of the enclosed CD-ROM4, and alignments_{—}136 of CD-Rom 5.

48. The method of claim 46, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

49. The method of claim 46, wherein said second database includes sequences of a type selected from the group consisting of expressed sequence tags, contigs, complementary DNA (cDNA) sequences, pre-messenger RNA (mRNA) sequences and mRNA sequences.

50. The method of claim 46, wherein an average sequence length of said expressed polynucleotide sequences of said second database is selected from a range of 0.02 to 0.8 Kb.

51. The method of claim 46, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

52. The method of claim 51, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

53. The method of claim 46 further comprising the step of testing the putative naturally occurring antisense transcripts for an ability to form said duplex with said at least one sense oriented polynucleotide sequence under physiological conditions.

54. The method of claim 46 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

55. A system for generating a database of a plurality of putative naturally occurring antisense transcripts, the system comprising a processing unit, said processing unit executing a software application configured for:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database.

56. The system of claim 55, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

57. The system of claim 55, wherein said second database includes sequences of a type selected from the group consisting of expressed sequence tags, contigs, complementary DNA (cDNA) sequences, pre-messenger RNA (mRNA) sequences and mRNA sequences.

58. The system of claim 55, wherein an average sequence length of said expressed polynucleotide sequences of said second database is selected from a range of 0.02 to 0.8 Kb.

59. The system of claim 55, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

60. The system of claim 59, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

61. The system of claim 55 further comprising the step of testing the putative naturally occurring antisense transcripts for an ability to form said duplex with said at least one sense oriented polynucleotide sequence under physiological conditions.

62. The system of claim 55 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

63. A method of identifying putative naturally occurring antisense transcripts, the method comprising screening a database of expressed polynucleotides sequences according to at least one sequence criterion, said at least one sequence criterion being selected to identify putative naturally occurring antisense transcripts.

64. The method of claim 63, wherein said database includes sequences of a type selected from the group consisting of expressed sequence tags, contigs, complementary DNA (cDNA) sequences, pre-messenger RNA (mRNA) sequences and mRNA sequences.

65. The method of claim 63, wherein an average sequence length of said expressed polynucleotide sequences of said second database is selected from a range of 0.02 to 0.8 Kb.

66. The method of claim 63, wherein said at least one sequence criterion is selected from the group consisting of sequence length, sequence annotation, sequence information, intron splice consensus site, intron sharing, sequence overlap, rare restriction site, poly(T) head, poly(A) tail, and poly(A) signal.

67. The method of claim 63 further comprising the step of testing the putative naturally occurring antisense transcripts for an ability to form a duplex with at least one sense oriented polynucleotide sequence under physiological conditions.

68. A method of quantifying at least one mRNA of interest in a biological sample, the method comprising:
(a) contacting the biological sample with at least one oligonucleotide capable of binding with the at least one mRNA of interest, wherein said at least one oligonucleotide is designed and configured so as to be complementary to a sequence region of the mRNA transcript of interest, said sequence region not being complementary with a naturally occurring antisense transcript; and
(b) detecting a level of binding between the at least one mRNA of interest and said at least one oligonucleotide to thereby quantify the at least one mRNA of interest in the biological sample.

69. The method of claim 68, wherein said at least one oligonucleotide is attached to a solid substrate.

70. The method of claim 69, wherein said solid substrate is configured as a microarray and whereas said at least one oligonucleotide includes a plurality of oligonucleotides each attached to said microarray in a regio-specific manner.

71. The method of claim 68, wherein said at least one oligonucleotide is labeled and whereas step (b) is effected by quantifying said label.

72. The method of claim 68, wherein a length of said at least one oligonucleotide is selected from a range of 15-200 nucleotides.

73. The method of claim 68, wherein said at least one oligonucleotide is a single stranded oligonucleotide.

74. The method of claim 68, wherein said at least one oligonucleotide is a double stranded oligonucleotide.

75. The method of claim 68, wherein a guanidine and cytosine content of said at least one oligonucleotide is at least 25%.

76. A method of quantifying the expression potential of at least one mRNA of interest in a biological sample, the method comprising:
(a) contacting the biological sample with at least one pair of oligonucleotides including a first oligonucleotide capable of binding the at least one mRNA of interest and a second oligonucleotide being capable of binding a naturally occurring antisense transcript complementary to the mRNA of interest; and
(b) detecting a level of binding between the at least one mRNA of interest and said first oligonucleotide and a level of binding between said naturally occurring antisense transcript complementary to the mRNA of interest and said second oligonucleotide to thereby quantify the expression potential of the at least one mRNA of interest in the biological sample.

77. The method of claim 76, wherein a length of each of said first and second oligonucleotides is selected from a range of 15-200 nucleotides

78. The method of claim 76, wherein said first and second oligonucleotides are single stranded oligonucleotides.

79. The method of claim 76, wherein said first and second oligonucleotides are double stranded oligonucleotide.

80. The method of claim 76, wherein a guanidine and cytosine content of each of said first and second oligonucleotides is at least 25%.

81. The method of claim 76, wherein said first and second oligonucleotides are labeled and whereas step (b) is effected by quantifying said label.

82. The method of claim 76, wherein said first and second oligonucleotides are attached to a solid substrate.

83. The method of claim 82, wherein said solid substrate is configured as a microarray and whereas each of said first and second oligonucleotides includes a plurality of oligonucleotides each attached to said microarray in a regio-specific manner.

84. A method of quantifying at least one naturally occurring antisense transcript of interest in a biological sample, the method comprising:
(a) contacting the biological sample with at least one oligonucleotide capable of binding with the at least one naturally occurring antisense transcript of interest, wherein said at least one oligonucleotide is designed and configured so as to be complementary to a sequence region of the naturally occurring antisense transcript of interest, said sequence region not being complementary with a naturally occurring mRNA transcript; and
(b) detecting a level of binding between the at least one naturally occurring antisense transcript of interest and said at least one oligonucleotide to thereby quantify the at least one naturally occurring antisense transcript of interest in the biological sample.

85. The method of claim 84, wherein said at least one oligonucleotide is attached to a solid substrate.

86. The method of claim 85, wherein said solid substrate is configured as a microarray and whereas said at least one oligonucleotide includes a plurality of oligonucleotides each attached to said microarray in a regio-specific manner.

87. The method of claim 84, wherein said at least one oligonucleotide is labeled and whereas step (b) is effected by quantifying said label.

88. The method of claim 84, wherein a length of said at least one oligonucleotide is selected from a range of 15-200 nucleotides.

89. The method of claim 84, wherein said at least one oligonucleotide is a single stranded oligonucleotide.

90. The method of claim 84, wherein said at least one oligonucleotide is a double stranded oligonucleotide.

91. The method of claim 84, wherein a guanidine and cytosine content of said at least one oligonucleotide is at least 25%.

92. A method of identifying a novel drug target, the method comprising:
(a) determining expression level of at least one naturally occurring antisense transcript of interest in cells characterized by an abnormal phenotype; and
(b) comparing said expression level of said at least one naturally occurring antisense transcript of interest in said cells characterized by an abnormal phenotype to an expression level of said at least one naturally occurring antisense transcript of interest in cells characterized by a normal phenotype, to thereby identify the novel drug target.

93. The method of claim 92, wherein said abnormal phenotype of said cells is selected from the group consisting of biochemical phenotype, morphological phenotype and nutritional phenotype.

94. The method of claim 92, wherein said determining expression level of at least one naturally occurring antisense transcript of interest is effected by at least one oligonucleotide designed and configured so as to be complementary to a sequence region of said at least one naturally occurring antisense transcript of interest, said sequence region not being complementary with a naturally occurring mRNA transcript.

95. The method of claim 94, wherein a length of said at least one oligonucleotide is selected from a range of 15-200 nucleotides.

96. The method of claim 94, wherein said at least one oligonucleotide is a single stranded oligonucleotide.

97. The method of claim 94, wherein said at least one oligonucleotide is a double stranded oligonucleotide.

98. The method of claim 94, wherein a guanidine and cytosine content of said at least one oligonucleotide is at least 25%.

99. The method of claim 94, wherein said at least one oligonucleotide is labeled and whereas step (b) is effected by quantifying said label.

100. The method of claim 94, wherein said at least one oligonucleotide is attached to a solid substrate.

101. The method of claim 100, wherein said solid substrate is configured as a microarray and whereas said at least one oligonucleotide includes a plurality of oligonucleotides each attached to said microarray in a regio-specific manner.

102. A method of treating or preventing a disease, condition or syndrome associated with an upregulation of a naturally occurring antisense transcript complementary to a naturally occurring mRNA transcript, the method comprising administering a therapeutically effective amount of an agent for regulating expression of the naturally occurring antisense transcript.

103. The method of claim 102, wherein said agent for regulating expression of the naturally occurring antisense transcript is at least one oligonucleotide designed and configured so as to hybridize to a sequence region of said at least one naturally occurring antisense transcript.

104. The method of claim 103, wherein said at least one oligonucleotide is a ribozyme.

105. The method of claim 103, wherein said at least one oligonucleotide is a sense transcript.

106. A method of diagnosing a disease, condition or syndrome associated with a substandard expression ratio of an mRNA of interest over a naturally occurring antisense transcript complementary to the mRNA of interest, the method comprising:
(a) quantifying expression level of the mRNA of interest and the naturally occurring antisense transcript complementary to the mRNA of interest;
(b) calculating the expression ratio of the mRNA of interest over the naturally occurring antisense transcript complementary to the mRNA of interest, thereby diagnosing the disease, condition or syndrome.

107. The method of claim 106, wherein quantifying said expression level of the mRNA of interest and the naturally occurring antisense transcript complementary to the mRNA of interest is effected by at least one pair of oligonucleotides including a first oligonucleotide capable of binding the mRNA of interest and a second oligonucleotide being capable of binding the naturally occurring antisense transcript complementary to the mRNA of interest.

108. The method of claim 107, wherein a length of each of said first and second oligonucleotides is selected from a range of 15-200 nucleotides

109. The method of claim 107, wherein said first and second oligonucleotides are single stranded oligonucleotides.

110. The method of claim 107, wherein said first and second oligonucleotides are double stranded oligonucleotides.

111. The method of claim 107, wherein a guanidine and cytosine content of each of said first and second oligonucleotides is at least 25%.

112. The method of claim 107, wherein said first and second oligonucleotides are labeled.

113. The method of claim 107, wherein said first and second oligonucleotides are attached to a solid substrate.

114. The method of claim 113, wherein said solid substrate is configured as a microarray and whereas each of said first and second oligonucleotides includes a plurality of oligonucleotides each attached to said microarray in a regio-specific manner.

115. A method of identifying co-regulated human polynucleotide sequences, the method comprising:
(a) computationally identifying non-human polynucleotide sequence pairs, each corresponding to an mRNA sequence and its naturally occurring antisense transcript;
(b) computationally identifying for each polynucleotide sequence of said polynucleotide sequence pairs a human orthologue polynucleotide sequence, thereby identifying human polynucleotide sequence pairs; and
(c) selecting from said human polynucleotide sequence pairs, specific polynucleotide sequence pairs having oppositely oriented polynucleotide sequences which are localized to a chromosome region, said specific polynucleotide sequence pairs being co-regulated human polynucleotide sequences.

116. The method of claim 115, wherein said specific polynucleotide sequence pairs are gapped by a distance not exceeding a predetermined value.

117. The method of claim 116, wherein said predetermined value does not exceed 10 Kb.

118. The method of claim 115, wherein step (a) is effected by:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database, thereby identifying said polynucleotide sequence pairs of mRNA sequences and naturally occurring antisense transcripts complementary to the mRNA sequences.

119. The method of claim 115, wherein step (b) is effected by a homology screening software application.

120. The method of claim 115, further comprising identifying oppositely oriented expressed sequences corresponding to the human co-regulated polynucleotide sequences.

121. A system for generating a database of co-regulated human polynucleotide sequences, the system comprising a processing unit, said processing unit executing a software application configured for:
(a) computationally identifying non-human polynucleotide sequence pairs, each corresponding to an mRNA sequence and its naturally occurring antisense transcript;
(b) computationally identifying for each polynucleotide sequence of said polynucleotide sequence pairs a human orthologue polynucleotide sequence, thereby identifying human polynucleotide sequence pairs;
(c) selecting from said human polynucleotide sequence pairs, specific polynucleotide sequence pairs having oppositely oriented polynucleotide sequences which are localized to a chromosome region, said specific polynucleotide sequence pairs being co-regulated human polynucleotide sequences; and
(d) storing the co-regulated human polynucleotide sequences to therevy generate the database of co-regulated human polynucleotide sequences

122. The system of claim 121, wherein said specific polynucleotide sequence pairs are gapped by a distance not exceeding a predetermined value.

123. The system of claim 122, wherein said predetermined value does not exceed 10 Kb.

124. The system of claim 121, wherein step (a) is effected by:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database, thereby identifying said polynucleotide sequence pairs of mRNA sequences and naturally occurring antisense transcripts complementary to the mRNA sequences.

125. The system of claim 121, wherein step (b) is effected by a homology screening software application.

126. The system of claim 121, further comprising identifying oppositely oriented expressed sequences corresponding to the human co-regulated polynucleotide sequences.

127. A computer readable storage medium comprising data stored in a retrievable manner, said data including sequence information of co-regulated human polynucleotide sequences as set forth in files seqs_{—}125 and/or seqs_{—}133 of enclosed CD-1, mouse_seqs, nuc_seqs_{—}136 and/or pep_seqs_{—}136 of enclosed CD-ROM4 and sequence annotations as set forth in the file annotations_{—}136 of enclosed CD-ROM4.

128. A method of modulating an activity or expression of a gene product, the method comprising upregulating or down regulating expression or activity of a naturally occurring antisense transcript of the gene product, thereby modulating the activity or expression of the gene product.

129. The method of claim 128, further comprising upregulating or down regulating expression or activity of the gene product.

130. An isolated polynucleotide comprising any of the nucleic acid sequences set forth in the file seqs_{—}125 or seqs_{—}133 of the enclosed CD-ROM1; or in the file nuc_seqs_{—}136 of the enclosed CD-ROMs 1-4.

131. An isolated polypeptide comprising any of the amino acid sequences set forth in the file pep_seqs_{—}136 of enclosed CD-ROM4.
